(19) United States
(12) Patent Application Publication
(10) Pub. No.: US 2024/0293412 A1
(43) Pub. Date: Sep. 5, 2024

(54) NAMPT ACTIVATORS FOR TREATING METABOLIC AND NEUROLOGICAL DISORDERS

(71) Applicants: The Board of Trustees of the University of Illinois, Urbana, IL (US); ARIZONA BOARD OF REGENTS ON BEHALF OF THE UNIVERSITY OF ARIZONA, TUCSON, AZ (US)

(72) Inventors: Gregory R THATCHER, Tucson, AZ (US); Rui XIONG, Chicago, IL (US); Kiira RATIA, Chicago, IL (US); Yangfeng LI, TUCSON, AZ (US); Zhengnan SHEN, Tucson, AZ (US)

(21) Appl. No.: 18/553,909

(22) PCT Filed: Apr. 7, 2022
(86) PCT No.: PCT/US2022/023880

§ 371 Date: Oct. 4, 2023

Related U.S. Application Data
(60) Provisional application No. 63/171,825, filed on Apr. 7, 2021.

(51) Int. Cl. A61K 31/519; A61K 31/55; C07D 487/04; C07D 491/048; C07D 495/04; C07D 519/00

(52) U.S. Cl. CPC A61K 31/519; A61K 31/55; C07D 487/04; C07D 491/048; C07D 495/04; C07D 519/00

(57) ABSTRACT

In accordance with the purpose(s) of the present disclosure, as embodied and broadly described herein, the disclosure, in one aspect, relates to compounds and methods for activating NAMPT in a subject. The compounds described herein are useful in treating a neurological disorder, disease, or condition. In another aspect, compounds described herein are useful in treating metabolic disorders and diabetes. Formula (I)

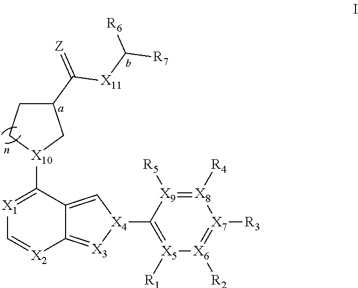

### CROSS REFERENCE TO RELATED APPLICATIONS

This application claims the benefit of U.S. Provisional Application No. 63/171,825 filed on Apr. 7, 2021, which is incorporated herein by reference in its entirety.

### BACKGROUND

Approximately 99% of all Alzheimer's and dementia (AD) cases are not early-onset, familial Alzheimer's and dementia (FAD). Aging is the single most significant risk factor for the development of Alzheimer's and dementia. “Anti-aging” treatments have entered mainstream science and represent an approach to Alzheimer's and Related Dementia (ADRD). The NIA Interventions Testing Program reported that mean or maximum lifespan in mice is significantly increased by interventions, including NAD-enhancing drugs¹⁻⁵. Demonstrated protective actions that combat the aging phenotype, include reductions in inflammation, oxidative stress, and metabolic dysfunction, and improved mitochondrial function.

Nicotinamide adenine dinucleotide (NAD⁺) levels and NAD⁺/NADH ratio decline in the aging brain, and NAD depletion is implicated in mitochondrial disorders and various age-related pathologies. Dietary supplementation with NAD-enhancing drugs (vitamin B₃ complex, NMN, NR, NAM, NAD) has been proposed to enhance lifespan (or healthspan) and reduce risk factors associated with AD, such as diabetes, obesity, and dyslipidemia.

The rate-limiting step in NAD synthesis is the NAMPT-catalyzed formation of NMN from salvage of nicotinamide (NAM), the product of NAD⁺ degradation by poly-ADP-ribose polymerases (PARPs), sirtuins (SIRTs), and CD38 (NAD nucleosidase) (FIG. 1). The ratio of oxidized to reduced NAD (NAD⁺/NADH) reflects cellular oxidative and mitochondrial stress, as NAD is an essential enzyme cofactor in electron and hydride transfer reactions, notably in mitochondria. NAD⁺ is catabolized as a substrate by PARPs, SIRTs, and CD38. These proteins are most closely linked with NAD depletion in physiology and pathophysiology,^{6,7} but NADase activity is present in ADP ribosyltransferases (ARTs) and SARM1.⁸⁻¹⁰ Intracellular NAMPT catalyzes the synthesis of NMN from NAM and PRPP (FIG. 1).^{11,12} Extracellular NAMPT (eNAMPT)¹³⁻¹⁵ is a plasma/tissue biomarker for some pathologies,¹⁶ including obesity and inflammation.^{17,18}

Aging significantly decreases NAMPT and NAD levels.¹⁹⁻²¹ NAMPT activity is viewed as key in cellular defense mechanisms controlling cell survival and maintaining metabolic homeostasis:²² NAMPT has regulatory actions on cellular metabolism, mitochondrial biogenesis, electron transport, ROS, energy production, and redox balance via NAD-dependent enzymes, such as NADPH oxidase. While in the context of cell survival, NAMPT mediates an adaptive response to inflammatory, oxidative, and genotoxic stress, a response which declines with age.^{23,24} In the aging brain, NAMPT is: (i) crucial in the proliferation, self-renewal, and differentiation of neural stem/progenitor cells (NSPCs);²⁵ (ii) the main supplier of NAD to NSPCs;²⁵ and (iii) critical for cognitive function.²⁶

In aged WT mice, NAMPT expression is most significantly decreased in the hippocampus and cortex.²⁷ NAMPT is mainly expressed in neurons and NSPCs, with hippocampal levels correlating with adult neurogenesis. Aging is a prime negative regulator of adult NSPC proliferation and knockout of NAMPT was observed to block neurogenesis and oligodendrogenesis.^{25,28,29} Sirtuins are NAD⁺-dependent and evidence suggests that NAMPT contributes to NSPC proliferation via the actions of SIRT1 and SIRT2; and to NSPC differentiation via SIRT1, SIRT2, and SIRT6.³⁰ NAMPT depletion causes perturbations in energy homeostasis and neuronal cell death¹³, and the NAMPT inhibitor FK866 causes a significant decrease in intracellular NAD, with the greatest depletion of NAD observed in the brain.³¹ The recent identification of SARM1 as an injury-activated NAD-depleting enzyme that causes axon degeneration has opened another mechanism whereby a N-PAM may support axonal and neuronal integrity.^{9,10,32,33}

### SUMMARY

In accordance with the purpose(s) of the present disclosure, as embodied and broadly described herein, the disclosure, in one aspect, relates to scaffold molecules that can activate NAMPT in a subject. In one aspect, the compounds have the Formula I

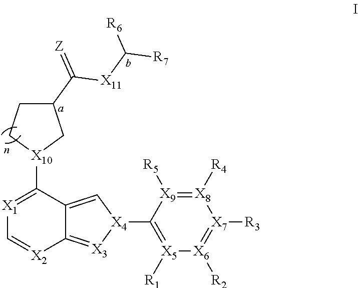

wherein X₁, X₂, X₃, and X₄ are N or CH;
X₅, X₆, X₇, X₈, and X₉ are N or C, wherein when X₅, X₆, X₇, X₈, or X₉ is N, R₁, R₂, R₃, R₄, R₅ and R₆ are not present;
X₁₀ is N or C(Y₁)₂;
X₁₁ is NY₁ or C(Y₁)₂;
Y₁ is independently selected from the group consisting of hydrogen, alkyl, alkoxy, haloalkyl, hydroxyalkyl, OH, CN, F, C, Br, and I;
   Z is O or S;
R₁, R₂, R₃, R₄, R₅ and R₆ are each independently selected from —H, —OH, a halogen, —CN, alkyl, alkynyl, C3-C6 cycloalkyl, a C1-C3 haloalkyl, and —C(O)—O-alkyl;
R₇ is independently selected from:

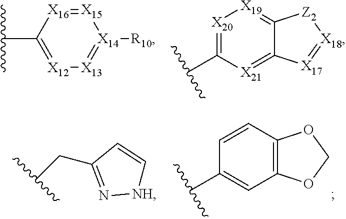

X₁₂, X₁₃, X₁₅, X₁₆, X₁₇, X₁₈, X₁₉, X₂₀, and X₂₁ are N or CY₂;
X₁₄ is N or C, wherein when X₁₄ is N, R₁₀ is not present;
Y₂ is selected from hydrogen, alkyl, alkoxy, haloalkyl, hydroxyalkyl, OH, CN, F, C, Br, and I;
Z₂ is S, NY₃ or O;
Y₃ is hydrogen or alky;
R₁₀ is independently selected from: hydrogen, alkyl, alkoxy, haloalkyl, hydroxyalkyl, OH, CN, F, C, Br, I, N(alkyl)₂, S-alkyl, S(O)-alkyl, and S(O)₂-alkyl;
n is an integer from 0 to 3; and
the stereochemistry at carbon a and carbon b is, independently, substantially R, substantially S, or racemic; or
a pharmaceutically acceptable salt thereof.

In another aspect, the compounds have the formula IV or V

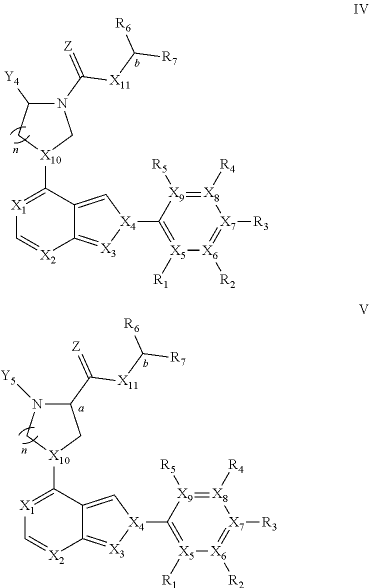

wherein X₁, X₂, X₃, and X₄ are N or CH;
X₅, X₆, X₇, X₈, and X₉ are N or C, wherein when X₅, X₆, X₇, X₈, or X₉ is N, R₁, R₂, R₃, R₄, R₅ and R₆ are not present;
X₁₀ is N or C(Y₁)₂;
X₁₁ is NY₁ or C(Y₁)₂;
Y₁, Y₄, and Y₅ are independently selected from the group consisting of hydrogen, alkyl, alkoxy, haloalkyl, hydroxyalkyl, OH, CN, F, Cl, Br, and I;
Z is O or S;
R₁, R₂, R₃, R₄, R₅ and R₆ are each independently selected from —H, —OH, a halogen, —CN, alkyl, alkynyl, C3-C6 cycloalkyl, a C1-C3 haloalkyl, and —C(O)—O-alkyl;
R₇ is independently selected from:

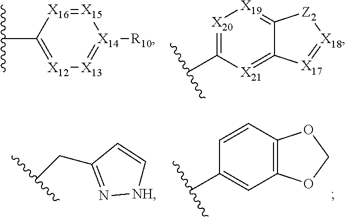

X₁₂, X₁₃, X₁₅, X₁₆, X₁₇, X₁₈, X₁₉, X₂₀, and X₂₁ are N or CY₂;
X₁₄ is N or C, wherein when X₁₄ is N, R₁₀ is not present;
Y₂ is selected from hydrogen, alkyl, alkoxy, haloalkyl, hydroxyalkyl, OH, CN, F, Cl, Br, and I;
Z₂ is S, NY₃ or O;
Y₃ is hydrogen or alkyl;
R₁₀ is independently selected from hydrogen, alkyl, alkoxy, haloalkyl, hydroxyalkyl, OH, CN, F, Cl, Br, I, N(alkyl)₂, S-alkyl, S(O)-alkyl, and S(O)₂-alkyl;
n is an integer from 0 to 3; and
the stereochemistry at carbon a and carbon b is, independently, substantially R, substantially S, or racemic; or
a pharmaceutically acceptable salt thereof.

In one aspect, the compounds described herein can activate NAMPT in a subject. In another aspect, the compounds described herein can increase NAD⁺ in a subject. In another aspect, the compounds described herein can treating a neurological disorder, diabetes, or metabolic disorder in a subject.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows the synthesis of NMN from NAM and PRPP by intracellular NAMPT catalysis.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay. For example, an IC₅₀ for an abietane described herein can be determined in an in vitro or cell-based assay system. Frequently, receptor assays make use of a suitable cell-line, e.g. a cell line that either expresses endogenously a target of interest, or has been transfected with a suitable expression vector that directs expression of a recombinant form of the target. For example, the IC₅₀ for a compound disclosed herein can be determined using cancer cells contacted with a compound described herein.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The position of a substituent can be defined relative to the positions of other substituents in an aromatic ring. For example, as shown below in relationship to the “R” group, a second substituent can be “ortho,” “para,” or “meta” to the R group, meaning that the second substituent is bonded to a carbon labeled ortho, para, or meta as indicated below. Combinations of ortho, para, and meta substituents relative to a given group or substituent are also envisioned and should be considered to be disclosed.

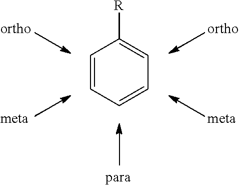

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A² where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹O A², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

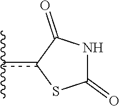

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

The terms “substantially R” and “substantially S” refer to the enantiomeric excess of one enantiomer over the other enantiomer. An enantiomer that is substantially R has a chiral center that has at least a 90% and up to 100% enantiomeric excess of the R enantiomer. An enantiomer that is substantially R has a chiral center that has at least a 90% and up to 100% enantiomeric excess of the R enantiomer. Enantiomeric excess is calculated by subtracting the amount of one enantiomer from the other enantiomer. A racemic compound is a compound with a chiral center having equal amounts of R and S enantiomer.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

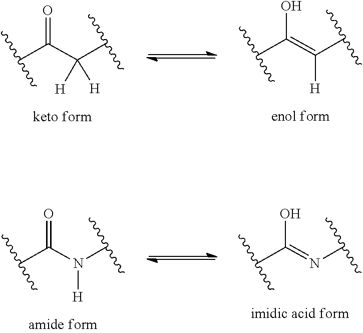 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

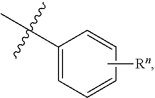

which is understood to be equivalent to a formula:

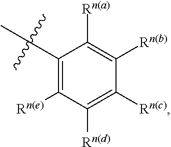

wherein n is typically an integer. That is, Rⁿ is understood to represent five independent substituents, R^{n(a)}, R^{n(b)}, R^{n(c)}, R^{n(d)} and R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intraarticular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-r interactions, anion-r interactions, polar r-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a hematological malignancy, breast cancer, and/or another solid malignancy. The effect can be therapeutic in terms of a partial or complete cure of a disease, condition, symptom or adverse effect attributed to the disease, disorder, or condition. The term “treatment” as used herein can include any treatment of a hematological malignancy, breast cancer, and/or another solid tumor in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C1-to-C6 alkyl esters and C5-to-C7 cycloalkyl esters, although C1-to-C4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C1-to-C6 alkyl amines and secondary C1-to-C6 dialkyl amines. In the case of secondary amines, the amine can also be in the form of a 5- or 6-membered heterocycle containing one nitrogen atom. Amides derived from ammonia, C1-to-C 3 alkyl primary amides and C1-to-C2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

Unless otherwise specified, temperatures referred to herein are based on atmospheric pressure (i.e. one atmosphere).

### Compounds and Methods of Making and Using the Compounds

In one aspect, disclosed herein is a compound having a structure according to Formula I:

In one aspect, the compounds have the Formula I

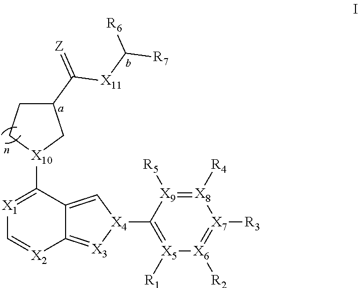

wherein X₁, X₂, X₃, and X₄ are N or CH;
X₅, X₆, X₇, X₈, and X₉ are N or C, wherein when X₅, X₆, X₇, X₈, or X₉ is N, R₁, R₂, R₃, R₄, R₅ and R₆ are not present;
X₁₀ is N or C(Y₁)₂;
X₁₁ is NY₁ or C(Y₁)₂;
Y₁ is independently selected from the group consisting of hydrogen, alkyl, alkoxy, haloalkyl, hydroxyalkyl, OH, CN, F, C, Br, and I;
   Z is O or S;
R₁, R₂, R₃, R₄, R₅ and R₆ are each independently selected from —H, —OH, a halogen, —CN, alkyl, alkynyl, C3-C6 cycloalkyl, a C1-C3 haloalkyl, and —C(O)—O-alkyl;
R₇ is independently selected from:

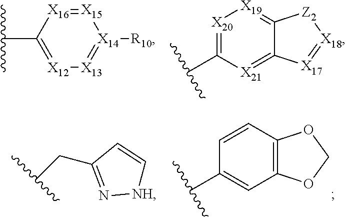

X₁₂, X₁₃, X₁₅, X₁₆, X₁₇, X₁₈, X₁₉, X₂₀, and X₂₁ are N or CY₂;
X₁₄ is N or C, wherein when X₁₄ is N, R₁₀ is not present;
Y₂ is selected from hydrogen, alkyl, alkoxy, haloalkyl, hydroxyalkyl, OH, CN, F, C, Br, and I;
Z₂ is S, NY₃ or O;
Y₃ is hydrogen or alky;
R₁₀ is independently selected from: hydrogen, alkyl, alkoxy, haloalkyl, hydroxyalkyl, OH, CN, F, Cl, Br, I, N(alkyl)₂, S-alkyl, S(O)-alkyl, and S(O)₂-alkyl;
n is an integer from 0 to 3; and
the stereochemistry at carbon a and carbon b is, independently, substantially R, substantially S, or racemic; or
a pharmaceutically acceptable salt thereof.

In another aspect, the compounds have the formula IV or V

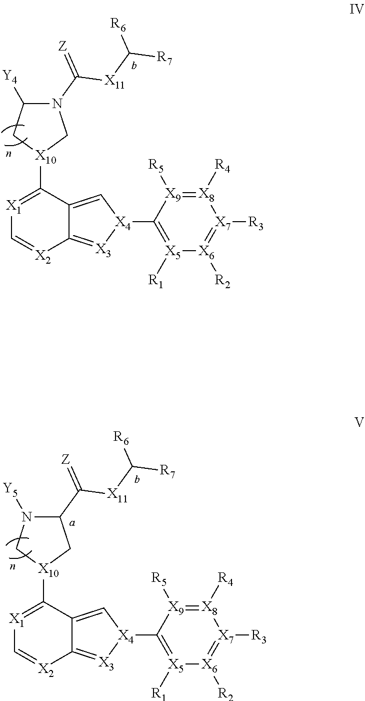

wherein X₁, X₂, X₃, and X₄ are N or CH;
X₅, X₆, X₇, X₈, and X₉ are N or C, wherein when X₅, X₆, X₇, X₈, or X₉ is N, R₁, R₂, R₃, R₄, R₅ and R₆ are not present;
X₁₀ is N or C(Y₁)₂;
X₁₁ is NY₁ or C(Y₁)₂;
Y₁, Y₄, and Y₅ are independently selected from the group consisting of hydrogen, alkyl, alkoxy, haloalkyl, hydroxyalkyl, OH, CN, F, Cl, Br, and I;
Z is O or S;
R₁, R₂, R₃, R₄, R₅ and R₆ are each independently selected from —H, —OH, a halogen, —CN, alkyl, alkynyl, C3-C6 cycloalkyl, a C1-C3 haloalkyl, and —C(O)—O-alkyl;
R₇ is independently selected from:

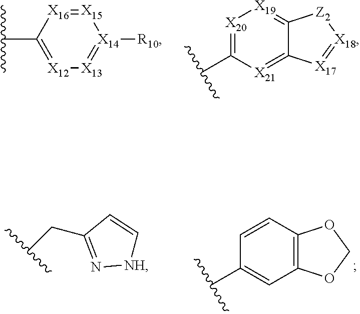

X₁₂, X₁₃, X₁₅, X₁₆, X₁₇, X₁₈, X₁₉, X₂₀, and X₂₁ are N or CY₂;
X₁₄ is N or C, wherein when X₁₄ is N, R₁₀ is not present;
Y₂ is selected from hydrogen, alkyl, alkoxy, haloalkyl, hydroxyalkyl, OH, CN, F, Cl, Br, and I;
Z₂ is S, NY₃ or O;
Y₃ is hydrogen or alkyl;
R₁₀ is independently selected from hydrogen, alkyl, alkoxy, haloalkyl, hydroxyalkyl, OH, CN, F, Cl, Br, I, N(alkyl)₂, S-alkyl, S(O)-alkyl, and S(O)₂-alkyl;
n is an integer from 0 to 3; and
the stereochemistry at carbon a and carbon b is, independently, substantially R, substantially S, or racemic; or
a pharmaceutically acceptable salt thereof.

In one aspect, X₁, X₂, X₃, X₄, and X₁₀ in Formulae I, IV, and V are each N. In another aspect, Z is O and X₁₁ is NH in Formulae I, IV, and V. In another aspect, n is 2 in Formulae I, IV, and V. In another aspect, X₅, X₆, X₇, X₈, and X₉ are each C in Formulae I, IV, and V. In another aspect, R₃ is a halogen, alkyl, or C1-C3 haloalkyl in Formulae I, IV, and V. In another aspect, R₆ is hydrogen in Formulae I, IV, and V.

In one aspect, R₇ in Formulae I, IV, and V is

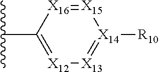

wherein X₁₂, X₁₃, X₁₅, and X₁₆, are each CY₂ and X₁₄ is C, and R₁₀ is F, Cl, Br, I or S-alkyl.

In one aspect, R₇ in Formulae I, IV, and V is

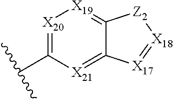

wherein Z₂ is S and each of X₁₇, X₁₈, X₁₉, X₂₀, and X₂₁ are CY₂.

In one aspect, the compound is Formula II

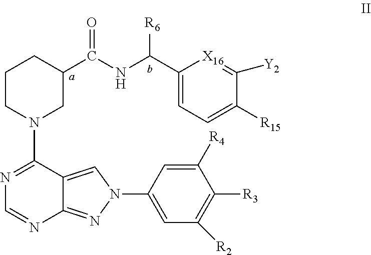

wherein X₁₆ is N or CH;
R₂, R₃, R₄, R₆, and Y₂ are independently selected from —H, —OH a halogen, —CN, alkynyl, C3-C6 cycloalkyl and C1-C3 haloalkyl; and
R₁₅ is selected from -SMe, -SEt, —I, —Br.

In one aspect, the compound is Formula III

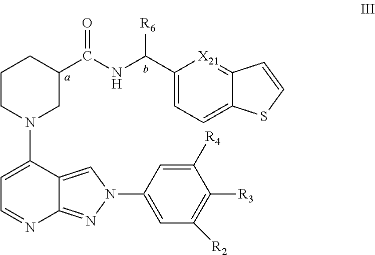

wherein X₂₁ is N or CH; and
R₂, R₃, R₄, and R₆ are independently selected from —H, —OH a halogen, —CN, alkynyl, C3-C6 cycloalkyl and C1-C3 haloalkyl; and

In one aspect, R₆ in Formulae I, IV, and V is hydrogen and the stereochemistry at carbon a is substantially R or substantially S.

In some aspects, the compound can be any one of compounds (1)-(403) as shown in Table 1.

TABLE 1
Structures and chemical characterization

| | | | LC-MS |
|---|---|---|---|
| Ex- | | | characterization |
| amples | Structure | Chemical name | (M + H⁺) |
| 1 | 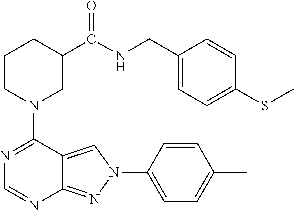 [SMILES: CSc1ccc(CNC(=O)C2CCCN(c3ncnc4nn(-c5ccc(C)cc5)cc34)C2)cc1] | N-(4-(methylthio)benzyl)- 1-(2-(p-tolyl)-2H- pyrazolo[3,4-d]pyrimidin- 4-yl)piperidine-3- carboxamide | 473.18 |
| 2 | 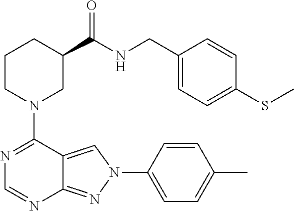 [SMILES: CSc1ccc(CNC(=O)[C@@H]2CCCN(c3ncnc4nn(-c5ccc(C)cc5)cc34)C2)cc1] | (R)-N-(4- (methylthio)benzyl)-1-(2- (p-tolyl)-2H-pyrazolo[3,4- d]pyrimidin-4- yl)piperidine-3- carboxamide | 473.23 |
| 3 | 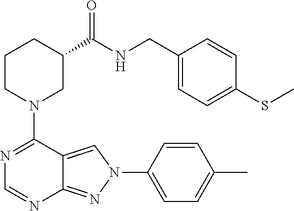 [SMILES: CSc1ccc(CNC(=O)[C@H]2CCCN(c3ncnc4nn(-c5ccc(C)cc5)cc34)C2)cc1] | (S)-N-(4- (methylthio)benzyl)-1-(2- (p-tolyl)-2H-pyrazolo[3,4- d]pyrimidin-4- yl)piperidine-3- carboxamide | 473.16 |
| 4 | 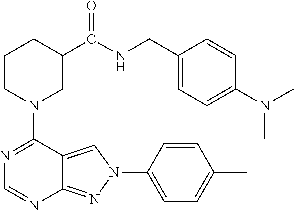 [SMILES: Cc1ccc(-n2cc3c(N4CCCC(C(=O)NCc5ccc(N(C)C)cc5)C4)ncnc3n2)cc1] | N-(4- (dimethylamino)benzyl)- 1-(2-(p-tolyl)-2H- pyrazolo[3,4-d]pyrimidin- 4-yl)piperidine-3- carboxamide | 470.24 |
| 5 | 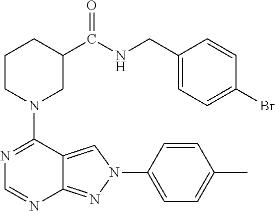 [SMILES: Cc1ccc(-n2cc3c(N4CCCC(C(=O)NCc5ccc(Br)cc5)C4)ncnc3n2)cc1] | N-(4-bromobenzyl)-1-(2- (p-tolyl)-2H-pyrazolo[3,4- d]pyrimidin-4- yl)piperidine-3- carboxamide | 505.07 |
| 6 | 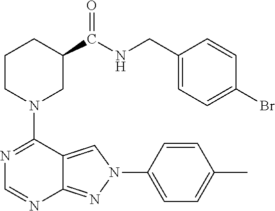 [SMILES: Cc1ccc(-n2cc3c(N4CCC[C@@H](C(=O)NCc5ccc(Br)cc5)C4)ncnc3n2)cc1] | (R)-N-(4-bromobenzyl)-1- (2-(p-tolyl)-2H- pyrazolo[3,4-d]pyrimidin- 4-yl)piperidine-3- carboxamide | 505.11 |
| 7 | 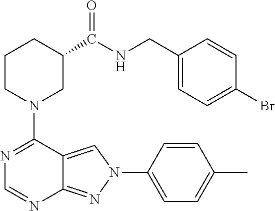 [SMILES: Cc1ccc(-n2cc3c(N4CCC[C@H](C(=O)NCc5ccc(Br)cc5)C4)ncnc3n2)cc1] | (S)-N-(4-bromobenzyl)-1- (2-(p-tolyl)-2H- pyrazolo[3,4-d]pyrimidin- 4-yl)piperidine-3- carboxamide | 505.09 |
| 8 | 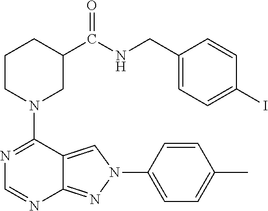 [SMILES: Cc1ccc(-n2cc3c(N4CCCC(C(=O)NCc5ccc(I)cc5)C4)ncnc3n2)cc1] | N-(4-iodobenzyl)-1-(2-(p- tolyl)-2H-pyrazolo[3,4- d]pyrimidin-4- yl)piperidine-3- carboxamide | 553.08 |
| 9 | 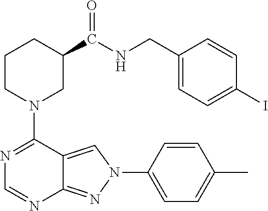 [SMILES: Cc1ccc(-n2cc3c(N4CCC[C@@H](C(=O)NCc5ccc(I)cc5)C4)ncnc3n2)cc1] | (R)-N-(4-iodobenzyl)-1- (2-(p-tolyl)-2H- pyrazolo[3,4-d]pyrimidin- 4-yl)piperidine-3- carboxamide | 553.05 |
| 10 | 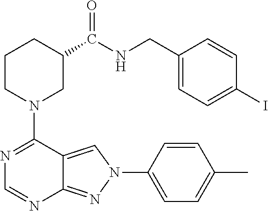 | (S)-N-(4-iodobenzyl)-1- (2-(p-tolyl)-2H- pyrazolo[3,4-d]pyrimidin- 4-yl)piperidine-3- carboxamide | 553.04 |
| 11 | 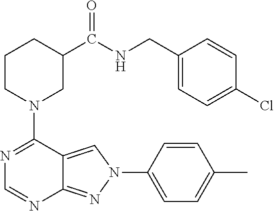 [SMILES: Cc1ccc(-n2cc3c(N4CCCC(C(=O)NCc5ccc(Cl)cc5)C4)ncnc3n2)cc1] | N-(4-chlorobenzyl)-1-(2- (p-tolyl)-2H-pyrazolo[3,4- d]pyrimidin-4- yl)piperidine-3- carboxamide | 461.16 |
| 12 | 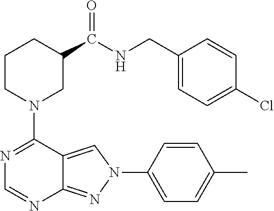 | (R)-N-(4-chlorobenzyl)-1- (2-(p-tolyl)-2H- pyrazolo[3,4-d]pyrimidin- 4-yl)piperidine-3- carboxamide | 461.27 |
| 13 | 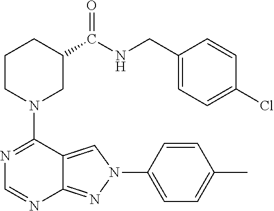 | (S)-N-(4-chlorobenzyl)-1- (2-(p-tolyl)-2H- pyrazolo[3,4-d]pyrimidin- 4-yl)piperidine-3- carboxamide | 461.24 |
| 14 | 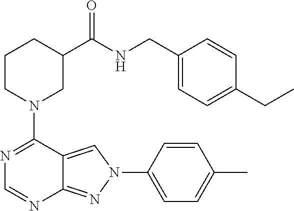 [SMILES: CCc1ccc(CNC(=O)C2CCCN(c3ncnc4nn(-c5ccc(C)cc5)cc34)C2)cc1] | N-(4-ethylbenzyl)-1-(2-(p- tolyl)-2H-pyrazolo[3,4- d]pyrimidin-4- yl)piperidine-3- carboxamide | 455.23 |
| 15 | 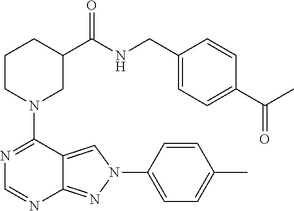 [SMILES: CC(=O)c1ccc(CNC(=O)C2CCCN(c3ncnc4nn(-c5ccc(C)cc5)cc34)C2)cc1] | N-(4-acetylbenzyl)-1-(2- (p-tolyl)-2H-pyrazolo[3,4- d]pyrimidin-4- yl)piperidine-3- carboxamide | 469.28 |
| 16 | 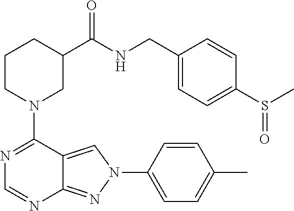 [SMILES: Cc1ccc(-n2cc3c(N4CCCC(C(=O)NCc5ccc(S(C)=O)cc5)C4)ncnc3n2)cc1] | N-(4- (methylsulfinyl)benzyl)-1- (2-(p-tolyl)-2H- pyrazolo[3,4-d]pyrimidin- 4-yl)piperidine-3- carboxamide | 489.68 |
| 17 | 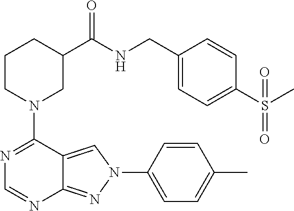 [SMILES: Cc1ccc(-n2cc3c(N4CCCC(C(=O)NCc5ccc(S(C)(=O)=O)cc5)C4)ncnc3n2)cc1] | N-(4- (methylsulfonyl)benzyl)- 1-(2-(p-tolyl)-2H- pyrazolo[3,4-d]pyrimidin- 4-yl)piperidine-3- carboxamide | 505.14 |
| 18 | 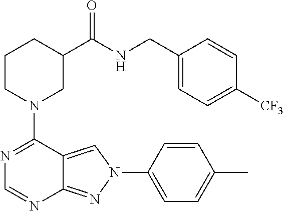 [SMILES: Cc1ccc(-n2cc3c(N4CCCC(C(=O)NCc5ccc(C(F)(F)F)cc5)C4)ncnc3n2)cc1] | 1-(2-(p-tolyl)-2H- pyrazolo[3,4-d]pyrimidin- 4-yl)-N-(4- (trifluoromethyl)benzyl) piperidine-3-carboxamide | 495.22 |
| 19 | 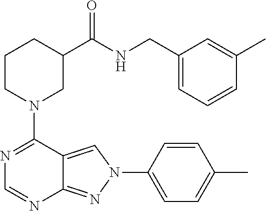 [SMILES: Cc1ccc(-n2cc3c(N4CCCC(C(=O)NCc5cccc(C)c5)C4)ncnc3n2)cc1] | N-(3-methylbenzyl)-1-(2- (p-tolyl)-2H-pyrazolo[3,4- d]pyrimidin-4- yl)piperidine-3- carboxamide | 441.24 |
| 20 | 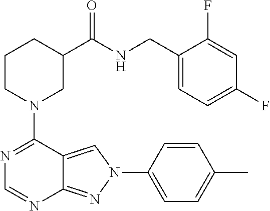 [SMILES: Cc1ccc(-n2cc3c(N4CCCC(C(=O)NCc5ccc(F)cc5F)C4)ncnc3n2)cc1] | N-(2,4-difluorobenzyl)-1- (2-(p-tolyl)-2H- pyrazolo[3,4-d]pyrimidin- 4-yl)piperidine-3- carboxamide | 463.46 |
| 21 | 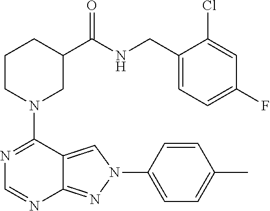 [SMILES: Cc1ccc(-n2cc3c(N4CCCC(C(=O)NCc5ccc(F)cc5Cl)C4)ncnc3n2)cc1] | N-(2-chloro-4- fluorobenzyl)-1-(2-(p- tolyl)-2H-pyrazolo[3,4- d]pyrimidin-4- yl)piperidine-3- carboxamide | 480.00 |
| 22 | 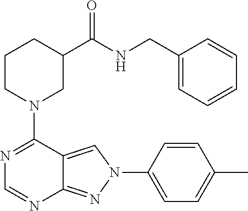 [SMILES: Cc1ccc(-n2cc3c(N4CCCC(C(=O)NCc5ccccc5)C4)ncnc3n2)cc1] | N-benzyl-1-(2-(p-tolyl)- 2H-pyrazolo[3,4- d]pyrimidin-4- yl)piperidine-3- carboxamide | 427.16 |
| 23 | 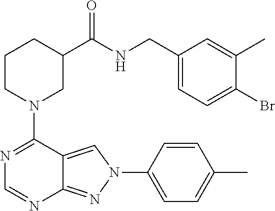 [SMILES: Cc1ccc(-n2cc3c(N4CCCC(C(=O)NCc5ccc(Br)c(C)c5)C4)ncnc3n2)cc1] | N-(4-bromo-3- methylbenzyl)-1-(2-(p- tolyl)-2H-pyrazolo[3,4- d]pyrimidin-4- yl)piperidine-3- carboxamide | 519.20 |
| 24 | 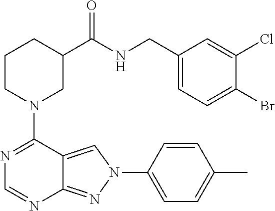 [SMILES: Cc1ccc(-n2cc3c(N4CCCC(C(=O)NCc5ccc(Br)c(Cl)c5)C4)ncnc3n2)cc1] | N-(4-bromo-3- chlorobenzyl)-1-(2-(p- tolyl)-2H-pyrazolo[3,4- d]pyrimidin-4- yl)piperidine-3- carboxamide | 539.12 |
| 25 | 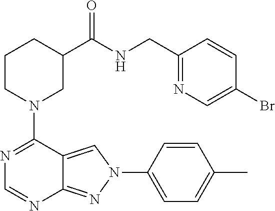 [SMILES: Cc1ccc(-n2cc3c(N4CCCC(C(=O)NCc5ccc(Br)cn5)C4)ncnc3n2)cc1] | N-((5-bromopyridin-2- yl)methyl)-1-(2-(p-tolyl)- 2H-pyrazolo[3,4- d]pyrimidin-4- yl)piperidine-3- carboxamide | 507.49 |
| 26 | 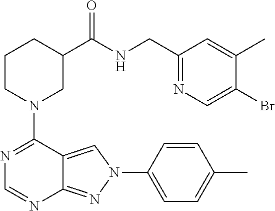 [SMILES: Cc1ccc(-n2cc3c(N4CCCC(C(=O)NCc5cc(C)c(Br)cn5)C4)ncnc3n2)cc1] | N-((5-bromo-4- methylpyridin-2- yl)methyl)-1-(2-(p-tolyl)- 2H-pyrazolo[3,4- d]pyrimidin-4- yl)piperidine-3- carboxamide | 520.13 |
| 27 | 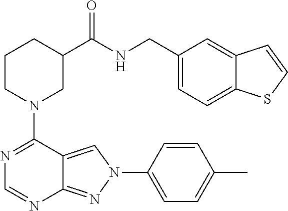 [SMILES: Cc1ccc(-n2cc3c(N4CCCC(C(=O)NCc5ccc6sccc6c5)C4)ncnc3n2)cc1] | N-(benzo[b]thiophen-5- ylmethyl)-1-(2-(p-tolyl)- 2H-pyrazolo[3,4- d]pyrimidin-4- yl)piperidine-3- carboxamide | 483.14 |
| 28 | 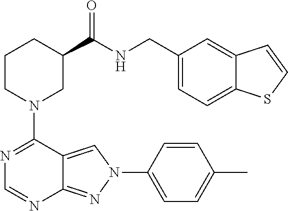 [SMILES: Cc1ccc(-n2cc3c(N4CCC[C@@H](C(=O)NCc5ccc6sccc6c5)C4)ncnc3n2)cc1] | (R)-N-(benzo[b]thiophen- 5-ylmethyl)-1-(2-(p-tolyl)- 2H-pyrazolo[3,4- d]pyrimidin-4- yl)piperidine-3- carboxamide | 483.23 |
| 29 | 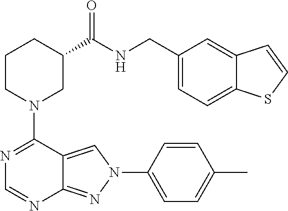 [SMILES: Cc1ccc(-n2cc3c(N4CCC[C@H](C(=O)NCc5ccc6sccc6c5)C4)ncnc3n2)cc1] | (S)-N-(benzo[b]thiophen- 5-ylmethyl)-1-(2-(p-tolyl)- 2H-pyrazolo[3,4- d]pyrimidin-4- yl)piperidine-3- carboxamide | 483.28 |
| 30 | 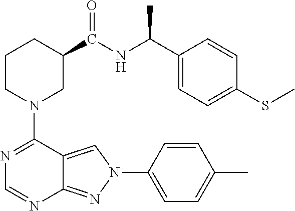 [SMILES: CSc1ccc([C@H](C)NC(=O)[C@@H]2CCCN(c3ncnc4nn(-c5ccc(C)cc5)cc34)C2)cc1] | (R)-N-((S)-1-(4- (methylthio)phenyl)ethyl)- 1-(2-(p-tolyl)-2H- pyrazolo[3,4-d]pyrimidin- 4-yl)piperidine-3- carboxamide | 487.21 |
| 31 | 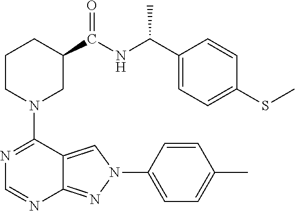 [SMILES: CSc1ccc([C@@H](C)NC(=O)[C@@H]2CCCN(c3ncnc4nn(-c5ccc(C)cc5)cc34)C2)cc1] | (R)-N-((R)-1-(4- (methylthio)phenyl)ethyl)- 1-(2-(p-tolyl)-2H- pyrazolo[3,4-d]pyrimidin- 4-yl)piperidine-3- carboxamide | 487.17 |
| 32 | 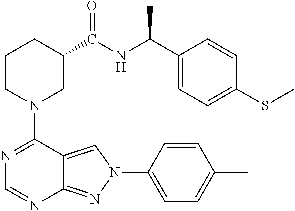 [SMILES: CSc1ccc([C@H](C)NC(=O)[C@H]2CCCN(c3ncnc4nn(-c5ccc(C)cc5)cc34)C2)cc1] | (S)-N-((S)-1-(4- (methylthio)phenyl)ethyl)- 1-(2-(p-tolyl)-2H- pyrazolo[3,4-d]pyrimidin- 4-yl)piperidine-3- carboxamide | 487.16 |
| 33 | 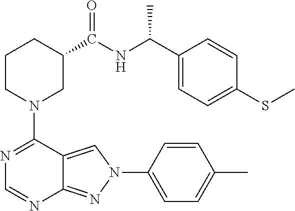 [SMILES: CSc1ccc([C@@H](C)NC(=O)[C@H]2CCCN(c3ncnc4nn(-c5ccc(C)cc5)cc34)C2)cc1] | (S)-N-((R)-1-(4- (methylthio)phenyl)ethyl)- 1-(2-(p-tolyl)-2H- pyrazolo[3,4-d]pyrimidin- 4-yl)piperidine-3- carboxamide | 487.28 |
| 34 | 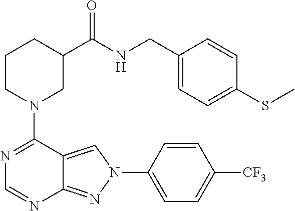 [SMILES: CSc1ccc(CNC(=O)C2CCCN(c3ncnc4nn(-c5ccc(C(F)(F)F)cc5)cc34)C2)cc1] | N-(4-(methylthio)benzyl)- 1-(2-(4- (trifluoromethyl)phenyl)- 2H-pyrazolo[3,4- d]pyrimidin-4- yl)piperidine-3- carboxamide | 527.13 |
| 35 | 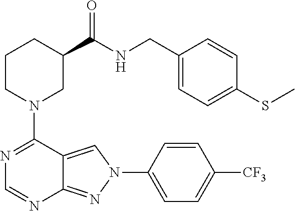 [SMILES: CSc1ccc(CNC(=O)[C@@H]2CCCN(c3ncnc4nn(-c5ccc(C(F)(F)F)cc5)cc34)C2)cc1] | (R)-N-(4- (methylthio)benzyl)-1-(2- (4- (trifluoromethyl)phenyl)- 2H-pyrazolo[3,4- d]pyrimidin-4- yl)piperidine-3- carboxamide | 527.14 |
| 36 | 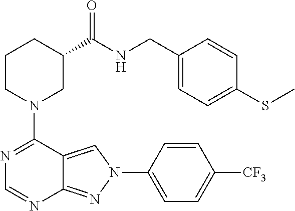 [SMILES: CSc1ccc(CNC(=O)[C@H]2CCCN(c3ncnc4nn(-c5ccc(C(F)(F)F)cc5)cc34)C2)cc1] | (S)-N-(4- (methylthio)benzyl)-1-(2- (4- (trifluoromethyl)phenyl)- 2H-pyrazolo[3,4- d]pyrimidin-4- yl)piperidine-3- carboxamide | 527.23 |
| 37 | 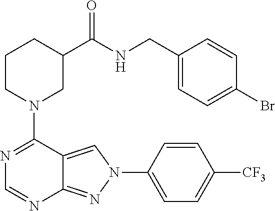 [SMILES: O=C(NCc1ccc(Br)cc1)C1CCCN(c2ncnc3nn(-c4ccc(C(F)(F)F)cc4)cc23)C1] | N-(4-bromobenzyl)-1-(2- (4- (trifluoromethyl)phenyl)- 2H-pyrazolo[3,4- d]pyrimidin-4- yl)piperidine-3- carboxamide | 559.04 |
| 38 | 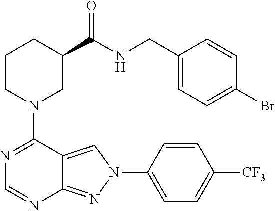 [SMILES: O=C(NCc1ccc(Br)cc1)[C@@H]1CCCN(c2ncnc3nn(-c4ccc(C(F)(F)F)cc4)cc23)C1] | (R)-N-(4-bromobenzyl)-1- (2-(4- (trifluoromethyl)phenyl)- 2H-pyrazolo[3,4- d]pyrimidin-4- yl)piperidine-3- carboxamide | 559.11 |
| 39 | 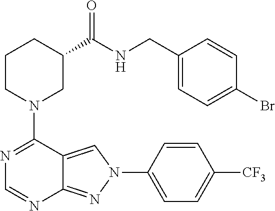 [SMILES: O=C(NCc1ccc(Br)cc1)[C@H]1CCCN(c2ncnc3nn(-c4ccc(C(F)(F)F)cc4)cc23)C1] | (S)-N-(4-bromobenzyl)-1- (2-(4- (trifluoromethyl)phenyl)- 2H-pyrazolo[3,4- d]pyrimidin-4- yl)piperidine-3- carboxamide | 559.05 |
| 40 | 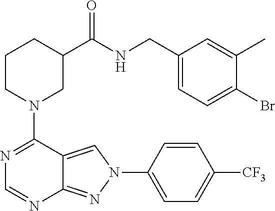 [SMILES: Cc1cc(CNC(=O)C2CCCN(c3ncnc4nn(-c5ccc(C(F)(F)F)cc5)cc34)C2)ccc1Br] | N-(4-bromo-3- methylbenzyl)-1-(2-(4- (trifluoromethyl)phenyl)- 2H-pyrazolo[3,4- d]pyrimidin-4- yl)piperidine-3- carboxamide | 573.14 |
| 41 | 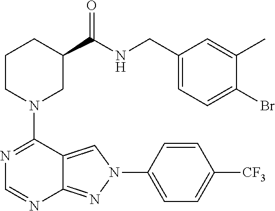 [SMILES: Cc1cc(CNC(=O)[C@@H]2CCCN(c3ncnc4nn(-c5ccc(C(F)(F)F)cc5)cc34)C2)ccc1Br] | (R)-N-(4-bromo-3- methylbenzyl)-1-(2-(4- (trifluoromethyl)phenyl)- 2H-pyrazolo[3,4- d]pyrimidin-4- yl)piperidine-3- carboxamide | 573.14 |
| 42 | 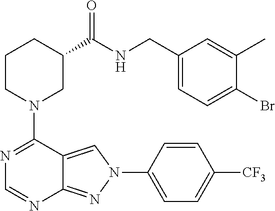 [SMILES: Cc1cc(CNC(=O)[C@H]2CCCN(c3ncnc4nn(-c5ccc(C(F)(F)F)cc5)cc34)C2)ccc1Br] | (S)-N-(4-bromo-3- methylbenzyl)-1-(2-(4- (trifluoromethyl)phenyl)- 2H-pyrazolo[3,4- d]pyrimidin-4- yl)piperidine-3- carboxamide | 573.11 |
| 43 | 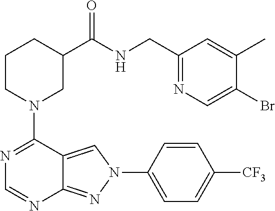 [SMILES: Cc1cc(CNC(=O)C2CCCN(c3ncnc4nn(-c5ccc(C(F)(F)F)cc5)cc34)C2)ncc1Br] | N-((5-bromo-4- methylpyridin-2- yl)methyl)-1-(2-(4- (trifluoromethyl)phenyl)- 2H-pyrazolo[3,4- d]pyrimidin-4- yl)piperidine-3- carboxamide | 574.09 |
| 44 | 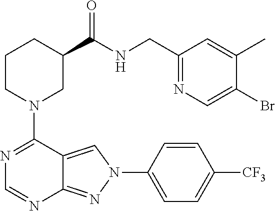 [SMILES: Cc1cc(CNC(=O)[C@@H]2CCCN(c3ncnc4nn(-c5ccc(C(F)(F)F)cc5)cc34)C2)ncc1Br] | (R)-N-((5-bromo-4- methylpyridin-2- yl)methyl)-1-(2-(4- (trifluoromethyl)phenyl)- 2H-pyrazolo[3,4- d]pyrimidin-4- yl)piperidine-3- carboxamide | 574.17 |
| 45 | 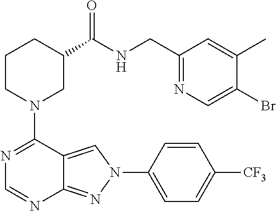 | (S)-N-((5-bromo-4- methylpyridin-2- yl)methyl)-1-(2-(4- (trifluoromethyl)phenyl)- 2H-pyrazolo[3,4- d]pyrimidin-4- yl)piperidine-3- carboxamide | 520.16 |
| 46 | 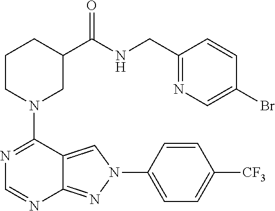 [SMILES: O=C(NCc1ccc(Br)cn1)C1CCCN(c2ncnc3nn(-c4ccc(C(F)(F)F)cc4)cc23)C1] | N-((5-bromopyridin-2- yl)methyl)-1-(2-(4- (trifluoromethyl)phenyl)- 2H-pyrazolo[3,4- d]pyrimidin-4- yl)piperidine-3- carboxamide | 560.11 |
| 47 | 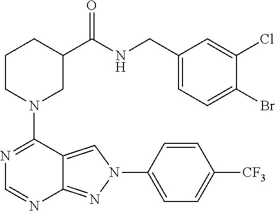 [SMILES: O=C(NCc1ccc(Br)c(Cl)c1)C1CCCN(c2ncnc3nn(-c4ccc(C(F)(F)F)cc4)cc23)C1] | N-(4-bromo-3- chlorobenzyl)-1-(2-(4- (trifluoromethyl)phenyl)- 2H-pyrazolo[3,4- d]pyrimidin-4- yl)piperidine-3- carboxamide | 593.18 |
| 48 | 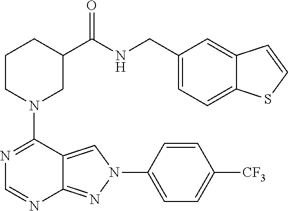 [SMILES: O=C(NCc1ccc2sccc2c1)C1CCCN(c2ncnc3nn(-c4ccc(C(F)(F)F)cc4)cc23)C1] | N-(benzo[b]thiophen-5- ylmethyl)-1-(2-(4- (trifluoromethyl)phenyl)- 2H-pyrazolo[3,4- d]pyrimidin-4- yl)piperidine-3- carboxamide | 537.12 |
| 49 | 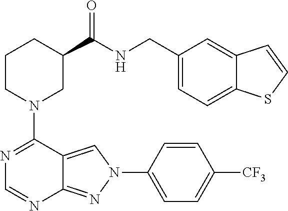 [SMILES: O=C(NCc1ccc2sccc2c1)[C@@H]1CCCN(c2ncnc3nn(-c4ccc(C(F)(F)F)cc4)cc23)C1] | (R)-N-(benzo[b]thiophen- 5-ylmethyl)-1-(2-(4- (trifluoromethyl)phenyl)- 2H-pyrazolo[3,4- d]pyrimidin-4- yl)piperidine-3- carboxamide | 537.16 |
| 50 | 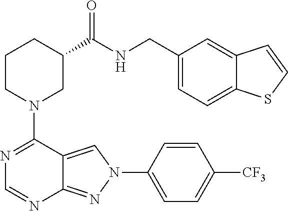 [SMILES: O=C(NCc1ccc2sccc2c1)[C@H]1CCCN(c2ncnc3nn(-c4ccc(C(F)(F)F)cc4)cc23)C1] | (S)-N-(benzo[b]thiophen- 5-ylmethyl)-1-(2-(4- (trifluoromethyl)phenyl)- 2H-pyrazolo[3,4- d]pyrimidin-4- yl)piperidine-3- carboxamide | 537.19 |
| 51 | 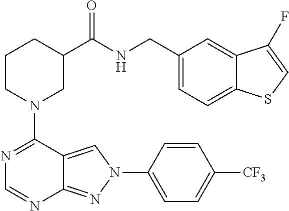 [SMILES: O=C(NCc1ccc2scc(F)c2c1)C1CCCN(c2ncnc3nn(-c4ccc(C(F)(F)F)cc4)cc23)C1] | N-((3- fluorobenzo[b]thiophen- 5-yl)methyl)-1-(2-(4- (trifluoromethyl)phenyl)- 2H-pyrazolo[3,4- d]pyrimidin-4- yl)piperidine-3- carboxamide | 555.15 |
| 52 | 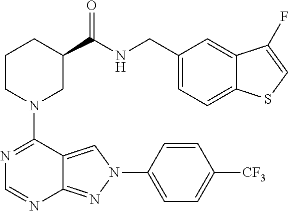 [SMILES: O=C(NCc1ccc2scc(F)c2c1)[C@@H]1CCCN(c2ncnc3nn(-c4ccc(C(F)(F)F)cc4)cc23)C1] | (R)-N-((3- fluorobenzo[b]thiophen- 5-yl)methyl)-1-(2-(4- (trifluoromethyl)phenyl)- 2H-pyrazolo[3,4- d]pyrimidin-4- yl)piperidine-3- carboxamide | 555.08 |
| 53 | 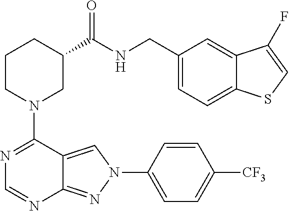 [SMILES: O=C(NCc1ccc2scc(F)c2c1)[C@H]1CCCN(c2ncnc3nn(-c4ccc(C(F)(F)F)cc4)cc23)C1] | (S)-N-((3- fluorobenzo[b]thiophen- 5-yl)methyl)-1-(2-(4- (trifluoromethyl)phenyl)- 2H-pyrazolo[3,4- d]pyrimidin-4- yl)piperidine-3- carboxamide | 555.11 |
| 54 | 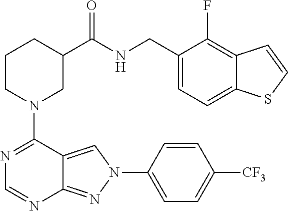 [SMILES: O=C(NCc1ccc2sccc2c1F)C1CCCN(c2ncnc3nn(-c4ccc(C(F)(F)F)cc4)cc23)C1] | N-((4- fluorobenzo[b]thiophen- 5-yl)methyl)-1-(2-(4- (trifluoromethyl)phenyl)- 2H-pyrazolo[3,4- d]pyrimidin-4- yl)piperidine-3- carboxamide | 555.19 |
| 55 | 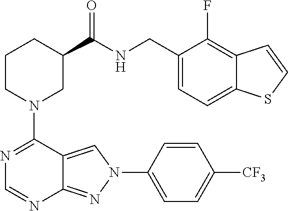 [SMILES: O=C(NCc1ccc2sccc2c1F)[C@@H]1CCCN(c2ncnc3nn(-c4ccc(C(F)(F)F)cc4)cc23)C1] | (R)-N-((4- fluorobenzo[b]thiophen- 5-yl)methyl)-1-(2-(4- (trifluoromethyl)phenyl)- 2H-pyrazolo[3,4- d]pyrimidin-4- yl)piperidine-3- carboxamide | 555.08 |
| 56 | 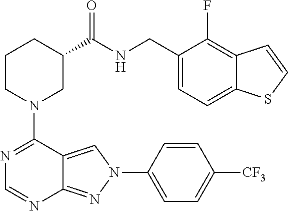 [SMILES: O=C(NCc1ccc2sccc2c1F)[C@H]1CCCN(c2ncnc3nn(-c4ccc(C(F)(F)F)cc4)cc23)C1] | (S)-N-((4- fluorobenzo[b]thiophen- 5-yl)methyl)-1-(2-(4- (trifluoromethyl)phenyl)- 2H-pyrazolo[3,4- d]pyrimidin-4- yl)piperidine-3- carboxamide | 555.08 |
| 57 | 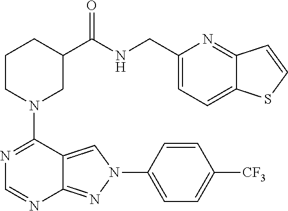 [SMILES: O=C(NCc1ccc2sccc2n1)C1CCCN(c2ncnc3nn(-c4ccc(C(F)(F)F)cc4)cc23)C1] | N-(thieno[3,2-b]pyridin-5- ylmethyl)-1-(2-(4- (trifluoromethyl)phenyl)- 2H-pyrazolo[3,4- d]pyrimidin-4- yl)piperidine-3- carboxamide | 538.17 |
| 58 | 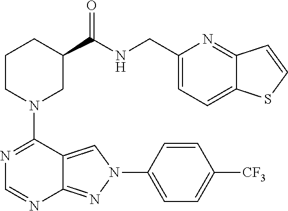 [SMILES: O=C(NCc1ccc2sccc2n1)[C@@H]1CCCN(c2ncnc3nn(-c4ccc(C(F)(F)F)cc4)cc23)C1] | (R)-N-(thieno[3,2- b]pyridin-5-ylmethyl)-1- (2-(4- (trifluoromethyl)phenyl)- 2H-pyrazolo[3,4- d]pyrimidin-4- yl)piperidine-3- carboxamide | 538.22 |
| 59 | 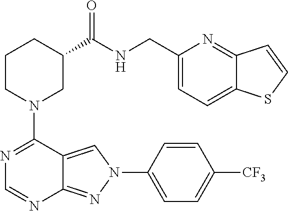 [SMILES: O=C(NCc1ccc2sccc2n1)[C@H]1CCCN(c2ncnc3nn(-c4ccc(C(F)(F)F)cc4)cc23)C1] | (S)-N-(thieno[3,2- b]pyridin-5-ylmethyl)-1- (2-(4- (trifluoromethyl)phenyl)- 2H-pyrazolo[3,4- d]pyrimidin-4- yl)piperidine-3- carboxamide | 538.24 |
| 60 | 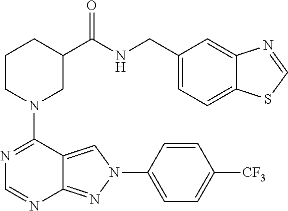 [SMILES: O=C(NCc1ccc2scnc2c1)C1CCCN(c2ncnc3nn(-c4ccc(C(F)(F)F)cc4)cc23)C1] | N-(benzo[d]thiazol-5- ylmethyl)-1-(2-(4- (trifluoromethyl)phenyl)- 2H-pyrazolo[3,4- d]pyrimidin-4- yl)piperidine-3- carboxamide | 538.17 |
| 61 | 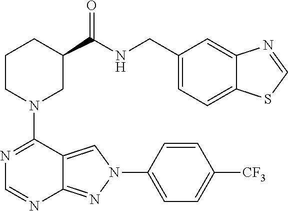 [SMILES: O=C(NCc1ccc2scnc2c1)[C@@H]1CCCN(c2ncnc3nn(-c4ccc(C(F)(F)F)cc4)cc23)C1] | (R)-N-(benzo[d]thiazol-5- ylmethyl)-1-(2-(4- (trifluoromethyl)phenyl)- 2H-pyrazolo[3,4- d]pyrimidin-4- yl)piperidine-3- carboxamide | 538.20 |
| 62 | 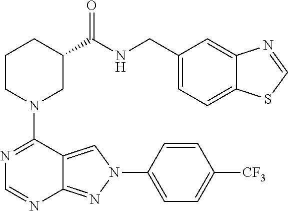 [SMILES: O=C(NCc1ccc2scnc2c1)[C@H]1CCCN(c2ncnc3nn(-c4ccc(C(F)(F)F)cc4)cc23)C1] | (S)-N-(benzo[d]thiazol-5- ylmethyl)-1-(2-(4- (trifluoromethyl)phenyl)- 2H-pyrazolo[3,4- d]pyrimidin-4- yl)piperidine-3- carboxamide | 538.14 |
| 63 | 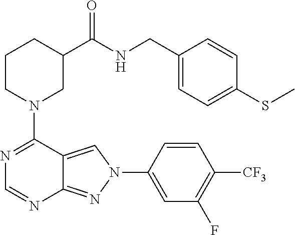 [SMILES: CSc1ccc(CNC(=O)C2CCCN(c3ncnc4nn(-c5ccc(C(F)(F)F)c(F)c5)cc34)C2)cc1] | 1-(2-(3-fluoro-4- (trifluoromethyl)phenyl)- 2H-pyrazolo[3,4- d]pyrimidin-4-yl)-N-(4- (methylthio)benzyl) piperidine-3-carboxamide | 545.19 |
| 64 | 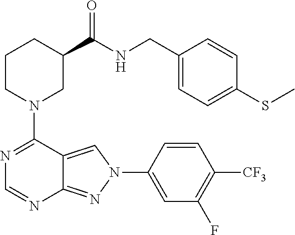 [SMILES: CSc1ccc(CNC(=O)[C@@H]2CCCN(c3ncnc4nn(-c5ccc(C(F)(F)F)c(F)c5)cc34)C2)cc1] | (R)-1-(2-(3-fluoro-4- (trifluoromethyl)phenyl)- 2H-pyrazolo[3,4- d]pyrimidin-4-yl)-N-(4- (methylthio)benzyl) piperidine-3-carboxamide | 545.25 |
| 65 | 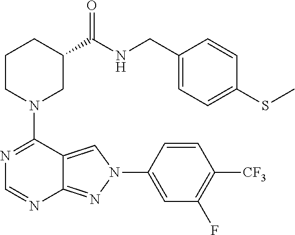 [SMILES: CSc1ccc(CNC(=O)[C@H]2CCCN(c3ncnc4nn(-c5ccc(C(F)(F)F)c(F)c5)cc34)C2)cc1] | (S)-1-(2-(3-fluoro-4- (trifluoromethyl)phenyl)- 2H-pyrazolo[3,4- d]pyrimidin-4-yl)-N-(4- (methylthio)benzyl) piperidine-3-carboxamide | 545.13 |
| 66 | 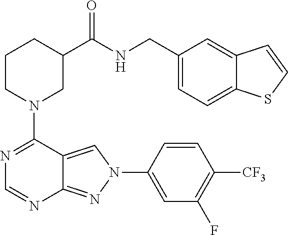 [SMILES: O=C(NCc1ccc2sccc2c1)C1CCCN(c2ncnc3nn(-c4ccc(C(F)(F)F)c(F)c4)cc23)C1] | N-(benzo[b]thiophen-5- ylmethyl)-1-(2-(3-fluoro-4- (trifluoromethyl)phenyl)- 2H-pyrazolo[3,4- d]pyrimidin-4- yl)piperidine-3- carboxamide | 555.20 |
| 67 | 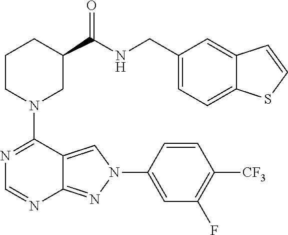 [SMILES: O=C(NCc1ccc2sccc2c1)[C@@H]1CCCN(c2ncnc3nn(-c4ccc(C(F)(F)F)c(F)c4)cc23)C1] | (R)-N-(benzo[b]thiophen- 5-ylmethyl)-1-(2-(3-fluoro- 4- (trifluoromethyl)phenyl)- 2H-pyrazolo[3,4- d]pyrimidin-4- yl)piperidine-3- carboxamide | 555.23 |
| 68 | 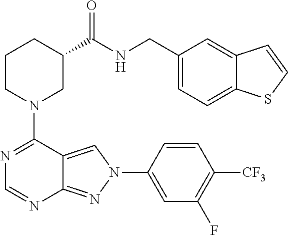 [SMILES: O=C(NCc1ccc2sccc2c1)[C@H]1CCCN(c2ncnc3nn(-c4ccc(C(F)(F)F)c(F)c4)cc23)C1] | (S)-N-(benzo[b]thiophen- 5-ylmethyl)-1-(2-(3-fluoro- 4- (trifluoromethyl)phenyl)- 2H-pyrazolo[3,4- d]pyrimidin-4- yl)piperidine-3- carboxamide | 555.09 |
| 69 | 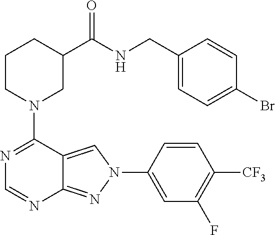 [SMILES: O=C(NCc1ccc(Br)cc1)C1CCCN(c2ncnc3nn(-c4ccc(C(F)(F)F)c(F)c4)cc23)C1] | N-(4-bromobenzyl)-1-(2- (3-fluoro-4- (trifluoromethyl)phenyl)- 2H-pyrazolo[3,4- d]pyrimidin-4- yl)piperidine-3- carboxamide | 577.05 |
| 70 | 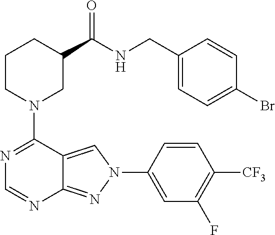 | (R)-N-(4-bromobenzyl)-1- (2-(3-fluoro-4- (trifluoromethyl)phenyl)- 2H-pyrazolo[3,4- d]pyrimidin-4- yl)piperidine-3- carboxamide | 577.02 |
| 71 | 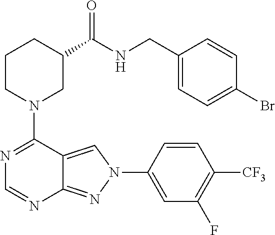 | (S)-N-(4-bromobenzyl)-1- (2-(3-fluoro-4- (trifluoromethyl)phenyl)- 2H-pyrazolo[3,4- d]pyrimidin-4- yl)piperidine-3- carboxamide | 577.02 |
| 72 | 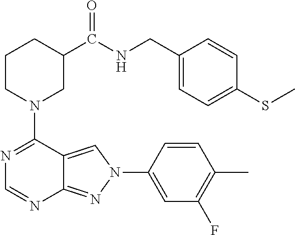 [SMILES: CSc1ccc(CNC(=O)C2CCCN(c3ncnc4nn(-c5ccc(C)c(F)c5)cc34)C2)cc1] | 1-(2-(3-fluoro-4- methylphenyl)-2H- pyrazolo[3,4-d]pyrimidin- 4-yl)-N-(4- (methylthio)benzyl) piperidine-3-carboxamide | 491.60 |
| 73 | 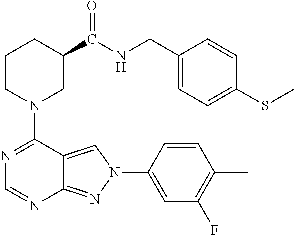 [SMILES: CSc1ccc(CNC(=O)[C@@H]2CCCN(c3ncnc4nn(-c5ccc(C)c(F)c5)cc34)C2)cc1] | (R)-1-(2-(3-fluoro-4- methylphenyl)-2H- pyrazolo[3,4-d]pyrimidin- 4-yl)-N-(4- (methylthio)benzyl) piperidine-3-carboxamide | 491.53 |
| 74 | 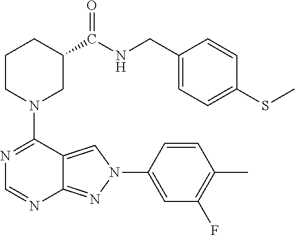 [SMILES: CSc1ccc(CNC(=O)[C@H]2CCCN(c3ncnc4nn(-c5ccc(C)c(F)c5)cc34)C2)cc1] | (S)-1-(2-(3-fluoro-4- methylphenyl)-2H- pyrazolo[3,4-d]pyrimidin- 4-yl)-N-(4- (methylthio)benzyl) piperidine-3-carboxamide | 491.59 |
| 75 | 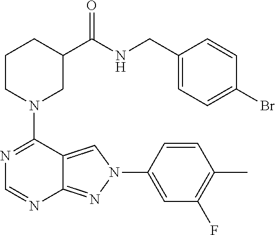 [SMILES: Cc1ccc(-n2cc3c(N4CCCC(C(=O)NCc5ccc(Br)cc5)C4)ncnc3n2)cc1F] | N-(4-bromobenzyl)-1-(2- (3-fluoro-4- methylphenyl)-2H- pyrazolo[3,4-d]pyrimidin- 4-yl)piperidine-3- carboxamide | 523.11 |
| 76 | 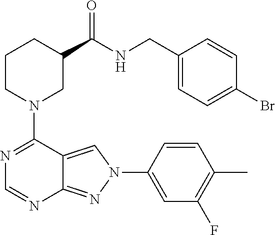 | (R)-N-(4-bromobenzyl)-1- (2-(3-fluoro-4- methylphenyl)-2H- pyrazolo[3,4-d]pyrimidin- 4-yl)piperidine-3- carboxamide | 523.17 |
| 77 | 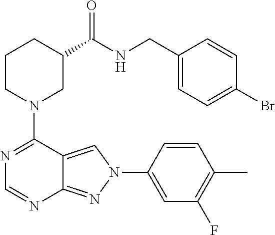 | (S)-N-(4-bromobenzyl)-1- (2-(3-fluoro-4- methylphenyl)-2H- pyrazolo[3,4-d]pyrimidin- 4-yl)piperidine-3- carboxamide | 523.10 |
| 78 | 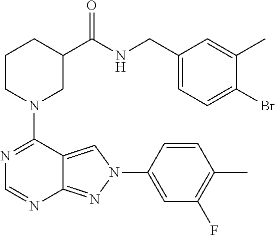 [SMILES: Cc1ccc(-n2cc3c(N4CCCC(C(=O)NCc5ccc(Br)c(C)c5)C4)ncnc3n2)cc1F] | N-(4-bromo-3- methylbenzyl)-1-(2-(3- fluoro-4-methylphenyl)- 2H-pyrazolo[3,4- d]pyrimidin-4- yl)piperidine-3- carboxamide | 537.20 |
| 79 | 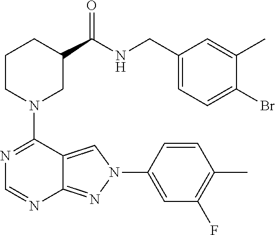 | (R)-N-(4-bromo-3- methylbenzyl)-1-(2-(3- fluoro-4-methylphenyl)- 2H-pyrazolo[3,4- d]pyrimidin-4- yl)piperidine-3- carboxamide | 537.09 |
| 80 | 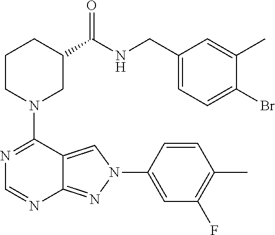 | (S)-N-(4-bromo-3- methylbenzyl)-1-(2-(3- fluoro-4-methylphenyl)- 2H-pyrazolo[3,4- d]pyrimidin-4- yl)piperidine-3- carboxamide | 537.21 |
| 81 | 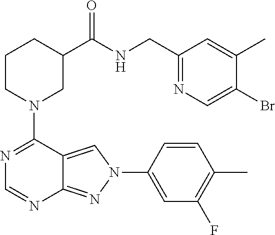 [SMILES: Cc1ccc(-n2cc3c(N4CCCC(C(=O)NCc5cc(C)c(Br)cn5)C4)ncnc3n2)cc1F] | N-((5-bromo-4- methylpyridin-2- yl)methyl)-1-(2-(3-fluoro- 4-methylphenyl)-2H- pyrazolo[3,4-d]pyrimidin- 4-yl)piperidine-3- carboxamide | 538.18 |
| 82 | 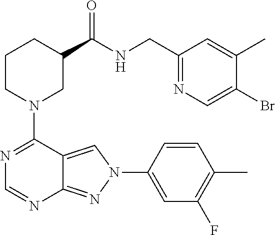 | (R)-N-((5-bromo-4- methylpyridin-2- yl)methyl)-1-(2-(3-fluoro- 4-methylphenyl)-2H- pyrazolo[3,4-d]pyrimidin- 4-yl)piperidine-3- carboxamide | 538.17 |
| 83 | 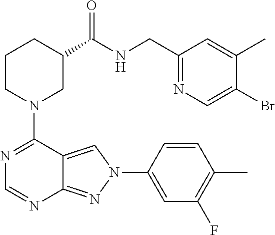 | (S)-N-((5-bromo-4- methylpyridin-2- yl)methyl)-1-(2-(3-fluoro- 4-methylphenyl)-2H- pyrazolo[3,4-d]pyrimidin- 4-yl)piperidine-3- carboxamide | 538.18 |
| 84 | 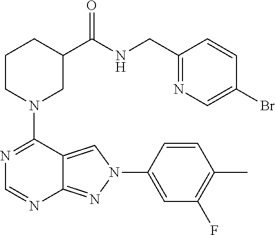 [SMILES: Cc1ccc(-n2cc3c(N4CCCC(C(=O)NCc5ccc(Br)cn5)C4)ncnc3n2)cc1F] | N-((5-bromopyridin-2- yl)methyl)-1-(2-(3-fluoro- 4-methylphenyl)-2H- pyrazolo[3,4-d]pyrimidin- 4-yl)piperidine-3- carboxamide | 524.11 |
| 85 | 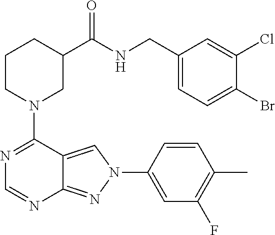 [SMILES: Cc1ccc(-n2cc3c(N4CCCC(C(=O)NCc5ccc(Br)c(Cl)c5)C4)ncnc3n2)cc1F] | N-(4-bromo-3- chlorobenzyl)-1-(2-(3- fluoro-4-methylphenyl)- 2H-pyrazolo[3,4- d]pyrimidin-4- yl)piperidine-3- carboxamide | 557.08 |
| 86 | 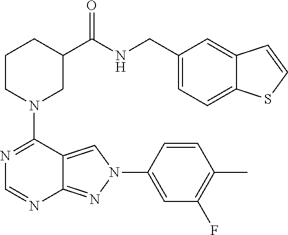 [SMILES: Cc1ccc(-n2cc3c(N4CCCC(C(=O)NCc5ccc6sccc6c5)C4)ncnc3n2)cc1F] | N-(benzo[b]thiophen-5- ylmethyl)-1-(2-(3-fluoro-4- methylphenyl)-2H- pyrazolo[3,4-d]pyrimidin- 4-yl)piperidine-3- carboxamide | 501.26 |
| 87 | 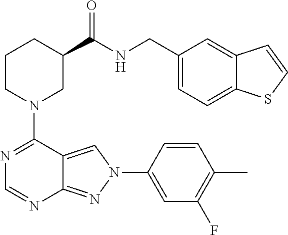 [SMILES: Cc1ccc(-n2cc3c(N4CCC[C@@H](C(=O)NCc5ccc6sccc6c5)C4)ncnc3n2)cc1F] | (R)-N-(benzo[b]thiophen- 5-ylmethyl)-1-(2-(3-fluoro- 4-methylphenyl)-2H- pyrazolo[3,4-d]pyrimidin- 4-yl)piperidine-3- carboxamide | 501.18 |
| 88 | 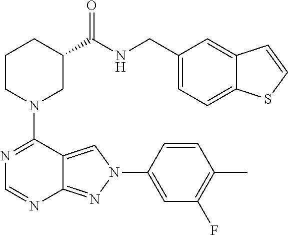 [SMILES: Cc1ccc(-n2cc3c(N4CCC[C@H](C(=O)NCc5ccc6sccc6c5)C4)ncnc3n2)cc1F] | (S)-N-(benzo[b]thiophen- 5-ylmethyl)-1-(2-(3-fluoro- 4-methylphenyl)-2H- pyrazolo[3,4-d]pyrimidin- 4-yl)piperidine-3- carboxamide | 501.12 |
| 89 | 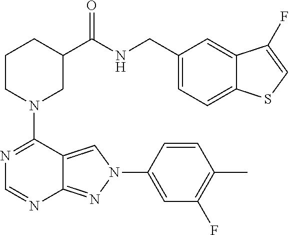 [SMILES: Cc1ccc(-n2cc3c(N4CCCC(C(=O)NCc5ccc6scc(F)c6c5)C4)ncnc3n2)cc1F] | 1-(2-(3-fluoro-4- methylphenyl)-2H- pyrazolo[3,4-d]pyrimidin- 4-yl)-N-((3- fluorobenzo[b]thiophen- 5-yl)methyl)piperidine-3- carboxamide | 520.63 |
| 90 | 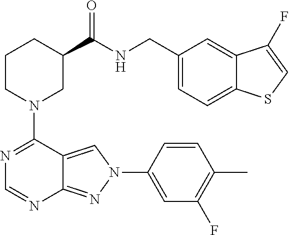 [SMILES: Cc1ccc(-n2cc3c(N4CCC[C@@H](C(=O)NCc5ccc6scc(F)c6c5)C4)ncnc3n2)cc1F] | (R)-1-(2-(3-fluoro-4- methylphenyl)-2H- pyrazolo[3,4-d]pyrimidin- 4-yl)-N-((3- fluorobenzo[b]thiophen- 5-yl)methyl)piperidine-3- carboxamide | 520.61 |
| 91 | 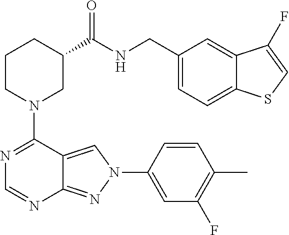 [SMILES: Cc1ccc(-n2cc3c(N4CCC[C@H](C(=O)NCc5ccc6scc(F)c6c5)C4)ncnc3n2)cc1F] | (S)-1-(2-(3-fluoro-4- methylphenyl)-2H- pyrazolo[3,4-d]pyrimidin- 4-yl)-N-((3- fluorobenzo[b]thiophen- 5-yl)methyl)piperidine-3- carboxamide | 520.62 |
| 92 | 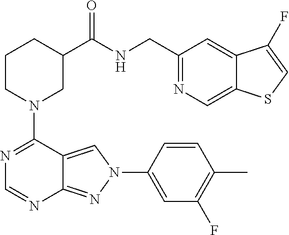 [SMILES: Cc1ccc(-n2cc3c(N4CCCC(C(=O)NCc5cc6c(F)csc6cn5)C4)ncnc3n2)cc1F] | 1-(2-(3-fluoro-4- methylphenyl)-2H- pyrazolo[3,4-d]pyrimidin- 4-yl)-N-((3- fluorothieno[2,3-c]pyridin- 5-yl)methyl)piperidine-3- carboxamide | 521.63 |
| 93 | 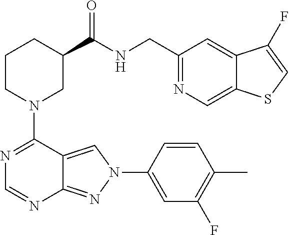 [SMILES: Cc1ccc(-n2cc3c(N4CCC[C@@H](C(=O)NCc5cc6c(F)csc6cn5)C4)ncnc3n2)cc1F] | (R)-1-(2-(3-fluoro-4- methylphenyl)-2H- pyrazolo[3,4-d]pyrimidin- 4-yl)-N-((3- fluorothieno[2,3-c]pyridin- 5-yl)methyl)piperidine-3- carboxamide | 521.57 |
| 94 | 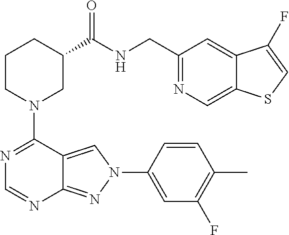 [SMILES: Cc1ccc(-n2cc3c(N4CCC[C@H](C(=O)NCc5cc6c(F)csc6cn5)C4)ncnc3n2)cc1F] | (S)-1-(2-(3-fluoro-4- methylphenyl)-2H- pyrazolo[3,4-d]pyrimidin- 4-yl)-N-((3- fluorothieno[2,3-c]pyridin- 5-yl)methyl)piperidine-3- carboxamide | 521.53 |
| 95 | 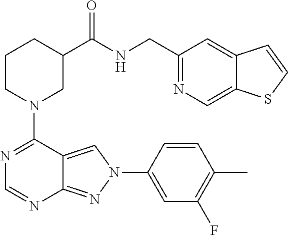 [SMILES: Cc1ccc(-n2cc3c(N4CCCC(C(=O)NCc5cc6ccsc6cn5)C4)ncnc3n2)cc1F] | 1-(2-(3-fluoro-4- methylphenyl)-2H- pyrazolo[3,4-d]pyrimidin- 4-yl)-N-(thieno[2,3- c]pyridin-5- ylmethyl)piperidine-3- carboxamide | 503.53 |
| 96 | 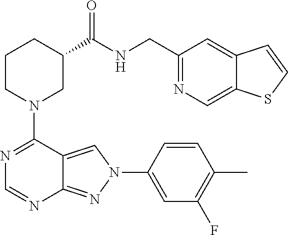 [SMILES: Cc1ccc(-n2cc3c(N4CCC[C@H](C(=O)NCc5cc6ccsc6cn5)C4)ncnc3n2)cc1F] | (S)-1-(2-(3-fluoro-4- methylphenyl)-2H- pyrazolo[3,4-d]pyrimidin- 4-yl)-N-(thieno[2,3- c]pyridin-5- ylmethyl)piperidine-3- carboxamide | 503.56 |
| 97 | 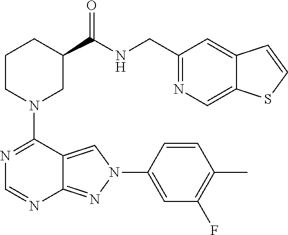 [SMILES: Cc1ccc(-n2cc3c(N4CCC[C@@H](C(=O)NCc5cc6ccsc6cn5)C4)ncnc3n2)cc1F] | (R)-1-(2-(3-fluoro-4- methylphenyl)-2H- pyrazolo[3,4-d]pyrimidin- 4-yl)-N-(thieno[2,3- c]pyridin-5- ylmethyl)piperidine-3- carboxamide | 503.64 |
| 98 | 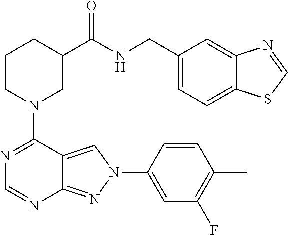 [SMILES: Cc1ccc(-n2cc3c(N4CCCC(C(=O)NCc5ccc6scnc6c5)C4)ncnc3n2)cc1F] | N-(benzo[d]thiazol-5- ylmethyl)-1-(2-(3-fluoro-4- methylphenyl)-2H- pyrazolo[3,4-d]pyrimidin- 4-yl)piperidine-3- carboxamide | 503.64 |
| 99 | 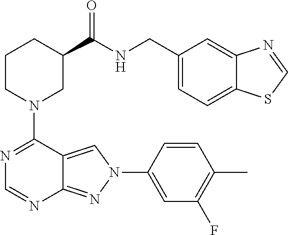 [SMILES: Cc1ccc(-n2cc3c(N4CCC[C@@H](C(=O)NCc5ccc6scnc6c5)C4)ncnc3n2)cc1F] | (R)-N-(benzo[d]thiazol-5- ylmethyl)-1-(2-(3-fluoro-4- methylphenyl)-2H- pyrazolo[3,4-d]pyrimidin- 4-yl)piperidine-3- carboxamide | 503.51 |
| 100 | 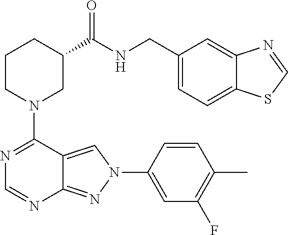 [SMILES: Cc1ccc(-n2cc3c(N4CCC[C@H](C(=O)NCc5ccc6scnc6c5)C4)ncnc3n2)cc1F] | (S)-N-(benzo[d]thiazol-5- ylmethyl)-1-(2-(3-fluoro-4- methylphenyl)-2H- pyrazolo[3,4-d]pyrimidin- 4-yl)piperidine-3- carboxamide | 503.67 |
| 101 | 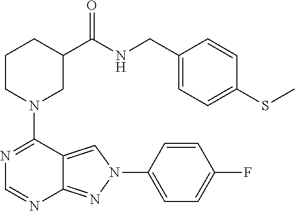 [SMILES: CSc1ccc(CNC(=O)C2CCCN(c3ncnc4nn(-c5ccc(F)cc5)cc34)C2)cc1] | 1-(2-(4-fluorophenyl)-2H- pyrazolo[3,4-d]pyrimidin- 4-yl)-N-(4- (methylthio)benzyl) piperidine-3-carboxamide | 477.19 |
| 102 | 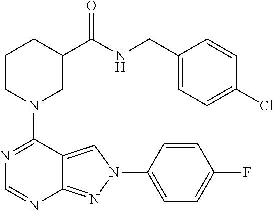 [SMILES: O=C(NCc1ccc(Cl)cc1)C1CCCN(c2ncnc3nn(-c4ccc(F)cc4)cc23)C1] | N-(4-chlorobenzyl)-1-(2- (4-fluorophenyl)-2H- pyrazolo[3,4-d]pyrimidin- 4-yl)piperidine-3- carboxamide | 465.20 |
| 103 | 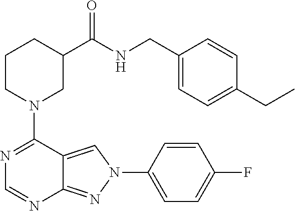 [SMILES: CCc1ccc(CNC(=O)C2CCCN(c3ncnc4nn(-c5ccc(F)cc5)cc34)C2)cc1] | N-(4-ethylbenzyl)-1-(2-(4- fluorophenyl)-2H- pyrazolo[3,4-d]pyrimidin- 4-yl)piperidine-3- carboxamide | 459.27 |
| 104 | 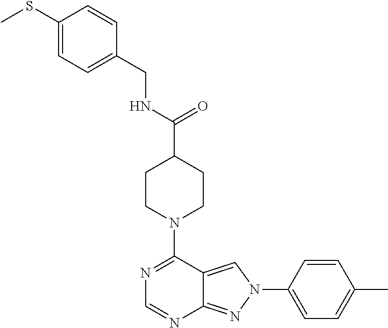 | N-(4-(methylthio)benzyl)- 1-(2-(p-tolyl)-2H- pyrazolo[3,4-d]pyrimidin- 4-yl)piperidine-4- carboxamide | 473.17 |
| 105 | 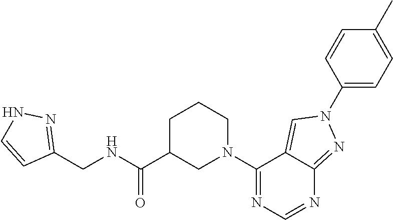 [SMILES: Cc1ccc(-n2cc3c(N4CCCC(C(=O)NCc5cc[nH]n5)C4)ncnc3n2)cc1] | N-((1H-pyrazol-3- yl)methyl)-1-(2-(p-tolyl)- 2H-pyrazolo[3,4- d]pyrimidin-4- yl)piperidine-3- carboxamide | 417.15 |
| 106 | 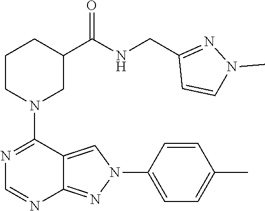 [SMILES: Cc1ccc(-n2cc3c(N4CCCC(C(=O)NCc5ccn(C)n5)C4)ncnc3n2)cc1] | N-((1-methyl-1H-pyrazol- 3-yl)methyl)-1-(2-(p-tolyl)- 2H-pyrazolo[3,4- d]pyrimidin-4- yl)piperidine-3- carboxamide | 431.30 |
| 107 | 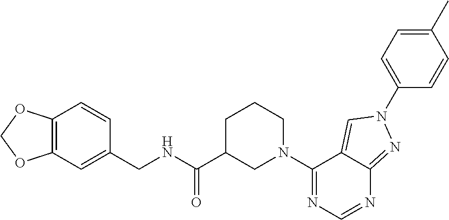 [SMILES: Cc1ccc(-n2cc3c(N4CCCC(C(=O)NCc5ccc6c(c5)OCO6)C4)ncnc3n2)cc1] | N-(benzo[d][1,3]dioxol-5- ylmethyl)-1-(2-(p-tolyl)- 2H-pyrazolo[3,4- d]pyrimidin-4- yl)piperidine-3- carboxamide | 471.17 |
| 108 | 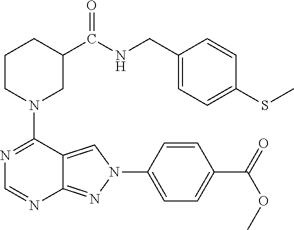 [SMILES: COC(=O)c1ccc(-n2cc3c(N4CCCC(C(=O)NCc5ccc(SC)cc5)C4)ncnc3n2)cc1] | methyl 4-(4-(3-((4- (methylthio)benzyl)carba- moyl)piperidin-1-yl)-2H- pyrazolo[3,4-d]pyrimidin- 2-yl)benzoate | 517.20 |
| 109 | 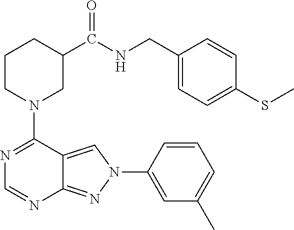 [SMILES: CSc1ccc(CNC(=O)C2CCCN(c3ncnc4nn(-c5cccc(C)c5)cc34)C2)cc1] | N-(4-(methylthio)benzyl)- 1-(2-(m-tolyl)-2H- pyrazolo[3,4-d]pyrimidin- 4-yl)piperidine-3- carboxamide | 473.16 |
| 110 | 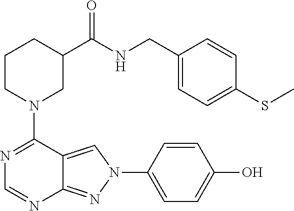 [SMILES: CSc1ccc(CNC(=O)C2CCCN(c3ncnc4nn(-c5ccc(O)cc5)cc34)C2)cc1] | 1-(2-(4-hydroxyphenyl)- 2H-pyrazolo[3,4- d]pyrimidin-4-yl)-N-(4- (methylthio)benzyl) piperidine-3-carboxamide | 475.16 |
| 111 | 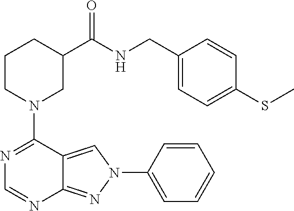 [SMILES: CSc1ccc(CNC(=O)C2CCCN(c3ncnc4nn(-c5ccccc5)cc34)C2)cc1] | N-(4-(methylthio)benzyl)- 1-(2-phenyl-2H- pyrazolo[3,4-d]pyrimidin- 4-yl)piperidine-3- carboxamide | 459.20 |
| 112 | 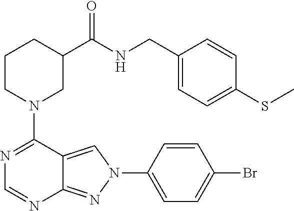 [SMILES: CSc1ccc(CNC(=O)C2CCCN(c3ncnc4nn(-c5ccc(Br)cc5)cc34)C2)cc1] | 1-(2-(4-bromophenyl)- 2H-pyrazolo[3,4- d]pyrimidin-4-yl)-N-(4- (methylthio)benzyl) piperidine-3-carboxamide | 537.14 |
| 113 | 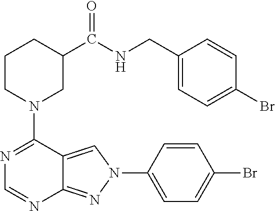 [SMILES: O=C(NCc1ccc(Br)cc1)C1CCCN(c2ncnc3nn(-c4ccc(Br)cc4)cc23)C1] | N-(4-bromobenzyl)-1-(2- (4-bromophenyl)-2H- pyrazolo[3,4-d]pyrimidin- 4-yl)piperidine-3- carboxamide | 568.96 |
| 114 | 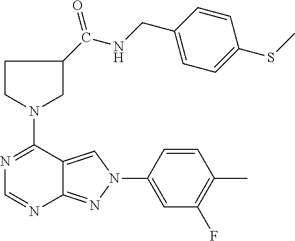 | 1-(2-(3-fluoro-4- methylphenyl)-2H- pyrazolo[3,4-d]pyrimidin- 4-yl)-N-(4- (methylthio)benzyl) pyrrolidine-3-carboxamide | 478.58 |
| 115 | 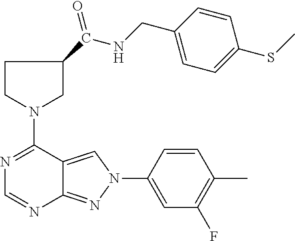 [SMILES: CSc1ccc(CNC(=O)[C@@H]2CCN(c3ncnc4nn(-c5ccc(C)c(F)c5)cc34)C2)cc1] | (R)-1-(2-(3-fluoro-4- methylphenyl)-2H- pyrazolo[3,4-d]pyrimidin- 4-yl)-N-(4- (methylthio)benzyl) pyrrolidine-3-carboxamide | 478.62 |
| 116 | 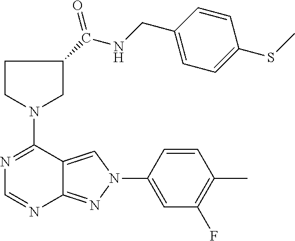 [SMILES: CSc1ccc(CNC(=O)[C@H]2CCN(c3ncnc4nn(-c5ccc(C)c(F)c5)cc34)C2)cc1] | (S)-1-(2-(3-fluoro-4- methylphenyl)-2H- pyrazolo[3,4-d]pyrimidin- 4-yl)-N-(4- (methylthio)benzyl) pyrrolidine-3-carboxamide | 478.61 |
| 117 | 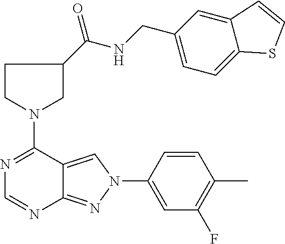 [SMILES: Cc1ccc(-n2cc3c(N4CCC(C(=O)NCc5ccc6sccc6c5)C4)ncnc3n2)cc1F] | N-(benzo[b]thiophen-5- ylmethyl)-1-(2-(3-fluoro-4- methylphenyl)-2H- pyrazolo[3,4-d]pyrimidin- 4-yl)pyrrolidine-3- carboxamide | 488.51 |
| 118 | 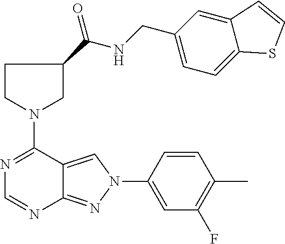 [SMILES: Cc1ccc(-n2cc3c(N4CC[C@@H](C(=O)NCc5ccc6sccc6c5)C4)ncnc3n2)cc1F] | (R)-N-(benzo[b]thiophen- 5-ylmethyl)-1-(2-(3-fluoro- 4-methylphenyl)-2H- pyrazolo[3,4-d]pyrimidin- 4-yl)pyrrolidine-3- carboxamide | 488.55 |
| 119 | 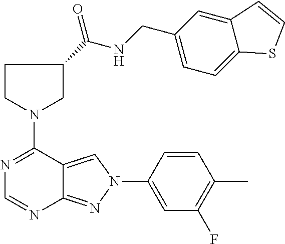 [SMILES: Cc1ccc(-n2cc3c(N4CC[C@H](C(=O)NCc5ccc6sccc6c5)C4)ncnc3n2)cc1F] | (S)-N-(benzo[b]thiophen- 5-ylmethyl)-1-(2-(3-fluoro- 4-methylphenyl)-2H- pyrazolo[3,4-d]pyrimidin- 4-yl)pyrrolidine-3- carboxamide | 488.61 |
| 120 | 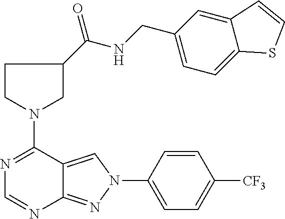 [SMILES: O=C(NCc1ccc2sccc2c1)C1CCN(c2ncnc3nn(-c4ccc(C(F)(F)F)cc4)cc23)C1] | N-(benzo[b]thiophen-5- ylmethyl)-1-(2-(4- (trifluoromethyl)phenyl)- 2H-pyrazolo[3,4- d]pyrimidin-4- yl)pyrrolidine-3- carboxamide | 524.63 |
| 121 | 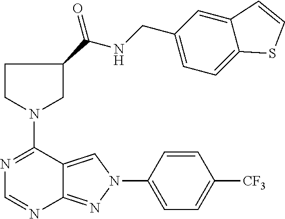 [SMILES: O=C(NCc1ccc2sccc2c1)[C@@H]1CCN(c2ncnc3nn(-c4ccc(C(F)(F)F)cc4)cc23)C1] | (R)-N-(benzo[b]thiophen- 5-ylmethyl)-1-(2-(4- (trifluoromethyl)phenyl)- 2H-pyrazolo[3,4- d]pyrimidin-4- yl)pyrrolidine-3- carboxamide | 524.56 |
| 122 | 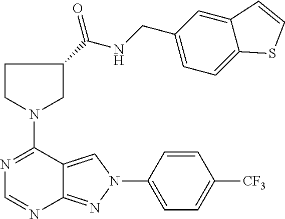 [SMILES: O=C(NCc1ccc2sccc2c1)[C@H]1CCN(c2ncnc3nn(-c4ccc(C(F)(F)F)cc4)cc23)C1] | (S)-N-(benzo[b]thiophen- 5-ylmethyl)-1-(2-(4- (trifluoromethyl)phenyl)- 2H-pyrazolo[3,4- d]pyrimidin-4- yl)pyrrolidine-3- carboxamide | 524.50 |
| 123 | 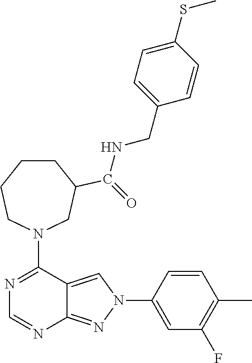 [SMILES: CSc1ccc(CNC(=O)C2CCCCN(c3ncnc4nn(-c5ccc(C)c(F)c5)cc34)C2)cc1] | 1-(2-(3-fluoro-4- methylphenyl)-2H- pyrazolo[3,4-d]pyrimidin- 4-yl)-N-(4- (methylthio)benzyl) azepane-3-carboxamide | 505.24 |
| 124 | 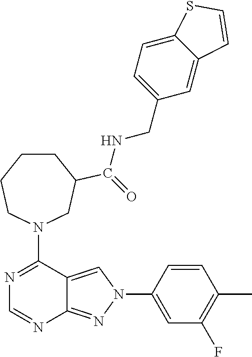 [SMILES: Cc1ccc(-n2cc3c(N4CCCCC(C(=O)NCc5ccc6sccc6c5)C4)ncnc3n2)cc1F] | N-(benzo[b]thiophen-5- ylmethyl)-1-(2-(3-fluoro-4- methylphenyl)-2H- pyrazolo[3,4-d]pyrimidin- 4-yl)azepane-3- carboxamide | 515.22 |
| 125 | 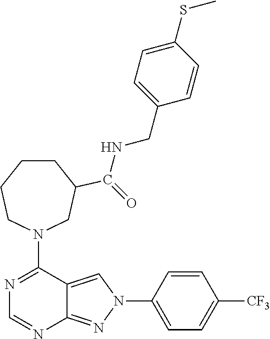 [SMILES: CSc1ccc(CNC(=O)C2CCCCN(c3ncnc4nn(-c5ccc(C(F)(F)F)cc5)cc34)C2)cc1] | N-(4-(methylthio)benzyl)- 1-(2-(4- (trifluoromethyl)phenyl)- 2H-pyrazolo[3,4- d]pyrimidin-4-yl)azepane- 3-carboxamide | 541.22 |
| 126 | 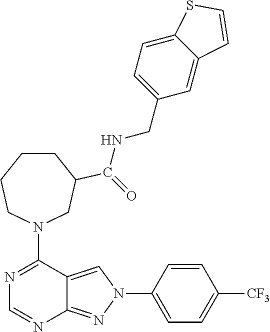 [SMILES: O=C(NCc1ccc2sccc2c1)C1CCCCN(c2ncnc3nn(-c4ccc(C(F)(F)F)cc4)cc23)C1] | N-(benzo[b]thiophen-5- ylmethyl)-1-(2-(4- (trifluoromethyl)phenyl)- 2H-pyrazolo[3,4- d]pyrimidin-4-yl)azepane- 3-carboxamide | 551.21 |
| 127 | 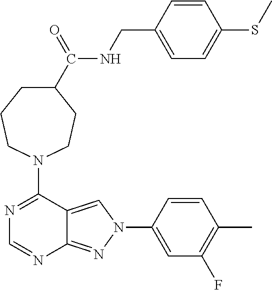 [SMILES: CSc1ccc(CNC(=O)C2CCCN(c3ncnc4nn(-c5ccc(C)c(F)c5)cc34)CC2)cc1] | 1-(2-(3-fluoro-4- methylphenyl)-2H- pyrazolo[3,4-d]pyrimidin- 4-yl)-N-(4- (methylthio)benzyl) azepane-4-carboxamide | 505.23 |
| 128 | 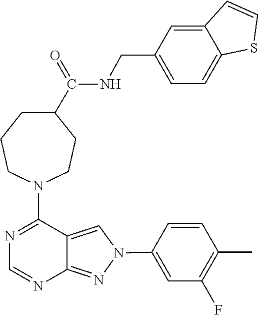 [SMILES: Cc1ccc(-n2cc3c(N4CCCC(C(=O)NCc5ccc6sccc6c5)CC4)ncnc3n2)cc1F] | N-(benzo[b]thiophen-5- ylmethyl)-1-(2-(3-fluoro-4- methylphenyl)-2H- pyrazolo[3,4-d]pyrimidin- 4-yl)azepane-4- carboxamide | 515.24 |
| 129 | 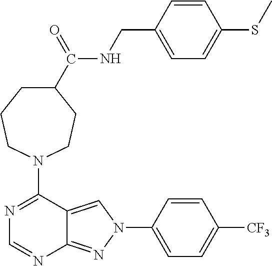 [SMILES: CSc1ccc(CNC(=O)C2CCCN(c3ncnc4nn(-c5ccc(C(F)(F)F)cc5)cc34)CC2)cc1] | N-(4-(methylthio)benzyl)- 1-(2-(4- (trifluoromethyl)phenyl)- 2H-pyrazolo[3,4- d]pyrimidin-4-yl)azepane- 4-carboxamide | 541.15 |
| 130 | 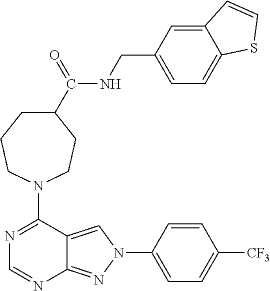 [SMILES: O=C(NCc1ccc2sccc2c1)C1CCCN(c2ncnc3nn(-c4ccc(C(F)(F)F)cc4)cc23)CC1] | N-(benzo[b]thiophen-5- ylmethyl)-1-(2-(4- (trifluoromethyl)phenyl)- 2H-pyrazolo[3,4- d]pyrimidin-4-yl)azepane- 4-carboxamide | 551.12 |
| 131 | 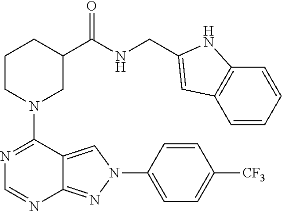 [SMILES: O=C(NCc1cc2ccccc2[nH]1)C1CCCN(c2ncnc3nn(-c4ccc(C(F)(F)F)cc4)cc23)C1] | N-((1H-indol-2-yl)methyl)- 1-(2-(4- (trifluoromethyl)phenyl)- 2H-pyrazolo[3,4- d]pyrimidin-4- yl)piperidine-3- carboxamide | 520.28 |
| 132 | 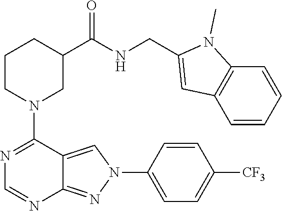 [SMILES: Cn1c(CNC(=O)C2CCCN(c3ncnc4nn(-c5ccc(C(F)(F)F)cc5)cc34)C2)cc2ccccc21] | N-((1-methyl-1H-indol-2- yl)methyl)-1-(2-(4- (trifluoromethyl)phenyl)- 2H-pyrazolo[3,4- d]pyrimidin-4- yl)piperidine-3- carboxamide | 534.17 |
| 133 | 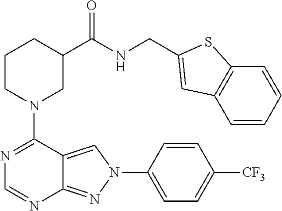 [SMILES: O=C(NCc1cc2ccccc2s1)C1CCCN(c2ncnc3nn(-c4ccc(C(F)(F)F)cc4)cc23)C1] | N-(benzo[b]thiophen-2- ylmethyl)-1-(2-(4- (trifluoromethyl)phenyl)- 2H-pyrazolo[3,4- d]pyrimidin-4- yl)piperidine-3- carboxamide | 537.33 |
| 134 | 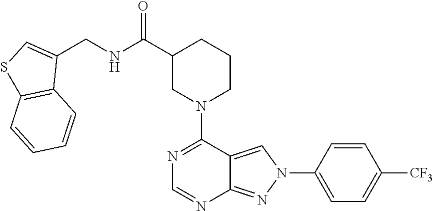 [SMILES: O=C(NCc1csc2ccccc12)C1CCCN(c2ncnc3nn(-c4ccc(C(F)(F)F)cc4)cc23)C1] | N-(benzo[b]thiophen-3- ylmethyl)-1-(2-(4- (trifluoromethyl)phenyl)- 2H-pyrazolo[3,4- d]pyrimidin-4- yl)piperidine-3- carboxamide | 537.20 |
| 135 | 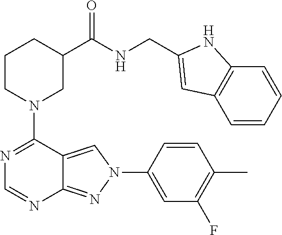 [SMILES: Cc1ccc(-n2cc3c(N4CCCC(C(=O)NCc5cc6ccccc6[nH]5)C4)ncnc3n2)cc1F] | N-((1H-indol-2-yl)methyl)- 1-(2-(3-fluoro-4- methylphenyl)-2H- pyrazolo[3,4-d]pyrimidin- 4-yl)piperidine-3- carboxamide | 484.23 |
| 136 | 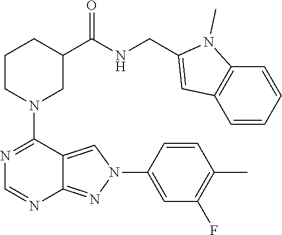 [SMILES: Cc1ccc(-n2cc3c(N4CCCC(C(=O)NCc5cc6ccccc6n5C)C4)ncnc3n2)cc1F] | 1-(2-(3-fluoro-4- methylphenyl)-2H- pyrazolo[3,4-d]pyrimidin- 4-yl)-N-((1-methyl-1H- indol-2- yl)methyl)piperidine-3- carboxamide | 498.23 |
| 137 | 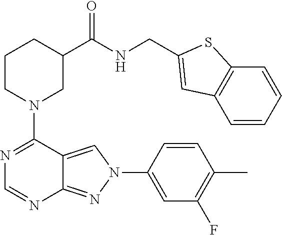 [SMILES: Cc1ccc(-n2cc3c(N4CCCC(C(=O)NCc5cc6ccccc6s5)C4)ncnc3n2)cc1F] | N-(benzo[b]thiophen-2- ylmethyl)-1-(2-(3-fluoro-4- methylphenyl)-2H- pyrazolo[3,4-d]pyrimidin- 4-yl)piperidine-3- carboxamide | 501.17 |
| 138 | 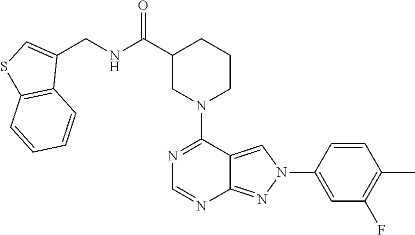 [SMILES: Cc1ccc(-n2cc3c(N4CCCC(C(=O)NCc5csc6ccccc56)C4)ncnc3n2)cc1F] | N-(benzo[b]thiophen-3- ylmethyl)-1-(2-(3-fluoro-4- methylphenyl)-2H- pyrazolo[3,4-d]pyrimidin- 4-yl)piperidine-3- carboxamide | 501.11 |
| 139 | 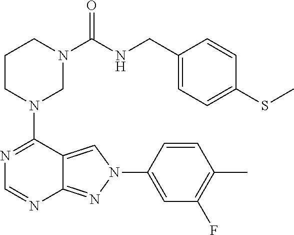 [SMILES: CSc1ccc(CNC(=O)N2CCCN(c3ncnc4nn(-c5ccc(C)c(F)c5)cc34)C2)cc1] | 3-(2-(3-fluoro-4- methylphenyl)-2H- pyrazolo[3,4-d]pyrimidin- 4-yl)-N-(4- (methylthio)benzyl) tetrahydropyrimidine- 1(2H)-carboxamide | 492.20 |
| 140 | 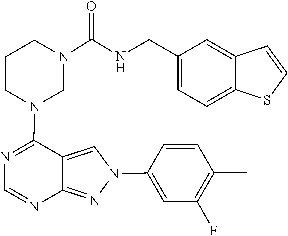 [SMILES: Cc1ccc(-n2cc3c(N4CCCN(C(=O)NCc5ccc6sccc6c5)C4)ncnc3n2)cc1F] | N-(benzo[b]thiophen-5- ylmethyl)-3-(2-(3-fluoro-4- methylphenyl)-2H- pyrazolo[3,4-d]pyrimidin- 4-yl)tetrahydropyrimidine- 1(2H)-carboxamide | 502.11 |
| 141 | 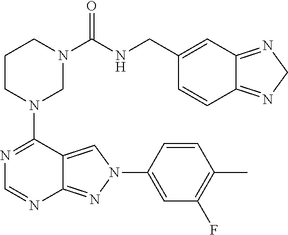 [SMILES: Cc1ccc(-n2cc3c(N4CCCN(C(=O)NCc5ccc6c(c5)=NCN=6)C4)ncnc3n2)cc1F] | N-((2H-benzo[d]imidazol- 5-yl)methyl)-3-(2-(3- fluoro-4-methylphenyl)- 2H-pyrazolo[3,4- d]pyrimidin-4- yl)tetrahydropyrimidine- 1(2H)-carboxamide | 486.19 |
| 142 | 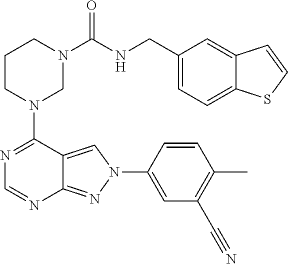 [SMILES: Cc1ccc(-n2cc3c(N4CCCN(C(=O)NCc5ccc6sccc6c5)C4)ncnc3n2)cc1C#N] | N-(benzo[b]thiophen-5- ylmethyl)-3-(2-(3-cyano- 4-methylphenyl)-2H- pyrazolo[3,4-d]pyrimidin- 4-yl)tetrahydropyrimidine- 1(2H)-carboxamide | 509.13 |
| 143 | 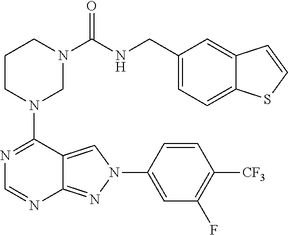 [SMILES: O=C(NCc1ccc2sccc2c1)N1CCCN(c2ncnc3nn(-c4ccc(C(F)(F)F)c(F)c4)cc23)C1] | N-(benzo[b]thiophen-5- ylmethyl)-3-(2-(3-fluoro-4- (trifluoromethyl)phenyl)- 2H-pyrazolo[3,4- d]pyrimidin-4- yl)tetrahydropyrimidine- 1(2H)-carboxamide | 556.17 |
| 144 | 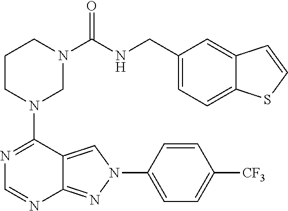 [SMILES: O=C(NCc1ccc2sccc2c1)N1CCCN(c2ncnc3nn(-c4ccc(C(F)(F)F)cc4)cc23)C1] | N-(benzo[b]thiophen-5- ylmethyl)-3-(2-(4- (trifluoromethyl)phenyl)- 2H-pyrazolo[3,4- d]pyrimidin-4- yl)tetrahydropyrimidine- 1(2H)-carboxamide | 538.20 |
| 145 | 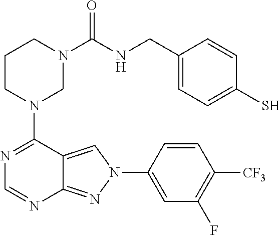 [SMILES: O=C(NCc1ccc(S)cc1)N1CCCN(c2ncnc3nn(-c4ccc(C(F)(F)F)c(F)c4)cc23)C1] | 3-(2-(3-fluoro-4- (trifluoromethyl)phenyl)- 2H-pyrazolo[3,4- d]pyrimidin-4-yl)-N-(4- mercaptobenzyl)tetra- hydropyrimidine-1(2H)- carboxamide | 532.10 |
| 146 | 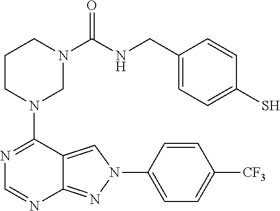 [SMILES: O=C(NCc1ccc(S)cc1)N1CCCN(c2ncnc3nn(-c4ccc(C(F)(F)F)cc4)cc23)C1] | N-(4-mercaptobenzyl)-3- (2-(4- (trifluoromethyl)phenyl)- 2H-pyrazolo[3,4- d]pyrimidin-4- yl)tetrahydropyrimidine- 1(2H)-carboxamide | 514.11 |
| 147 | 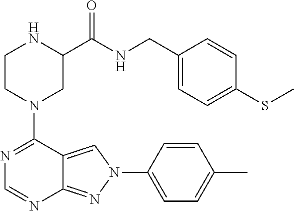 [SMILES: CSc1ccc(CNC(=O)C2CN(c3ncnc4nn(-c5ccc(C)cc5)cc34)CCN2)cc1] | N-(4-(methylthio)benzyl)- 4-(2-(p-tolyl)-2H- pyrazolo[3,4-d]pyrimidin- 4-yl)piperazine-2- carboxamide | 474.60 |
| 148 | 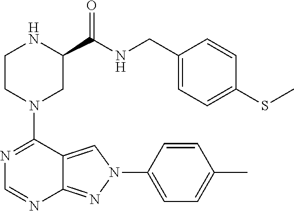 [SMILES: CSc1ccc(CNC(=O)[C@H]2CN(c3ncnc4nn(-c5ccc(C)cc5)cc34)CCN2)cc1] | (R)-N-(4- (methylthio)benzyl)-4-(2- (p-tolyl)-2H-pyrazolo[3,4- d]pyrimidin-4- yl)piperazine-2- carboxamide | 474.30 |
| 149 | 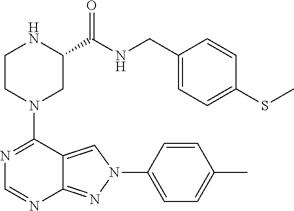 [SMILES: CSc1ccc(CNC(=O)[C@@H]2CN(c3ncnc4nn(-c5ccc(C)cc5)cc34)CCN2)cc1] | (S)-N-(4- (methylthio)benzyl)-4-(2- (p-tolyl)-2H-pyrazolo[3,4- d]pyrimidin-4- yl)piperazine-2- carboxamide | 474.54 |
| 150 | 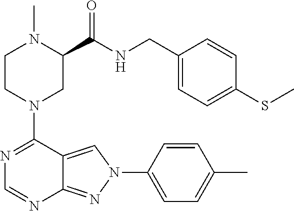 [SMILES: CSc1ccc(CNC(=O)[C@H]2CN(c3ncnc4nn(-c5ccc(C)cc5)cc34)CCN2C)cc1] | (R)-1-methyl-N-(4- (methylthio)benzyl)-4-(2- (p-tolyl)-2H-pyrazolo[3,4- d]pyrimidin-4- yl)piperazine-2- carboxamide | 488.41 |
| 151 | 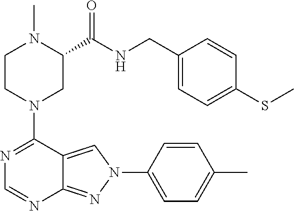 [SMILES: CSc1ccc(CNC(=O)[C@@H]2CN(c3ncnc4nn(-c5ccc(C)cc5)cc34)CCN2C)cc1] | (S)-1-methyl-N-(4- (methylthio)benzyl)-4-(2- (p-tolyl)-2H-pyrazolo[3,4- d]pyrimidin-4- yl)piperazine-2- carboxamide | 488.44 |
| 152 | 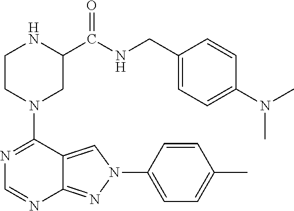 [SMILES: Cc1ccc(-n2cc3c(N4CCNC(C(=O)NCc5ccc(N(C)C)cc5)C4)ncnc3n2)cc1] | N-(4- (dimethylamino)benzyl)- 4-(2-(p-tolyl)-2H- pyrazolo[3,4-d]pyrimidin- 4-yl)piperazine-2- carboxamide | 471.23 |
| 153 | 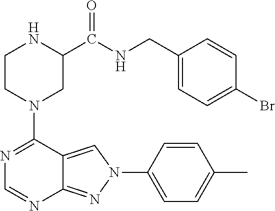 [SMILES: Cc1ccc(-n2cc3c(N4CCNC(C(=O)NCc5ccc(Br)cc5)C4)ncnc3n2)cc1] | N-(4-bromobenzyl)-4-(2- (p-tolyl)-2H-pyrazolo[3,4- d]pyrimidin-4- yl)piperazine-2- carboxamide | 506.23 |
| 154 | 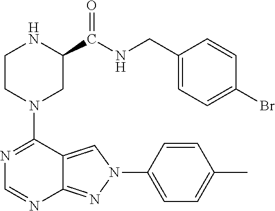 [SMILES: Cc1ccc(-n2cc3c(N4CCN[C@@H](C(=O)NCc5ccc(Br)cc5)C4)ncnc3n2)cc1] | (R)-N-(4-bromobenzyl)-4- (2-(p-tolyl)-2H- pyrazolo[3,4-d]pyrimidin- 4-yl)piperazine-2- carboxamide | 506.34 |
| 155 | 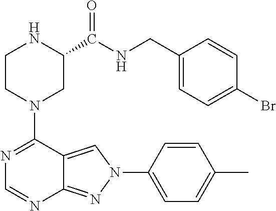 [SMILES: Cc1ccc(-n2cc3c(N4CCN[C@H](C(=O)NCc5ccc(Br)cc5)C4)ncnc3n2)cc1] | (S)-N-(4-bromobenzyl)-4- (2-(p-tolyl)-2H- pyrazolo[3,4-d]pyrimidin- 4-yl)piperazine-2- carboxamide | 506.22 |
| 156 | 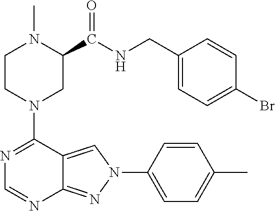 [SMILES: Cc1ccc(-n2cc3c(N4CCN(C)[C@@H](C(=O)NCc5ccc(Br)cc5)C4)ncnc3n2)cc1] | (R)-N-(4-bromobenzyl)-1- methyl-4-(2-(p-tolyl)-2H- pyrazolo[3,4-d]pyrimidin- 4-yl)piperazine-2- carboxamide | 520.27 |
| 157 | 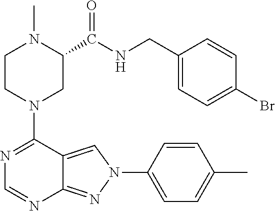 [SMILES: Cc1ccc(-n2cc3c(N4CCN(C)[C@H](C(=O)NCc5ccc(Br)cc5)C4)ncnc3n2)cc1] | (S)-N-(4-bromobenzyl)-1- methyl-4-(2-(p-tolyl)-2H- pyrazolo[3,4-d]pyrimidin- 4-yl)piperazine-2- carboxamide | 520.31 |
| 158 | 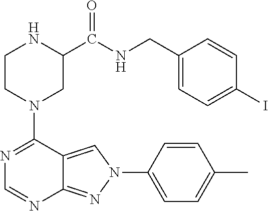 [SMILES: Cc1ccc(-n2cc3c(N4CCNC(C(=O)NCc5ccc(I)cc5)C4)ncnc3n2)cc1] | N-(4-iodobenzyl)-4-(2-(p- tolyl)-2H-pyrazolo[3,4- d]pyrimidin-4- yl)piperazine-2- carboxamide | 554.14 |
| 159 | 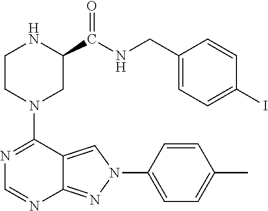 [SMILES: Cc1ccc(-n2cc3c(N4CCN[C@@H](C(=O)NCc5ccc(I)cc5)C4)ncnc3n2)cc1] | (R)-N-(4-iodobenzyl)-4- (2-(p-tolyl)-2H- pyrazolo[3,4-d]pyrimidin- 4-yl)piperazine-2- carboxamide | 554.20 |
| 160 | 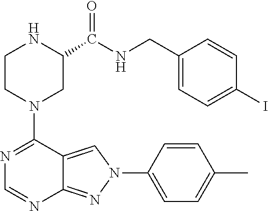 | (R)-N-(4-iodobenzyl)-4- (2-(p-tolyl)-2H- pyrazolo[3,4-d]pyrimidin- 4-yl)piperazine-2- carboxamide | 554.20 |
| 161 | 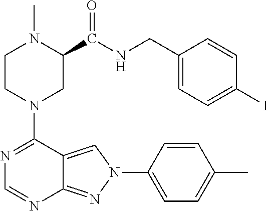 [SMILES: Cc1ccc(-n2cc3c(N4CCN(C)[C@@H](C(=O)NCc5ccc(I)cc5)C4)ncnc3n2)cc1] | (R)-N-(4-iodobenzyl)-1- methyl-4-(2-(p-tolyl)-2H- pyrazolo[3,4-d]pyrimidin- 4-yl)piperazine-2- carboxamide | 568.19 |
| 162 | 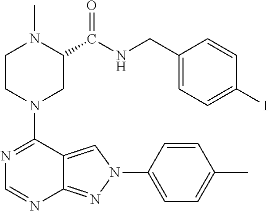 | (S)-N-(4-iodobenzyl)-1- methyl-4-(2-(p-tolyl)-2H- pyrazolo[3,4-d]pyrimidin- 4-yl)piperazine-2- carboxamide | 568.22 |
| 163 | 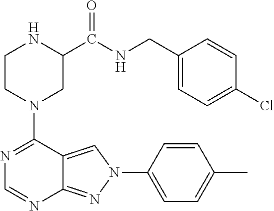 [SMILES: Cc1ccc(-n2cc3c(N4CCNC(C(=O)NCc5ccc(Cl)cc5)C4)ncnc3n2)cc1] | N-(4-chlorobenzyl)-4-(2- (p-tolyl)-2H-pyrazolo[3,4- d]pyrimidin-4- yl)piperazine-2- carboxamide | 462.31 |
| 164 | 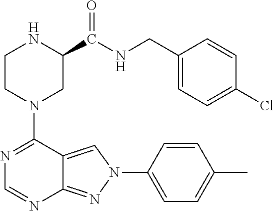 [SMILES: Cc1ccc(-n2cc3c(N4CCN[C@@H](C(=O)NCc5ccc(Cl)cc5)C4)ncnc3n2)cc1] | (R)-N-(4-chlorobenzyl)-4- (2-(p-tolyl)-2H- pyrazolo[3,4-d]pyrimidin- 4-yl)piperazine-2- carboxamide | 462.28 |
| 165 | 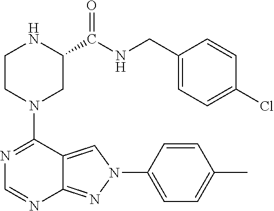 [SMILES: Cc1ccc(-n2cc3c(N4CCN[C@H](C(=O)NCc5ccc(Cl)cc5)C4)ncnc3n2)cc1] | (S)-N-(4-chlorobenzyl)-4- (2-(p-tolyl)-2H- pyrazolo[3,4-d]pyrimidin- 4-yl)piperazine-2- carboxamide | 462.23 |
| 166 | 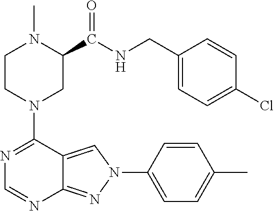 [SMILES: Cc1ccc(-n2cc3c(N4CCN(C)[C@@H](C(=O)NCc5ccc(Cl)cc5)C4)ncnc3n2)cc1] | (R)-N-(4-chlorobenzyl)-1- methyl-4-(2-(p-tolyl)-2H- pyrazolo[3,4-d]pyrimidin- 4-yl)piperazine-2- carboxamide | 476.25 |
| 167 | 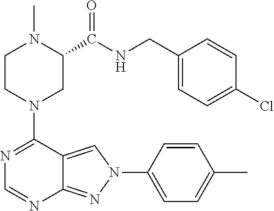 [SMILES: Cc1ccc(-n2cc3c(N4CCN(C)[C@H](C(=O)NCc5ccc(Cl)cc5)C4)ncnc3n2)cc1] | (S)-N-(4-chlorobenzyl)-1- methyl-4-(2-(p-tolyl)-2H- pyrazolo[3,4-d]pyrimidin- 4-yl)piperazine-2- carboxamide | 476.31 |
| 168 | 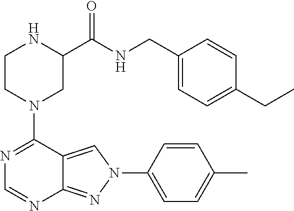 [SMILES: CCc1ccc(CNC(=O)C2CN(c3ncnc4nn(-c5ccc(C)cc5)cc34)CCN2)cc1] | N-(4-ethylbenzyl)-4-(2-(p- tolyl)-2H-pyrazolo[3,4- d]pyrimidin-4- yl)piperazine-2- carboxamide | 456.27 |
| 169 | 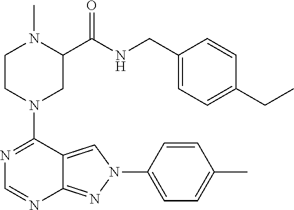 [SMILES: CCc1ccc(CNC(=O)C2CN(c3ncnc4nn(-c5ccc(C)cc5)cc34)CCN2C)cc1] | N-(4-ethylbenzyl)-1- methyl-4-(2-(p-tolyl)-2H- pyrazolo[3,4-d]pyrimidin- 4-yl)piperazine-2- carboxamide | 470.33 |
| 170 | 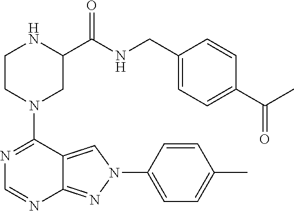 [SMILES: CC(=O)c1ccc(CNC(=O)C2CN(c3ncnc4nn(-c5ccc(C)cc5)cc34)CCN2)cc1] | N-(4-acetylbenzyl)-4-(2- (p-tolyl)-2H-pyrazolo[3,4- d]pyrimidin-4- yl)piperazine-2- carboxamide | 470.31 |
| 171 | 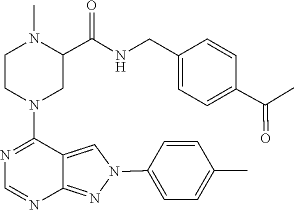 [SMILES: CC(=O)c1ccc(CNC(=O)C2CN(c3ncnc4nn(-c5ccc(C)cc5)cc34)CCN2C)cc1] | N-(4-acetylbenzyl)-1- methyl-4-(2-(p-tolyl)-2H- pyrazolo[3,4-d]pyrimidin- 4-yl)piperazine-2- carboxamide | 484.37 |
| 172 | 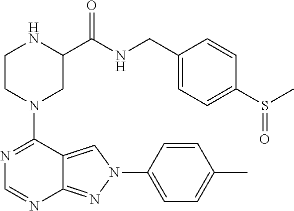 [SMILES: Cc1ccc(-n2cc3c(N4CCNC(C(=O)NCc5ccc(S(C)=O)cc5)C4)ncnc3n2)cc1] | N-(4- (methylsulfinyl)benzyl)-4- (2-(p-tolyl)-2H- pyrazolo[3,4-d]pyrimidin- 4-yl)piperazine-2- carboxamide | 489.68 |
| 173 | 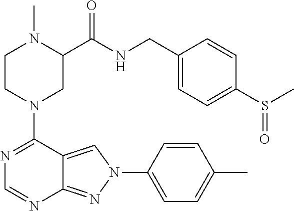 [SMILES: Cc1ccc(-n2cc3c(N4CCN(C)C(C(=O)NCc5ccc(S(C)=O)cc5)C4)ncnc3n2)cc1] | 1-methyl-N-(4- (methylsulfinyl)benzyl)-4- (2-(p-tolyl)-2H- pyrazolo[3,4-d]pyrimidin- 4-yl)piperazine-2- carboxamide | 504.27 |
| 174 | 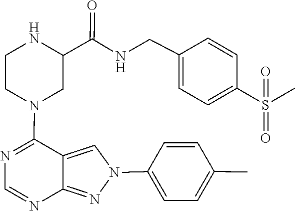 [SMILES: Cc1ccc(-n2cc3c(N4CCNC(C(=O)NCc5ccc(S(C)(=O)=O)cc5)C4)ncnc3n2)cc1] | N-(4- (methylsulfonyl)benzyl)- 4-(2-(p-tolyl)-2H- pyrazolo[3,4-d]pyrimidin- 4-yl)piperazine-2- carboxamide | 506.17 |
| 175 | 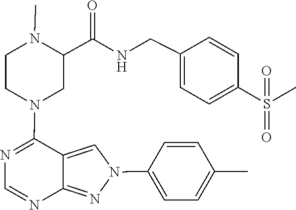 [SMILES: Cc1ccc(-n2cc3c(N4CCN(C)C(C(=O)NCc5ccc(S(C)(=O)=O)cc5)C4)ncnc3n2)cc1] | 1-methyl-N-(4- (methylsulfonyl)benzyl)- 4-(2-(p-tolyl)-2H- pyrazolo[3,4-d]pyrimidin- 4-yl)piperazine-2- carboxamide | 520.33 |
| 176 | 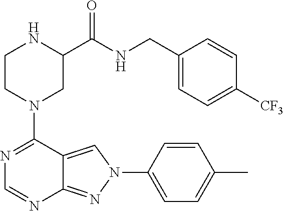 [SMILES: Cc1ccc(-n2cc3c(N4CCNC(C(=O)NCc5ccc(C(F)(F)F)cc5)C4)ncnc3n2)cc1] | 4-(2-(p-tolyl)-2H- pyrazolo[3,4-d]pyrimidin- 4-yl)-N-(4- (trifluoromethyl)benzyl) piperazine-2-carboxamide | 496.22 |
| 177 | 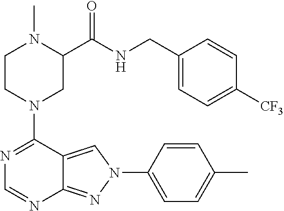 [SMILES: Cc1ccc(-n2cc3c(N4CCN(C)C(C(=O)NCc5ccc(C(F)(F)F)cc5)C4)ncnc3n2)cc1] | 1-methyl-4-(2-(p-tolyl)- 2H-pyrazolo[3,4- d]pyrimidin-4-yl)-N-(4- (trifluoromethyl)benzyl) piperazine-2-carboxamide | 510.29 |
| 178 | 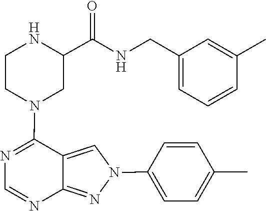 [SMILES: Cc1ccc(-n2cc3c(N4CCNC(C(=O)NCc5cccc(C)c5)C4)ncnc3n2)cc1] | N-(3-methylbenzyl)-4-(2- (p-tolyl)-2H-pyrazolo[3,4- d]pyrimidin-4- yl)piperazine-2- carboxamide | 442.27 |
| 179 | 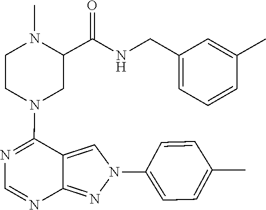 [SMILES: Cc1ccc(-n2cc3c(N4CCN(C)C(C(=O)NCc5cccc(C)c5)C4)ncnc3n2)cc1] | 1-methyl-N-(3- methylbenzyl)-4-(2-(p- tolyl)-2H-pyrazolo[3,4- d]pyrimidin-4- yl)piperazine-2- carboxamide | 456.37 |
| 180 | 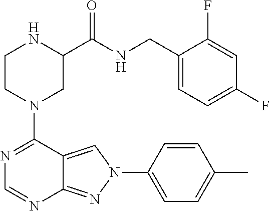 [SMILES: Cc1ccc(-n2cc3c(N4CCNC(C(=O)NCc5ccc(F)cc5F)C4)ncnc3n2)cc1] | N-(2,4-difluorobenzyl)-4- (2-(p-tolyl)-2H- pyrazolo[3,4-d]pyrimidin- 4-yl)piperazine-2- carboxamide | 464.37 |
| 181 | 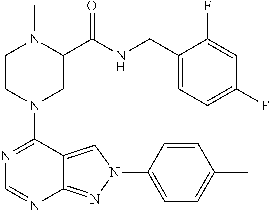 [SMILES: Cc1ccc(-n2cc3c(N4CCN(C)C(C(=O)NCc5ccc(F)cc5F)C4)ncnc3n2)cc1] | N-(2,4-difluorobenzyl)-1- methyl-4-(2-(p-tolyl)-2H- pyrazolo[3,4-d]pyrimidin- 4-yl)piperazine-2- carboxamide | 478.24 |
| 182 | 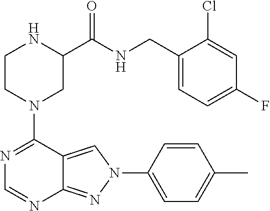 [SMILES: Cc1ccc(-n2cc3c(N4CCNC(C(=O)NCc5ccc(F)cc5Cl)C4)ncnc3n2)cc1] | N-(2-chloro-4- fluorobenzyl)-4-(2-(p- tolyl)-2H-pyrazolo[3,4- d]pyrimidin-4- yl)piperazine-2- carboxamide | 480.29 |
| 183 | 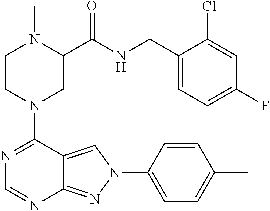 [SMILES: Cc1ccc(-n2cc3c(N4CCN(C)C(C(=O)NCc5ccc(F)cc5Cl)C4)ncnc3n2)cc1] | N-(2-chloro-4- fluorobenzyl)-1-methyl-4- (2-(p-tolyl)-2H- pyrazolo[3,4-d]pyrimidin- 4-yl)piperazine-2- carboxamide | 494.37 |
| 184 | 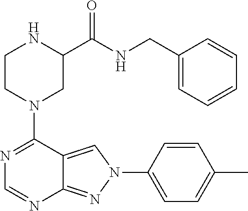 [SMILES: Cc1ccc(-n2cc3c(N4CCNC(C(=O)NCc5ccccc5)C4)ncnc3n2)cc1] | N-benzyl-4-(2-(p-tolyl)- 2H-pyrazolo[3,4- d]pyrimidin-4- yl)piperazine-2- carboxamide | 428.16 |
| 185 | 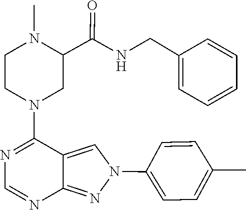 [SMILES: Cc1ccc(-n2cc3c(N4CCN(C)C(C(=O)NCc5ccccc5)C4)ncnc3n2)cc1] | N-benzyl-1-methyl-4-(2- (p-tolyl)-2H-pyrazolo[3,4- d]pyrimidin-4- yl)piperazine-2- carboxamide | 442.37 |
| 186 | 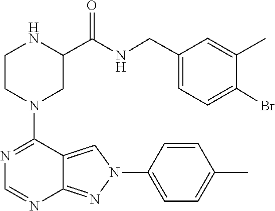 [SMILES: Cc1ccc(-n2cc3c(N4CCNC(C(=O)NCc5ccc(Br)c(C)c5)C4)ncnc3n2)cc1] | N-(4-bromo-3- methylbenzyl)-4-(2-(p- tolyl)-2H-pyrazolo[3,4- d]pyrimidin-4- yl)piperazine-2- carboxamide | 520.21 |
| 187 | 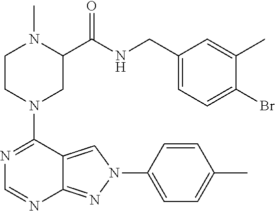 [SMILES: Cc1ccc(-n2cc3c(N4CCN(C)C(C(=O)NCc5ccc(Br)c(C)c5)C4)ncnc3n2)cc1] | N-(4-bromo-3- methylbenzyl)-1-methyl- 4-(2-(p-tolyl)-2H- pyrazolo[3,4-d]pyrimidin- 4-yl)piperazine-2- carboxamide | 534.17 |
| 188 | 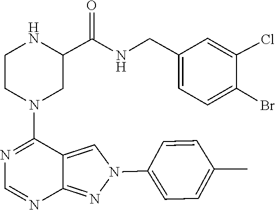 [SMILES: Cc1ccc(-n2cc3c(N4CCNC(C(=O)NCc5ccc(Br)c(Cl)c5)C4)ncnc3n2)cc1] | N-(4-bromo-3- chlorobenzyl)-4-(2-(p- tolyl)-2H-pyrazolo[3,4- d]pyrimidin-4- yl)piperazine-2- carboxamide | 540.06 |
| 189 | 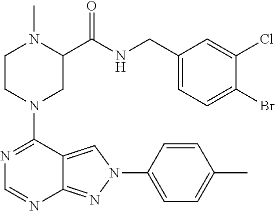 [SMILES: Cc1ccc(-n2cc3c(N4CCN(C)C(C(=O)NCc5ccc(Br)c(Cl)c5)C4)ncnc3n2)cc1] | N-(4-bromo-3- chlorobenzyl)-1-methyl-4- (2-(p-tolyl)-2H- pyrazolo[3,4-d]pyrimidin- 4-yl)piperazine-2- carboxamide | 554.10 |
| 190 | 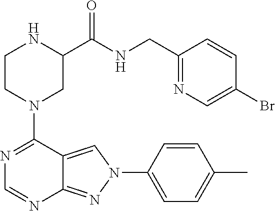 [SMILES: Cc1ccc(-n2cc3c(N4CCNC(C(=O)NCc5ccc(Br)cn5)C4)ncnc3n2)cc1] | N-((5-bromopyridin-2- yl)methyl)-4-(2-(p-tolyl)- 2H-pyrazolo[3,4- d]pyrimidin-4- yl)piperazine-2- carboxamide | 507.08 |
| 191 | 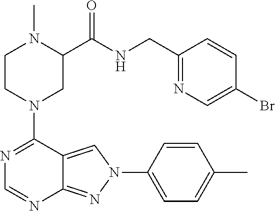 [SMILES: Cc1ccc(-n2cc3c(N4CCN(C)C(C(=O)NCc5ccc(Br)cn5)C4)ncnc3n2)cc1] | N-((5-bromopyridin-2- yl)methyl)-1-methyl-4-(2- (p-tolyl)-2H-pyrazolo[3,4- d]pyrimidin-4- yl)piperazine-2- carboxamide | 521.37 |
| 192 | 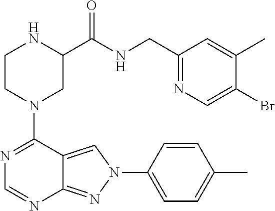 [SMILES: Cc1ccc(-n2cc3c(N4CCNC(C(=O)NCc5cc(C)c(Br)cn5)C4)ncnc3n2)cc1] | N-((5-bromo-4- methylpyridin-2- yl)methyl)-4-(2-(p-tolyl)- 2H-pyrazolo[3,4- d]pyrimidin-4- yl)piperazine-2- carboxamide | 521.33 |
| 193 | 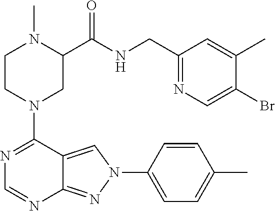 [SMILES: Cc1ccc(-n2cc3c(N4CCN(C)C(C(=O)NCc5cc(C)c(Br)cn5)C4)ncnc3n2)cc1] | N-((5-bromo-4- methylpyridin-2- yl)methyl)-1-methyl-4-(2- (p-tolyl)-2H-pyrazolo[3,4- d]pyrimidin-4- yl)piperazine-2- carboxamide | 535.12 |
| 194 | 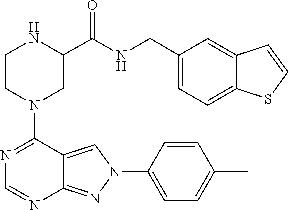 [SMILES: Cc1ccc(-n2cc3c(N4CCNC(C(=O)NCc5ccc6sccc6c5)C4)ncnc3n2)cc1] | N-(benzo[b]thiophen-5- ylmethyl)-4-(2-(p-tolyl)- 2H-pyrazolo[3,4- d]pyrimidin-4- yl)piperazine-2- carboxamide | 484.07 |
| 195 | 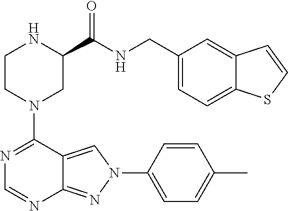 [SMILES: Cc1ccc(-n2cc3c(N4CCN[C@@H](C(=O)NCc5ccc6sccc6c5)C4)ncnc3n2)cc1] | (R)-N-(benzo[b]thiophen- 5-ylmethyl)-4-(2-(p-tolyl)- 2H-pyrazolo[3,4- d]pyrimidin-4- yl)piperazine-2- carboxamide | 484.11 |
| 196 | 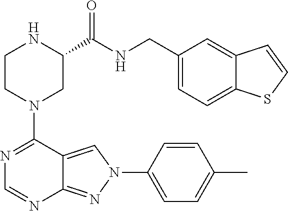 [SMILES: Cc1ccc(-n2cc3c(N4CCN[C@H](C(=O)NCc5ccc6sccc6c5)C4)ncnc3n2)cc1] | (S)-N-(benzo[b]thiophen- 5-ylmethyl)-4-(2-(p-tolyl)- 2H-pyrazolo[3,4- d]pyrimidin-4- yl)piperazine-2- carboxamide | 484.21 |
| 197 | 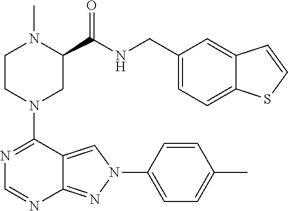 [SMILES: Cc1ccc(-n2cc3c(N4CCN(C)[C@@H](C(=O)NCc5ccc6sccc6c5)C4)ncnc3n2)cc1] | (R)-N-(benzo[b]thiophen- 5-ylmethyl)-1-methyl-4- (2-(p-tolyl)-2H- pyrazolo[3,4-d]pyrimidin- 4-yl)piperazine-2- carboxamide | 498.33 |
| 198 | 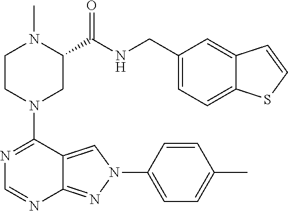 [SMILES: Cc1ccc(-n2cc3c(N4CCN(C)[C@H](C(=O)NCc5ccc6sccc6c5)C4)ncnc3n2)cc1] | (S)-N-(benzo[b]thiophen- 5-ylmethyl)-1-methyl-4- (2-(p-tolyl)-2H- pyrazolo[3,4-d]pyrimidin- 4-yl)piperazine-2- carboxamide | 498.27 |
| 199 | 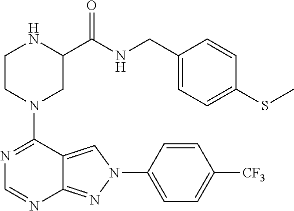 [SMILES: CSc1ccc(CNC(=O)C2CN(c3ncnc4nn(-c5ccc(C(F)(F)F)cc5)cc34)CCN2)cc1] | N-(4-(methylthio)benzyl)- 4-(2-(4- (trifluoromethyl)phenyl)- 2H-pyrazolo[3,4- d]pyrimidin-4- yl)piperazine-2- carboxamide | 528.29 |
| 200 | 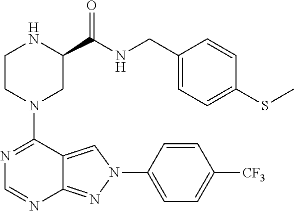 [SMILES: CSc1ccc(CNC(=O)[C@H]2CN(c3ncnc4nn(-c5ccc(C(F)(F)F)cc5)cc34)CCN2)cc1] | (R)-N-(4- (methylthio)benzyl)-4-(2- (4- (trifluoromethyl)phenyl)- 2H-pyrazolo[3,4- d]pyrimidin-4- yl)piperazine-2- carboxamide | 528.21 |
| 201 | 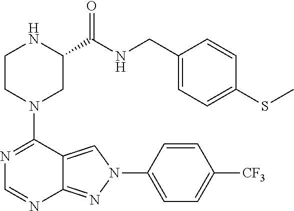 [SMILES: CSc1ccc(CNC(=O)[C@@H]2CN(c3ncnc4nn(-c5ccc(C(F)(F)F)cc5)cc34)CCN2)cc1] | (S)-N-(4- (methylthio)benzyl)-4-(2- (4- (trifluoromethyl)phenyl)- 2H-pyrazolo[3,4- d]pyrimidin-4- yl)piperazine-2- carboxamide | 528.22 |
| 202 | 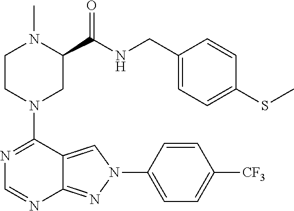 [SMILES: CSc1ccc(CNC(=O)[C@H]2CN(c3ncnc4nn(-c5ccc(C(F)(F)F)cc5)cc34)CCN2C)cc1] | (R)-1-methyl-N-(4- (methylthio)benzyl)-4-(2- (4- (trifluoromethyl)phenyl)- 2H-pyrazolo[3,4- d]pyrimidin-4- yl)piperazine-2- carboxamide | 542.33 |
| 203 | 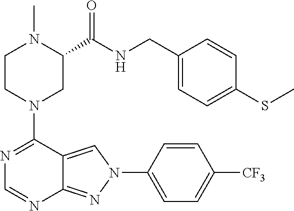 [SMILES: CSc1ccc(CNC(=O)[C@@H]2CN(c3ncnc4nn(-c5ccc(C(F)(F)F)cc5)cc34)CCN2C)cc1] | (S)-1-methyl-N-(4- (methylthio)benzyl)-4-(2- (4- (trifluoromethyl)phenyl)- 2H-pyrazolo[3,4- d]pyrimidin-4- yl)piperazine-2- carboxamide | 542.23 |
| 204 | 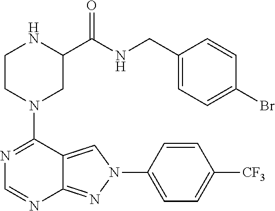 [SMILES: O=C(NCc1ccc(Br)cc1)C1CN(c2ncnc3nn(-c4ccc(C(F)(F)F)cc4)cc23)CCN1] | N-(4-bromobenzyl)-4-(2- (4- (trifluoromethyl)phenyl)- 2H-pyrazolo[3,4- d]pyrimidin-4- yl)piperazine-2- carboxamide | 560.37 |
| 205 | 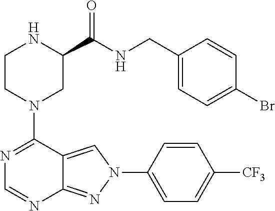 [SMILES: O=C(NCc1ccc(Br)cc1)[C@H]1CN(c2ncnc3nn(-c4ccc(C(F)(F)F)cc4)cc23)CCN1] | (R)-N-(4-bromobenzyl)-4- (2-(4- (trifluoromethyl)phenyl)- 2H-pyrazolo[3,4- d]pyrimidin-4- yl)piperazine-2- carboxamide | 560.11 |
| 206 | 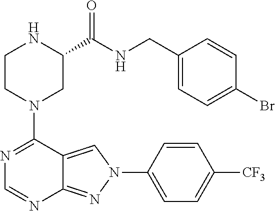 [SMILES: O=C(NCc1ccc(Br)cc1)[C@@H]1CN(c2ncnc3nn(-c4ccc(C(F)(F)F)cc4)cc23)CCN1] | (S)-N-(4-bromobenzyl)-4- (2-(4- (trifluoromethyl)phenyl)- 2H-pyrazolo[3,4- d]pyrimidin-4- yl)piperazine-2- carboxamide | 560.17 |
| 207 | 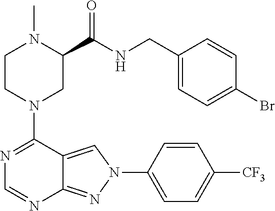 [SMILES: CN1CCN(c2ncnc3nn(-c4ccc(C(F)(F)F)cc4)cc23)C[C@@H]1C(=O)NCc1ccc(Br)cc1] | (R)-N-(4-bromobenzyl)-1- methyl-4-(2-(4- (trifluoromethyl)phenyl)- 2H-pyrazolo[3,4- d]pyrimidin-4- yl)piperazine-2- carboxamide | 574.13 |
| 208 | 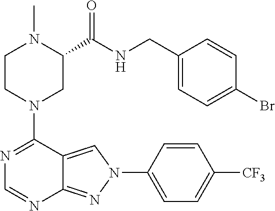 [SMILES: CN1CCN(c2ncnc3nn(-c4ccc(C(F)(F)F)cc4)cc23)C[C@H]1C(=O)NCc1ccc(Br)cc1] | (S)-N-(4-bromobenzyl)-1- methyl-4-(2-(4- (trifluoromethyl)phenyl)- 2H-pyrazolo[3,4- d]pyrimidin-4- yl)piperazine-2- carboxamide | 574.21 |
| 209 | 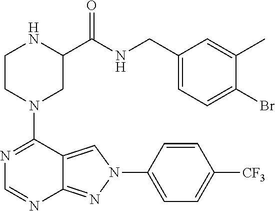 [SMILES: Cc1cc(CNC(=O)C2CN(c3ncnc4nn(-c5ccc(C(F)(F)F)cc5)cc34)CCN2)ccc1Br] | N-(4-bromo-3- methylbenzyl)-4-(2-(4- (trifluoromethyl)phenyl)- 2H-pyrazolo[3,4- d]pyrimidin-4- yl)piperazine-2- carboxamide | 574.11 |
| 210 | 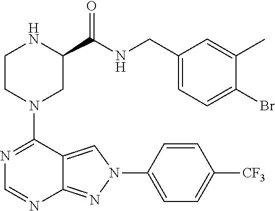 [SMILES: Cc1cc(CNC(=O)[C@H]2CN(c3ncnc4nn(-c5ccc(C(F)(F)F)cc5)cc34)CCN2)ccc1Br] | (R)-N-(4-bromo-3- methylbenzyl)-4-(2-(4- (trifluoromethyl)phenyl)- 2H-pyrazolo[3,4- d]pyrimidin-4- yl)piperazine-2- carboxamide | 574.17 |
| 211 | 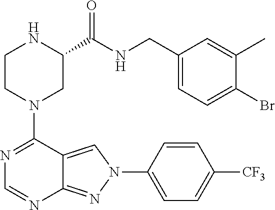 [SMILES: Cc1cc(CNC(=O)[C@@H]2CN(c3ncnc4nn(-c5ccc(C(F)(F)F)cc5)cc34)CCN2)ccc1Br] | (S)-N-(4-bromo-3- methylbenzyl)-4-(2-(4- (trifluoromethyl)phenyl)- 2H-pyrazolo[3,4- d]pyrimidin-4- yl)piperazine-2- carboxamide | 574.23 |
| 212 | 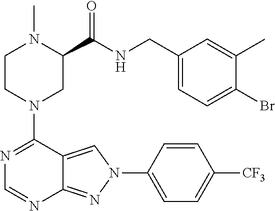 [SMILES: Cc1cc(CNC(=O)[C@H]2CN(c3ncnc4nn(-c5ccc(C(F)(F)F)cc5)cc34)CCN2C)ccc1Br] | (R)-N-(4-bromo-3- methylbenzyl)-1-methyl- 4-(2-(4- (trifluoromethyl)phenyl)- 2H-pyrazolo[3,4- d]pyrimidin-4- yl)piperazine-2- carboxamide | 588.41 |
| 213 | 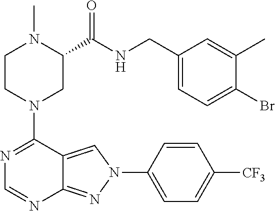 [SMILES: Cc1cc(CNC(=O)[C@@H]2CN(c3ncnc4nn(-c5ccc(C(F)(F)F)cc5)cc34)CCN2C)ccc1Br] | (S)-N-(4-bromo-3- methylbenzyl)-1-methyl- 4-(2-(4- (trifluoromethyl)phenyl)- 2H-pyrazolo[3,4- d]pyrimidin-4- yl)piperazine-2- carboxamide | 588.17 |
| 214 | 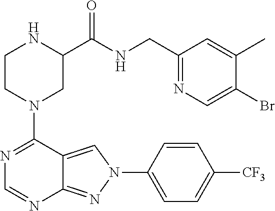 [SMILES: Cc1cc(CNC(=O)C2CN(c3ncnc4nn(-c5ccc(C(F)(F)F)cc5)cc34)CCN2)ncc1Br] | N-((5-bromo-4- methylpyridin-2- yl)methyl)-4-(2-(4- (trifluoromethyl)phenyl)- 2H-pyrazolo[3,4- d]pyrimidin-4- yl)piperazine-2- carboxamide | 575.33 |
| 215 | 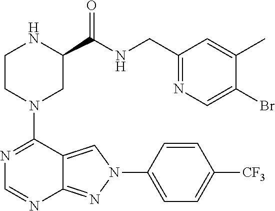 [SMILES: Cc1cc(CNC(=O)[C@H]2CN(c3ncnc4nn(-c5ccc(C(F)(F)F)cc5)cc34)CCN2)ncc1Br] | (R)-N-((5-bromo-4- methylpyridin-2- yl)methyl)-4-(2-(4- (trifluoromethyl)phenyl)- 2H-pyrazolo[3,4- d]pyrimidin-4- yl)piperazine-2- carboxamide | 575.27 |
| 216 | 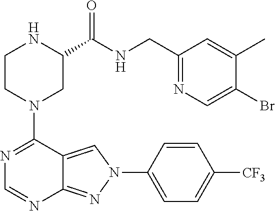 | (S)-N-((5-bromo-4- methylpyridin-2- yl)methyl)-4-(2-(4- (trifluoromethyl)phenyl)- 2H-pyrazolo[3,4- d]pyrimidin-4- yl)piperazine-2- carboxamide | 575.12 |
| 217 | 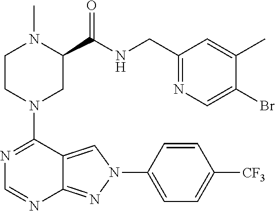 [SMILES: Cc1cc(CNC(=O)[C@H]2CN(c3ncnc4nn(-c5ccc(C(F)(F)F)cc5)cc34)CCN2C)ncc1Br] | (R)-N-((5-bromo-4- methylpyridin-2- yl)methyl)-1-methyl-4-(2- (4- (trifluoromethyl)phenyl)- 2H-pyrazolo[3,4- d]pyrimidin-4- yl)piperazine-2- carboxamide | 589.27 |
| 218 | 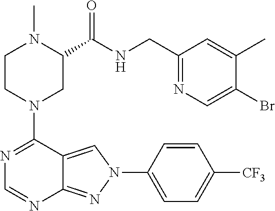 | (S)-N-((5-bromo-4- methylpyridin-2- yl)methyl)-1-methyl-4-(2- (4- (trifluoromethyl)phenyl)- 2H-pyrazolo[3,4- d]pyrimidin-4- yl)piperazine-2- carboxamide | 589.24 |
| 219 | 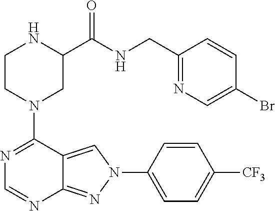 [SMILES: O=C(NCc1ccc(Br)cn1)C1CN(c2ncnc3nn(-c4ccc(C(F)(F)F)cc4)cc23)CCN1] | N-((5-bromopyridin-2- yl)methyl)-4-(2-(4- (trifluoromethyl)phenyl)- 2H-pyrazolo[3,4- d]pyrimidin-4- yl)piperazine-2- carboxamide | 561.01 |
| 220 | 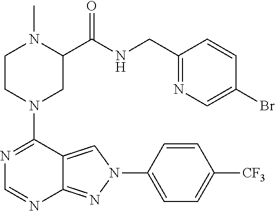 [SMILES: CN1CCN(c2ncnc3nn(-c4ccc(C(F)(F)F)cc4)cc23)CC1C(=O)NCc1ccc(Br)cn1] | N-((5-bromopyridin-2- yl)methyl)-1-methyl-4-(2- (4- (trifluoromethyl)phenyl)- 2H-pyrazolo[3,4- d]pyrimidin-4- yl)piperazine-2- carboxamide | 575.21 |
| 221 | 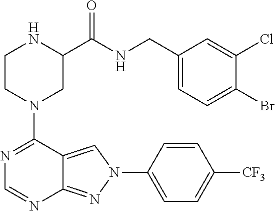 [SMILES: O=C(NCc1ccc(Br)c(Cl)c1)C1CN(c2ncnc3nn(-c4ccc(C(F)(F)F)cc4)cc23)CCN1] | N-(4-bromo-3- chlorobenzyl)-4-(2-(4- (trifluoromethyl)phenyl)- 2H-pyrazolo[3,4- d]pyrimidin-4- yl)piperazine-2- carboxamide | 594.13 |
| 222 | 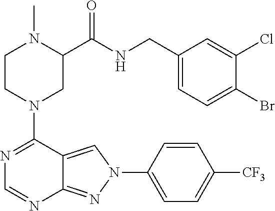 [SMILES: CN1CCN(c2ncnc3nn(-c4ccc(C(F)(F)F)cc4)cc23)CC1C(=O)NCc1ccc(Br)c(Cl)c1] | N-(4-bromo-3- chlorobenzyl)-1-methyl-4- (2-(4- (trifluoromethyl)phenyl)- 2H-pyrazolo[3,4- d]pyrimidin-4- yl)piperazine-2- carboxamide | 608.17 |
| 223 | 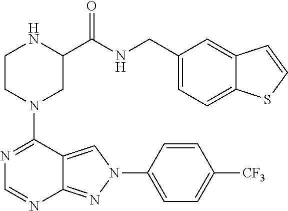 [SMILES: O=C(NCc1ccc2sccc2c1)C1CN(c2ncnc3nn(-c4ccc(C(F)(F)F)cc4)cc23)CCN1] | N-(benzo[b]thiophen-5- ylmethyl)-4-(2-(4- (trifluoromethyl)phenyl)- 2H-pyrazolo[3,4- d]pyrimidin-4- yl)piperazine-2- carboxamide | 537.12 |
| 224 | 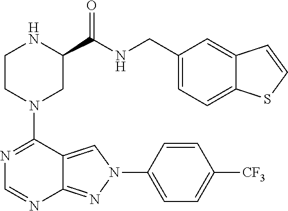 [SMILES: O=C(NCc1ccc2sccc2c1)[C@H]1CN(c2ncnc3nn(-c4ccc(C(F)(F)F)cc4)cc23)CCN1] | (R)-N-(benzo[b]thiophen- 5-ylmethyl)-4-(2-(4- (trifluoromethyl)phenyl)- 2H-pyrazolo[3,4- d]pyrimidin-4- yl)piperazine-2- carboxamide | 538.20 |
| 225 | 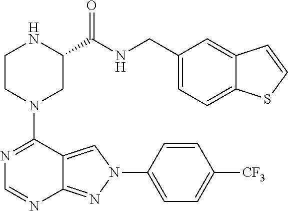 [SMILES: O=C(NCc1ccc2sccc2c1)[C@@H]1CN(c2ncnc3nn(-c4ccc(C(F)(F)F)cc4)cc23)CCN1] | (S)-N-(benzo[b]thiophen- 5-ylmethyl)-4-(2-(4- (trifluoromethyl)phenyl)- 2H-pyrazolo[3,4- d]pyrimidin-4- yl)piperazine-2- carboxamide | 538.17 |
| 226 | 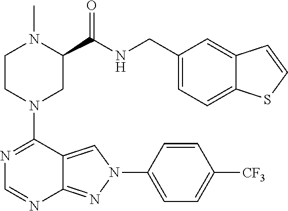 [SMILES: CN1CCN(c2ncnc3nn(-c4ccc(C(F)(F)F)cc4)cc23)C[C@@H]1C(=O)NCc1ccc2sccc2c1] | (R)-N-(benzo[b]thiophen- 5-ylmethyl)-1-methyl-4- (2-(4- (trifluoromethyl)phenyl)- 2H-pyrazolo[3,4- d]pyrimidin-4- yl)piperazine-2- carboxamide | 552.31 |
| 227 | 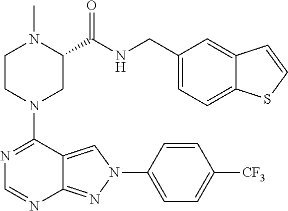 [SMILES: CN1CCN(c2ncnc3nn(-c4ccc(C(F)(F)F)cc4)cc23)C[C@H]1C(=O)NCc1ccc2sccc2c1] | (S)-N-(benzo[b]thiophen- 5-ylmethyl)-1-methyl-4- (2-(4- (trifluoromethyl)phenyl)- 2H-pyrazolo[3,4- d]pyrimidin-4- yl)piperazine-2- carboxamide | 552.37 |
| 228 | 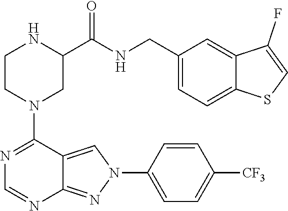 [SMILES: O=C(NCc1ccc2scc(F)c2c1)C1CN(c2ncnc3nn(-c4ccc(C(F)(F)F)cc4)cc23)CCN1] | N-((3- fluorobenzo[b]thiophen- 5-yl)methyl)-4-(2-(4- (trifluoromethyl)phenyl)- 2H-pyrazolo[3,4- d]pyrimidin-4- yl)piperazine-2- carboxamide | 556.11 |
| 229 | 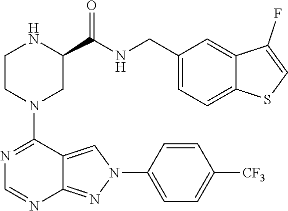 [SMILES: O=C(NCc1ccc2scc(F)c2c1)[C@H]1CN(c2ncnc3nn(-c4ccc(C(F)(F)F)cc4)cc23)CCN1] | (R)-N-((3- fluorobenzo[b]thiophen- 5-yl)methyl)-4-(2-(4- (trifluoromethyl)phenyl)- 2H-pyrazolo[3,4- d]pyrimidin-4- yl)piperazine-2- carboxamide | 556.07 |
| 230 | 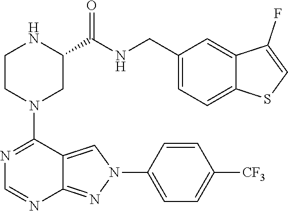 [SMILES: O=C(NCc1ccc2scc(F)c2c1)[C@@H]1CN(c2ncnc3nn(-c4ccc(C(F)(F)F)cc4)cc23)CCN1] | (S)-N-((3- fluorobenzo[b]thiophen- 5-yl)methyl)-4-(2-(4- (trifluoromethyl)phenyl)- 2H-pyrazolo[3,4- d]pyrimidin-4- yl)piperazine-2- carboxamide | 556.19 |
| 231 | 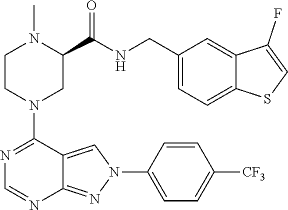 [SMILES: CN1CCN(c2ncnc3nn(-c4ccc(C(F)(F)F)cc4)cc23)C[C@@H]1C(=O)NCc1ccc2scc(F)c2c1] | (R)-N-((3- fluorobenzo[b]thiophen- 5-yl)methyl)-1-methyl-4- (2-(4- (trifluoromethyl)phenyl)- 2H-pyrazolo[3,4- d]pyrimidin-4- yl)piperazine-2- carboxamide | 570.24 |
| 232 | 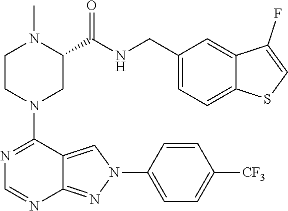 [SMILES: CN1CCN(c2ncnc3nn(-c4ccc(C(F)(F)F)cc4)cc23)C[C@H]1C(=O)NCc1ccc2scc(F)c2c1] | (S)-N-((3- fluorobenzo[b]thiophen- 5-yl)methyl)-1-methyl-4- (2-(4- (trifluoromethyl)phenyl)- 2H-pyrazolo[3,4- d]pyrimidin-4- yl)piperazine-2- carboxamide | 574.10 |
| 233 | 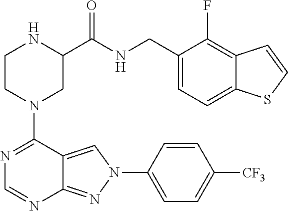 [SMILES: O=C(NCc1ccc2sccc2c1F)C1CN(c2ncnc3nn(-c4ccc(C(F)(F)F)cc4)cc23)CCN1] | N-((4- fluorobenzo[b]thiophen- 5-yl)methyl)-4-(2-(4- (trifluoromethyl)phenyl)- 2H-pyrazolo[3,4- d]pyrimidin-4- yl)piperazine-2- carboxamide | 556.25 |
| 234 | 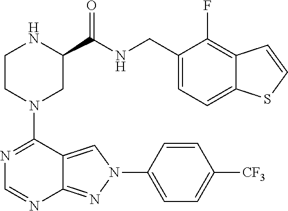 [SMILES: O=C(NCc1ccc2sccc2c1F)[C@H]1CN(c2ncnc3nn(-c4ccc(C(F)(F)F)cc4)cc23)CCN1] | (R)-N-((4- fluorobenzo[b]thiophen- 5-yl)methyl)-4-(2-(4- (trifluoromethyl)phenyl)- 2H-pyrazolo[3,4- d]pyrimidin-4- yl)piperazine-2- carboxamide | 556.12 |
| 235 | 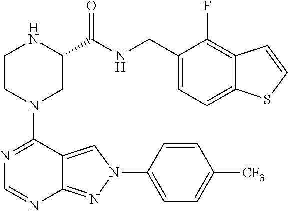 [SMILES: O=C(NCc1ccc2sccc2c1F)[C@@H]1CN(c2ncnc3nn(-c4ccc(C(F)(F)F)cc4)cc23)CCN1] | (S)-N-((4- fluorobenzo[b]thiophen- 5-yl)methyl)-4-(2-(4- (trifluoromethyl)phenyl)- 2H-pyrazolo[3,4- d]pyrimidin-4- yl)piperazine-2- carboxamide | 556.17 |
| 236 | 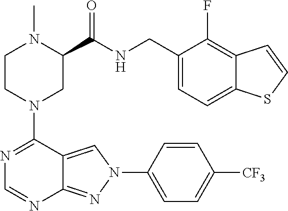 [SMILES: CN1CCN(c2ncnc3nn(-c4ccc(C(F)(F)F)cc4)cc23)C[C@@H]1C(=O)NCc1ccc2sccc2c1F] | (R)-N-((4- fluorobenzo[b]thiophen- 5-yl)methyl)-1-methyl-4- (2-(4- (trifluoromethyl)phenyl)- 2H-pyrazolo[3,4- d]pyrimidin-4- yl)piperazine-2- carboxamide | 570.35 |
| 237 | 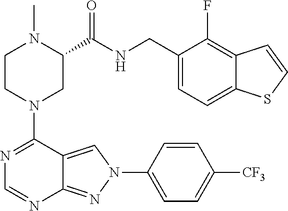 [SMILES: CN1CCN(c2ncnc3nn(-c4ccc(C(F)(F)F)cc4)cc23)C[C@H]1C(=O)NCc1ccc2sccc2c1F] | (S)-N-((4- fluorobenzo[b]thiophen- 5-yl)methyl)-1-methyl-4- (2-(4- (trifluoromethyl)phenyl)- 2H-pyrazolo[3,4- d]pyrimidin-4- yl)piperazine-2- carboxamide | 570.29 |
| 238 | 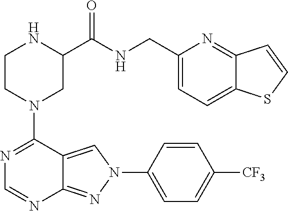 [SMILES: O=C(NCc1ccc2sccc2n1)C1CN(c2ncnc3nn(-c4ccc(C(F)(F)F)cc4)cc23)CCN1] | N-(thieno[3,2-b]pyridin-5- ylmethyl)-4-(2-(4- (trifluoromethyl)phenyl)- 2H-pyrazolo[3,4- d]pyrimidin-4- yl)piperazine-2- carboxamide | 539.20 |
| 239 | 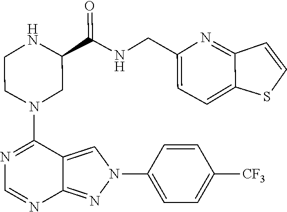 [SMILES: O=C(NCc1ccc2sccc2n1)[C@H]1CN(c2ncnc3nn(-c4ccc(C(F)(F)F)cc4)cc23)CCN1] | (R)-N-(thieno[3,2- b]pyridin-5-ylmethyl)-4- (2-(4- (trifluoromethyl)phenyl)- 2H-pyrazolo[3,4- d]pyrimidin-4- yl)piperazine-2- carboxamide | 539.13 |
| 240 | 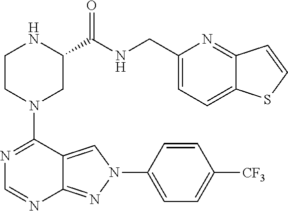 [SMILES: O=C(NCc1ccc2sccc2n1)[C@@H]1CN(c2ncnc3nn(-c4ccc(C(F)(F)F)cc4)cc23)CCN1] | (S)-N-(thieno[3,2- b]pyridin-5-ylmethyl)-4- (2-(4- (trifluoromethyl)phenyl)- 2H-pyrazolo[3,4- d]pyrimidin-4- yl)piperazine-2- carboxamide | 539.09 |
| 241 | 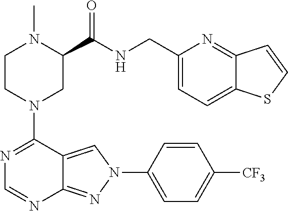 [SMILES: CN1CCN(c2ncnc3nn(-c4ccc(C(F)(F)F)cc4)cc23)C[C@@H]1C(=O)NCc1ccc2sccc2n1] | (R)-1-methyl-N- (thieno[3,2-b]pyridin-5- ylmethyl)-4-(2-(4- (trifluoromethyl)phenyl)- 2H-pyrazolo[3,4- d]pyrimidin-4- yl)piperazine-2- carboxamide | 553.19 |
| 242 | 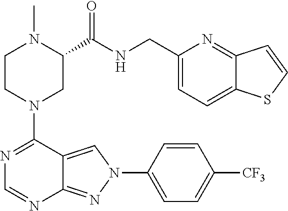 [SMILES: CN1CCN(c2ncnc3nn(-c4ccc(C(F)(F)F)cc4)cc23)C[C@H]1C(=O)NCc1ccc2sccc2n1] | (S)-1-methyl-N- (thieno[3,2-b]pyridin-5- ylmethyl)-4-(2-(4- (trifluoromethyl)phenyl)- 2H-pyrazolo[3,4- d]pyrimidin-4- yl)piperazine-2- carboxamide | 553.21 |
| 243 | 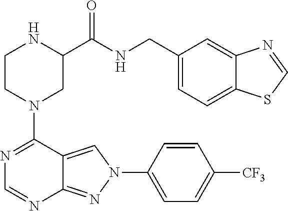 [SMILES: O=C(NCc1ccc2scnc2c1)C1CN(c2ncnc3nn(-c4ccc(C(F)(F)F)cc4)cc23)CCN1] | N-(benzo[d]thiazol-5- ylmethyl)-4-(2-(4- (trifluoromethyl)phenyl)- 2H-pyrazolo[3,4- d]pyrimidin-4- yl)piperazine-2- carboxamide | 539.27 |
| 244 | 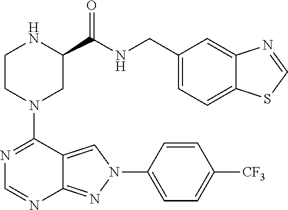 [SMILES: O=C(NCc1ccc2scnc2c1)[C@H]1CN(c2ncnc3nn(-c4ccc(C(F)(F)F)cc4)cc23)CCN1] | (R)-N-(benzo[d]thiazol-5- ylmethyl)-4-(2-(4- (trifluoromethyl)phenyl)- 2H-pyrazolo[3,4- d]pyrimidin-4- yl)piperazine-2- carboxamide | 539.33 |
| 245 | 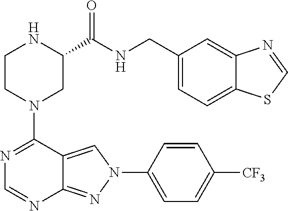 [SMILES: O=C(NCc1ccc2scnc2c1)[C@@H]1CN(c2ncnc3nn(-c4ccc(C(F)(F)F)cc4)cc23)CCN1] | (S)-N-(benzo[d]thiazol-5- ylmethyl)-1-(2-(4- (trifluoromethyl)phenyl)- 2H-pyrazolo[3,4- d]pyrimidin-4- yl)piperidine-3- carboxamide | 539.22 |
| 246 | 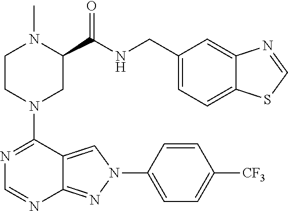 [SMILES: CN1CCN(c2ncnc3nn(-c4ccc(C(F)(F)F)cc4)cc23)C[C@@H]1C(=O)NCc1ccc2scnc2c1] | (R)-N-(benzo[d]thiazol-5- ylmethyl)-1-methyl-4-(2- (4- (trifluoromethyl)phenyl)- 2H-pyrazolo[3,4- d]pyrimidin-4- yl)piperazine-2- carboxamide | 553.14 |
| 247 | 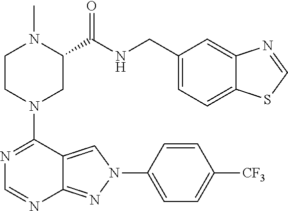 [SMILES: CN1CCN(c2ncnc3nn(-c4ccc(C(F)(F)F)cc4)cc23)C[C@H]1C(=O)NCc1ccc2scnc2c1] | (S)-N-(benzo[d]thiazol-5- ylmethyl)-1-methyl-4-(2- (4- (trifluoromethyl)phenyl)- 2H-pyrazolo[3,4- d]pyrimidin-4- yl)piperazine-2- carboxamide | 553.12 |
| 248 | 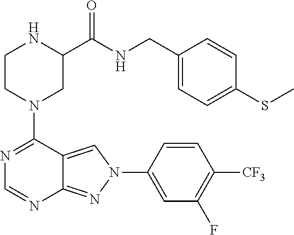 [SMILES: CSc1ccc(CNC(=O)C2CN(c3ncnc4nn(-c5ccc(C(F)(F)F)c(F)c5)cc34)CCN2)cc1] | 4-(2-(3-fluoro-4- (trifluoromethyl)phenyl)- 2H-pyrazolo[3,4- d]pyrimidin-4-yl)-N-(4- (methylthio)benzyl) piperazine-2-carboxamide | 546.11 |
| 249 | 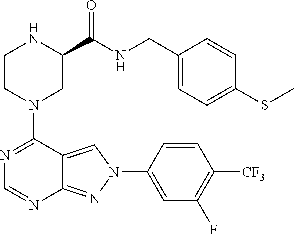 [SMILES: CSc1ccc(CNC(=O)[C@H]2CN(c3ncnc4nn(-c5ccc(C(F)(F)F)c(F)c5)cc34)CCN2)cc1] | (R)-4-(2-(3-fluoro-4- (trifluoromethyl)phenyl)- 2H-pyrazolo[3,4- d]pyrimidin-4-yl)-N-(4- (methylthio)benzyl) piperazine-2-carboxamide | 546.16 |
| 250 | 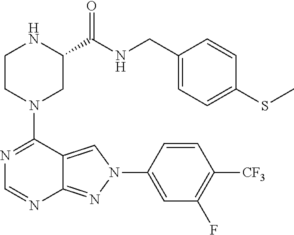 [SMILES: CSc1ccc(CNC(=O)[C@@H]2CN(c3ncnc4nn(-c5ccc(C(F)(F)F)c(F)c5)cc34)CCN2)cc1] | (S)-4-(2-(3-fluoro-4- (trifluoromethyl)phenyl)- 2H-pyrazolo[3,4- d]pyrimidin-4-yl)-N-(4- (methylthio)benzyl) piperazine-2-carboxamide | 546.20 |
| 251 | 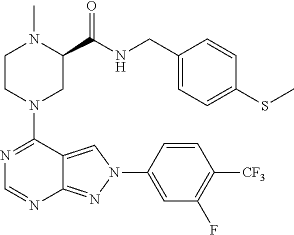 [SMILES: CSc1ccc(CNC(=O)[C@H]2CN(c3ncnc4nn(-c5ccc(C(F)(F)F)c(F)c5)cc34)CCN2C)cc1] | (R)-4-(2-(3-fluoro-4- (trifluoromethyl)phenyl)- 2H-pyrazolo[3,4- d]pyrimidin-4-yl)-1- methyl-N-(4- (methylthio)benzyl) piperazine-2-carboxamide | 560.14 |
| 252 | 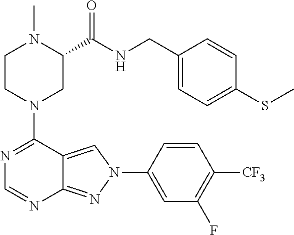 [SMILES: CSc1ccc(CNC(=O)[C@@H]2CN(c3ncnc4nn(-c5ccc(C(F)(F)F)c(F)c5)cc34)CCN2C)cc1] | (S)-4-(2-(3-fluoro-4- (trifluoromethyl)phenyl)- 2H-pyrazolo[3,4- d]pyrimidin-4-yl)-1- methyl-N-(4- (methylthio)benzyl) piperazine-2-carboxamide | 560.13 |
| 253 | 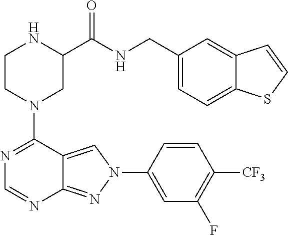 [SMILES: O=C(NCc1ccc2sccc2c1)C1CN(c2ncnc3nn(-c4ccc(C(F)(F)F)c(F)c4)cc23)CCN1] | N-(benzo[b]thiophen-5- ylmethyl)-4-(2-(3-fluoro-4- (trifluoromethyl)phenyl)- 2H-pyrazolo[3,4- d]pyrimidin-4- yl)piperazine-2- carboxamide | 556.07 |
| 254 | 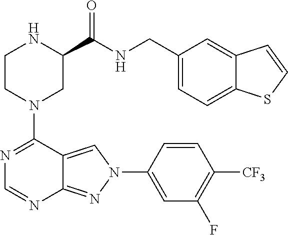 [SMILES: O=C(NCc1ccc2sccc2c1)[C@H]1CN(c2ncnc3nn(-c4ccc(C(F)(F)F)c(F)c4)cc23)CCN1] | (R)-N-(benzo[b]thiophen- 5-ylmethyl)-4-(2-(3-fluoro- 4- (trifluoromethyl)phenyl)- 2H-pyrazolo[3,4- d]pyrimidin-4- yl)piperazine-2- carboxamide | 556.11 |
| 255 | 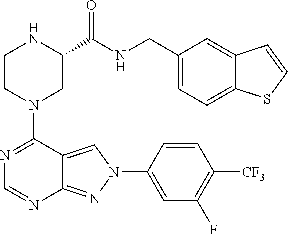 [SMILES: O=C(NCc1ccc2sccc2c1)[C@@H]1CN(c2ncnc3nn(-c4ccc(C(F)(F)F)c(F)c4)cc23)CCN1] | (S)-N-(benzo[b]thiophen- 5-ylmethyl)-4-(2-(3-fluoro- 4- (trifluoromethyl)phenyl)- 2H-pyrazolo[3,4- d]pyrimidin-4- yl)piperazine-2- carboxamide | 556.09 |
| 256 | 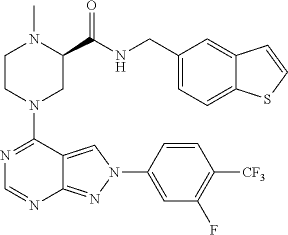 [SMILES: CN1CCN(c2ncnc3nn(-c4ccc(C(F)(F)F)c(F)c4)cc23)C[C@@H]1C(=O)NCc1ccc2sccc2c1] | (R)-N-(benzo[b]thiophen- 5-ylmethyl)-4-(2-(3-fluoro- 4- (trifluoromethyl)phenyl)- 2H-pyrazolo[3,4- d]pyrimidin-4-yl)-1- methylpiperazine-2- carboxamide | 570.11 |
| 257 | 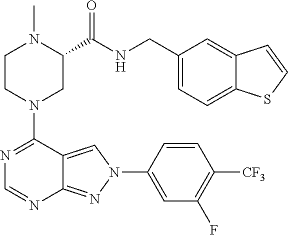 [SMILES: CN1CCN(c2ncnc3nn(-c4ccc(C(F)(F)F)c(F)c4)cc23)C[C@H]1C(=O)NCc1ccc2sccc2c1] | (S)-N-(benzo[b]thiophen- 5-ylmethyl)-4-(2-(3-fluoro- 4- (trifluoromethyl)phenyl)- 2H-pyrazolo[3,4- d]pyrimidin-4-yl)-1- methylpiperazine-2- carboxamide | 570.08 |
| 258 | 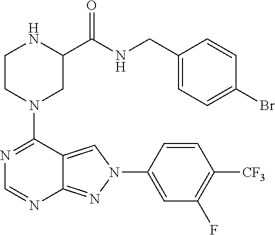 [SMILES: O=C(NCc1ccc(Br)cc1)C1CN(c2ncnc3nn(-c4ccc(C(F)(F)F)c(F)c4)cc23)CCN1] | N-(4-bromobenzyl)-4-(2- (3-fluoro-4- (trifluoromethyl)phenyl)- 2H-pyrazolo[3,4- d]pyrimidin-4- yl)piperazine-2- carboxamide | 578.12 |
| 259 | 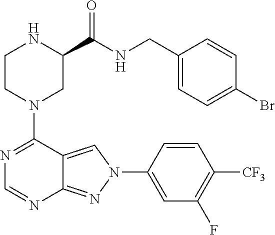 [SMILES: O=C(NCc1ccc(Br)cc1)[C@H]1CN(c2ncnc3nn(-c4ccc(C(F)(F)F)c(F)c4)cc23)CCN1] | (R)-N-(4-bromobenzyl)-4- (2-(3-fluoro-4- (trifluoromethyl)phenyl)- 2H-pyrazolo[3,4- d]pyrimidin-4- yl)piperazine-2- carboxamide | 578.00 |
| 260 | 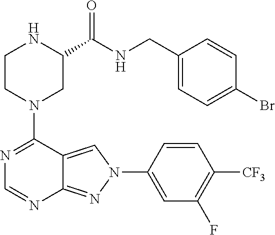 | (S)-N-(4-bromobenzyl)-4- (2-(3-fluoro-4- (trifluoromethyl)phenyl)- 2H-pyrazolo[3,4- d]pyrimidin-4- yl)piperazine-2- carboxamide | 577.02 |
| 261 | 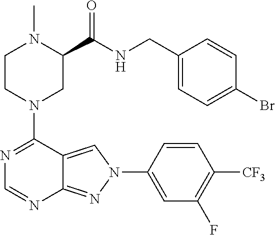 [SMILES: CN1CCN(c2ncnc3nn(-c4ccc(C(F)(F)F)c(F)c4)cc23)C[C@@H]1C(=O)NCc1ccc(Br)cc1] | (R)-N-(4-bromobenzyl)-4- (2-(3-fluoro-4- (trifluoromethyl)phenyl)- 2H-pyrazolo[3,4- d]pyrimidin-4-yl)-1- methylpiperazine-2- carboxamide | 592.20 |
| 262 | 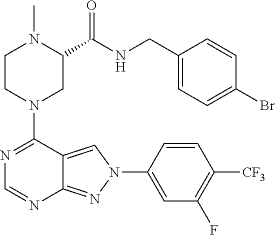 | (S)-N-(4-bromobenzyl)-4- (2-(3-fluoro-4- (trifluoromethyl)phenyl)- 2H-pyrazolo[3,4- d]pyrimidin-4-yl)-1- methylpiperazine-2- carboxamide | 592.12 |
| 263 | 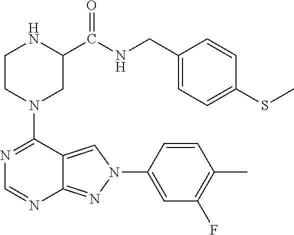 [SMILES: CSc1ccc(CNC(=O)C2CN(c3ncnc4nn(-c5ccc(C)c(F)c5)cc34)CCN2)cc1] | 4-(2-(3-fluoro-4- methylphenyl)-2H- pyrazolo[3,4-d]pyrimidin- 4-yl)-N-(4- (methylthio)benzyl) piperazine-2-carboxamide | 492.13 |
| 264 | 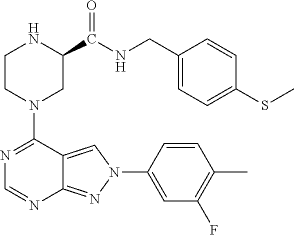 [SMILES: CSc1ccc(CNC(=O)[C@H]2CN(c3ncnc4nn(-c5ccc(C)c(F)c5)cc34)CCN2)cc1] | (R)-4-(2-(3-fluoro-4- methylphenyl)-2H- pyrazolo[3,4-d]pyrimidin- 4-yl)-N-(4- (methylthio)benzyl) piperazine-2-carboxamide | 492.17 |
| 265 | 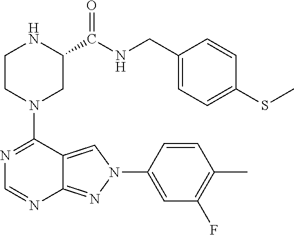 [SMILES: CSc1ccc(CNC(=O)[C@@H]2CN(c3ncnc4nn(-c5ccc(C)c(F)c5)cc34)CCN2)cc1] | (S)-4-(2-(3-fluoro-4- methylphenyl)-2H- pyrazolo[3,4-d]pyrimidin- 4-yl)-N-(4- (methylthio)benzyl) piperazine-2-carboxamide | 492.08 |
| 266 | 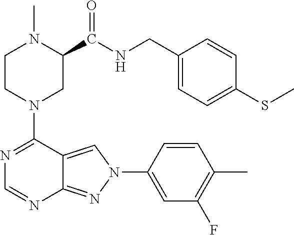 [SMILES: CSc1ccc(CNC(=O)[C@H]2CN(c3ncnc4nn(-c5ccc(C)c(F)c5)cc34)CCN2C)cc1] | (R)-4-(2-(3-fluoro-4- methylphenyl)-2H- pyrazolo[3,4-d]pyrimidin- 4-yl)-1-methyl-N-(4- (methylthio)benzyl) piperazine-2-carboxamide | 506.32 |
| 267 | 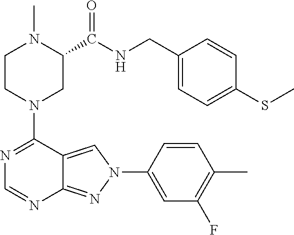 [SMILES: CSc1ccc(CNC(=O)[C@@H]2CN(c3ncnc4nn(-c5ccc(C)c(F)c5)cc34)CCN2C)cc1] | (S)-4-(2-(3-fluoro-4- methylphenyl)-2H- pyrazolo[3,4-d]pyrimidin- 4-yl)-1-methyl-N-(4- (methylthio)benzyl) piperazine-2-carboxamide | 506.27 |
| 268 | 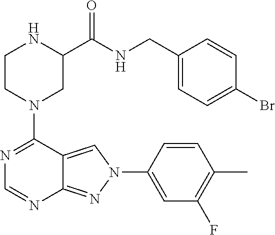 [SMILES: Cc1ccc(-n2cc3c(N4CCNC(C(=O)NCc5ccc(Br)cc5)C4)ncnc3n2)cc1F] | N-(4-bromobenzyl)-4-(2- (3-fluoro-4- methylphenyl)-2H- pyrazolo[3,4-d]pyrimidin- 4-yl)piperazine-2- carboxamide | 524.02 |
| 269 | 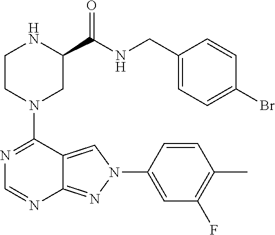 [SMILES: Cc1ccc(-n2cc3c(N4CCN[C@@H](C(=O)NCc5ccc(Br)cc5)C4)ncnc3n2)cc1F] | (R)-N-(4-bromobenzyl)-4- (2-(3-fluoro-4- methylphenyl)-2H- pyrazolo[3,4-d]pyrimidin- 4-yl)piperazine-2- carboxamide | 524.10 |
| 270 | 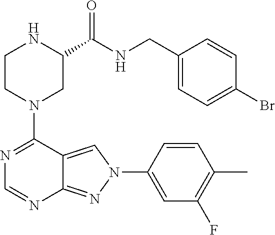 | (S)-N-(4-bromobenzyl)-4- (2-(3-fluoro-4- methylphenyl)-2H- pyrazolo[3,4-d]pyrimidin- 4-yl)piperazine-2- carboxamide | 524.10 |
| 271 | 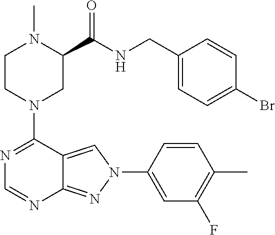 [SMILES: Cc1ccc(-n2cc3c(N4CCN(C)[C@@H](C(=O)NCc5ccc(Br)cc5)C4)ncnc3n2)cc1F] | (R)-N-(4-bromobenzyl)-4- (2-(3-fluoro-4- methylphenyl)-2H- pyrazolo[3,4-d]pyrimidin- 4-yl)-1-methylpiperazine- 2-carboxamide | 538.41 |
| 272 | 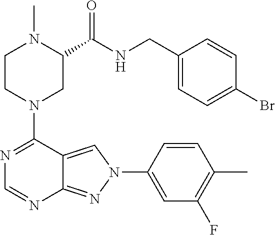 | (R)-N-(4-bromobenzyl)-4- (2-(3-fluoro-4- methylphenyl)-2H- pyrazolo[3,4-d]pyrimidin- 4-yl)-1-methylpiperazine- 2-carboxamide | 538.27 |
| 273 | 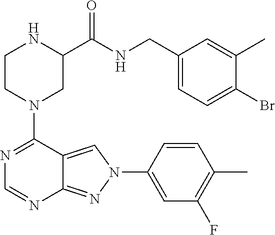 [SMILES: Cc1ccc(-n2cc3c(N4CCNC(C(=O)NCc5ccc(Br)c(C)c5)C4)ncnc3n2)cc1F] | N-(4-bromo-3- methylbenzyl)-4-(2-(3- fluoro-4-methylphenyl)- 2H-pyrazolo[3,4- d]pyrimidin-4- yl)piperazine-2- carboxamide | 538.19 |
| 274 | 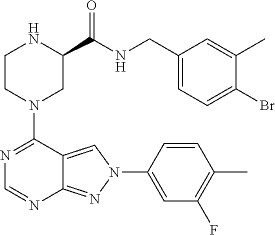 [SMILES: Cc1ccc(-n2cc3c(N4CCN[C@@H](C(=O)NCc5ccc(Br)c(C)c5)C4)ncnc3n2)cc1F] | (R)-N-(4-bromo-3- methylbenzyl)-4-(2-(3- fluoro-4-methylphenyl)- 2H-pyrazolo[3,4- d]pyrimidin-4- yl)piperazine-2- carboxamide | 538.13 |
| 275 | 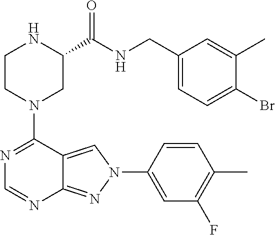 [SMILES: Cc1ccc(-n2cc3c(N4CCN[C@H](C(=O)NCc5ccc(Br)c(C)c5)C4)ncnc3n2)cc1F] | (S)-N-(4-bromo-3- methylbenzyl)-4-(2-(3- fluoro-4-methylphenyl)- 2H-pyrazolo[3,4- d]pyrimidin-4- yl)piperazine-2- carboxamide | 538.10 |
| 276 | 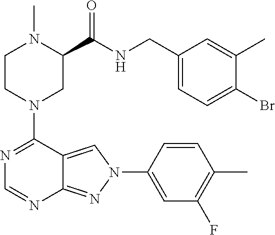 [SMILES: Cc1ccc(-n2cc3c(N4CCN(C)[C@@H](C(=O)NCc5ccc(Br)c(C)c5)C4)ncnc3n2)cc1F] | (R)-N-(4-bromo-3- methylbenzyl)-4-(2-(3- fluoro-4-methylphenyl)- 2H-pyrazolo[3,4- d]pyrimidin-4-yl)-1- methylpiperazine-2- carboxamide | 552.16 |
| 277 | 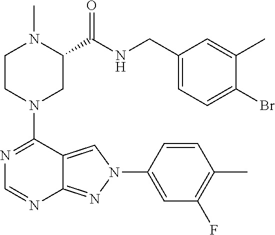 [SMILES: Cc1ccc(-n2cc3c(N4CCN(C)[C@H](C(=O)NCc5ccc(Br)c(C)c5)C4)ncnc3n2)cc1F] | (S)-N-(4-bromo-3- methylbenzyl)-4-(2-(3- fluoro-4-methylphenyl)- 2H-pyrazolo[3,4- d]pyrimidin-4-yl)-1- methylpiperazine-2- carboxamide | 552.12 |
| 278 | 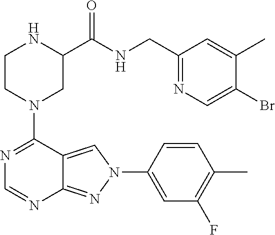 [SMILES: Cc1ccc(-n2cc3c(N4CCNC(C(=O)NCc5cc(C)c(Br)cn5)C4)ncnc3n2)cc1F] | N-((5-bromo-4- methylpyridin-2- yl)methyl)-4-(2-(3-fluoro- 4-methylphenyl)-2H- pyrazolo[3,4-d]pyrimidin- 4-yl)piperazine-2- carboxamide | 538.10 |
| 279 | 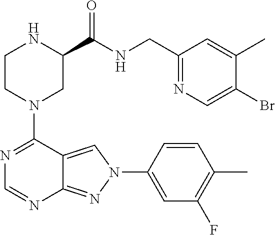 [SMILES: Cc1ccc(-n2cc3c(N4CCN[C@@H](C(=O)NCc5cc(C)c(Br)cn5)C4)ncnc3n2)cc1F] | (R)-N-((5-bromo-4- methylpyridin-2- yl)methyl)-4-(2-(3-fluoro- 4-methylphenyl)-2H- pyrazolo[3,4-d]pyrimidin- 4-yl)piperazine-2- carboxamide | 538.16 |
| 280 | 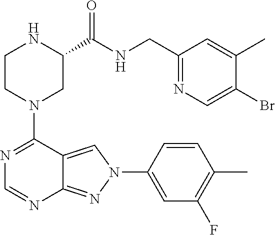 [SMILES: Cc1ccc(-n2cc3c(N4CCN[C@H](C(=O)NCc5cc(C)c(Br)cn5)C4)ncnc3n2)cc1F] | (S)-N-((5-bromo-4- methylpyridin-2- yl)methyl)-4-(2-(3-fluoro- 4-methylphenyl)-2H- pyrazolo[3,4-d]pyrimidin- 4-yl)piperazine-2- carboxamide | 538.11 |
| 281 | 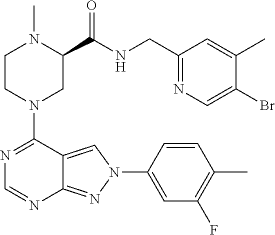 [SMILES: Cc1ccc(-n2cc3c(N4CCN(C)[C@@H](C(=O)NCc5cc(C)c(Br)cn5)C4)ncnc3n2)cc1F] | (R)-N-((5-bromo-4- methylpyridin-2- yl)methyl)-4-(2-(3-fluoro- 4-methylphenyl)-2H- pyrazolo[3,4-d]pyrimidin- 4-yl)-1-methylpiperazine- 2-carboxamide | 553.07 |
| 282 | 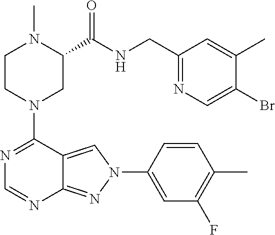 [SMILES: Cc1ccc(-n2cc3c(N4CCN(C)[C@H](C(=O)NCc5cc(C)c(Br)cn5)C4)ncnc3n2)cc1F] | (S)-N-((5-bromo-4- methylpyridin-2- yl)methyl)-4-(2-(3-fluoro- 4-methylphenyl)-2H- pyrazolo[3,4-d]pyrimidin- 4-yl)-1-methylpiperazine- 2-carboxamide | 553.11 |
| 283 | 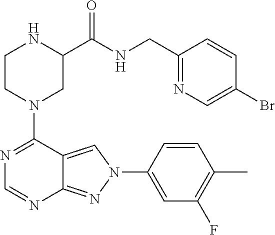 [SMILES: Cc1ccc(-n2cc3c(N4CCNC(C(=O)NCc5ccc(Br)cn5)C4)ncnc3n2)cc1F] | N-((5-bromopyridin-2- yl)methyl)-4-(2-(3-fluoro- 4-methylphenyl)-2H- pyrazolo[3,4-d]pyrimidin- 4-yl)piperazine-2- carboxamide | 524.11 |
| 284 | 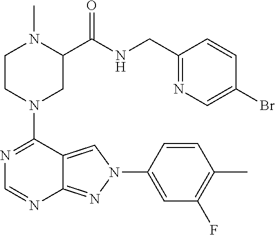 [SMILES: Cc1ccc(-n2cc3c(N4CCN(C)C(C(=O)NCc5ccc(Br)cn5)C4)ncnc3n2)cc1F] | N-((5-bromopyridin-2- yl)methyl)-4-(2-(3-fluoro- 4-methylphenyl)-2H- pyrazolo[3,4-d]pyrimidin- 4-yl)-1-methylpiperazine- 2-carboxamide | 537.99 |
| 285 | 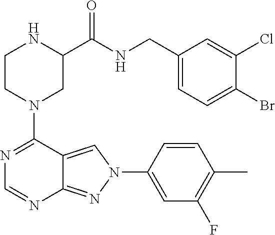 [SMILES: Cc1ccc(-n2cc3c(N4CCNC(C(=O)NCc5ccc(Br)c(Cl)c5)C4)ncnc3n2)cc1F] | N-(4-bromo-3- chlorobenzyl)-4-(2-(3- fluoro-4-methylphenyl)- 2H-pyrazolo[3,4- d]pyrimidin-4- yl)piperazine-2- carboxamide | 558.09 |
| 286 | 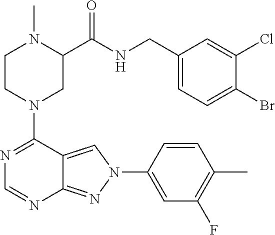 [SMILES: Cc1ccc(-n2cc3c(N4CCN(C)C(C(=O)NCc5ccc(Br)c(Cl)c5)C4)ncnc3n2)cc1F] | N-(4-bromo-3- chlorobenzyl)-4-(2-(3- fluoro-4-methylphenyl)- 2H-pyrazolo[3,4- d]pyrimidin-4-yl)-1- methylpiperazine-2- carboxamide | 572.10 |
| 287 | 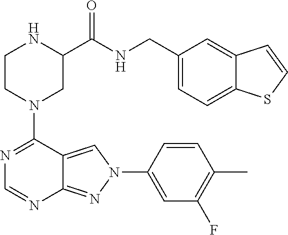 [SMILES: Cc1ccc(-n2cc3c(N4CCNC(C(=O)NCc5ccc6sccc6c5)C4)ncnc3n2)cc1F] | N-(benzo[b]thiophen-5- ylmethyl)-4-(2-(3-fluoro-4- methylphenyl)-2H- pyrazolo[3,4-d]pyrimidin- 4-yl)piperazine-2- carboxamide | 502.19 |
| 288 | 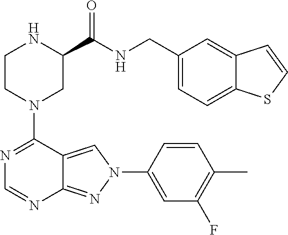 [SMILES: Cc1ccc(-n2cc3c(N4CCN[C@@H](C(=O)NCc5ccc6sccc6c5)C4)ncnc3n2)cc1F] | (R)-N-(benzo[b]thiophen- 5-ylmethyl)-4-(2-(3-fluoro- 4-methylphenyl)-2H- pyrazolo[3,4-d]pyrimidin- 4-yl)piperazine-2- carboxamide | 502.17 |
| 289 | 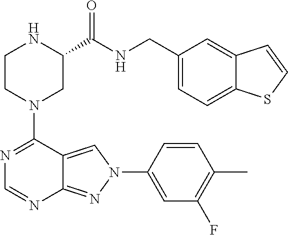 [SMILES: Cc1ccc(-n2cc3c(N4CCN[C@H](C(=O)NCc5ccc6sccc6c5)C4)ncnc3n2)cc1F] | (S)-N-(benzo[b]thiophen- 5-ylmethyl)-4-(2-(3-fluoro- 4-methylphenyl)-2H- pyrazolo[3,4-d]pyrimidin- 4-yl)piperazine-2- carboxamide | 502.09 |
| 290 | 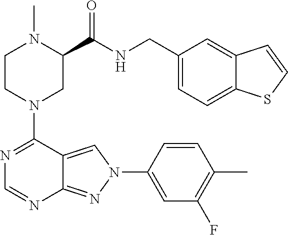 [SMILES: Cc1ccc(-n2cc3c(N4CCN(C)[C@@H](C(=O)NCc5ccc6sccc6c5)C4)ncnc3n2)cc1F] | (R)-N-(benzo[b]thiophen- 5-ylmethyl)-4-(2-(3-fluoro- 4-methylphenyl)-2H- pyrazolo[3,4-d]pyrimidin- 4-yl)-1-methylpiperazine- 2-carboxamide | 516.17 |
| 291 | 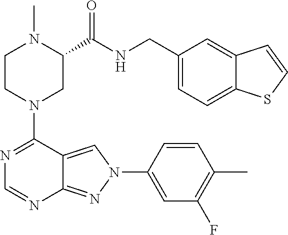 [SMILES: Cc1ccc(-n2cc3c(N4CCN(C)[C@H](C(=O)NCc5ccc6sccc6c5)C4)ncnc3n2)cc1F] | (S)-N-(benzo[b]thiophen- 5-ylmethyl)-4-(2-(3-fluoro- 4-methylphenyl)-2H- pyrazolo[3,4-d]pyrimidin- 4-yl)-1-methylpiperazine- 2-carboxamide | 516.16 |
| 292 | 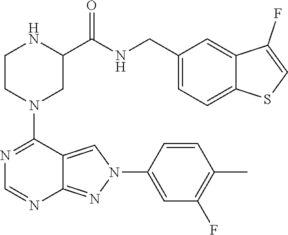 [SMILES: Cc1ccc(-n2cc3c(N4CCNC(C(=O)NCc5ccc6scc(F)c6c5)C4)ncnc3n2)cc1F] | 4-(2-(3-fluoro-4- methylphenyl)-2H- pyrazolo[3,4-d]pyrimidin- 4-yl)-N-((3- fluorobenzo[b]thiophen- 5-yl)methyl)piperazine-2- carboxamide | 520.19 |
| 293 | 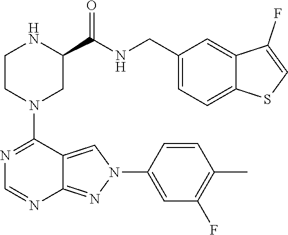 [SMILES: Cc1ccc(-n2cc3c(N4CCN[C@@H](C(=O)NCc5ccc6scc(F)c6c5)C4)ncnc3n2)cc1F] | (R)-4-(2-(3-fluoro-4- methylphenyl)-2H- pyrazolo[3,4-d]pyrimidin- 4-yl)-N-((3- fluorobenzo[b]thiophen- 5-yl)methyl)piperazine-2- carboxamide | 520.16 |
| 294 | 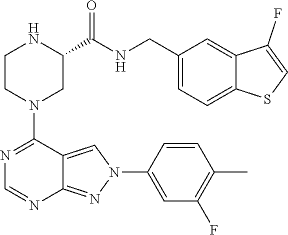 [SMILES: Cc1ccc(-n2cc3c(N4CCN[C@H](C(=O)NCc5ccc6scc(F)c6c5)C4)ncnc3n2)cc1F] | (S)-4-(2-(3-fluoro-4- methylphenyl)-2H- pyrazolo[3,4-d]pyrimidin- 4-yl)-N-((3- fluorobenzo[b]thiophen- 5-yl)methyl)piperazine-2- carboxamide | 520.16 |
| 295 | 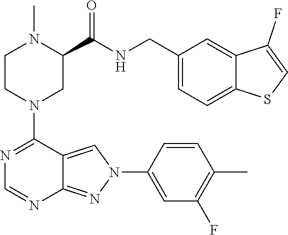 [SMILES: Cc1ccc(-n2cc3c(N4CCN(C)[C@@H](C(=O)NCc5ccc6scc(F)c6c5)C4)ncnc3n2)cc1F] | (R)-4-(2-(3-fluoro-4- methylphenyl)-2H- pyrazolo[3,4-d]pyrimidin- 4-yl)-N-((3- fluorobenzo[b]thiophen- 5-yl)methyl)-1- methylpiperazine-2- carboxamide | 534.13 |
| 296 | 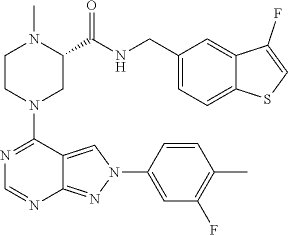 [SMILES: Cc1ccc(-n2cc3c(N4CCN(C)[C@H](C(=O)NCc5ccc6scc(F)c6c5)C4)ncnc3n2)cc1F] | (S)-4-(2-(3-fluoro-4- methylphenyl)-2H- pyrazolo[3,4-d]pyrimidin- 4-yl)-N-((3- fluorobenzo[b]thiophen- 5-yl)methyl)-1- methylpiperazine-2- carboxamide | 534.09 |
| 297 | 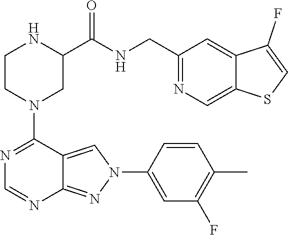 [SMILES: Cc1ccc(-n2cc3c(N4CCNC(C(=O)NCc5cc6c(F)csc6cn5)C4)ncnc3n2)cc1F] | 4-(2-(3-fluoro-4- methylphenyl)-2H- pyrazolo[3,4-d]pyrimidin- 4-yl)-N-((3- fluorothieno[2,3-c]pyridin- 5-yl)methyl)piperazine-2- carboxamide | 521.06 |
| 298 | 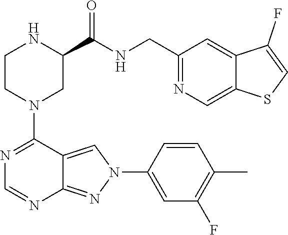 [SMILES: Cc1ccc(-n2cc3c(N4CCN[C@@H](C(=O)NCc5cc6c(F)csc6cn5)C4)ncnc3n2)cc1F] | (R)-4-(2-(3-fluoro-4- methylphenyl)-2H- pyrazolo[3,4-d]pyrimidin- 4-yl)-N-((3- fluorothieno[2,3-c]pyridin- 5-yl)methyl)piperazine-2- carboxamide | 521.10 |
| 299 | 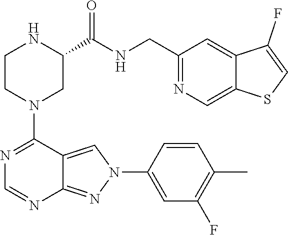 [SMILES: Cc1ccc(-n2cc3c(N4CCN[C@H](C(=O)NCc5cc6c(F)csc6cn5)C4)ncnc3n2)cc1F] | (S)-1-(2-(3-fluoro-4- methylphenyl)-2H- pyrazolo[3,4-d]pyrimidin- 4-yl)-N-((3- fluorothieno[2,3-c]pyridin- 5-yl)methyl)piperidine-3- carboxamide | 521.13 |
| 300 | 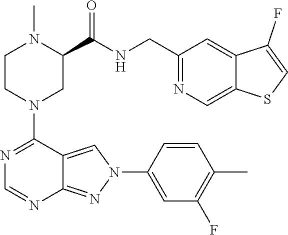 [SMILES: Cc1ccc(-n2cc3c(N4CCN(C)[C@@H](C(=O)NCc5cc6c(F)csc6cn5)C4)ncnc3n2)cc1F] | (R)-4-(2-(3-fluoro-4- methylphenyl)-2H- pyrazolo[3,4-d]pyrimidin- 4-yl)-N-((3- fluorothieno[2,3-c]pyridin- 5-yl)methyl)-1- methylpiperazine-2- carboxamide | 534.17 |
| 301 | 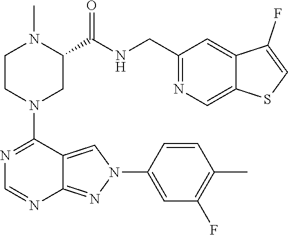 [SMILES: Cc1ccc(-n2cc3c(N4CCN(C)[C@H](C(=O)NCc5cc6c(F)csc6cn5)C4)ncnc3n2)cc1F] | (S)-4-(2-(3-fluoro-4- methylphenyl)-2H- pyrazolo[3,4-d]pyrimidin- 4-yl)-N-((3- fluorothieno[2,3-c]pyridin- 5-yl)methyl)-1- methylpiperazine-2- carboxamide | 534.11 |
| 302 | 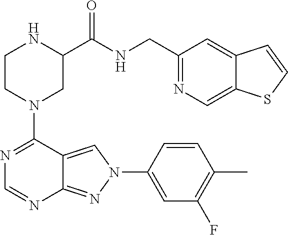 [SMILES: Cc1ccc(-n2cc3c(N4CCNC(C(=O)NCc5cc6ccsc6cn5)C4)ncnc3n2)cc1F] | 4-(2-(3-fluoro-4- methylphenyl)-2H- pyrazolo[3,4-d]pyrimidin- 4-yl)-N-(thieno[2,3- c]pyridin-5- ylmethyl)piperazine-2- carboxamide | 503.53 |
| 303 | 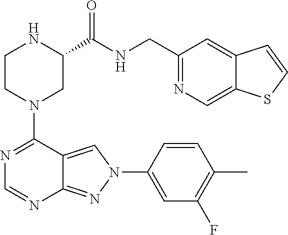 [SMILES: Cc1ccc(-n2cc3c(N4CCN[C@H](C(=O)NCc5cc6ccsc6cn5)C4)ncnc3n2)cc1F] | (S)-4-(2-(3-fluoro-4- methylphenyl)-2H- pyrazolo[3,4-d]pyrimidin- 4-yl)-N-(thieno[2,3- c]pyridin-5- ylmethyl)piperazine-2- carboxamide | 503.37 |
| 304 | 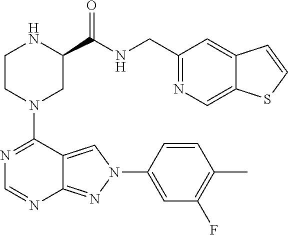 [SMILES: Cc1ccc(-n2cc3c(N4CCN[C@@H](C(=O)NCc5cc6ccsc6cn5)C4)ncnc3n2)cc1F] | (R)-4-(2-(3-fluoro-4- methylphenyl)-2H- pyrazolo[3,4-d]pyrimidin- 4-yl)-N-(thieno[2,3- c]pyridin-5- ylmethyl)piperazine-2- carboxamide | 503.21 |
| 305 | 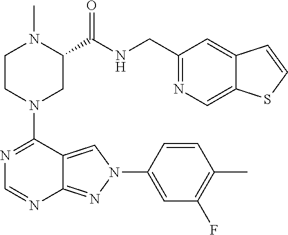 [SMILES: Cc1ccc(-n2cc3c(N4CCN(C)[C@H](C(=O)NCc5cc6ccsc6cn5)C4)ncnc3n2)cc1F] | (S)-4-(2-(3-fluoro-4- methylphenyl)-2H- pyrazolo[3,4-d]pyrimidin- 4-yl)-1-methyl-N- (thieno[2,3-c]pyridin-5- ylmethyl)piperazine-2- carboxamide | 517.20 |
| 306 | 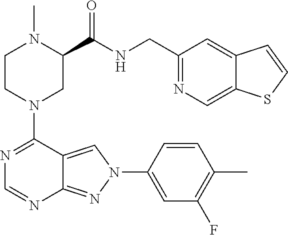 [SMILES: Cc1ccc(-n2cc3c(N4CCN(C)[C@@H](C(=O)NCc5cc6ccsc6cn5)C4)ncnc3n2)cc1F] | (R)-4-(2-(3-fluoro-4- methylphenyl)-2H- pyrazolo[3,4-d]pyrimidin- 4-yl)-1-methyl-N- (thieno[2,3-c]pyridin-5- ylmethyl)piperazine-2- carboxamide | 517.16 |
| 307 | 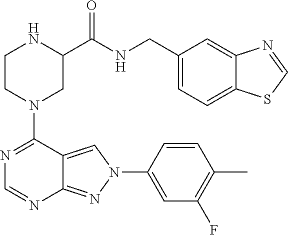 [SMILES: Cc1ccc(-n2cc3c(N4CCNC(C(=O)NCc5ccc6scnc6c5)C4)ncnc3n2)cc1F] | N-(benzo[d]thiazol-5- ylmethyl)-4-(2-(3-fluoro-4- methylphenyl)-2H- pyrazolo[3,4-d]pyrimidin- 4-yl)piperazine-2- carboxamide | 503.17 |
| 308 | 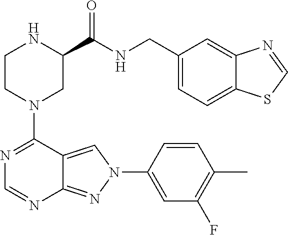 [SMILES: Cc1ccc(-n2cc3c(N4CCN[C@@H](C(=O)NCc5ccc6scnc6c5)C4)ncnc3n2)cc1F] | (R)-N-(benzo[d]thiazol-5- ylmethyl)-4-(2-(3-fluoro-4- methylphenyl)-2H- pyrazolo[3,4-d]pyrimidin- 4-yl)piperazine-2- carboxamide | 503.16 |
| 309 | 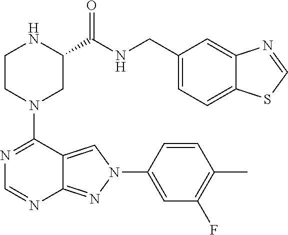 [SMILES: Cc1ccc(-n2cc3c(N4CCN[C@H](C(=O)NCc5ccc6scnc6c5)C4)ncnc3n2)cc1F] | (S)-N-(benzo[d]thiazol-5- ylmethyl)-4-(2-(3-fluoro-4- methylphenyl)-2H- pyrazolo[3,4-d]pyrimidin- 4-yl)piperazine-2- carboxamide | 503.17 |
| 310 | 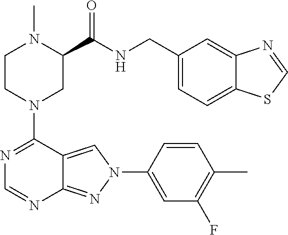 [SMILES: Cc1ccc(-n2cc3c(N4CCN(C)[C@@H](C(=O)NCc5ccc6scnc6c5)C4)ncnc3n2)cc1F] | (R)-N-(benzo[d]thiazol-5- ylmethyl)-4-(2-(3-fluoro-4- methylphenyl)-2H- pyrazolo[3,4-d]pyrimidin- 4-yl)-1-methylpiperazine- 2-carboxamide | 517.08 |
| 311 | 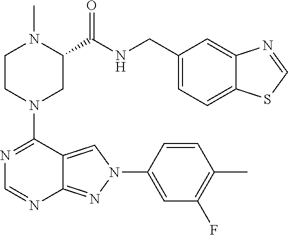 [SMILES: Cc1ccc(-n2cc3c(N4CCN(C)[C@H](C(=O)NCc5ccc6scnc6c5)C4)ncnc3n2)cc1F] | (S)-N-(benzo[d]thiazol-5- ylmethyl)-4-(2-(3-fluoro-4- methylphenyl)-2H- pyrazolo[3,4-d]pyrimidin- 4-yl)-1-methylpiperazine- 2-carboxamide | 517.12 |
| 312 | 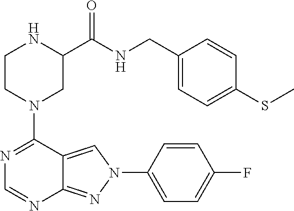 [SMILES: CSc1ccc(CNC(=O)C2CN(c3ncnc4nn(-c5ccc(F)cc5)cc34)CCN2)cc1] | 4-(2-(4-fluorophenyl)-2H- pyrazolo[3,4-d]pyrimidin- 4-yl)-N-(4- (methylthio)benzyl) piperazine-2-carboxamide | 478.12 |
| 313 | 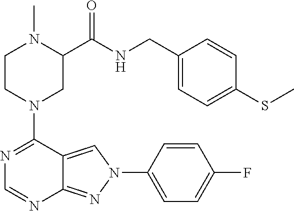 [SMILES: CSc1ccc(CNC(=O)C2CN(c3ncnc4nn(-c5ccc(F)cc5)cc34)CCN2C)cc1] | 4-(2-(4-fluorophenyl)-2H- pyrazolo[3,4-d]pyrimidin- 4-yl)-1-methyl-N-(4- (methylthio)benzyl) piperazine-2-carboxamide | 492.17 |
| 314 | 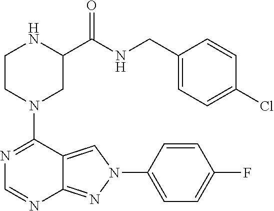 [SMILES: O=C(NCc1ccc(Cl)cc1)C1CN(c2ncnc3nn(-c4ccc(F)cc4)cc23)CCN1] | N-(4-chlorobenzyl)-4-(2- (4-fluorophenyl)-2H- pyrazolo[3,4-d]pyrimidin- 4-yl)piperazine-2- carboxamide | 466.16 |
| 315 | 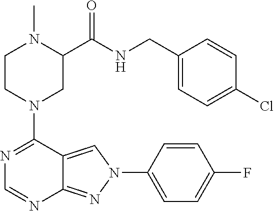 [SMILES: CN1CCN(c2ncnc3nn(-c4ccc(F)cc4)cc23)CC1C(=O)NCc1ccc(Cl)cc1] | N-(4-chlorobenzyl)-4-(2- (4-fluorophenyl)-2H- pyrazolo[3,4-d]pyrimidin- 4-yl)-1-methylpiperazine- 2-carboxamide | 479.13 |
| 316 | 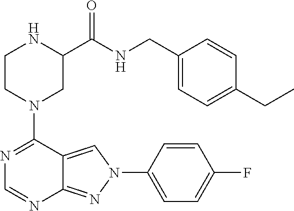 [SMILES: CCc1ccc(CNC(=O)C2CN(c3ncnc4nn(-c5ccc(F)cc5)cc34)CCN2)cc1] | N-(4-ethylbenzyl)-4-(2-(4- fluorophenyl)-2H- pyrazolo[3,4-d]pyrimidin- 4-yl)piperazine-2- carboxamide | 460.22 |
| 317 | 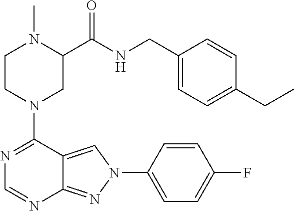 [SMILES: CCc1ccc(CNC(=O)C2CN(c3ncnc4nn(-c5ccc(F)cc5)cc34)CCN2C)cc1] | N-(4-ethylbenzyl)-4-(2-(4- fluorophenyl)-2H- pyrazolo[3,4-d]pyrimidin- 4-yl)-1-methylpiperazine- 2-carboxamide | 474.13 |
| 318 | 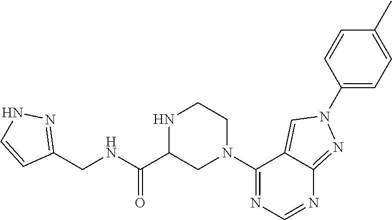 [SMILES: Cc1ccc(-n2cc3c(N4CCNC(C(=O)NCc5cc[nH]n5)C4)ncnc3n2)cc1] | N-((1H-pyrazol-3- yl)methyl)-4-(2-(p-tolyl)- 2H-pyrazolo[3,4- d]pyrimidin-4- yl)piperazine-2- carboxamide | 417.16 |
| 319 | 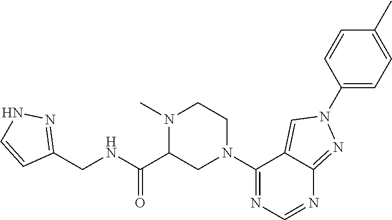 [SMILES: Cc1ccc(-n2cc3c(N4CCN(C)C(C(=O)NCc5cc[nH]n5)C4)ncnc3n2)cc1] | N-((1H-pyrazol-3- yl)methyl)-1-methyl-4-(2- (p-tolyl)-2H-pyrazolo[3,4- d]pyrimidin-4- yl)piperazine-2- carboxamide | 431.10 |
| 320 | 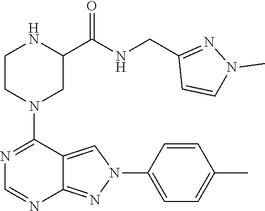 [SMILES: Cc1ccc(-n2cc3c(N4CCNC(C(=O)NCc5ccn(C)n5)C4)ncnc3n2)cc1] | N-((1-methyl-1H-pyrazol- 3-yl)methyl)-4-(2-(p-tolyl)- 2H-pyrazolo[3,4- d]pyrimidin-4- yl)piperazine-2- carboxamide | 432.28 |
| 321 | 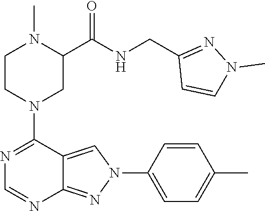 [SMILES: Cc1ccc(-n2cc3c(N4CCN(C)C(C(=O)NCc5ccn(C)n5)C4)ncnc3n2)cc1] | 1-methyl-N-((1-methyl- 1H-pyrazol-3-yl)methyl)- 4-(2-(p-tolyl)-2H- pyrazolo[3,4-d]pyrimidin- 4-yl)piperazine-2- carboxamide | 446.15 |
| 322 | 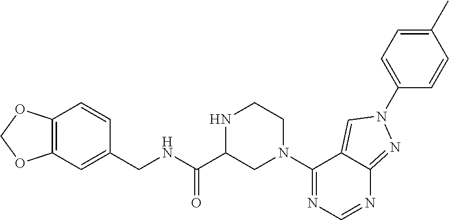 [SMILES: Cc1ccc(-n2cc3c(N4CCNC(C(=O)NCc5ccc6c(c5)OCO6)C4)ncnc3n2)cc1] | N-(benzo[d][1,3]dioxol-5- ylmethyl)-4-(2-(p-tolyl)- 2H-pyrazolo[3,4- d]pyrimidin-4- yl)piperazine-2- carboxamide | 472.12 |
| 323 | 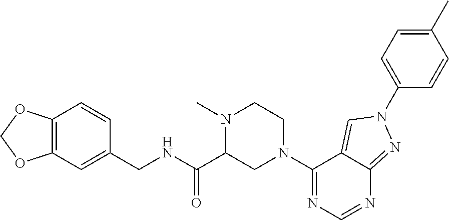 [SMILES: Cc1ccc(-n2cc3c(N4CCN(C)C(C(=O)NCc5ccc6c(c5)OCO6)C4)ncnc3n2)cc1] | N-(benzo[d][1,3]dioxol-5- ylmethyl)-1-methyl-4-(2- (p-tolyl)-2H-pyrazolo[3,4- d]pyrimidin-4- yl)piperazine-2- carboxamide | 486.27 |
| 324 | 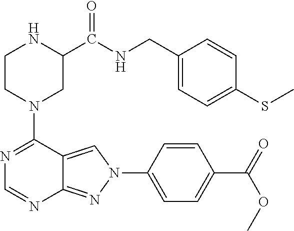 [SMILES: COC(=O)c1ccc(-n2cc3c(N4CCNC(C(=O)NCc5ccc(SC)cc5)C4)ncnc3n2)cc1] | methyl 4-(4-(3-((4- (methylthio)benzyl)carba- moyl)piperazin-1-yl)-2H- pyrazolo[3,4-d]pyrimidin- 2-yl)benzoate | 518.23 |
| 325 | 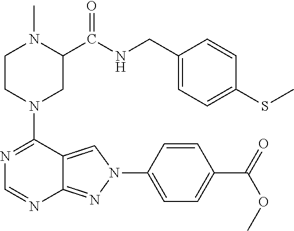 [SMILES: COC(=O)c1ccc(-n2cc3c(N4CCN(C)C(C(=O)NCc5ccc(SC)cc5)C4)ncnc3n2)cc1] | methyl 4-(4-(4-methyl-3- ((4-(methylthio) benzyl)carbamoyl) piperazin-1-yl)-2H- pyrazolo[3,4-d]pyrimidin- 2-yl)benzoate | 532.16 |
| 326 | 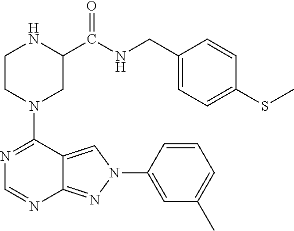 [SMILES: CSc1ccc(CNC(=O)C2CN(c3ncnc4nn(-c5cccc(C)c5)cc34)CCN2)cc1] | N-(4-(methylthio)benzyl)- 4-(2-(m-tolyl)-2H- pyrazolo[3,4-d]pyrimidin- 4-yl)piperazine-2- carboxamide | 474.13 |
| 327 | 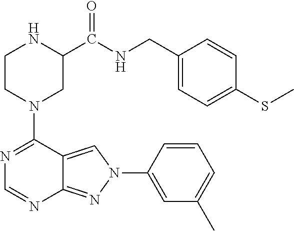 [SMILES: CSc1ccc(CNC(=O)C2CN(c3ncnc4nn(-c5cccc(C)c5)cc34)CCN2)cc1] | 1-methyl-N-(4- (methylthio)benzyl)-4-(2- (m-tolyl)-2H-pyrazolo[3,4- d]pyrimidin-4- yl)piperazine-2- carboxamide | 488.17 |
| 328 | 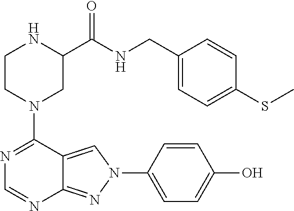 [SMILES: CSc1ccc(CNC(=O)C2CN(c3ncnc4nn(-c5ccc(O)cc5)cc34)CCN2)cc1] | 4-(2-(4-hydroxyphenyl)- 2H-pyrazolo[3,4- d]pyrimidin-4-yl)-N-(4- (methylthio)benzyl) piperazine-2-carboxamide | 476.17 |
| 329 | 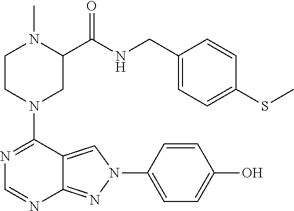 [SMILES: CSc1ccc(CNC(=O)C2CN(c3ncnc4nn(-c5ccc(O)cc5)cc34)CCN2C)cc1] | 4-(2-(4-hydroxyphenyl)- 2H-pyrazolo[3,4- d]pyrimidin-4-yl)-1- methyl-N-(4- (methylthio)benzyl) piperazine-2-carboxamide | 490.13 |
| 330 | 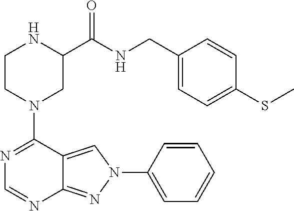 [SMILES: CSc1ccc(CNC(=O)C2CN(c3ncnc4nn(-c5ccccc5)cc34)CCN2)cc1] | N-(4-(methylthio)benzyl)- 4-(2-phenyl-2H- pyrazolo[3,4-d]pyrimidin- 4-yl)piperazine-2- carboxamide | 460.20 |
| 331 | 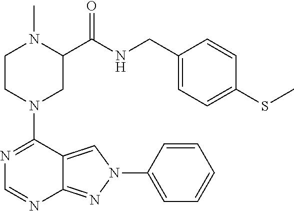 [SMILES: CSc1ccc(CNC(=O)C2CN(c3ncnc4nn(-c5ccccc5)cc34)CCN2C)cc1] | 1-methyl-N-(4- (methylthio)benzyl)-4-(2- phenyl-2H-pyrazolo[3,4- d]pyrimidin-4- yl)piperazine-2- carboxamide | 474.09 |
| 332 | 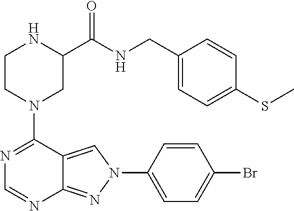 [SMILES: CSc1ccc(CNC(=O)C2CN(c3ncnc4nn(-c5ccc(Br)cc5)cc34)CCN2)cc1] | 4-(2-(4-bromophenyl)- 2H-pyrazolo[3,4- d]pyrimidin-4-yl)-N-(4- (methylthio)benzyl) piperazine-2-carboxamide | 538.06 |
| 333 | 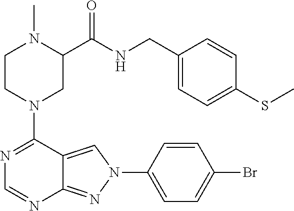 [SMILES: CSc1ccc(CNC(=O)C2CN(c3ncnc4nn(-c5ccc(Br)cc5)cc34)CCN2C)cc1] | 4-(2-(4-bromophenyl)- 2H-pyrazolo[3,4- d]pyrimidin-4-yl)-1- methyl-N-(4- (methylthio)benzyl) piperazine-2-carboxamide | 552.17 |
| 334 | 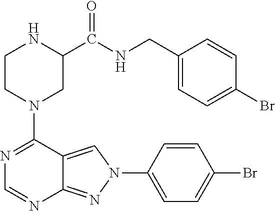 [SMILES: O=C(NCc1ccc(Br)cc1)C1CN(c2ncnc3nn(-c4ccc(Br)cc4)cc23)CCN1] | N-(4-bromobenzyl)-4-(2- (4-bromophenyl)-2H- pyrazolo[3,4-d]pyrimidin- 4-yl)piperazine-2- carboxamide | 570.08 |
| 335 | 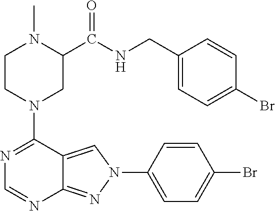 [SMILES: CN1CCN(c2ncnc3nn(-c4ccc(Br)cc4)cc23)CC1C(=O)NCc1ccc(Br)cc1] | N-(4-bromobenzyl)-4-(2- (4-bromophenyl)-2H- pyrazolo[3,4-d]pyrimidin- 4-yl)-1-methylpiperazine- 2-carboxamide | 584.17 |
| 336 | 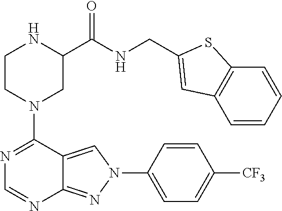 [SMILES: O=C(NCc1cc2ccccc2s1)C1CN(c2ncnc3nn(-c4ccc(C(F)(F)F)cc4)cc23)CCN1] | N-(benzo[b]thiophen-2- ylmethyl)-4-(2-(4- (trifluoromethyl)phenyl)- 2H-pyrazolo[3,4- d]pyrimidin-4- yl)piperazine-2- carboxamide | 538.22 |
| 337 | 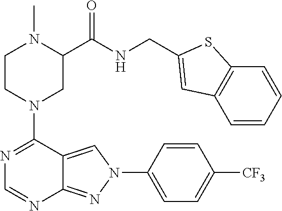 [SMILES: CN1CCN(c2ncnc3nn(-c4ccc(C(F)(F)F)cc4)cc23)CC1C(=O)NCc1cc2ccccc2s1] | N-(benzo[b]thiophen-2- ylmethyl)-1-methyl-4-(2- (4- (trifluoromethyl)phenyl)- 2H-pyrazolo[3,4- d]pyrimidin-4- yl)piperazine-2- carboxamide | 552.17 |
| 338 | 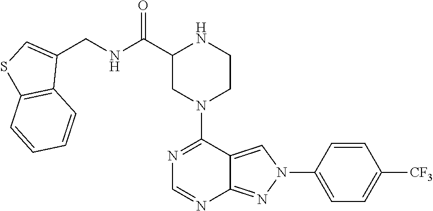 [SMILES: O=C(NCc1csc2ccccc12)C1CN(c2ncnc3nn(-c4ccc(C(F)(F)F)cc4)cc23)CCN1] | N-(benzo[b]thiophen-3- ylmethyl)-4-(2-(4- (trifluoromethyl)phenyl)- 2H-pyrazolo[3,4- d]pyrimidin-4- yl)piperazine-2- carboxamide | 538.11 |
| 339 | 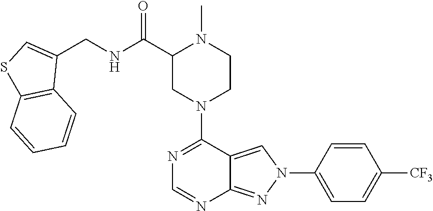 [SMILES: CN1CCN(c2ncnc3nn(-c4ccc(C(F)(F)F)cc4)cc23)CC1C(=O)NCc1csc2ccccc12] | N-(benzo[b]thiophen-3- ylmethyl)-1-methyl-4-(2- (4- (trifluoromethyl)phenyl)- 2H-pyrazolo[3,4- d]pyrimidin-4- yl)piperazine-2- carboxamide | 552.13 |
| 340 | 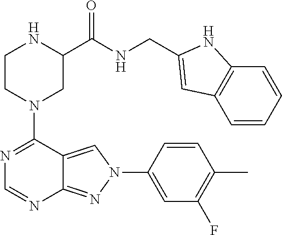 [SMILES: Cc1ccc(-n2cc3c(N4CCNC(C(=O)NCc5cc6ccccc6[nH]5)C4)ncnc3n2)cc1F] | N-((1H-indol-2-yl)methyl)- 4-(2-(3-fluoro-4- methylphenyl)-2H- pyrazolo[3,4-d]pyrimidin- 4-yl)piperazine-2- carboxamide | 485.14 |
| 341 | 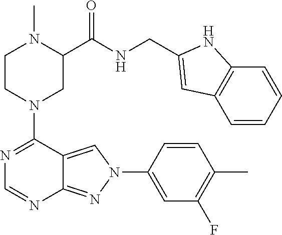 [SMILES: Cc1ccc(-n2cc3c(N4CCN(C)C(C(=O)NCc5cc6ccccc6[nH]5)C4)ncnc3n2)cc1F] | N-((1H-indol-2-yl)methyl)- 4-(2-(3-fluoro-4- methylphenyl)-2H- pyrazolo[3,4-d]pyrimidin- 4-yl)-1-methylpiperazine- 2-carboxamide | 499.22 |
| 342 | 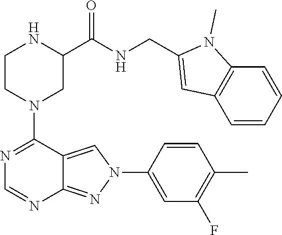 [SMILES: Cc1ccc(-n2cc3c(N4CCNC(C(=O)NCc5cc6ccccc6n5C)C4)ncnc3n2)cc1F] | 4-(2-(3-fluoro-4- methylphenyl)-2H- pyrazolo[3,4-d]pyrimidin- 4-yl)-N-((1-methyl-1H- indol-2- yl)methyl)piperazine-2- carboxamide | 499.15 |
| 343 | 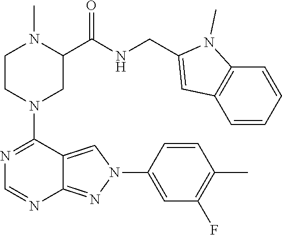 [SMILES: Cc1ccc(-n2cc3c(N4CCN(C)C(C(=O)NCc5cc6ccccc6n5C)C4)ncnc3n2)cc1F] | 4-(2-(3-fluoro-4- methylphenyl)-2H- pyrazolo[3,4-d]pyrimidin- 4-yl)-1-methyl-N-((1- methyl-1H-indol-2- yl)methyl)piperazine-2- carboxamide | 513.20 |
| 344 | 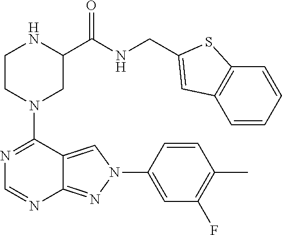 [SMILES: Cc1ccc(-n2cc3c(N4CCNC(C(=O)NCc5cc6ccccc6s5)C4)ncnc3n2)cc1F] | N-(benzo[b]thiophen-2- ylmethyl)-4-(2-(3-fluoro-4- methylphenyl)-2H- pyrazolo[3,4-d]pyrimidin- 4-yl)piperazine-2- carboxamide | 502.11 |
| 345 | 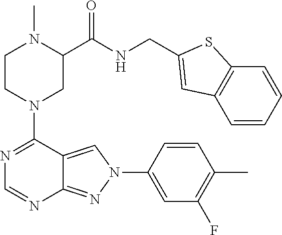 [SMILES: Cc1ccc(-n2cc3c(N4CCN(C)C(C(=O)NCc5cc6ccccc6s5)C4)ncnc3n2)cc1F] | N-(benzo[b]thiophen-2- ylmethyl)-4-(2-(3-fluoro-4- methylphenyl)-2H- pyrazolo[3,4-d]pyrimidin- 4-yl)-1-methylpiperazine- 2-carboxamide | 516.08 |
| 346 | 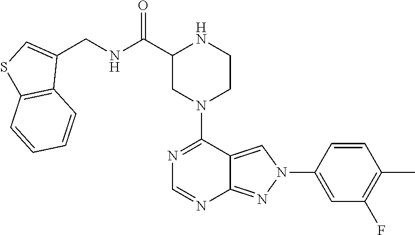 [SMILES: Cc1ccc(-n2cc3c(N4CCNC(C(=O)NCc5csc6ccccc56)C4)ncnc3n2)cc1F] | N-(benzo[b]thiophen-3- ylmethyl)-4-(2-(3-fluoro-4- methylphenyl)-2H- pyrazolo[3,4-d]pyrimidin- 4-yl)piperazine-2- carboxamide | 501.13 |
| 347 | 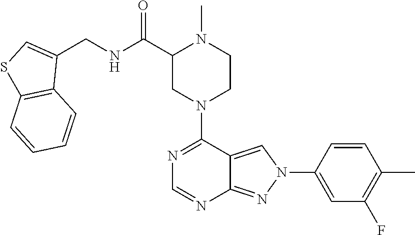 [SMILES: Cc1ccc(-n2cc3c(N4CCN(C)C(C(=O)NCc5csc6ccccc56)C4)ncnc3n2)cc1F] | N-(benzo[b]thiophen-3- ylmethyl)-4-(2-(3-fluoro-4- methylphenyl)-2H- pyrazolo[3,4-d]pyrimidin- 4-yl)-1-methylpiperazine- 2-carboxamide | 516.22 |
| 348 | 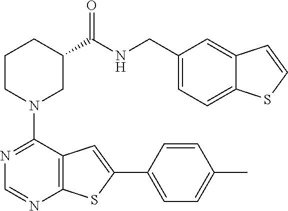 [SMILES: Cc1ccc(-c2cc3c(N4CCC[C@H](C(=O)NCc5ccc6sccc6c5)C4)ncnc3s2)cc1] | (S)-N-(benzo[b]thiophen- 5-ylmethyl)-1-(6-(p- tolyl)thieno[2,3- d]pyrimidin-4- yl)piperidine-3- carboxamide | 499.13 |
| 349 | 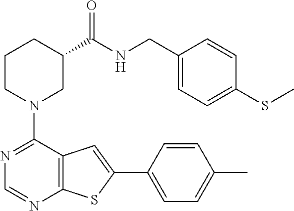 [SMILES: CSc1ccc(CNC(=O)[C@H]2CCCN(c3ncnc4sc(-c5ccc(C)cc5)cc34)C2)cc1] | (S)-N-(4- (methylthio)benzyl)-1-(6- (p-tolyl)thieno[2,3- d]pyrimidin-4- yl)piperidine-3- carboxamide | 489.12 |
| 350 | 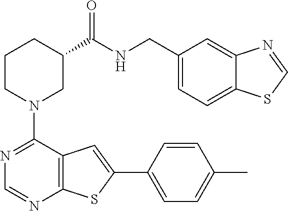 [SMILES: Cc1ccc(-c2cc3c(N4CCC[C@H](C(=O)NCc5ccc6scnc6c5)C4)ncnc3s2)cc1] | (S)-N-(benzo[d]thiazol-5- ylmethyl)-1-(6-(p- tolyl)thieno[2,3- d]pyrimidin-4- yl)piperidine-3- carboxamide | 500.11 |
| 351 | 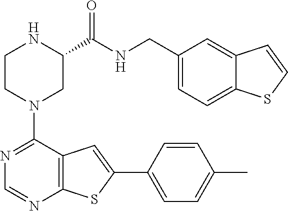 [SMILES: Cc1ccc(-c2cc3c(N4CCN[C@H](C(=O)NCc5ccc6sccc6c5)C4)ncnc3s2)cc1] | (S)-N-(benzo[b]thiophen- 5-ylmethyl)-4-(6-(p- tolyl)thieno[2,3- d]pyrimidin-4- yl)piperazine-2- carboxamide | 500.21 |
| 352 | 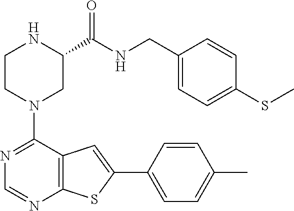 [SMILES: CSc1ccc(CNC(=O)[C@@H]2CN(c3ncnc4sc(-c5ccc(C)cc5)cc34)CCN2)cc1] | (S)-N-(4- (methylthio)benzyl)-4-(6- (p-tolyl)thieno[2,3- d]pyrimidin-4- yl)piperazine-2- carboxamide | 450.19 |
| 353 | 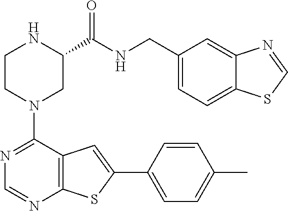 [SMILES: Cc1ccc(-c2cc3c(N4CCN[C@H](C(=O)NCc5ccc6scnc6c5)C4)ncnc3s2)cc1] | (S)-N-(benzo[d]thiazol-5- ylmethyl)-4-(6-(p- tolyl) thieno[2,3- d]pyrimidin-4- yl)piperazine-2- carboxamide | 501.11 |
| 354 | 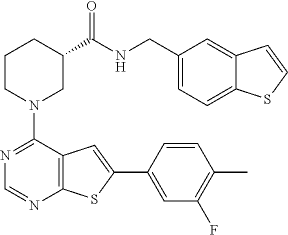 [SMILES: Cc1ccc(-c2cc3c(N4CCC[C@H](C(=O)NCc5ccc6sccc6c5)C4)ncnc3s2)cc1F] | (S)-N-(benzo[b]thiophen- 5-ylmethyl)-1-(6-(3-fluoro- 4- methylphenyl)thieno[2,3- d]pyrimidin-4- yl)piperidine-3- carboxamide | 517.11 |
| 355 | 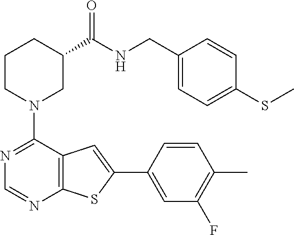 [SMILES: CSc1ccc(CNC(=O)[C@H]2CCCN(c3ncnc4sc(-c5ccc(C)c(F)c5)cc34)C2)cc1] | (S)-1-(6-(3-fluoro-4- methylphenyl)thieno[2,3- d]pyrimidin-4-yl)-N-(4- (methylthio)benzyl) piperidine-3-carboxamide | 507.17 |
| 356 | 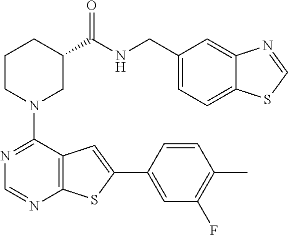 [SMILES: Cc1ccc(-c2cc3c(N4CCC[C@H](C(=O)NCc5ccc6scnc6c5)C4)ncnc3s2)cc1F] | (S)-N-(benzo[d]thiazol-5- ylmethyl)-1-(6-(3-fluoro-4- methylphenyl)thieno[2,3- d]pyrimidin-4- yl)piperidine-3- carboxamide | 518.09 |
| 357 | 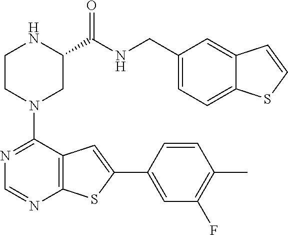 [SMILES: Cc1ccc(-c2cc3c(N4CCN[C@H](C(=O)NCc5ccc6sccc6c5)C4)ncnc3s2)cc1F] | (S)-N-(benzo[b]thiophen- 5-ylmethyl)-4-(6-(3-fluoro- 4- methylphenyl)thieno[2,3- d]pyrimidin-4- yl)piperazine-2- carboxamide | 518.13 |
| 358 | 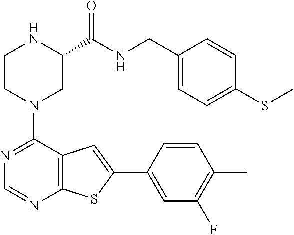 [SMILES: CSc1ccc(CNC(=O)[C@@H]2CN(c3ncnc4sc(-c5ccc(C)c(F)c5)cc34)CCN2)cc1] | (S)-4-(6-(3-fluoro-4- methylphenyl)thieno[2,3- d]pyrimidin-4-yl)-N-(4- (methylthio)benzyl) piperazine-2-carboxamide | 508.14 |
| 359 | 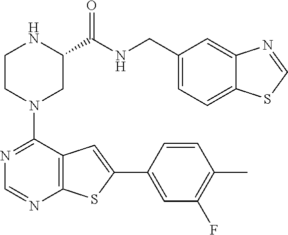 [SMILES: Cc1ccc(-c2cc3c(N4CCN[C@H](C(=O)NCc5ccc6scnc6c5)C4)ncnc3s2)cc1F] | (S)-N-(benzo[d]thiazol-5- ylmethyl)-4-(6-(3-fluoro-4- methylphenyl)thieno[2,3- d]pyrimidin-4- yl)piperazine-2- carboxamide | 519.18 |
| 360 | 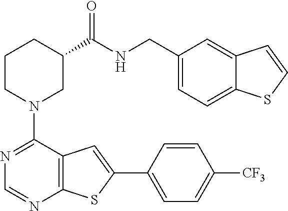 [SMILES: O=C(NCc1ccc2sccc2c1)[C@H]1CCCN(c2ncnc3sc(-c4ccc(C(F)(F)F)cc4)cc23)C1] | (S)-N-(benzo[b]thiophen- 5-ylmethyl)-1-(6-(4- (trifluoromethyl)phenyl) thieno[2,3-d]pyrimidin-4- yl)piperidine-3- carboxamide | 553.19 |
| 361 | 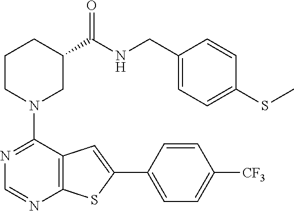 [SMILES: CSc1ccc(CNC(=O)[C@H]2CCCN(c3ncnc4sc(-c5ccc(C(F)(F)F)cc5)cc34)C2)cc1] | (S)-N-(4- (methylthio)benzyl)-1-(6- (4- (trifluoromethyl)phenyl) thieno[2,3-d]pyrimidin-4- yl)piperidine-3- carboxamide | 543.17 |
| 362 | 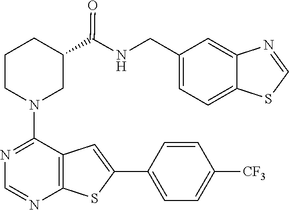 [SMILES: O=C(NCc1ccc2scnc2c1)[C@H]1CCCN(c2ncnc3sc(-c4ccc(C(F)(F)F)cc4)cc23)C1] | (S)-N-(benzo[d]thiazol-5- ylmethyl)-1-(6-(4- (trifluoromethyl)phenyl) thieno[2,3-d]pyrimidin-4- yl)piperidine-3- carboxamide | 554.08 |
| 363 | 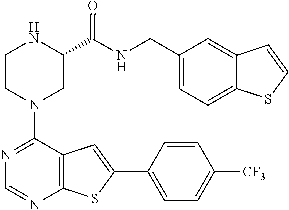 [SMILES: O=C(NCc1ccc2sccc2c1)[C@@H]1CN(c2ncnc3sc(-c4ccc(C(F)(F)F)cc4)cc23)CCN1] | (S)-N-(benzo[b]thiophen- 5-ylmethyl)-4-(6-(4- (trifluoromethyl)phenyl) thieno[2,3-d]pyrimidin-4- yl)piperazine-2- carboxamide | 554.01 |
| 364 | 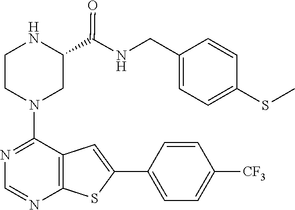 [SMILES: CSc1ccc(CNC(=O)[C@@H]2CN(c3ncnc4sc(-c5ccc(C(F)(F)F)cc5)cc34)CCN2)cc1] | (S)-N-(4- (methylthio)benzyl)-4-(6- (4- (trifluoromethyl)phenyl) thieno[2,3-d]pyrimidin-4- yl)piperazine-2- carboxamide | 544.17 |
| 365 | 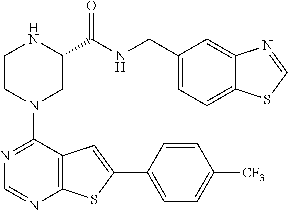 [SMILES: O=C(NCc1ccc2scnc2c1)[C@@H]1CN(c2ncnc3sc(-c4ccc(C(F)(F)F)cc4)cc23)CCN1] | (S)-N-(benzo[d]thiazol-5- ylmethyl)-4-(6-(4- (trifluoromethyl)phenyl) thieno[2,3-d]pyrimidin-4- yl)piperazine-2- carboxamide | 555.06 |
| 366 | 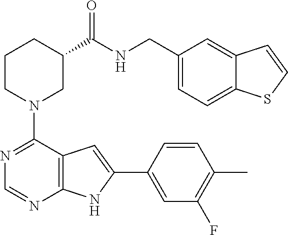 [SMILES: Cc1ccc(-c2cc3c(N4CCC[C@H](C(=O)NCc5ccc6sccc6c5)C4)ncnc3[nH]2)cc1F] | (S)-N-(benzo[b]thiophen- 5-ylmethyl)-1-(6-(3-fluoro- 4-methylphenyl)-7H- pyrrolo[2,3-d]pyrimidin-4- yl)piperidine-3- carboxamide | 500.17 |
| 367 | 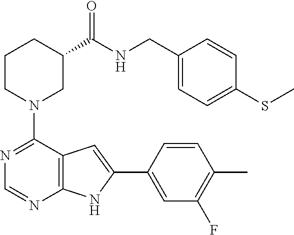 [SMILES: CSc1ccc(CNC(=O)[C@H]2CCCN(c3ncnc4[nH]c(-c5ccc(C)c(F)c5)cc34)C2)cc1] | (S)-1-(6-(3-fluoro-4- methylphenyl)-7H- pyrrolo[2,3-d]pyrimidin-4- yl)-N-(4- (methylthio)benzyl) piperidine-3-carboxamide | 490.25 |
| 368 | 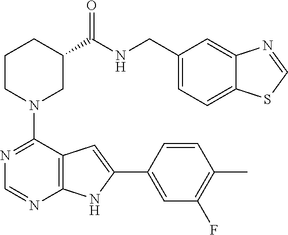 [SMILES: Cc1ccc(-c2cc3c(N4CCC[C@H](C(=O)NCc5ccc6scnc6c5)C4)ncnc3[nH]2)cc1F] | (S)-N-(benzo[d]thiazol-5- ylmethyl)-1-(6-(3-fluoro-4- methylphenyl)-7H- pyrrolo[2,3-d]pyrimidin-4- yl)piperidine-3- carboxamide | 501.24 |
| 369 | 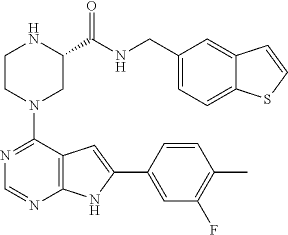 [SMILES: Cc1ccc(-c2cc3c(N4CCN[C@H](C(=O)NCc5ccc6sccc6c5)C4)ncnc3[nH]2)cc1F] | (S)-N-(benzo[b]thiophen- 5-ylmethyl)-4-(6-(3-fluoro- 4-methylphenyl)-7H- pyrrolo[2,3-d]pyrimidin-4- yl)piperazine-2- carboxamide | 501.21 |
| 370 | 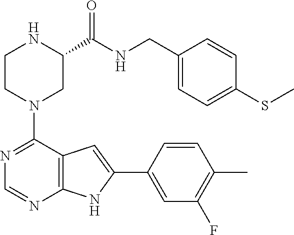 [SMILES: CSc1ccc(CNC(=O)[C@@H]2CN(c3ncnc4[nH]c(-c5ccc(C)c(F)c5)cc34)CCN2)cc1] | (S)-4-(6-(3-fluoro-4- methylphenyl)-7H- pyrrolo[2,3-d]pyrimidin-4- yl)-N-(4- (methylthio)benzyl) piperazine-2-carboxamide | 491.13 |
| 371 | 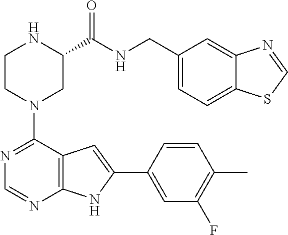 [SMILES: Cc1ccc(-c2cc3c(N4CCN[C@H](C(=O)NCc5ccc6scnc6c5)C4)ncnc3[nH]2)cc1F] | (S)-N-(benzo[d]thiazol-5- ylmethyl)-4-(6-(3-fluoro-4- methylphenyl)-7H- pyrrolo[2,3-d]pyrimidin-4- yl)piperazine-2- carboxamide | 502.10 |
| 372 | 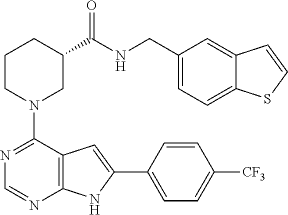 [SMILES: O=C(NCc1ccc2sccc2c1)[C@H]1CCCN(c2ncnc3[nH]c(-c4ccc(C(F)(F)F)cc4)cc23)C1] | (S)-N-(benzo[b]thiophen- 5-ylmethyl)-1-(6-(4- (trifluoromethyl)phenyl)- 7H-pyrrolo[2,3- d]pyrimidin-4- yl)piperidine-3- carboxamide | 536.13 |
| 373 | 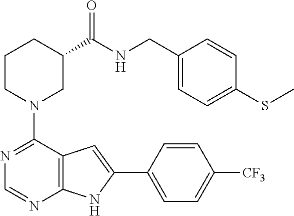 [SMILES: CSc1ccc(CNC(=O)[C@H]2CCCN(c3ncnc4[nH]c(-c5ccc(C(F)(F)F)cc5)cc34)C2)cc1] | (S)-N-(4- (methylthio)benzyl)-1-(6- (4- (trifluoromethyl)phenyl)- 7H-pyrrolo[2,3- d]pyrimidin-4- yl)piperidine-3- carboxamide | 526.17 |
| 374 | 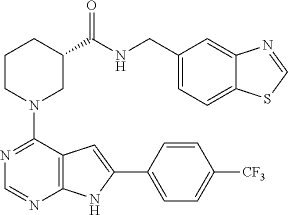 [SMILES: O=C(NCc1ccc2scnc2c1)[C@H]1CCCN(c2ncnc3[nH]c(-c4ccc(C(F)(F)F)cc4)cc23)C1] | (S)-N-(benzo[d]thiazol-5- ylmethyl)-1-(6-(4- (trifluoromethyl)phenyl)- 7H-pyrrolo[2,3- d]pyrimidin-4- yl)piperidine-3- carboxamide | 537.20 |
| 375 | 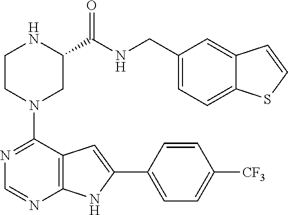 [SMILES: O=C(NCc1ccc2sccc2c1)[C@@H]1CN(c2ncnc3[nH]c(-c4ccc(C(F)(F)F)cc4)cc23)CCN1] | (S)-N-(benzo[b]thiophen- 5-ylmethyl)-4-(6-(4- (trifluoromethyl)phenyl)- 7H-pyrrolo[2,3- d]pyrimidin-4- yl)piperazine-2- carboxamide | 537.11 |
| 376 | 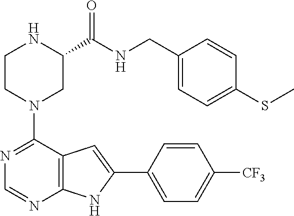 [SMILES: CSc1ccc(CNC(=O)[C@@H]2CN(c3ncnc4[nH]c(-c5ccc(C(F)(F)F)cc5)cc34)CCN2)cc1] | (S)-N-(4- (methylthio)benzyl)-4-(6- (4- (trifluoromethyl)phenyl)- 7H-pyrrolo[2,3- d]pyrimidin-4- yl)piperazine-2- carboxamide | 527.12 |
| 377 | 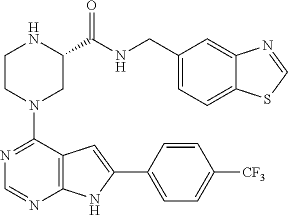 [SMILES: O=C(NCc1ccc2scnc2c1)[C@@H]1CN(c2ncnc3[nH]c(-c4ccc(C(F)(F)F)cc4)cc23)CCN1] | (S)-N-(benzo[d]thiazol-5- ylmethyl)-4-(6-(4- (trifluoromethyl)phenyl)- 7H-pyrrolo[2,3- d]pyrimidin-4- yl)piperazine-2- carboxamide | 538.17 |
| 378 | 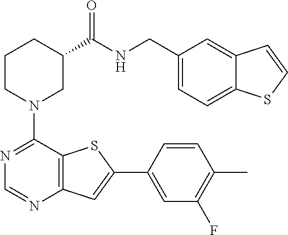 [SMILES: Cc1ccc(-c2cc3ncnc(N4CCC[C@H](C(=O)NCc5ccc6sccc6c5)C4)c3s2)cc1F] | (S)-N-(benzo[b]thiophen- 5-ylmethyl)-1-(6-(3-fluoro- 4- methylphenyl)thieno[3,2- d]pyrimidin-4- yl)piperidine-3- carboxamide | 517.19 |
| 379 | 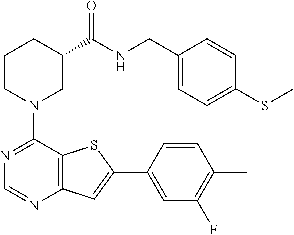 [SMILES: CSc1ccc(CNC(=O)[C@H]2CCCN(c3ncnc4cc(-c5ccc(C)c(F)c5)sc34)C2)cc1] | (S)-1-(6-(3-fluoro-4- methylphenyl)thieno[3,2- d]pyrimidin-4-yl)-N-(4- (methylthio)benzyl) piperidine-3-carboxamide | 507.11 |
| 380 | 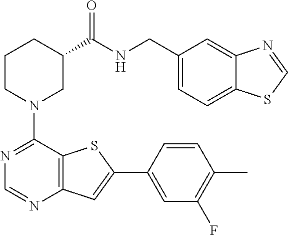 [SMILES: Cc1ccc(-c2cc3ncnc(N4CCC[C@H](C(=O)NCc5ccc6scnc6c5)C4)c3s2)cc1F] | (S)-N-(benzo[d]thiazol-5- ylmethyl)-1-(6-(3-fluoro-4- methylphenyl)thieno[3,2- d]pyrimidin-4- yl)piperidine-3- carboxamide | 518.12 |
| 381 | 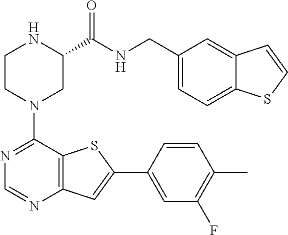 [SMILES: Cc1ccc(-c2cc3ncnc(N4CCN[C@H](C(=O)NCc5ccc6sccc6c5)C4)c3s2)cc1F] | (S)-N-(benzo[b]thiophen- 5-ylmethyl)-4-(6-(3-fluoro- 4- methylphenyl)thieno[3,2- d]pyrimidin-4- yl)piperazine-2- carboxamide | 518.10 |
| 382 | 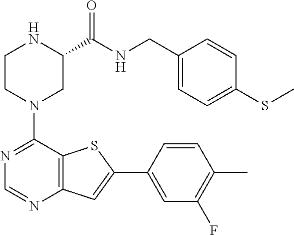 [SMILES: CSc1ccc(CNC(=O)[C@@H]2CN(c3ncnc4cc(-c5ccc(C)c(F)c5)sc34)CCN2)cc1] | (S)-4-(6-(3-fluoro-4- methylphenyl)thieno[3,2- d]pyrimidin-4-yl)-N-(4- (methylthio)benzyl) piperazine-2-carboxamide | 508.13 |
| 383 | 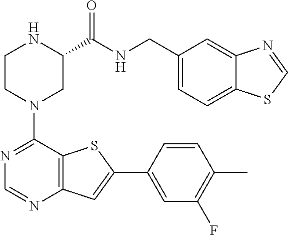 [SMILES: Cc1ccc(-c2cc3ncnc(N4CCN[C@H](C(=O)NCc5ccc6scnc6c5)C4)c3s2)cc1F] | (S)-N-(benzo[d]thiazol-5- ylmethyl)-4-(6-(3-fluoro-4- methylphenyl)thieno[3,2- d]pyrimidin-4- yl)piperazine-2- carboxamide | 519.17 |
| 384 | 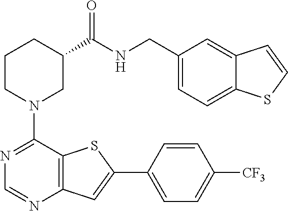 [SMILES: O=C(NCc1ccc2sccc2c1)[C@H]1CCCN(c2ncnc3cc(-c4ccc(C(F)(F)F)cc4)sc23)C1] | (S)-N-(benzo[b]thiophen- 5-ylmethyl)-1-(6-(4- (trifluoromethyl)phenyl) thieno[3,2-d]pyrimidin-4- yl)piperidine-3- carboxamide | 553.18 |
| 385 | 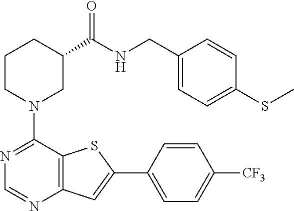 [SMILES: CSc1ccc(CNC(=O)[C@H]2CCCN(c3ncnc4cc(-c5ccc(C(F)(F)F)cc5)sc34)C2)cc1] | (S)-N-(4- (methylthio)benzyl)-1-(6- (4- (trifluoromethyl)phenyl) thieno[3,2-d]pyrimidin-4- yl)piperidine-3- carboxamide | 443.09 |
| 386 | 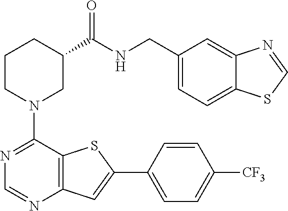 [SMILES: O=C(NCc1ccc2scnc2c1)[C@H]1CCCN(c2ncnc3cc(-c4ccc(C(F)(F)F)cc4)sc23)C1] | (S)-N-(benzo[d]thiazol-5- ylmethyl)-1-(6-(4- (trifluoromethyl)phenyl) thieno[3,2-d]pyrimidin-4- yl)piperidine-3- carboxamide | 554.18 |
| 387 | 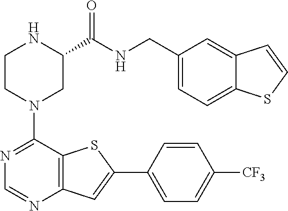 [SMILES: O=C(NCc1ccc2sccc2c1)[C@@H]1CN(c2ncnc3cc(-c4ccc(C(F)(F)F)cc4)sc23)CCN1] | (S)-N-(benzo[b]thiophen- 5-ylmethyl)-4-(6-(4- (trifluoromethyl)phenyl) thieno[3,2-d]pyrimidin-4- yl)piperazine-2- carboxamide | 554.08 |
| 388 | 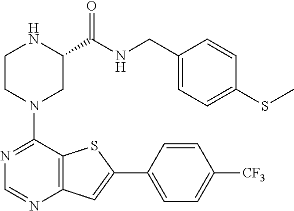 [SMILES: CSc1ccc(CNC(=O)[C@@H]2CN(c3ncnc4cc(-c5ccc(C(F)(F)F)cc5)sc34)CCN2)cc1] | (S)-N-(4- (methylthio)benzyl)-4-(6- (4- (trifluoromethyl)phenyl) thieno[3,2-d]pyrimidin-4- yl)piperazine-2- carboxamide | 544.17 |
| 389 | 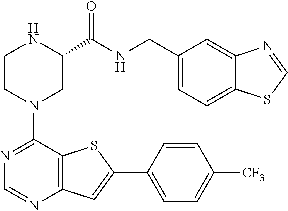 [SMILES: O=C(NCc1ccc2scnc2c1)[C@@H]1CN(c2ncnc3cc(-c4ccc(C(F)(F)F)cc4)sc23)CCN1] | (S)-N-(benzo[d]thiazol-5- ylmethyl)-4-(6-(4- (trifluoromethyl)phenyl) thieno[3,2-d]pyrimidin-4- yl)piperazine-2- carboxamide | 555.10 |
| 390 | 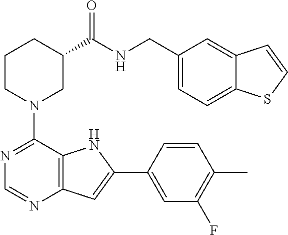 [SMILES: Cc1ccc(-c2cc3ncnc(N4CCC[C@H](C(=O)NCc5ccc6sccc6c5)C4)c3[nH]2)cc1F] | (S)-N-(benzo[b]thiophen- 5-ylmethyl)-1-(6-(3-fluoro- 4-methylphenyl)-5H- pyrrolo[3,2-d]pyrimidin-4- yl)piperidine-3- carboxamide | 500.19 |
| 391 | 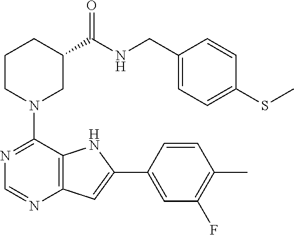 [SMILES: CSc1ccc(CNC(=O)[C@H]2CCCN(c3ncnc4cc(-c5ccc(C)c(F)c5)[nH]c34)C2)cc1] | (S)-1-(6-(3-fluoro-4- methylphenyl)-5H- pyrrolo[3,2-d]pyrimidin-4- yl)-N-(4- (methylthio)benzyl) piperidine-3-carboxamide | 490.18 |
| 392 | 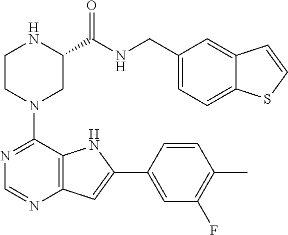 [SMILES: Cc1ccc(-c2cc3ncnc(N4CCN[C@H](C(=O)NCc5ccc6sccc6c5)C4)c3[nH]2)cc1F] | (S)-N-(benzo[b]thiophen- 5-ylmethyl)-4-(6-(3-fluoro- 4-methylphenyl)-5H- pyrrolo[3,2-d]pyrimidin-4- yl)piperazine-2- carboxamide | 501.11 |
| 393 | 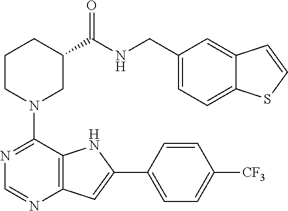 [SMILES: O=C(NCc1ccc2sccc2c1)[C@H]1CCCN(c2ncnc3cc(-c4ccc(C(F)(F)F)cc4)[nH]c23)C1] | (S)-N-(benzo[b]thiophen- 5-ylmethyl)-1-(6-(3-fluoro- 4-methylphenyl)-5H- pyrrolo[3,2-d]pyrimidin-4- yl)piperidine-3- carboxamide | 536.17 |
| 394 | 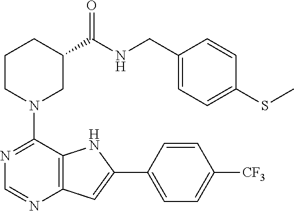 [SMILES: CSc1ccc(CNC(=O)[C@H]2CCCN(c3ncnc4cc(-c5ccc(C(F)(F)F)cc5)[nH]c34)C2)cc1] | (S)-N-(4- (methylthio)benzyl)-1-(6- (4- (trifluoromethyl)phenyl)- 5H-pyrrolo[3,2- d]pyrimidin-4- yl)piperidine-3- carboxamide | 526.13 |
| 395 | 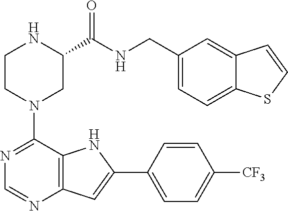 [SMILES: O=C(NCc1ccc2sccc2c1)[C@@H]1CN(c2ncnc3cc(-c4ccc(C(F)(F)F)cc4)[nH]c23)CCN1] | (S)-N-(benzo[b]thiophen- 5-ylmethyl)-4-(6-(4- (trifluoromethyl)phenyl)- 5H-pyrrolo[3,2- d]pyrimidin-4- yl)piperazine-2- carboxamide | 537.11 |
| 396 | 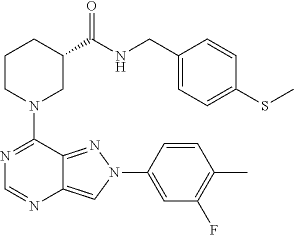 | (S)-1-(2-(3-fluoro-4- methylphenyl)-2H- pyrazolo[4,3-d]pyrimidin- 7-yl)-N-(4- (methylthio)benzyl) piperidine-3-carboxamide | 491.20 |
| 397 | 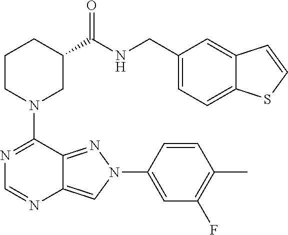 | (S)-N-(benzo[b]thiophen- 5-ylmethyl)-1-(2-(3-fluoro- 4-methylphenyl)-2H- pyrazolo[4,3-d]pyrimidin- 7-yl)piperidine-3- carboxamide | 501.11 |
| 398 | 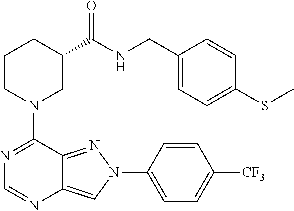 [SMILES: CSc1ccc(CNC(=O)[C@H]2CCCN(c3ncnc4cn(-c5ccc(C(F)(F)F)cc5)nc34)C2)cc1] | (S)-N-(4- (methylthio)benzyl)-1-(2- (4- (trifluoromethyl)phenyl)- 2H-pyrazolo[4,3- d]pyrimidin-7- yl)piperidine-3- carboxamide | 527.10 |
| 399 | 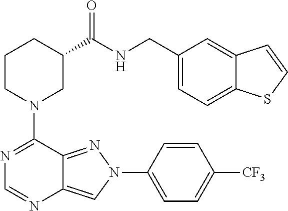 | (S)-N-(benzo[b]thiophen- 5-ylmethyl)-1-(2-(4- (trifluoromethyl)phenyl)- 2H-pyrazolo[4,3- d]pyrimidin-7- yl)piperidine-3- carboxamide | 537.12 |
| 400 | 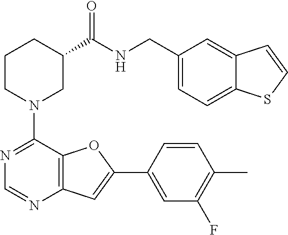 [SMILES: Cc1ccc(-c2cc3ncnc(N4CCC[C@H](C(=O)NCc5ccc6sccc6c5)C4)c3o2)cc1F] | (S)-N-(benzo[b]thiophen- 5-ylmethyl)-1-(6-(3-fluoro- 4-methylphenyl)furo[3,2- d]pyrimidin-4- yl)piperidine-3- carboxamide | 501.10 |
| 401 | 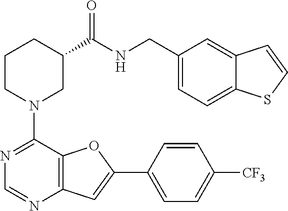 [SMILES: O=C(NCc1ccc2sccc2c1)[C@H]1CCCN(c2ncnc3cc(-c4ccc(C(F)(F)F)cc4)oc23)C1] | (S)-N-(benzo[b]thiophen- 5-ylmethyl)-1-(6-(4- (trifluoromethyl)phenyl) furo[3,2-d]pyrimidin-4- yl)piperidine-3- carboxamide | 537.11 |
| 402 | 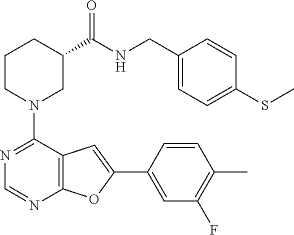 [SMILES: CSc1ccc(CNC(=O)[C@H]2CCCN(c3ncnc4oc(-c5ccc(C)c(F)c5)cc34)C2)cc1] | (S)-1-(6-(3-fluoro-4- methylphenyl)furo[2,3- d]pyrimidin-4-yl)-N-(4- (methylthio)benzyl) piperidine-3-carboxamide | 491.12 |
| 403 | 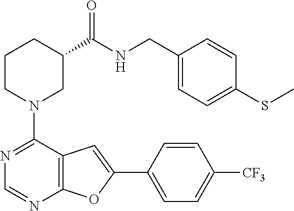 [SMILES: CSc1ccc(CNC(=O)[C@H]2CCCN(c3ncnc4oc(-c5ccc(C(F)(F)F)cc5)cc34)C2)cc1] | (S)-N-(4- (methylthio)benzyl)-1-(6- (4- (trifluoromethyl)phenyl) furo[2,3-d]pyrimidin-4- yl)piperidine-3- carboxamide | 527.11 |

The compounds described herein can be made by using techniques and methods generally known in the art. Exemplary methods for producing the compounds described herein, as well as characterization information, are provided in the Examples.

### Therapeutic Applications

Methods for Treatment of Neurological Disorders. Metabolic Disorders, or Diabetes

In one aspect, disclosed herein is a method for the treatment of a neurological disorder, disorder, or condition in a subject, the method including the step of administering to the subject a therapeutically effective amount of at least one disclosed compound, or a pharmaceutically acceptable salt thereof, or the disclosed pharmaceutical composition. In some aspects, the subject is a human. In another aspect, the subject has been diagnosed with a need for treatment of the neurological disorder, disorder, or condition prior to the administering step. In some aspects, the method further includes the step of identifying a subject in need of treatment of the neurological disorder, disorder, or condition.

In one aspect, the disease, disorder, or condition is Parkinson's disease, Alzheimer's disease, Down syndrome, spinocerebellar ataxia, amyotrophic lateral sclerosis, Huntington's disease, stroke, radiation therapy, chronic stress, abuse of a neuro-active drug, retinal degeneration, spinal cord injury, peripheral nerve injury chemotherapeutic agent mediated cardiotoxicity, schizophrenia, major depression, bipolar disorder, normal aging, epilepsy, traumatic brain injury and/or a visual symptom associated therewith, post-traumatic stress disorder, physiological weight loss associated with various conditions, cognitive decline and/or general frailty associated with normal aging and/or chemotherapy, chemotherapy induced neuropathy, concussive injury, crush injury, peripheral neuropathy, diabetic neuropathy, post-traumatic headache, multiple sclerosis, retinal degeneration and dystrophy (such as Leber congenital amaurosis, retinitis pigmentosa, cone-rod dystrophy, microphthalmia, anophthalmia, myopia, and hyperopia), spinal cord injury, traumatic spinal cord injury, peripheral nerve injury (such as peripheral nerve crush injury, neonatal brachial plexus palsy, and traumatic facial nerve palsy), retinal neuronal death related diseases (such as glaucoma and age related macular degeneration, diabetic retinopathy, retinal blood vessel occlusions, retinal medication toxicity (such as what amino glycosides or plaquenil can cause), retinal trauma (e.g., post-surgical), retinal infections, choroidal dystrophies, retinal pigmentary retinopathies, inflammatory and cancer mediated auto immune diseases that result in retinal neuronal cell death), Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy (olivopontocerebellar atrophy), Hereditary spastic paraparesis, Friedreich ataxia, Non-Friedreich ataxia, Spinocerebellar atrophies, Amyloidoses, Metabolic-related (e.g., Diabetes) neurodegenerative disorders, Toxin-related neurodegenerative disorders, Multiple sclerosis, Charcot Marie Tooth, Diabetic neuropathy, Metabolic neuropathies, Endocrine neuropathies, Orthostatic hypotension, Creutzfeldt-Jacob Disease, Primary progressive aphasia, Frontotemporal Lobar Degeneration, Cortical blindness, Shy-Drager Syndrome (Multiple, System Atrophy with Orthostatic Hypotension), Diffuse cerebral cortical atrophy of non-Alzheimer type, Lewy-body dementia, Pick disease (lobar atrophy), Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, and Tourette syndrome.

In some aspects, the compound described herein can enhance the cognitive performance of a subject in need thereof. In some aspects, the compounds can treat or prevent a metabolic disorder. In another aspect, the compounds can treat or prevent a prevent obesity or an insulin resistance disorder, such as diabetes in a subject. In some aspects, the compound is a bioenergetic agent for restoring the quality of aged cells.

In some aspects, the free compound or pharmaceutical formulation thereof (discussed in greater detail elsewhere herein) can have an IC₅₀ that is less than 0.001, less than 0.01, less than 0.1, less than 1 μM, less than 3 μM, and/or less than 5 μM. In some aspects, the free compound or pharmaceutical formulation thereof (discussed in greater detail elsewhere herein) can have an IC₅₀ ranging from 0.0001 μM to 0.001 μM, from 0.001 to 0.01 μM, from 0.01 μM to 0.1 μM, 0.1 μM to 1 μM, 1 μM to 2 μM, 2 μM to 3 μM, 3 μM to 4 μM, or 4 μM to 5 μM. In some aspects, the free compound or pharmaceutical formulation thereof (discussed in greater detail elsewhere herein) can have an EC₅₀ for activating NAMPT ranging from 0.0001 μM to 0.001 μM, from 0.001 to 0.01 μM, from 0.01 μM to 0.1 μM, 0.1 μM to 1 μM, 1 μM to 2 μM, 2 μM to 3 μM, 3 μM to 4 μM, or 4 μM to 5 μM. In other aspects, the compounds described herein can increase the production of NAD⁺. In one aspect, the compounds described herein can increase the production of NAD⁺ by at least 2-fold or more, at least 3-fold, at least 4-fold, or at least 5-fold.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethylcellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In one aspect, an appropriate dosage level will generally be about 0.01 to 1000 mg of a compound described herein per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

### Kits

In a further aspect, the present disclosure relates to kits comprising (a) at least one disclosed compound, or a pharmaceutically acceptable salt, hydrate, solvate, or polymorph thereof and one of and (b) instructions for treating a neurological disease, disorder, or condition.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A compound of Formula I

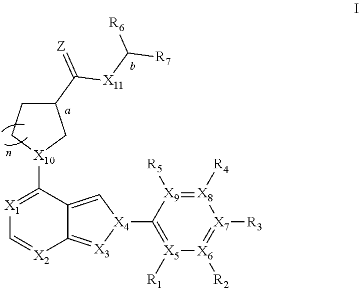

wherein X₁, X₂, X₃, and X₄ are N or CH;
X₅, X₆, X₇, X₈, and X₉ are N or C, wherein when X₅, X₆, X₇, X₈, or X₉ is N, R₁, R₂, R₃, R₄, R₅ and R₆ are not present;
X₁₀ is N or C(Y₁)₂;
X₁₁ is NY₁ or C(Y₁)₂;
Y₁ is independently selected from the group consisting of hydrogen, alkyl, alkoxy, haloalkyl, hydroxyalkyl, OH, CN, F, Cl, Br, and I;
Z is O or S;
R₁, R₂, R₃, R₄, R₅ and R₆ are each independently selected from —H, —OH, a halogen, —CN, alkyl, alkynyl, C3-C6 cycloalkyl, a C1-C3 haloalkyl, and —C(O)—O-alkyl;
R₇ is independently selected from:

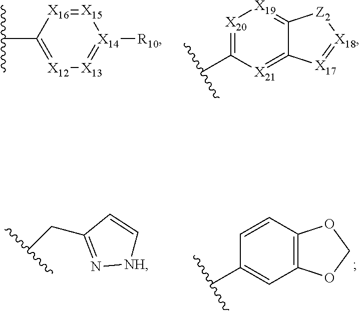

X₁₂, X₁₃, X₁₅, X₁₆, X₁₇, X₁₈, X₁₉, X₂₀, and X₂₁ are N or CY₂;
X₁₄ is N or C, wherein when X₁₄ is N, R₁₀ is not present;
Y₂ is selected from hydrogen, alkyl, alkoxy, haloalkyl, hydroxyalkyl, OH, CN, F, Cl, Br, and I;
Z₂ is S, NY₃ or O;
Y₃ is hydrogen or alky;
R₁₀ is independently selected from: hydrogen, alkyl, alkoxy, haloalkyl, hydroxyalkyl, OH, CN, F, Cl, Br, I, N(alkyl)₂, S-alkyl, S(O)-alkyl, and S(O)₂-alkyl;
n is an integer from 0 to 3; and
the stereochemistry at carbon a and carbon b is, independently, substantially R, substantially S, or racemic; or
a pharmaceutically acceptable salt thereof.

Aspect 2. The compound of Aspect 1, wherein X₁, X₂, X₃, X₄, and X₁₁ are each N.

Aspect 3. The compound of Aspect 1 or 2, wherein Z is O and X₁₁ is NH.

Aspect 4. The compound of any one of Aspects 1-3, wherein n is 2.

Aspect 5. The compound of any one of Aspects 1-4, wherein X₅, X₆, X₇, X₈, and X₉ are each C.

Aspect 6. The compound of any one of Aspects 1-5, wherein R₃ is a halogen, alkyl, or C1-C3 haloalkyl.

Aspect 7. The compound of any one of Aspects 1-6, wherein R₆ is hydrogen.

Aspect 8. The compound of any one of Aspects 1-7, wherein R₇ is

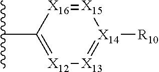

Aspect 9. The compound of Aspect 8, wherein X₁₂, X₁₃, X₁₅, and X₁₆, are each CY₂ and X₁₄ is C.

Aspect 10. The compound of Aspect 8 or 9, wherein R₁₀ is F, Cl, Br, I or S-alkyl.

Aspect 11. The compound of any one of Aspects 1-7, wherein R₇ is

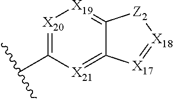

Aspect 12. The compound of Aspect 11, wherein Z₂ is S and each of X₁₇, X₁₈, X₁₉, X₂₀, and X₂₁ are CY₂.

Aspect 13. The compound of Aspect 1, wherein the compound is Formula II

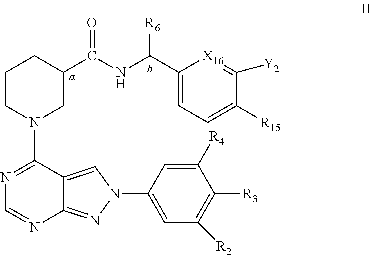

wherein X₁₆ is N or CH;
R₂, R₃, R₄, R₆, and Y₂ are independently selected from —H, —OH a halogen, —CN, alkynyl,
C3-C6 cycloalkyl and C1-C3 haloalkyl; and
R₁₅ is selected from -SMe, -SEt, —I, —Br.

Aspect 14. The compound of Aspect 1, wherein the compound is Formula III

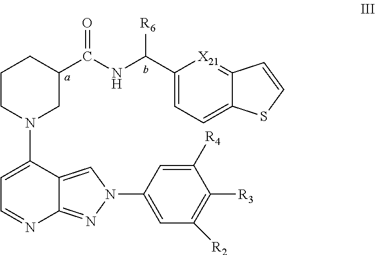

wherein X₂₁ is N or CH; and
R₂, R₃, R₄, and R₆ are independently selected from —H, —OH a halogen, —CN, alkynyl,
C3-C6 cycloalkyl and C1-C3 haloalkyl.

Aspect 15. The compound of any one of Aspects 1-14, wherein R₆ is hydrogen and the stereochemistry at carbon a is substantially R or substantially S.

Aspect 16. A compound of Formula IV or V

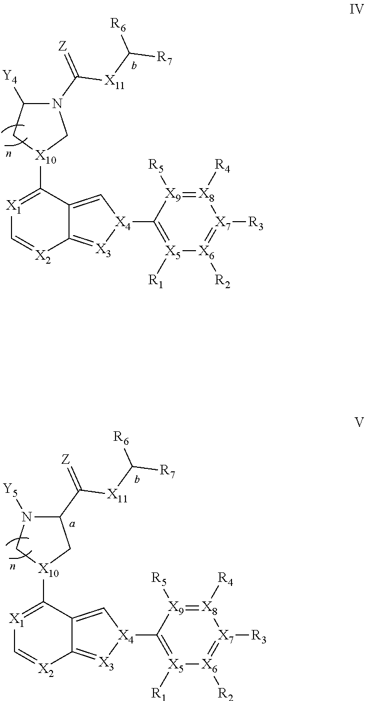

wherein X₁, X₂, X₃, and X₄ are N or CH;
X₅, X₆, X₇, X₈, and X₉ are N or C, wherein when X₅, X₆, X₇, X₈, or X₉ is N, R₁, R₂, R₃, R₄, R₅ and R₆ are not present;
X₁₀ is N or C(Y₁)₂;
X₁₁ is NY₁ or C(Y₁)₂;
Y₁, Y₄, and Y₅ are independently selected from the group consisting of hydrogen, alkyl, alkoxy, haloalkyl, hydroxyalkyl, OH, CN, F, Cl, Br, and I;
Z is O or S;
R₁, R₂, R₃, R₄, R₅ and R₆ are each independently selected from —H, —OH, a halogen, —CN, alkyl, alkynyl, C3-C6 cycloalkyl, a C1-C3 haloalkyl, and —C(O)—O-alkyl;
R₇ is independently selected from:

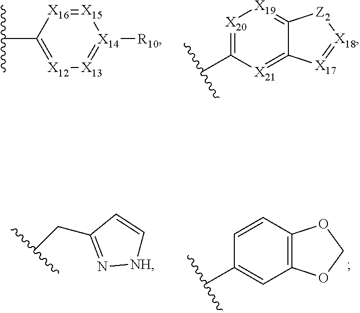

X₁₂, X₁₃, X₁₅, X₁₆, X₁₇, X₁₈, X₁₉, X₂₀, and X₂₁ are N or CY₂;
X₁₄ is N or C, wherein when X₁₄ is N, R₁₀ is not present;
Y₂ is selected from hydrogen, alkyl, alkoxy, haloalkyl, hydroxyalkyl, OH, CN, F, Cl, Br, and I;
Z₂ is S, NY₃ or O;
Y₃ is hydrogen or alkyl;
R₁₀ is independently selected from hydrogen, alkyl, alkoxy, haloalkyl, hydroxyalkyl, OH, CN, F, Cl, Br, I, N(alkyl)₂, S-alkyl, S(O)-alkyl, and S(O)₂-alkyl;
n is an integer from 0 to 3; and
the stereochemistry at carbon a and carbon b is, independently, substantially R, substantially S, or racemic; or
a pharmaceutically acceptable salt thereof.

Aspect 17. The compound of Aspect 16, wherein n is 2, X₁, X₂, X₃, X₄, and X₁₀ are each N, Z is O, and X₁₁ is NH.

Aspect 18. A method for activating NAMPT in a subject, the method comprising administering to the subject the compound of any one of Aspects 1-17.

Aspect 19. A method for increasing NAD⁺ in a subject, the method comprising administering to the subject the compound of any one of Aspects 1-17.

Aspect 20. A method for treating a neurological disorder, diabetes, or a metabolic disorder in a subject, the method comprising administering to the subject the compound of any one of Aspects 1-17.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### General Synthetic Scheme

The compounds described herein, including compounds of general formula (I) and specific examples, can be prepared by methodologies in the reaction schemes depicted in schemes 1-8.

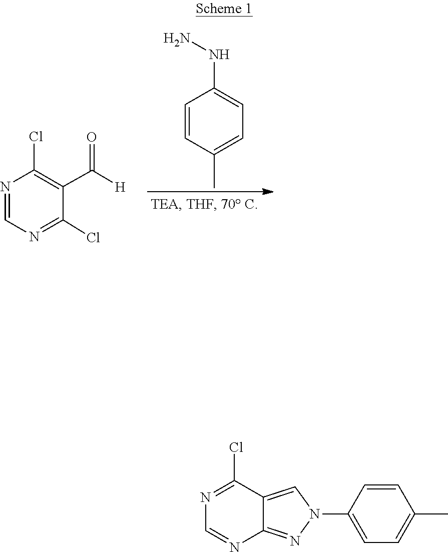

To a 100 mL flask was added 4,6-dichloropyrimidine-5-carboxaldehyde (500 mg, 2.83 mmol), p-tolylhydrazine hydrochloride (449 mg, 2.83 mmol), THF (20 mL) and triethylamine (1.2 mL, 8.49 mmol). The reaction was stirred under a nitrogen atmosphere, at 0° C. for 30 min and then heated to 65° C. for 50 min. The solvent was removed under vacuum and the crude product was purified by flash chromatography on silica to give the product 240 mg (35%).

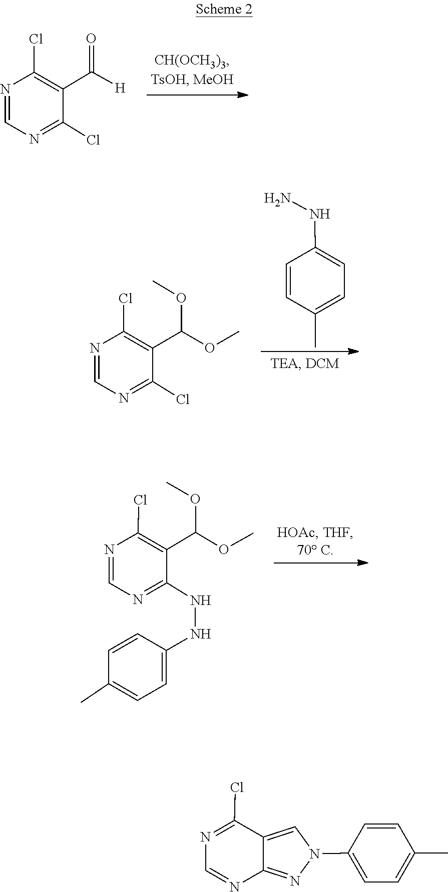

To a solution of 4,6-dichloropyrimidine-5-carboxaldehyde (500 mg, 2.83 mmol), CH(OCH₃)₃(464 μL, 4.25 mmol) in MeOH (10 mL) was added TsOH·H₂O (53 mg, 0.28 mmol) and stirred at room temperature for 1 h. Upon completion, the reaction mass was quenched with TEA (1 mL), diluted with EA (100 mL) and washed with water (100 mL) and brine (100 mL). The organic layer was dried over Na₂SO₄ and concentrated in vacuo to give the product without further purification.

4,6-dichloro-5-(dimethoxymethyl)pyrimidine (2.83 mmol) which was generated from precious step and p-tolylhydrazine hydrochloride (538 mg, 3.40 mmol) was dissolved in TEA/DCM (1 mL/4 mL). The reaction was stirred at room temperature for 2 h and then remove the solvent under vacuum to give the crude compound. The crude product was purified by flash chromatography (ethyl acetate/hexane gradient) to give the yellow solid (795 mg, yield 91% for 2 steps).

To a solution of 4-chloro-5-(dimethoxymethyl)-6-(2-(p-tolyl)hydrazineyl)pyrimidine (500 mg, 1.62 mmol) in THF (5 mL) was added HOAc (1 mL). The reaction was stirred at room temperature for half an hour and then warm up to 70° C. for another 2 h. After the reaction completed, the solvent was removed under vacuum and the crude product was purified by flash chromatography on silica to give the product 166 mg (yield 42%)

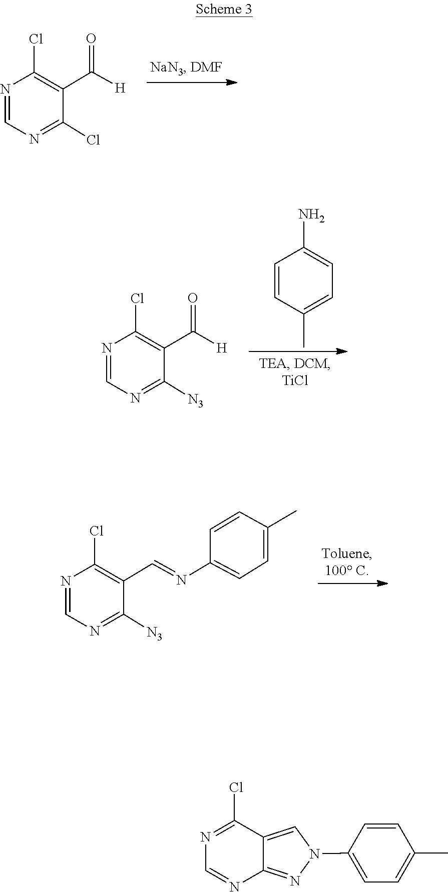

A solution of 4,6-dichloropyrimidine-5-carboxaldehyde (500 mg, 2.83 mmol) in dimethylformamide (5 mL) was combined with a small excess of sodium azide (188 mg, 2.90 mmol) and stirred at room temperature for 2 h. Then the reaction mixture was poured into ice/water (50 mL), the obtained precipitate filtered by suction and purified by flash chromatography on silica to give the product 447 mg (yield 82%)

To a solution of 4-azido-6-chloropyrimidine-5-carbaldehyde (200 mg, 1.09 mmol), p-toluidine (129 mg, 1.20 mmol) and TEA (456 μL, 3.27 mmol) in DCM (5 mL) was slowly added titanium tetrachloride (1 M in DCM, 650 μL, 0.65 mmol). The reaction was stirred at room temperature for 18 h, and then removed the solvent. The residue was diluted with EA (100 mL) and washed with water (100 mL) and brine (100 mL). The organic layer was dried over Na₂SO₄ and concentrated in vacuo to give the product which can be directly used for the next step without further purification.

(E)-1-(4-azido-6-chloropyrimidin-5-yl)-N-(p-tolyl)methanimine (1.09 mmol) which was generated from previous step was dissolved in toluene (5 mL) and heated to 100° C. for 10 h. Upon the reaction finished, the solvent was removed under high vacuum and the residue was purified by flash chromatography on silica to give the product 164 mg (yield 67% for 2 steps)

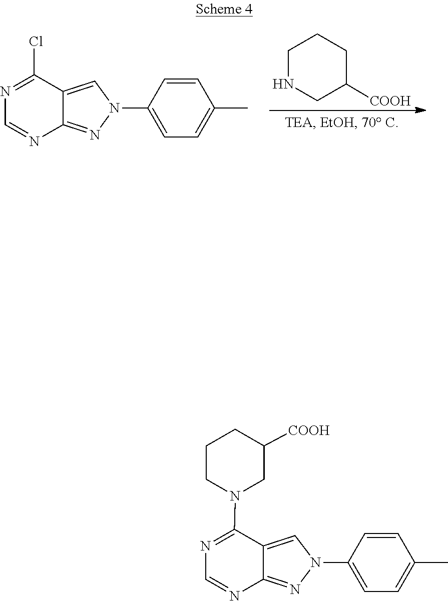 [SMILES: Cc1ccc(-n2cc3c(Cl)ncnc3n2)cc1 ; O=C(O)C1CCCNC1 ; Cc1ccc(-n2cc3c(N4CCCC(C(=O)O)C4)ncnc3n2)cc1]

A solution of 4-chloro-2-(p-tolyl)-2H-pyrazolo[3,4-d]pyrimidine (100 mg, 0.41 mmol) and piperidine-3-carboxylic acid (64 mg, 0.49 mmol) in TEA/EtOH (0.5 mL/2 mL) was stirred at 70° C. for 1 h. After removing the solvent, the residue was purified by flash chromatography on silica to give the product 128 mg (yield 92%)

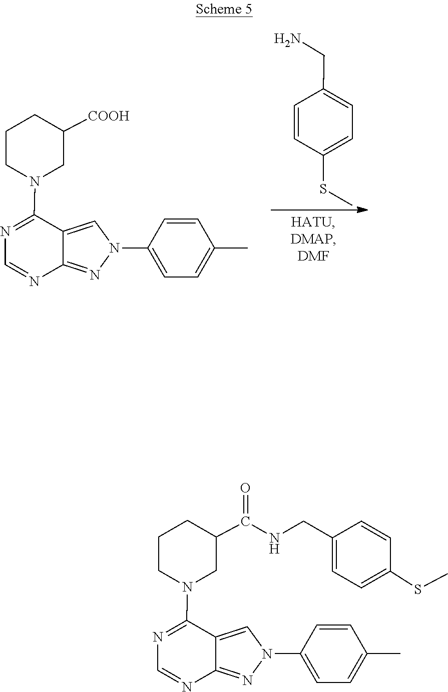

A solution of (50 mg, 0.15 mmol), 4-(methylsulfanyl)benzylamine (34 mg, 0.23 mmol), HATU (87 mg, 0.23 mmol) and DMAP (73 mg, 0.6 mmol) in DMF (2 mL) was stirred at room temperature for 2 h. After the reaction finished, the complex was diluted with EA (50 mL) and washed with water (50 mL) and brine (50 mL). The organic layer was dried over Na₂SO₄ and concentrated in vacuo. The crude compound was purified with prep-HPLC to give the product 98 mg (yield 90%, purity>98%)

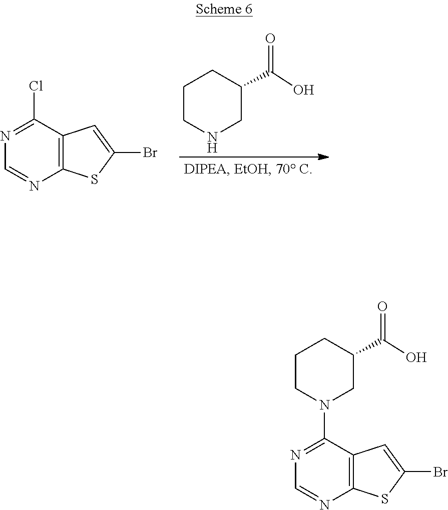 [SMILES: Clc1ncnc2sc(Br)cc12 ; O=C(O)[C@H]1CCCNC1 ; O=C(O)[C@H]1CCCN(c2ncnc3sc(Br)cc23)C1]

A solution of 6-bromo-4-chlorothieno[2,3-d]pyrimidine (100 mg, 0.40 mmol) and (S)-piperidine-3-carboxylic acid (78 mg, 0.60 mmol) in DIPEA/EtOH (0.5 mL/2 mL) was stirred at 70° C. for 1 h. After removing the solvent, the residue was purified by flash chromatography on silica to give the product 132 mg (yield 97%)

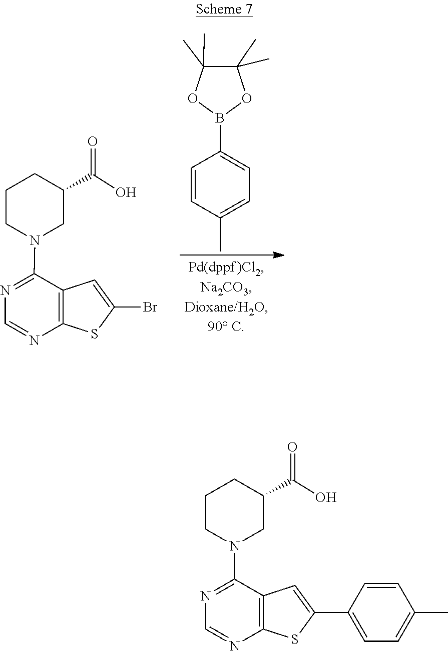

A flask fitted with a rubber septum was charged with (S)-1-(6-bromothieno[2,3-d]pyrimidin-4-yl)piperidine-3-carboxylic acid (34 mg, 0.10 mmol), 4,4,5,5-tetramethyl-2-(p-tolyl)-1,3,2-dioxaborolane (33 mg, 0.15 mmol), Pd(dppf)Cl₂ (8 mg, 0.01 mmol), Na₂CO₃ (32 mg, 0.3 mmol), Dioxane/H₂O (2 mL/0.2 mL) and then purged with argon. The mixture was stirred at 95° C. overnight. The reaction mixture was then cooled to room temperature, diluted with ethyl acetate (20 mL), filtered through celite and concentrated in vacuo. The purification by Prep-HPLC afforded the product 29 mg (yield 83%)

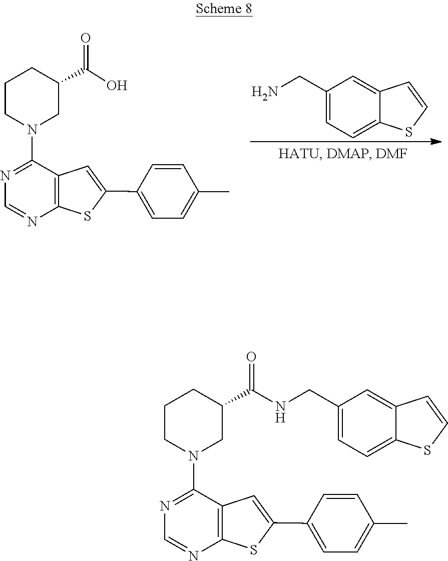 [SMILES: Cc1ccc(-c2cc3c(N4CCC[C@H](C(=O)O)C4)ncnc3s2)cc1 ; NCc1ccc2sccc2c1 ; Cc1ccc(-c2cc3c(N4CCC[C@H](C(=O)NCc5ccc6sccc6c5)C4)ncnc3s2)cc1]

A solution of (35 mg, 0.10 mmol), 4-(methylsulfanyl)benzylamine (24 mg, 0.15 mmol), HATU (46 mg, 0.12 mmol) and DMAP (37 mg, 0.30 mmol) in DMF (2 mL) was stirred at room temperature for 2 h. After the reaction finished, the complex was diluted with EA (50 mL) and washed with water (50 mL) and brine (50 mL). The organic layer was dried over Na₂SO₄ and concentrated in vacuo. The crude compound was purified with prep-HPLC to give the product 45 mg (yield 90%, purity>98%)

### Exemplary Compounds

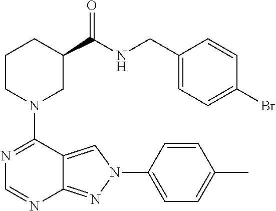 [SMILES: Cc1ccc(-n2cc3c(N4CCC[C@@H](C(=O)NCc5ccc(Br)cc5)C4)ncnc3n2)cc1]

(R)—N-(4-bromobenzyl)-1-(2-(p-tolyl)-2H-pyrazolo[3,4-d]pyrimidin-4-yl)piperidine-3-carboxamide. White solid (yield 82%): [α]_{D}²⁵=−98.4 (c 0.5, MeOH); ¹H NMR (400 MHz, DMSO-d₆) δ 9.27 (s, 1H), 8.55-8.47 (m, 1H), 8.27 (s, 1H), 8.14 (s, 1H), 8.01-7.92 (m, 2H), 7.55-7.45 (m, 1H), 7.44-7.36 (m, 2H), 7.27-7.10 (m, 2H), 5.06-4.45 (m, 2H), 4.31-4.19 (m, 2H), 3.23-3.02 (m, 2H), 2.47 (s, 1H), 2.39 (s, 3H), 1.97-1.76 (m, 3H), 1.71-1.48 (m, 1H); ¹³C NMR (100 MHz, DMSO-d₆) δ 172.58, 163.06, 160.86, 157.45, 155.84, 138.98, 137.73, 137.12, 131.15, 130.00, 129.42, 129.32, 123.39, 120.11, 119.75, 102.25, 47.68, 46.80, 42.18, 41.33, 27.62, 24.44, 20.58; HRMS (ESI) calcd for C₂₅H₂₆BrN₆O [M+H]⁺ 505.1346, found 505.1353.

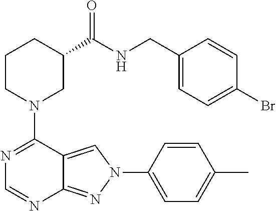 [SMILES: Cc1ccc(-n2cc3c(N4CCC[C@H](C(=O)NCc5ccc(Br)cc5)C4)ncnc3n2)cc1]

(S)—N-(4-bromobenzyl)-1-(2-(p-tolyl)-2H-pyrazolo[3,4-d]pyrimidin-4-yl)piperidine-3-carboxamide. White solid (yield 78%): [α]_{D}²⁵=+90.4 (c 0.7, MeOH); ¹H NMR (400 MHz, DMSO-d₆) δ 9.27 (s, 1H), 8.50 (t, J=6.0 Hz, 1H), 8.27 (s, 1H), 8.14 (s, 1H), 8.03-7.92 (m, 2H), 7.55-7.44 (m, 1H), 7.40 (d, J=8.4 Hz, 2H), 7.21 (d, J=8.4 Hz, 2H), 5.03-4.50 (m, 2H), 4.29-4.19 (m, 2H), 3.21-3.01 (m, 2H), 2.48-2.45 (m, 1H), 2.39 (s, 3H), 1.98-1.78 (m, 3H), 1.62-1.48 (m, 1H); ¹³C NMR (100 MHz, DMSO-d₆) δ 172.60, 163.06, 160.86, 157.45, 155.84, 138.83, 137.73, 137.19, 131.15, 130.00, 129.42, 129.32, 123.39, 120.11, 119.75, 102.25, 47.78, 46.55, 42.18, 41.33, 27.62, 24.44, 20.59; HRMS (ESI) calcd for C₂₅H₂₆BrN₆O [M+H]⁺ 505.1346, found 505.1353.

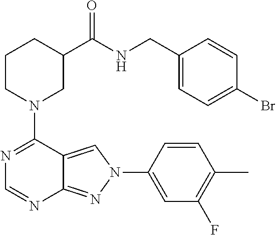 [SMILES: Cc1ccc(-n2cc3c(N4CCCC(C(=O)NCc5ccc(Br)cc5)C4)ncnc3n2)cc1F]

N-(4-bromobenzyl)-1-(2-(3-fluoro-4-methylphenyl)-2H-pyrazolo[3,4-d]pyrimidin-4-yl)piperidine-3-carboxamide. Light brown solid (yield 78%): ¹H NMR (400 MHz, DMSO-d₆) δ 9.34 (s, 1H), 8.56-8.45 (m, 1H), 8.29 (s, 1H), 7.97 (dd, J=11.0, 2.2 Hz, 1H), 7.87 (dd, J=8.3, 2.3 Hz, 1H), 7.56-7.42 (m, 3H), 7.27-7.17 (m, 2H), 5.12-4.40 (m, 2H), 4.34-4.18 (m, 2H), 3.30-3.05 (m, 2H), 2.48 (s, 1H), 2.32 (s, 3H), 2.02-1.78 (m, 3H), 1.63-1.48 (m, 1H); ¹³C NMR (100 MHz, DMSO-d₆) δ 172.56, 160.70, 160.69 (d, J=243.3 Hz), 156.74 (d, J=135.9 Hz), 138.95, 138.46 (d, J=10.2 Hz), 132.43 (d, J=5.7 Hz), 131.12, 129.42, 124.14 (d, J=17.3 Hz), 123.91, 119.76, 115.74, 107.12 (d, J=27.8 Hz), 102.45, 46.95, 46.15, 42.11, 41.35, 27.62, 24.34, 13.87; HRMS (ESI) calcd for C₂₅H₂₅BrFN₆O [M+H]⁺ 523.1252, found 523.1258.

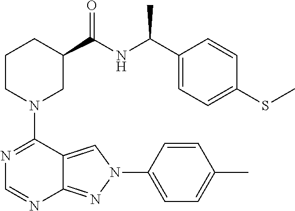 [SMILES: CSc1ccc([C@H](C)NC(=O)[C@@H]2CCCN(c3ncnc4nn(-c5ccc(C)cc5)cc34)C2)cc1]

(R)—N—((S)-1-(4-(methylthio)phenyl)ethyl)-1-(2-(p-tolyl)-2H-pyrazolo[3,4-d]pyrimidin-4-yl)piperidine-3-carboxamide. White solid (yield 87%): [α]_{D}²⁵=−91.2 (c 0.3, MeOH); ¹H NMR (400 MHz, DMSO-d₆) δ 9.30 (s, 1H), 8.39 (d, J=8.0 Hz, 1H), 8.28 (s, 1H), 8.07-7.96 (m, 2H), 7.40 (d, J=8.3 Hz, 2H), 7.26-7.16 (m, 4H), 4.88 (p, J=7.1 Hz, 1H), 4.62-4.41 (m, 2H), 3.27-3.02 (m, 2H), 2.47 (s, 1H), 2.44 (s, 3H), 2.39 (s, 3H), 1.93-1.67 (m, 3H), 1.58-1.45 (m, 1H), 1.32 (d, J=7.1 Hz, 3H); ¹³C NMR (100 MHz, DMSO-d₆) δ 171.59, 160.87, 157.43, 155.88, 141.67, 137.73, 137.12, 135.97, 130.00, 126.50, 126.12, 123.43, 120.09, 102.24, 47.23, 46.89, 46.42, 42.01, 27.71, 24.33, 22.43, 20.57, 14.96; HRMS (ESI) calcd for C₂₇H₃₁N₆OS [M+H]⁺ 487.2275, found 487.2277.

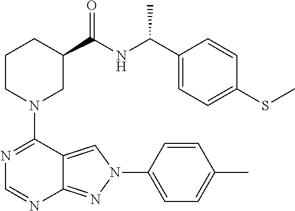 [SMILES: CSc1ccc([C@@H](C)NC(=O)[C@@H]2CCCN(c3ncnc4nn(-c5ccc(C)cc5)cc34)C2)cc1]

(R)—N—((R)-1-(4-(methylthio)phenyl)ethyl)-1-(2-(p-tolyl)-2H-pyrazolo[3,4-d]pyrimidin-4-yl)piperidine-3-carboxamide. White solid (yield 84%): [α]_{D}²⁵=−32.0 (c 0.4, MeOH); ¹H NMR (400 MHz, DMSO-d₆) δ 9.29 (s, 1H), 8.37 (d, J=8.0 Hz, 1H), 8.27 (s, 1H), 7.97 (d, J=8.1 Hz, 2H), 7.40 (d, J=8.3 Hz, 2H), 7.28-7.21 (m, 2H), 7.20-7.11 (m, 2H), 4.89 (p, J=7.1 Hz, 1H), 4.70-4.31 (m, 2H), 3.46-3.33 (m, 2H), 2.48-2.47 (m, 1H), 2.44 (s, 3H), 2.40 (s, 3H), 1.88 (d, J=25.1 Hz, 3H), 1.53 (d, J=12.9 Hz, 1H), 1.31 (d, J=7.0 Hz, 3H); ¹³C NMR (100 MHz, DMSO-d₆) δ 171.56, 160.87, 157.44, 155.87, 142.36, 137.73, 137.11, 136.05, 130.01, 126.65, 126.04, 123.38, 120.08, 102.25, 47.11, 46.91, 46.25, 42.13, 27.53, 24.59, 22.20, 20.58, 14.91; HRMS (ESI) calcd for C₂₇H₃₁N₆OS [M+H]⁺ 487.2275, found 487.2280.

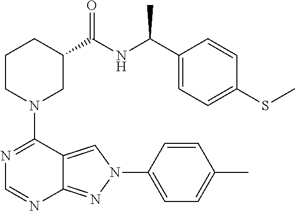 [SMILES: CSc1ccc([C@H](C)NC(=O)[C@H]2CCCN(c3ncnc4nn(-c5ccc(C)cc5)cc34)C2)cc1]

(S)—N—((S)-1-(4-(methylthio)phenyl)ethyl)-1-(2-(p-tolyl)-2H-pyrazolo[3,4-d]pyrimidin-4-yl)piperidine-3-carboxamide. White solid (yield 91%): [α]_{D}²⁵=+29.1 (c 0.3, MeOH); ¹H NMR (400 MHz, DMSO-d₆) δ 9.29 (s, 1H), 8.38 (d, J=8.0 Hz, 1H), 8.27 (s, 1H), 7.97 (d, J=8.1 Hz, 2H), 7.40 (d, J=8.3 Hz, 2H), 7.28-7.10 (m, 4H), 4.98-4.83 (m, 1H), 4.79-4.33 (m, 2H), 3.30-3.12 (m, 2H), 2.48-2.46 (m, 1H), 2.44 (s, 3H), 2.40 (s, 3H), 1.96-1.77 (m, 3H), 1.61-1.46 (m, 1H), 1.31 (d, J=7.0 Hz, 3H). ¹³C NMR (100 MHz, DMSO-d₆) δ 171.57, 160.89, 157.45, 155.89, 141.33, 137.74, 137.13, 136.06, 130.03, 126.67, 126.05, 123.40, 120.09, 102.26, 47.08, 46.48, 45.93, 41.98, 27.55, 24.16, 22.21, 20.60, 14.92; HRMS (ESI) calcd for C₂₇H₃₁N₆OS [M+H]⁺ 487.2275, found 487.2280.

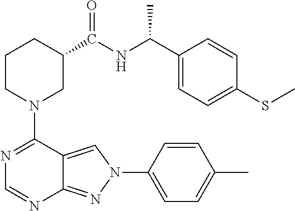 [SMILES: CSc1ccc([C@@H](C)NC(=O)[C@H]2CCCN(c3ncnc4nn(-c5ccc(C)cc5)cc34)C2)cc1]

(S)—N—((R)-1-(4-(methylthio)phenyl)ethyl)-1-(2-(p-tolyl)-2H-pyrazolo[3,4-d]pyrimidin-4-yl)piperidine-3-carboxamide. White solid (yield 88%): [α]_{D}²⁵=+87.7 (c 0.4, MeOH); ¹H NMR (400 MHz, DMSO-d₆) δ 9.30 (s, 1H), 8.38 (d, J=8.0 Hz, 1H), 8.28 (s, 1H), 8.04-7.97 (m, 2H), 7.40 (d, J=8.3 Hz, 2H), 7.29-7.16 (m, 4H), 4.88 (p, J=7.1 Hz, 1H), 4.72-4.37 (m, 2H), 3.52-3.16 (m, 2H), 2.48-2.47 (m, 1H), 2.45 (s, 3H), 2.39 (s, 3H), 1.96-1.68 (m, 3H), 1.60-1.44 (m, 1H), 1.32 (d, J=7.1 Hz, 3H); ¹³C NMR (100 MHz, DMSO-d₆) δ 171.57, 160.86, 157.42, 155.86, 141.66, 137.69, 137.11, 135.95, 129.97, 126.48, 126.10, 123.41, 120.07, 102.22, 47.20, 46.64, 46.04, 42.02, 27.69, 24.33, 22.40, 20.54, 14.94; HRMS (ESI) calcd for C₂₇H₃₁N₆OS [M+H]⁺ 487.2275, found 487.2280.

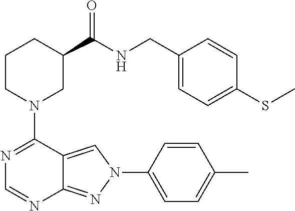 [SMILES: CSc1ccc(CNC(=O)[C@@H]2CCCN(c3ncnc4nn(-c5ccc(C)cc5)cc34)C2)cc1]

(R)—N-(4-(methylthio)benzyl)-1-(2-(p-tolyl)-2H-pyrazolo[3,4-d]pyrimidin-4-yl)piperidine-3-carboxamide. White solid (yield 84%): [α]_{D}²⁵=−87.8 (c 0.7, MeOH); ¹H NMR (400 MHz, DMSO-d₆) δ 9.27 (s, 1H), 8.46 (t, J=6.0 Hz, 1H), 8.27 (s, 1H), 7.97 (d, J=8.2 Hz, 2H), 7.39 (d, J=8.2 Hz, 2H), 7.19 (s, 4H), 5.03-4.47 (m, 2H), 4.33-4.16 (m, 2H), 3.43-3.25 (m, 2H), 2.47-2.46 (m, 1H), 2.44 (s, 3H), 2.39 (s, 3H), 2.00-1.75 (m, 3H), 1.61-1.46 (m, 1H); ¹³C NMR (100 MHz, DMSO-d₆) δ 172.43, 160.86, 157.43, 155.83, 137.68, 137.11, 136.27, 136.22, 129.97, 127.90, 126.09, 123.34, 120.07, 102.24, 46.72, 45.32, 42.11, 41.50, 27.62, 24.35, 20.56, 14.93; HRMS (ESI) calcd for C₂₆H₂₉N₆OS [M+H]⁺ 473.2118, found 473.2125.

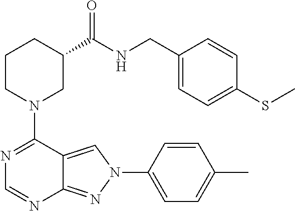 [SMILES: CSc1ccc(CNC(=O)[C@H]2CCCN(c3ncnc4nn(-c5ccc(C)cc5)cc34)C2)cc1]

(S)—N-(4-(methylthio)benzyl)-1-(2-(p-tolyl)-2H-pyrazolo[3,4-d]pyrimidin-4-yl)piperidine-3-carboxamide. White solid (yield 87%): [α]_{D}²⁵=+94.5 (c 0.5, MeOH); ¹H NMR (400 MHz, DMSO-d₆) δ 9.27 (s, 1H), 8.45 (t, J=5.7 Hz, 1H), 8.27 (s, 1H), 7.97 (d, J=8.1 Hz, 2H), 7.39 (d, J=8.2 Hz, 2H), 7.26-7.04 (m, 4H), 5.02-4.43 (m, 2H), 4.31-4.17 (m, 2H), 3.56-3.14 (m, 2H), 2.47-2.46 (m, 1H), 2.44 (s, 3H), 2.39 (s, 3H), 1.98-1.77 (m, 3H), 1.61-1.46 (m, 1H). ¹³C NMR (100 MHz, DMSO-d₆) δ 172.90, 161.33, 157.90, 156.31, 138.15, 137.58, 136.75, 136.69, 130.44, 128.37, 126.57, 123.81, 120.54, 102.72, 47.19, 46.55, 42.64, 41.97, 28.10, 24.84, 21.03, 15.41; HRMS (ESI) calcd for C₂₆H₂₉N₆OS [M+H]⁺ 473.2118, found 473.2127.

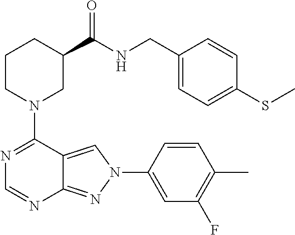 [SMILES: CSc1ccc(CNC(=O)[C@@H]2CCCN(c3ncnc4nn(-c5ccc(C)c(F)c5)cc34)C2)cc1]

(R)-1-(2-(3-fluoro-4-methylphenyl)-2H-pyrazolo[3,4-d]pyrimidin-4-yl)-N-(4-(methylthio)benzyl)piperidine-3-carboxamide. Light brown solid (yield 84%): [α]_{D}²⁵=−94.2 (c 1.2, MeOH); ¹H NMR (400 MHz, DMSO-d₆) δ 9.35 (s, 1H), 8.47-8.40 (m, 1H), 8.28 (s, 1H), 7.98 (dd, J=11.0, 2.3 Hz, 1H), 7.88 (dd, J=8.3, 2.2 Hz, 1H), 7.51 (t, J=8.4 Hz, 1H), 7.24-7.16 (m, 4H), 5.08-4.39 (m, 2H), 4.31-4.18 (m, 2H), 3.44-3.24 (m, 2H), 2.49-2.47 (m, 1H), 2.44 (s, 3H), 2.31 (d, J=1.8 Hz, 3H), 1.99-1.77 (m, 3H), 1.61-1.48 (m, 1H); ¹³C NMR (100 MHz, DMSO-d₆) δ 172.41, 160.87, 160.68 (d, J=243.3 Hz), 157.42, 156.16, 138.48 (d, J=10.4 Hz), 136.28, 136.20, 132.43 (d, J=6.1 Hz), 127.90, 126.07, 124.10 (d, J=17.3 Hz), 123.85, 115.73 (d, J=3.3 Hz), 107.12 (d, J=27.8 Hz), 102.47, 46.89, 46.35, 42.18, 41.50, 27.62, 24.24, 14.91, 13.86; HRMS (ESI) calcd for C₂₆H₂₈FN₆OS [M+H]⁺ 491.2024, found 491.2039.

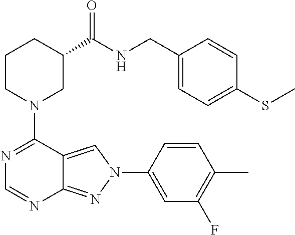 [SMILES: CSc1ccc(CNC(=O)[C@H]2CCCN(c3ncnc4nn(-c5ccc(C)c(F)c5)cc34)C2)cc1]

(S)-1-(2-(3-fluoro-4-methylphenyl)-2H-pyrazolo[3,4-d]pyrimidin-4-yl)-N-(4-(methylthio)benzyl)piperidine-3-carboxamide. Light brown solid (yield 87%): [α]_{D}²⁵=+84.2 (c 0.7, MeOH); ¹H NMR (400 MHz, DMSO-d₆) δ 9.34 (s, 1H), 8.49-8.39 (m, 1H), 8.28 (s, 1H), 7.98 (dd, J=11.0, 2.3 Hz, 1H), 7.88 (dd, J=8.2, 2.2 Hz, 1H), 7.50 (t, J=8.4 Hz, 1H), 7.23-7.15 (m, 4H), 5.07-4.43 (m, 2H), 4.30-4.17 (m, 2H), 3.37 (s, 2H), 2.48-2.46 (m, 1H), 2.44 (s, 3H), 2.31 (d, J=1.8 Hz, 3H), 2.02-1.76 (m, 3H), 1.62-1.47 (m, 1H); ¹³C NMR (100 MHz, DMSO-d₆) δ 172.41, 160.86, 160.67 (d, J=243.5 Hz), 157.41, 156.14, 138.47 (d, J=10.2 Hz), 136.28, 136.19, 132.41 (d, J=5.9 Hz), 127.90, 126.07, 124.08 (d, J=17.5 Hz), 123.82, 115.71 (d, J=2.6 Hz), 107.09 (d, J=27.7 Hz), 102.46, 46.57, 45.94, 42.21, 41.50, 27.63, 24.33, 14.91, 13.83; HRMS (ESI) calcd for C₂₆H₂₈FN₆OS [M+H]⁺ 491.2024, found 491.2032.

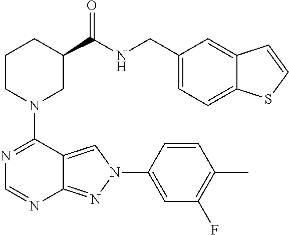 [SMILES: Cc1ccc(-n2cc3c(N4CCC[C@@H](C(=O)NCc5ccc6sccc6c5)C4)ncnc3n2)cc1F]

(R)—N-(benzo[b]thiophen-5-ylmethyl)-1-(2-(3-fluoro-4-methylphenyl)-2H-pyrazolo[3,4-d]pyrimidin-4-yl)piperidine-3-carboxamide. Light brown solid (yield 84%): [α]_{D}²⁵=−90.4 (c 1.0, MeOH); ¹H NMR (400 MHz, Chloroform-d) δ 8.35 (s, 1H), 8.22 (s, 1H), 7.77 (d, J=8.3 Hz, 1H), 7.67 (d, J=1.7 Hz, 1H), 7.55 (dd, J=10.2, 2.1 Hz, 1H), 7.49-7.42 (m, 1H), 7.42 (d, J=5.4 Hz, 1H), 7.28-7.18 (m, 4H), 4.55 (d, J=5.5 Hz, 2H), 4.33-4.02 (m, 3H), 3.66-3.47 (m, 1H), 2.73-2.63 (m, 1H), 2.32 (d, J=1.9 Hz, 3H), 2.26-2.14 (m, 1H), 2.07-1.98 (m, 1H), 1.87-1.77 (m, 1H), 1.75-1.62 (m, 1H); ¹³C NMR (100 MHz, DMSO-d₆) δ 172.54, 160.80, 160.73 (d, J=242.3 Hz), 157.48, 156.15, 139.57, 138.50 (d, J=10.2 Hz), 137.70, 135.73, 132.48 (d, J=5.8 Hz), 127.80, 124.27, 124.10, 123.96, 123.84, 122.47, 122.11, 115.80 (d, J=2.8 Hz), 107.20 (d, J=27.6 Hz), 102.51, 46.88, 46.17, 42.25, 42.08, 27.70, 24.33, 13.89; HRMS (ESI) calcd for C₂₇H₂₆FN₆OS [M+H]⁺ 501.1867, found 501.1868.

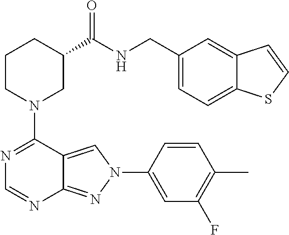 [SMILES: Cc1ccc(-n2cc3c(N4CCC[C@H](C(=O)NCc5ccc6sccc6c5)C4)ncnc3n2)cc1F]

(S)—N-(benzo[b]thiophen-5-ylmethyl)-1-(2-(3-fluoro-4-methylphenyl)-2H-pyrazolo[3,4-d]pyrimidin-4-yl)piperidine-3-carboxamide. Light brown solid (yield 87%): [α]_{D}²⁵=+82.7 (c 2.1, MeOH); ¹H NMR (400 MHz, DMSO-d₆) δ 9.35 (s, 1H), 8.58-8.47 (m, 1H), 8.28 (s, 1H), 8.00-7.82 (m, 3H), 7.77-7.72 (m, 2H), 7.49 (t, J=8.4 Hz, 1H), 7.43-7.37 (m, 1H), 7.27 (dd, J=8.4, 1.6 Hz, 1H), 5.07-4.46 (m, 2H), 4.40 (d, J=5.8 Hz, 2H), 3.32-3.09 (m, 2H), 2.55-2.52 (m, 1H), 2.31 (d, J=1.8 Hz, 3H), 2.03-1.79 (m, 3H), 1.63-1.46 (m, 1H); ¹³C NMR (100 MHz, DMSO-d₆) δ 172.54, 160.80, 160.72 (d, J=243.4 Hz), 157.48, 156.15, 139.58, 138.50 (d, J=10.3 Hz), 137.68, 135.74, 132.48 (d, J=5.8 Hz), 127.80, 124.27, 124.10, 123.96, 123.84, 122.47, 122.11, 115.80 (d, J=2.8 Hz), 107.20 (d, J=27.6 Hz), 102.51, 46.88, 46.23, 42.25, 42.08, 27.70, 24.30, 13.90; HRMS (ESI) calcd for C₂₇H₂₆FN₆OS [M+H]⁺ 501.1867, found 501.1876.

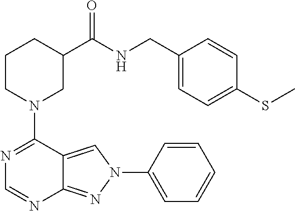 [SMILES: CSc1ccc(CNC(=O)C2CCCN(c3ncnc4nn(-c5ccccc5)cc34)C2)cc1]

N-(4-(methylthio)benzyl)-1-2-phenyl-2H-pyrazolo[3,4-d]pyrimidin-4-yl)piperidine-3-carboxamide. White solid (yield 72%): ¹H NMR (400 MHz, Chloroform-d) δ 8.47 (s, 1H), 8.31 (s, 1H), 7.90 (d, J=8.0 Hz, 2H), 7.58-7.50 (m, 2H), 7.46-7.41 (m, 1H), 7.20-7.12 (m, 4H), 4.38 (qd, J=14.6, 5.6 Hz, 2H), 4.26-4.16 (m, 3H), 3.75-3.62 (m, 1H), 2.68-2.58 (m, 1H), 2.45 (s, 3H), 2.28-2.20 (m, 1H), 2.05-1.95 (m, 1H), 1.84-1.67 (m, 2H); HRMS (ESI) calcd for C₂₅H₂₇N₆OS [M+H]⁺ 459.1962, found 459.1967.

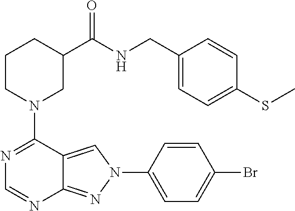 [SMILES: CSc1ccc(CNC(=O)C2CCCN(c3ncnc4nn(-c5ccc(Br)cc5)cc34)C2)cc1]

1-(2-(4-bromophenyl)-2H-pyrazolo[3,4-d]pyrimidin-4-yl)-N-(4-(methylthio)benzyl)piperidine-3-carboxamide. White solid (yield 87%): ¹H NMR (400 MHz, DMSO-d₆) δ 9.37 (s, 1H), 8.48-8.41 (m, 1H), 8.28 (s, 1H), 8.08 (d, J=8.7 Hz, 2H), 7.88-7.73 (m, 2H), 7.25-7.11 (m, 4H), 5.08-4.44 (m, 2H), 4.29-4.16 (m, 2H), 3.59-3.17 (m, 2H), 2.54-2.52 (m, 1H), 2.44 (s, 3H), 2.00-1.74 (m, 3H), 1.59-1.47 (m, 1H); ¹³C NMR (100 MHz, DMSO-d₆) δ 172.40, 160.98, 157.43, 156.18, 138.54, 136.27, 136.20, 132.46, 127.90, 126.07, 123.95, 122.08, 120.76, 102.57, 47.06, 46.32, 42.21, 41.49, 27.59, 24.33, 14.93; HRMS (ESI) calcd for C₂₅H₂₅BrN₆OS [M+H]⁺ 537.1067, found 537.1070.

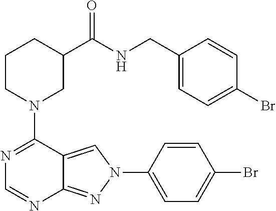 [SMILES: O=C(NCc1ccc(Br)cc1)C1CCCN(c2ncnc3nn(-c4ccc(Br)cc4)cc23)C1]

N-(4-bromobenzyl)-1-(2-(4-bromophenyl)-2H-pyrazolo[3,4-d]pyrimidin-4-yl)piperidine-3-carboxamide. Light brown solid (yield 83%): ¹H NMR (400 MHz, DMSO-d₆) δ 9.37 (s, 1H), 8.54-8.49 (m, 1H), 8.28 (s, 1H), 8.08 (d, J=8.5 Hz, 2H), 7.88-7.74 (m, 2H), 7.48 (d, J=8.0 Hz, 2H), 7.26-7.16 (m, 2H), 5.07-4.43 (m, 2H), 4.25 (d, J=5.9 Hz, 2H), 3.61-3.36 (m, 2H), 2.44 (s, 1H), 2.05-1.75 (m, 3H), 1.61-1.44 (m, 1H); ¹³C NMR (100 MHz, DMSO-d₆) δ 172.52, 160.99, 157.44, 156.19, 138.97, 138.54, 132.47, 131.13, 129.42, 123.97, 122.10, 120.78, 119.74, 102.58, 46.82, 46.38, 42.07, 41.32, 27.60, 24.34; HRMS (ESI) calcd for C₂₄H₂₃Br₂N₆O [M+H]⁺ 569.0295, found 569.0288.

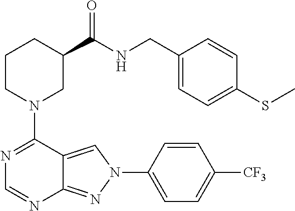 [SMILES: CSc1ccc(CNC(=O)[C@@H]2CCCN(c3ncnc4nn(-c5ccc(C(F)(F)F)cc5)cc34)C2)cc1]

(R)—N-(4-(methylthio)benzyl)-1-(2-(4-(trifluoromethyl)phenyl)-2H-pyrazolo[3,4-d]pyrimidin-4-yl)piperidine-3-carboxamide. Light brown solid (yield 84%): [α]_{D}²⁵=−94.4 (c 0.7, MeOH); ¹H NMR (400 MHz, Chloroform-d) δ 8.57 (s, 1H), 8.32 (s, 1H), 8.06 (d, J=8.4 Hz, 2H), 7.80 (d, J=8.5 Hz, 2H), 7.19-7.13 (m, 4H), 4.46-4.34 (m, 2H), 4.28-4.09 (m, 2H), 3.66 (d, J=3.8 Hz, 2H), 2.67-2.59 (m, 1H), 2.45 (s, 3H), 2.28-2.15 (m, 1H), 2.08-1.98 (m, 1H), 1.91-1.67 (m, 2H); ¹³C NMR (100 MHz, DMSO-d₆) δ 172.38, 161.14, 157.51, 156.53, 142.15, 136.28, 136.20, 127.94 (q, J=49.3 Hz), 127.92, 126.90, 126.06, 124.63, 123.97 (q, J=273.2 Hz), 120.63, 102.87, 47.01, 46.42, 42.18, 41.50, 27.59, 24.46, 14.90; HRMS (ESI) calcd for C₂₆H₂₆F₃N₆OS [M+H]⁺ 527.1835, found 527.1841.

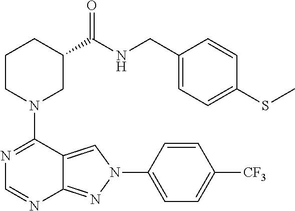 [SMILES: CSc1ccc(CNC(=O)[C@H]2CCCN(c3ncnc4nn(-c5ccc(C(F)(F)F)cc5)cc34)C2)cc1]

(S)—N-(4-(methylthio)benzyl)-1-(2-(4-(trifluoromethyl)phenyl)-2H-pyrazolo[3,4-d]pyrimidin-4-yl)piperidine-3-carboxamide. Light brown solid (yield 84%): [α]_{D}²⁵=+81.7 (c 0.3, MeOH); ¹H NMR (400 MHz, DMSO-d₆) δ 9.49 (s, 1H), 8.48-8.42 (m, 1H), 8.36 (d, J=8.4 Hz, 2H), 8.30 (s, 1H), 7.99 (d, J=8.5 Hz, 2H), 7.29-7.15 (m, 4H), 5.03-4.46 (m, 2H), 4.32-4.16 (m, 2H), 3.22-3.02 (m, 2H), 2.47-2.40 (m, 4H), 2.00-1.78 (m, 3H), 1.62-1.49 (m, 1H); ¹³C NMR (100 MHz, DMSO-d₆) δ 172.39, 161.12, 157.51, 156.51, 142.15, 136.28, 136.20, 127.94 (q, J=49.2 Hz), 127.92, 126.90, 126.06, 124.63, 123.97 (q, J=272.1 Hz), 120.63, 102.87, 46.99, 46.02, 42.18, 41.50, 27.59, 24.45, 14.90; HRMS (ESI) calcd for C₂₆H₂₆F₃N₆OS [M+H]⁺ 527.1835, found 527.1844.

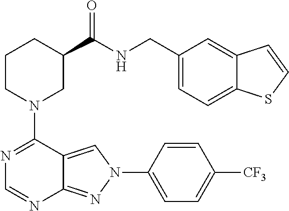 [SMILES: O=C(NCc1ccc2sccc2c1)[C@@H]1CCCN(c2ncnc3nn(-c4ccc(C(F)(F)F)cc4)cc23)C1]

(R)—N-(benzo[b]thiophen-5-ylmethyl)-1-(2-(4-(trifluoromethyl)phenyl)-2H-pyrazolo[3,4-d]pyrimidin-4-yl)piperidine-3-carboxamide. Light brown solid (yield 87%): [α]_{D}²⁵=−80.4 (c 0.8, MeOH); ¹H NMR (400 MHz, Chloroform-d) δ 8.55 (s, 1H), 8.31 (s, 1H), 8.03 (d, J=8.4 Hz, 2H), 7.83-7.75 (m, 3H), 7.68 (s, 1H), 7.44 (d, J=5.5 Hz, 1H), 7.26-7.17 (m, 2H), 4.61-4.50 (m, 2H), 4.33-4.11 (m, 3H), 3.71-3.59 (m, 1H), 2.69-2.61 (m, 1H), 2.22 (s, 1H), 2.09-2.00 (m, 1H), 1.90-1.81 (m, 1H), 1.78-1.67 (m, 1H); ¹³C NMR (100 MHz, DMSO-d₆) δ 172.39, 161.10, 157.50, 156.49, 142.13, 139.52, 137.63, 135.71, δ 128.08 (q, J=33.1 Hz), 127.75, 126.88, 124.63, 124.07, 123.97 (q, J=271.4 Hz), 123.79, 122.43, 122.08, 120.62, 102.87, 47.03, 46.37, 42.24, 42.03, 27.64, 24.66; HRMS (ESI) calcd for C₂₇H₂₄F₃N₆OS [M+H]⁺ 537.1679, found 537.1685.

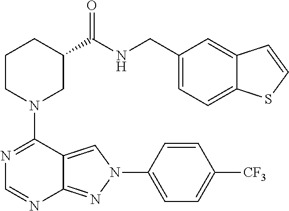 [SMILES: O=C(NCc1ccc2sccc2c1)[C@H]1CCCN(c2ncnc3nn(-c4ccc(C(F)(F)F)cc4)cc23)C1]

(S)—N-(benzo[b]thiophen-5-ylmethyl)-1-(2-(4-(trifluoromethyl)phenyl)-2H-pyrazolo[3,4-d]pyrimidin-4-yl)piperidine-3-carboxamide. Light brown solid (yield 87%): [α]_{D}²⁵=+83.4 (c 0.5, MeOH); ¹H NMR (400 MHz, DMSO-d₆) δ 9.48 (s, 1H), 8.54 (t, J=5.5 Hz, 1H), 8.34 (d, J=8.3 Hz, 2H), 8.30 (s, 1H), 8.01-7.88 (m, 3H), 7.79-7.70 (m, 2H), 7.48-7.37 (m, 1H), 7.28 (d, J=8.3 Hz, 1H), 5.13-4.52 (m, 2H), 4.41 (d, J=5.7 Hz, 2H), 3.48-3.37 (m, 2H), 2.64-2.54 (m, 1H), 2.08-1.77 (m, 3H), 1.56 (d, J=12.1 Hz, 1H); ¹³C NMR (100 MHz, DMSO-d₆) δ 172.39, 161.12, 157.51, 156.50, 142.13, 139.52, 137.63, 135.71, δ 128.01 (q, J=32.5 Hz), 127.75, 126.88, 124.62, 124.07, 123.97 (q, J=271.5 Hz), 123.79, 122.43, 122.08, 120.62, 102.87, 47.00, 46.39, 42.24, 42.03, 27.63, 24.66; HRMS (ESI) calcd for C₂₇H₂₄F₃N₆OS [M+H]⁺ 537.1679, found 537.1686.

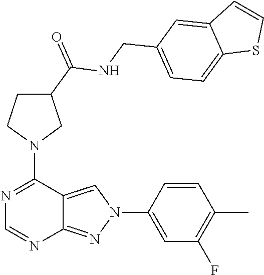 [SMILES: Cc1ccc(-n2cc3c(N4CCC(C(=O)NCc5ccc6sccc6c5)C4)ncnc3n2)cc1F]

N-(benzo[b]thiophen-5-ylmethyl)-1-(2-(3-fluoro-4-methylphenyl)-2H-pyrazolo[3,4-d]pyrimidin-4-yl)pyrrolidine-3-carboxamide. Light brown solid (yield 71%): ¹H NMR (400 MHz, DMSO-d₆) δ 9.22 (d, J=14.7 Hz, 1H), 8.79-8.64 (m, 1H), 8.25 (s, 1H), 8.04-7.92 (m, 2H), 7.94-7.85 (m, 1H), 7.82-7.73 (m, 2H), 7.54-7.44 (m, 1H), 7.44 (dd, J=7.0, 5.4 Hz, 1H), 7.30 (ddd, J=10.1, 8.3, 1.7 Hz, 1H), 4.55-4.38 (m, 2H), 4.15-3.63 (m, 4H), 3.26-3.12 (m, 1H), 2.44-2.05 (m, 5H); ¹³C NMR (100 MHz, DMSO-d₆) δ 171.90, 160.69 (d, J=243.4 Hz), 160.18, 156.61, 155.88, 139.57, 138.58 (d, J=10.2 Hz), 137.70, 135.61 (d, J=15.3 Hz), 132.39, 127.83, 124.13, 124.04, 123.94, 123.82, 122.49, 122.10 (d, J=11.5 Hz), 115.56, 106.93 (d, J=27.7 Hz), 103.49, 50.35, 47.99, 43.70, 41.96, 29.09, 13.83; HRMS (ESI) calcd for C₂₆H₂₄FN₆OS [M+H]⁺ 487.1711, found 487.1720.

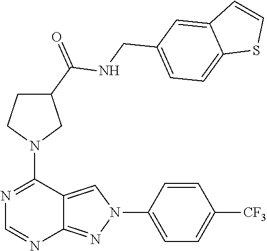 [SMILES: O=C(NCc1ccc2sccc2c1)C1CCN(c2ncnc3nn(-c4ccc(C(F)(F)F)cc4)cc23)C1]

N-(benzo[b]thiophen-5-ylmethyl)-1-(2-(4-(trifluoromethyl)phenyl)-2H-pyrazolo[3,4-d]pyrimidin-4-yl)pyrrolidine-3-carboxamide. Light brown solid (yield 77%): ¹H NMR (400 MHz, DMSO-d₆) δ 9.36 (d, J=13.7 Hz, 1H), 8.80-8.65 (m, 1H), 8.38 (dd, J=8.7, 3.5 Hz, 2H), 8.27 (s, 1H), 8.02-7.92 (m, 3H), 7.82-7.73 (m, 2H), 7.48-7.40 (m, 1H), 7.35-7.25 (m, 1H), 4.51-4.39 (m, 2H), 4.17-3.66 (m, 4H), 3.24-3.19 (m, 1H), 2.42-2.07 (m, 2H); ¹³C NMR (100 MHz, DMSO-d₆) δ 172.37, 160.94, 157.44, 156.44, 142.71, 140.05, 138.19, 136.16, 128.32 (q, J=32.6 Hz), 128.31, 127.35, 125.39, 124.65, 124.47 (q, J=272.2 Hz), 122.52, 120.91, 104.40, 50.91, 47.76, 44.18, 42.42, 29.58; HRMS (ESI) calcd for C₂₆H₂₂F₃N₆OS [M+H]⁺ 523.1522, found 523.1527.

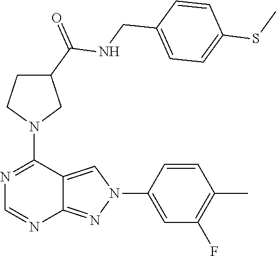 [SMILES: CSc1ccc(CNC(=O)C2CCN(c3ncnc4nn(-c5ccc(C)c(F)c5)cc34)C2)cc1]

1-(2-(3-fluoro-4-methylphenyl)-2H-pyrazolo[3,4-d]pyrimidin-4-yl)-N-(4-(methylthio)benzyl)pyrrolidine-3-carboxamide. Light brown solid (yield 80%): ¹H NMR (400 MHz, DMSO-d₆) δ 9.21 (d, J=13.1 Hz, 1H), 8.70-8.58 (m, 1H), 8.24 (s, 1H), 8.03-7.95 (m, 1H), 7.92-7.87 (m, 1H), 7.54-7.46 (m, 1H), 7.27-7.19 (m, 4H), 4.36-4.22 (m, 2H), 4.11-3.62 (m, 4H), 3.24-3.12 (m, 1H), 2.45 (s, 3H), 2.37-2.06 (m, 5H); ¹³C NMR (100 MHz, DMSO-d₆) δ 171.88, 160.70 (d, J=243.0 Hz), 160.17, 156.60, 155.87, 138.57 (d, J=10.3 Hz), 136.34, 136.19, 132.38, 127.89, 126.13, 124.11, 123.85 (d, J=17.4 Hz), 115.53 (d, J=4.7 Hz), 106.93 (d, J=27.7 Hz), 103.49, 50.33, 47.97, 43.65, 41.93, 29.07, 14.93, 13.83; HRMS (ESI) calcd for C₂₅H₂₆FN₆OS [M+H]⁺ 477.1867, found 477.1876.

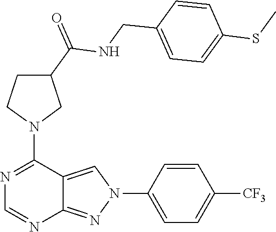 [SMILES: CSc1ccc(CNC(=O)C2CCN(c3ncnc4nn(-c5ccc(C(F)(F)F)cc5)cc34)C2)cc1]

N-(4-(methylthio)benzyl)-1-(2-(4-(trifluoromethyl)phenyl)-2H-pyrazolo[3,4-d]pyrimidin-4-yl)pyrrolidine-3-carboxamide. Light brown solid (yield 74%): ¹H NMR (400 MHz, DMSO-d₆) δ 9.35 (d, J=12.5 Hz, 1H), 8.73-8.57 (m, 1H), 8.41-8.35 (m, 2H), 8.27 (s, 1H), 7.97 (dd, J=8.7, 4.5 Hz, 2H), 7.29-7.20 (m, 4H), 4.36-4.22 (m, 2H), 4.13-3.65 (m, 4H), 3.22-3.15 (m, 1H), 2.45 (s, 3H), 2.41-2.03 (m, 2H); ¹³C NMR (100 MHz, DMSO-d₆) δ 172.34, 160.93, 157.43, 156.43, 142.70, 136.82, 136.51, 128.37, 128.16, 127.34, 126.61, 125.38, 124.47 (q, J=271.9 Hz), 120.90, 104.39, 50.89, 48.51, 44.13, 42.39, 29.56, 15.42; HRMS (ESI) calcd for C₂₅H₂₄F₃N₆OS [M+H]⁺ 513.1679, found 513.1683.

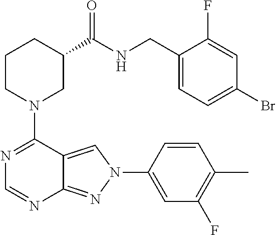 [SMILES: Cc1ccc(-n2cc3c(N4CCC[C@H](C(=O)NCc5ccc(Br)cc5F)C4)ncnc3n2)cc1F]

(S)—N-(4-bromo-2-fluorobenzyl)-1-(2-(3-fluoro-4-methylphenyl)-2H-pyrazolo[3,4-d]pyrimidin-4-yl)piperidine-3-carboxamide. Light brown solid (yield 74%): [α]_{D}²⁵=+86.7 (c 0.5, MeOH); ¹H NMR (400 MHz, DMSO-d₆) δ 9.32 (s, 1H), 8.50 (t, J=5.8 Hz, 1H), 8.27 (s, 1H), 7.96 (dd, J=11.0, 2.2 Hz, 1H), 7.87 (dd, J=8.3, 2.2 Hz, 1H), 7.54-7.46 (m, 2H), 7.39-7.22 (m, 2H), 5.06-4.39 (m, 2H), 4.34-4.20 (m, 2H), 3.52 (s, 2H), 2.55 (s, 1H), 2.31 (s, 3H), 1.98-1.76 (m, 3H), 1.61-1.48 (m, 1H); ¹³C NMR (100 MHz, DMSO-d₆) δ 172.63, 160.84, 160.65 (d, J=243.4 Hz), 159.88 (d, J=249.6 Hz), 157.40, 156.11, 138.45 (d, J=10.4 Hz), 132.38 (d, J=6.1 Hz), 131.07 (d, J=5.0 Hz), 127.40, 125.68 (d, J=14.7 Hz), 124.07 (d, J=17.1 Hz), 123.79, 120.07 (d, J=9.8 Hz), 118.43 (d, J=25.0 Hz), 115.69, 107.08 (d, J=28.0 Hz), 102.43, 46.60, 45.87, 41.97, 35.52, 27.55, 24.11, 13.83; HRMS (ESI) calcd for C₂₅H₂₄BrF₂N₆O [M+H]⁺ 541.1158, found 541.1166.

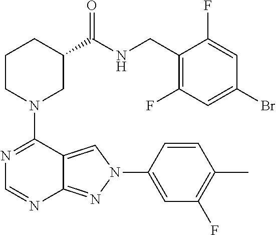 [SMILES: Cc1ccc(-n2cc3c(N4CCC[C@H](C(=O)NCc5c(F)cc(Br)cc5F)C4)ncnc3n2)cc1F]

(S)—N-(4-bromo-2,6-difluorobenzyl)-1-(2-(3-fluoro-4-methylphenyl)-2H-pyrazolo[3,4-d]pyrimidin-4-yl)piperidine-3-carboxamide. Light brown solid (yield 83%): [α]_{D}²⁵=+84.5 (c 0.7, MeOH); ¹H NMR (400 MHz, DMSO-d₆) δ 9.28 (s, 1H), 8.39 (t, J=5.3 Hz, 1H), 8.25 (s, 1H), 7.94 (d, J=11.0 Hz, 1H), 7.85 (d, J=8.0 Hz, 1H), 7.53-7.47 (m, 1H), 7.44-7.38 (m, 2H), 4.96-4.45 (m, 2H), 4.33-4.23 (m, 2H), 3.41-3.33 (m, 2H), 2.46-2.40 (m, 1H), 2.31 (s, 3H), 1.91-1.73 (m, 3H), 1.57-1.45 (m, 1H); ¹³C NMR (100 MHz, DMSO-d₆) δ 172.23, 163.41, 161.00 (d, J=251.6 Hz), 160.91 (d, J=251.8 Hz), 160.82, 160.66 (d, J=243.3 Hz), 157.37, 156.09, 138.45 (d, J=10.2 Hz), 132.38 (d, J=6.0 Hz), 124.07 (d, J=17.5 Hz), 123.74, 120.58, 115.69, 115.26 (d, J=28.2 Hz), 114.05, 107.07 (d, J=27.5 Hz), 102.40, 47.50, 46.43, 41.78, 30.46, 27.45, 24.26, 13.84; HRMS (ESI) calcd for C₂₅H₂₃BrF₃N₆O [M+H]⁺ 559.1063, found 559.1070.

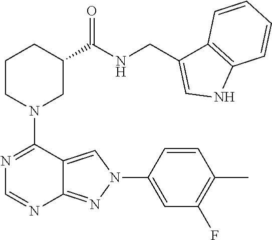 [SMILES: Cc1ccc(-n2cc3c(N4CCC[C@H](C(=O)NCc5c[nH]c6ccccc56)C4)ncnc3n2)cc1F]

(S)—N-((1H-indol-3-yl)methyl)-1-(2-(3-fluoro-4-methylphenyl)-2H-pyrazolo[3,4-d]pyrimidin-4-yl)piperidine-3-carboxamide. Light brown solid (yield 83%): [α]_{D}²⁵=+88.2 (c 0.4, MeOH); ¹H NMR (400 MHz, DMSO-d₆) δ 10.91 (s, 1H), 9.31 (s, 1H), 8.27-8.20 (m, 2H), 7.96 (dd, J=11.0, 2.2 Hz, 1H), 7.88-7.81 (m, 1H), 7.56 (d, J=7.9 Hz, 1H), 7.52-7.46 (m, 1H), 7.35 (d, J=8.1 Hz, 1H), 7.25 (d, J=2.2 Hz, 1H), 7.10-7.03 (m, 1H), 6.99-6.93 (m, 1H), 5.00-4.34 (m, 4H), 3.26-3.10 (m, 2H), 2.48-2.40 (m, 1H), 2.30 (s, 3H), 1.93-1.78 (m, 3H), 1.57-1.44 (m, 1H); ¹³C NMR (101 MHz, DMSO-d₆) δ 172.12, 160.83, 160.67 (d, J=243.3 Hz), 157.37, 156.11, 138.46 (d, J=10.4 Hz), 136.34, 132.38 (d, J=5.9 Hz), 126.45, 124.04 (d, J=17.2 Hz), 123.77, 123.72, 121.11, 118.65, 118.45, 115.68, 111.42, 107.10 (d, J=27.7 Hz), 102.44, 47.24, 46.22, 42.15, 33.98, 27.74, 24.63, 13.82; HRMS (ESI) calcd for C₂₇H₂₇FN₇O [M+H]⁺ 484.2256, found 484.2261.

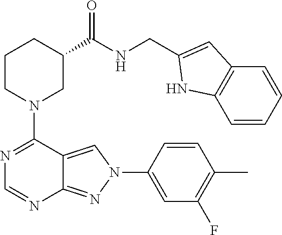 [SMILES: Cc1ccc(-n2cc3c(N4CCC[C@H](C(=O)NCc5cc6ccccc6[nH]5)C4)ncnc3n2)cc1F]

(S)—N-((1H-indol-2-yl)methyl)-1-(2-(3-fluoro-4-methylphenyl)-2H-pyrazolo[3,4-d]pyrimidin-4-yl)piperidine-3-carboxamide. Light brown solid (yield 84%): [α]_{D}²⁵=+79.2 (c 0.4, MeOH); ¹H NMR (400 MHz, DMSO-d₆) δ 10.95 (d, J=2.1 Hz, 1H), 9.35 (s, 1H), 8.49 (t, J=5.5 Hz, 1H), 8.28 (s, 1H), 7.98 (dd, J=11.0, 2.2 Hz, 1H), 7.87 (d, J=8.2 Hz, 1H), 7.50-7.40 (m, 2H), 7.34-7.28 (m, 1H), 7.05-6.92 (m, 2H), 6.26 (dd, J=2.1, 0.9 Hz, 1H), 5.17-4.33 (m, 4H), 3.79-3.46 (m, 2H), 2.58-2.52 (m, 1H), 2.31 (d, J=1.8 Hz, 3H), 2.01-1.79 (m, 3H), 1.60-1.47 (m, 1H); ¹³C NMR (101 MHz, DMSO-d₆) δ 172.48, 160.87, 160.67 (d, J=243.5 Hz), 157.41, 156.15, 138.47 (d, J=10.2 Hz), 136.94, 136.13, 132.41 (d, J=6.1 Hz), 127.85, 124.08 (d, J=17.4 Hz), 123.82, 120.60, 119.49, 118.78, 115.70, 111.02, 107.13 (d, J=27.7 Hz), 102.47, 99.11, 47.09, 46.45, 42.23, 36.05, 27.68, 24.38, 13.86; HRMS (ESI) calcd for C₂₇H₂₇FN₇O [M+H]⁺ 484.2256, found 484.2261.

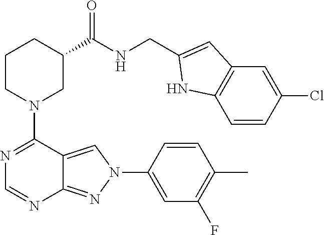 [SMILES: Cc1ccc(-n2cc3c(N4CCC[C@H](C(=O)NCc5cc6cc(Cl)ccc6[nH]5)C4)ncnc3n2)cc1F]

(S)—N-((5-chloro-1H-indol-2-yl)methyl)-1-(2-(3-fluoro-4-methylphenyl)-2H-pyrazolo[3,4-d]pyrimidin-4-yl)piperidine-3-carboxamide. Light brown solid (yield 74%): [α]_{D}²⁵=+89.3 (c 0.7, MeOH); ¹H NMR (400 MHz, DMSO-d₆) δ 11.19 (s, 1H), 9.34 (s, 1H), 8.52 (t, J=5.6 Hz, 1H), 8.28 (s, 1H), 7.97 (dd, J=11.0, 2.2 Hz, 1H), 7.86 (d, J=8.3 Hz, 1H), 7.51-7.42 (m, 2H), 7.35-7.29 (m, 1H), 7.05-7.00 (m, 1H), 6.26 (d, J=1.9 Hz, 1H), 5.13-4.29 (m, 4H), 3.81-3.49 (m, 2H), 2.57-2.52 (m, 1H), 2.30 (s, 3H), 2.04-1.80 (m, 3H), 1.62-1.47 (m, 1H); ¹³C NMR (100 MHz, DMSO-d₆) δ 172.56, 160.87, 160.67 (d, J=243.4 Hz), 157.42, 156.14, 139.01, 138.47 (d, J=10.5 Hz), 134.59, 132.40 (d, J=6.2 Hz), 129.06, 124.08 (d, J=17.1 Hz), 123.80, 123.35, 120.49, 118.64, 115.69 (d, J=3.5 Hz), 112.48, 107.12 (d, J=27.6 Hz), 102.47, 98.91, 46.84, 46.17, 42.20, 35.97, 27.64, 24.63, 13.86; HRMS (ESI) calcd for C₂₇H₂₅ClFN₇O [M+H]⁺ 518.1866, found 518.1870.

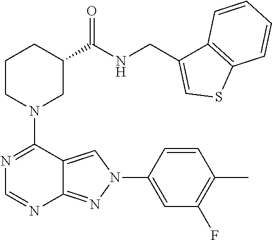 [SMILES: Cc1ccc(-n2cc3c(N4CCC[C@H](C(=O)NCc5csc6ccccc56)C4)ncnc3n2)cc1F]

(S)—N-(benzo[b]thiophen-3-ylmethyl)-1-(2-(3-fluoro-4-methylphenyl)-2H-pyrazolo[3,4-d]pyrimidin-4-yl)piperidine-3-carboxamide. Light brown solid (yield 78%): [α]_{D}²⁵=+90.1 (c 0.8, MeOH); ¹H NMR (400 MHz, DMSO-d₆) δ 9.32 (s, 1H), 8.48 (t, J=5.7 Hz, 1H), 8.26 (s, 1H), 8.01-7.94 (m, 2H), 7.90-7.82 (m, 2H), 7.55 (s, 1H), 7.53-7.47 (m, 1H), 7.41-7.34 (m, 2H), 5.09-4.35 (m, 4H), 3.93-3.55 (m, 2H), 2.58-2.53 (m, 1H), 2.31 (s, 3H), 1.99-1.79 (m, 3H), 1.60-1.45 (m, 1H); ¹³C NMR (100 MHz, DMSO-d₆) δ 172.46, 160.83, 160.66 (d, J=243.3 Hz), 157.40, 156.09, 139.87, 138.46 (d, J=10.4 Hz), 137.69, 133.81, 132.38 (d, J=6.0 Hz), 124.41, 124.14, 124.08, 124.02, 123.78, 122.92, 121.83, 115.68, 107.09 (d, J=27.6 Hz), 102.44, 46.97, 46.00, 42.10, 36.44, 27.67, 24.54, 13.83; HRMS (ESI) calcd for C₂₇H₂₆FN₆OS [M+H]⁺ 501.1867, found 501.1873.

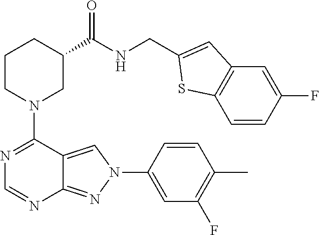 [SMILES: Cc1ccc(-n2cc3c(N4CCC[C@H](C(=O)NCc5cc6cc(F)ccc6s5)C4)ncnc3n2)cc1F]

(S)-1-(2-(3-fluoro-4-methylphenyl)-2H-pyrazolo[3,4-d]pyrimidin-4-yl)-N-((5-fluorobenzo[b]thiophen-2-yl)methyl)piperidine-3-carboxamide. [α]_{D}²⁵=+84.4 (c 0.4, MeOH); Light brown solid (yield 87%): ¹H NMR (400 MHz, DMSO-d₆) δ 9.32 (s, 1H), 8.74 (t, J=5.9 Hz, 1H), 8.28 (s, 1H), 8.00-7.82 (m, 3H), 7.56 (d, J=9.9 Hz, 1H), 7.51-7.42 (m, 1H), 7.29-7.24 (m, 1H), 7.21-7.13 (m, 1H), 5.10-4.40 (m, 4H), 3.55-3.35 (m, 2H), 2.58-2.54 (m, 1H), 2.29 (d, J=1.8 Hz, 3H), 2.01-1.80 (m, 3H), 1.62-1.50 (m, 1H); ¹³C NMR (101 MHz, DMSO-d₆) δ 172.55, 160.84, 160.65 (d, J=243.5 Hz), 160.16 (d, J=238.6 Hz), 157.42, 156.11, 146.90, 140.40 (d, J=9.8 Hz), 138.44 (d, J=10.2 Hz), 134.61, 132.35 (d, J=5.9 Hz), 124.13, 124.00, 123.77, 121.16 (d, J=4.2 Hz), 115.68, 112.41 (d, J=25.0 Hz), 108.59 (d, J=23.2 Hz), 107.08 (d, J=27.7 Hz), 102.45, 46.98, 46.29, 42.00, 37.92, 27.54, 24.41, 13.80; HRMS (ESI) calcd for C₂₇H₂₅F₂N₆OS [M+H]⁺ 519.1773, found 519.1778.

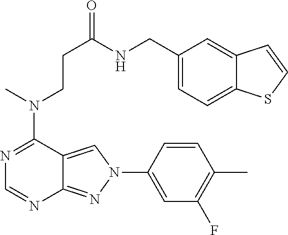 [SMILES: Cc1ccc(-n2cc3c(N(C)CCC(=O)NCc4ccc5sccc5c4)ncnc3n2)cc1F]

N-(benzo[b]thiophen-5-ylmethyl)-3-((2-(3-fluoro-4-methylphenyl)-2H-pyrazolo[3,4-d]pyrimidin-4-yl)(methyl)amino)propenamide. White solid (yield 88%): ¹H NMR (400 MHz, DMSO-d₆) δ 9.21 (s, 1H), 8.56-8.50 (m, 1H), 8.27 (s, 1H), 8.02-7.81 (m, 3H), 7.76-7.68 (m, 2H), 7.54-7.46 (m, 1H), 7.39-7.34 (m, 1H), 7.23 (dd, J=8.3, 1.7 Hz, 1H), 4.39 (d, J=5.8 Hz, 2H), 4.15-4.00 (m, 2H), 3.45 (s, 3H), 2.70-2.55 (m, 2H), 2.30 (s, 3H); ¹³C NMR (100 MHz, DMSO-d₆) δ 170.24, 160.69 (d, J=243.6 Hz), 160.59, 157.99, 156.22, 139.51, 138.52 (d, J=10.3 Hz), 137.60, 135.68, 132.44 (d, J=6.1 Hz), 127.75, 124.70, 124.09, 123.72, 122.36, 122.02, 115.61, 107.03 (d, J=27.5 Hz), 103.26, 47.83, 46.86, 42.16, 33.31, 13.85; HRMS (ESI) calcd for C₂₅H₂₄FN₆OS [M+H]⁺ 475.1711, found 475.1715.

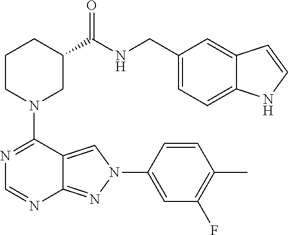 [SMILES: Cc1ccc(-n2cc3c(N4CCC[C@H](C(=O)NCc5ccc6[nH]ccc6c5)C4)ncnc3n2)cc1F]

(S)—N-((1H-indol-5-yl)methyl)-1-(2-(3-fluoro-4-methylphenyl)-2H-pyrazolo[3,4-d]pyrimidin-4-yl)piperidine-3-carboxamide. [α]_{D}²⁵=+91.1 (c 0.5, MeOH); Light brown solid (yield 87%): ¹H NMR (400 MHz, DMSO-d₆) δ 11.03 (s, 1H), 9.34 (s, 1H), 8.45-8.36 (m, 1H), 8.27 (s, 1H), 7.98 (dd, J=11.0, 2.2 Hz, 1H), 7.86 (d, J=8.3 Hz, 1H), 7.51-7.44 (m, 1H), 7.43-7.40 (m, 1H), 7.35-7.29 (m, 2H), 7.00 (dd, J=8.3, 1.7 Hz, 1H), 6.38-6.33 (m, 1H), 5.15-4.27 (m, 4H), 3.23-3.05 (m, 2H), 2.48-2.42 (m, 1H), 2.31 (d, J=1.8 Hz, 3H), 1.98-1.79 (m, 3H), 1.61-1.47 (m, 1H); ¹³C NMR (100 MHz, DMSO-d₆) δ 172.19, 160.86, 160.68 (d, J=243.0 Hz), 157.41, 156.15, 138.47 (d, J=10.0 Hz), 135.00, 132.40 (d, J=6.2 Hz), 129.54, 127.55, 125.55, 124.07 (d, J=17.0 Hz), 123.84, 120.99, 118.78, 115.69 (d, J=2.6 Hz), 111.22, 107.11 (d, J=27.8 Hz), 102.46, 100.83, 46.43, 45.93, 42.62, 42.15, 27.71, 24.68, 13.85; HRMS (ESI) calcd for C₂₇H₂₇FN₇O [M+H]⁺ 484.2256, found 484.2261.

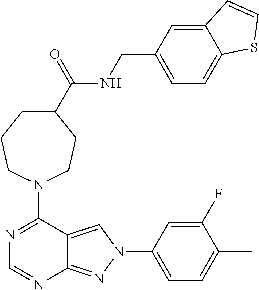 [SMILES: Cc1ccc(-n2cc3c(N4CCCC(C(=O)NCc5ccc6sccc6c5)CC4)ncnc3n2)cc1F]

N-(benzo[b]thiophen-5-ylmethyl)-1-(2-(3-fluoro-4-methylphenyl)-2H-pyrazolo[3,4-d]pyrimidin-4-yl)azepane-4-carboxamide. Light brown solid (yield 88%): ¹H NMR (400 MHz, DMSO-d₆) δ 9.19 (s, 1H), 8.33 (t, J=6.0 Hz, 1H), 8.26 (s, 1H), 7.98 (d, J=11.1 Hz, 1H), 7.94-7.87 (m, 2H), 7.75-7.69 (m, 2H), 7.53-7.48 (m, 1H), 7.41 (dd, J=11.4, 5.4 Hz, 1H), 7.23 (d, J=8.3 Hz, 1H), 4.39-4.32 (m, 2H), 4.29-4.15 (m, 1H), 4.13-4.03 (m, 1H), 4.00-3.64 (m, 2H), 2.40-2.28 (m, 4H), 2.23-1.52 (m, 6H); ¹³C NMR (101 MHz, DMSO-d₆) δ 175.13, 160.68 (d, J=243.2 Hz), 160.68, 157.65, 156.27, 139.51, 138.51 (d, J=10.5 Hz), 137.54, 135.97 (d, J=3.8 Hz), 132.40 (d, J=5.9 Hz), 127.71, 124.28 (d, J=9.1 Hz), 124.05, 123.98, 123.81, 122.39, 121.94, 115.69 (d, J=3.5 Hz), 107.06 (d, J=27.8 Hz), 102.76, 46.54, 44.74, 41.92, 30.36, 30.23, 25.80, 13.83 (d, J=2.9 Hz); HRMS (ESI) calcd for C₂₈H₂₈FN₆OS [M+H]⁺ 515.2024, found 515.2031.

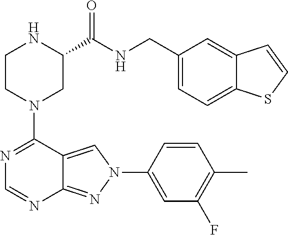 [SMILES: Cc1ccc(-n2cc3c(N4CCN[C@H](C(=O)NCc5ccc6sccc6c5)C4)ncnc3n2)cc1F]

(S)—N-(benzo[b]thiophen-5-ylmethyl)-4-(2-(3-fluoro-4-methylphenyl)-2H-pyrazolo[3,4-d]pyrimidin-4-yl)piperazine-2-carboxamide. White solid (74%): [α]_{D}²⁵=+78.8 (c 1.2, MeOH); ¹H NMR (400 MHz, Methanol-d₄) δ 8.99 (s, 1H), 8.25 (s, 1H), 7.78-7.59 (m, 4H), 7.51 (d, J=5.4 Hz, 1H), 7.37-7.30 (m, 1H), 7.26-7.18 (m, 2H), 4.74-4.47 (m, 3H), 4.30-4.18 (m, 1H), 4.03-3.79 (m, 3H), 3.40-3.32 (m, 1H), 3.22-3.10 (m, 1H), 2.31 (d, J=2.0 Hz, 3H); ¹³C NMR (100 MHz, Methanol-d₄) δ 171.18, 162.58 (d, J=245.0 Hz), 161.08, 159.46, 156.82, 141.31, 140.12, 139.80 (d, J=9.5 Hz), 135.76, 133.45 (d, J=5.7 Hz), 128.17, 126.52 (d, J=17.5 Hz), 125.13, 125.00, 124.64, 123.53, 123.45, 116.84 (d, J=3.6 Hz), 108.69 (d, J=28.0 Hz), 103.79, 58.10, 46.41, 46.20, 44.26, 43.53, 14.19 (d, J=3.2 Hz); HRMS (ESI) calcd for C₂₆H₂₅FN₇OS [M+H]⁺ 502.1820, found 502.1829.

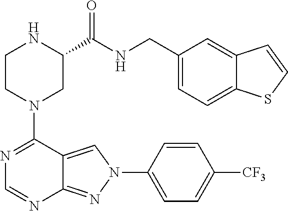 [SMILES: O=C(NCc1ccc2sccc2c1)[C@@H]1CN(c2ncnc3nn(-c4ccc(C(F)(F)F)cc4)cc23)CCN1]

(S)—N-(benzo[b]thiophen-5-ylmethyl)-4-(2-(4-(trifluoromethyl)phenyl)-2H-pyrazolo[3,4-d]pyrimidin-4-yl)piperazine-2-carboxamide. Light brown solid (yield 84%): [α]_{D}²⁵=+84.5 (c 0.4, MeOH); ¹H NMR (400 MHz, Methanol-d₄) δ 9.12 (s, 1H), 8.26 (s, 1H), 8.12 (d, J=8.4 Hz, 2H), 7.80 (d, J=8.4 Hz, 2H), 7.74-7.63 (m, 2H), 7.49 (d, J=5.4 Hz, 1H), 7.22-7.12 (m, 2H), 4.76-4.45 (m, 3H), 4.34-4.19 (m, 1H), 4.05-3.81 (m, 3H), 3.40-3.33 (m, 1H), 3.24-3.14 (m, 1H); ¹³C NMR (100 MHz, Methanol-d₄) δ 170.73, 161.38, 159.56, 157.19, 143.33, 141.28, 140.11, 135.72, 131.17 (q, J=32.9 Hz), 128.20, 127.95, 127.91, 125.53, 125.24 (q, J=271.4 Hz), 125.13, 124.63, 123.54, 123.44, 121.90, 104.18, 57.97, 47.31, 46.08, 44.27, 43.41; HRMS (ESI) calcd for C₂₆H₂₃F₃N₇OS [M+H]⁺ 538.1631, found 538.1634.

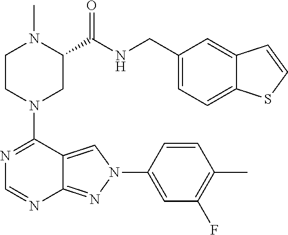 [SMILES: Cc1ccc(-n2cc3c(N4CCN(C)[C@H](C(=O)NCc5ccc6sccc6c5)C4)ncnc3n2)cc1F]

(S)—N-(benzo[b]thiophen-5-ylmethyl)-4-(2-(3-fluoro-4-methylphenyl)-2H-pyrazolo[3,4-d]pyrimidin-4-yl)-1-methylpiperazine-2-carboxamide. Light brown solid (yield 80%): [α]_{D}²⁵=+87.2 (c 0.6, MeOH); ¹H NMR (400 MHz, Methanol-d₄) δ 8.97 (s, 1H), 8.27 (s, 1H), 8.15 (s, 1H), 7.75-7.61 (m, 4H), 7.50 (d, J=5.4 Hz, 1H), 7.41-7.31 (m, 1H), 7.26-7.14 (m, 2H), 4.70-4.27 (m, 4H), 3.93-3.82 (m, 1H), 3.75-3.64 (m, 1H), 3.17-3.07 (m, 1H), 3.08-2.98 (m, 1H), 2.68-2.56 (m, 1H), 2.39 (s, 3H), 2.32 (s, 3H); ¹³C NMR (101 MHz, Methanol-d₄) δ 172.96, 162.61 (d, J=245.1 Hz), 160.80, 159.36, 156.80, 141.28, 140.06, 139.88 (d, J=9.6 Hz), 136.04, 133.70-133.03 (m), 128.14, 126.50 (d, J=17.2 Hz), 125.15, 124.65, 123.60, 123.42, 116.91, 108.76 (d, J=28.3 Hz), 103.85, 68.05, 52.79, 47.39, 46.27, 44.06, 43.92, 14.20; HRMS (ESI) calcd for C₂₇H₂₇FN₆OS [M+H]⁺ 516.1976, found 516.1984.

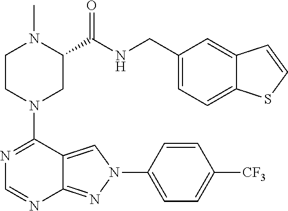 [SMILES: CN1CCN(c2ncnc3nn(-c4ccc(C(F)(F)F)cc4)cc23)C[C@H]1C(=O)NCc1ccc2sccc2c1]

(S)—N-(benzo[b]thiophen-5-ylmethyl)-1-methyl-4-(2-(4-(trifluoromethyl)phenyl)-2H-pyrazolo[3,4-d]pyrimidin-4-yl)piperazine-2-carboxamide. Light brown solid (yield 84%): [α]_{D}²⁵=+82.1 (c 0.6, MeOH); ¹H NMR (400 MHz, Methanol-d₄) δ 9.07 (s, 1H), 8.29 (s, 1H), 8.17-8.10 (m, 3H), 7.82 (d, J=8.4 Hz, 2H), 7.71-7.67 (m, 2H), 7.48 (d, J=5.5 Hz, 1H), 7.23-7.17 (m, 2H), 4.69-4.29 (m, 4H), 3.98-3.86 (m, 1H), 3.77-3.65 (m, 1H), 3.18-3.10 (m, 1H), 3.09-3.02 (m, 1H), 2.68-2.59 (m, 1H), 2.40 (s, 3H); ¹³C NMR (100 MHz, Methanol-d₄) δ 172.95, 161.21, 159.45, 157.25, 143.42, 141.27, 140.05, 136.07, 131.13 (q, J=33.1 Hz), 128.15, 127.94, 127.90, 125.57, 125.28 (q, J=271.1 Hz), 125.15, 124.64, 123.60, 123.40, 121.92, 104.29, 68.02, 52.72, 47.03, 46.37, 44.05, 43.93; HRMS (ESI) calcd for C₂₇H₂₅F₃N₇OS [M+H]⁺ 552.1788, found 552.1789.

### NAMPT Coupled Enzyme Activity Assay

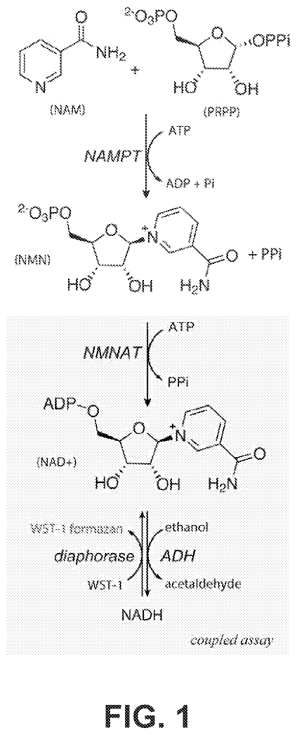

The NAMPT enzyme assay is based on conditions from Burgos and Schramm 34 and adapted to include a cycling reaction to quantitate NAD⁺ production colorimetrically (FIG. 1). The assay follows the NAMPT-catalyzed production of NMN from substrates NAM and PRPP by coupling NMN formation to the NMNAT reaction, which produces NAD⁺ from NMN and ATP. The NAD⁺ is then cycled by alcohol dehydrogenase (ADH) and diaphorase to continuously produce WST-1 formazan, which can be detected at 450 nm. Assays are performed at 25° C. in clear 384-well plates, with a final assay volume of 30 uL, and contain the following: 50 mM HEPES, pH 7.5, 5 mM MgCl₂, 50 mM NaCl, 0.01% Triton-X 100, 2.5 mM ATP, 40 uM PRPP, 1-500 uM NAM, 1.5 uL WST-1 (Roche Cell Proliferation Reagent), 1 U/mL ADH, 0.083 U/mL diaphorase, 1.5% ethanol, 1% DMSO, 30 nM NAMPT, and 7.4 nM purified human NMNAT1. All assay reagents were acquired from Sigma-Aldrich unless otherwise specified. N-terminal His₆-NMNAT1 and C-terminal His₆-NAMPT were overexpressed and purified by metal chelate affinity chromatography. Following assay assembly, well signals were measured continuously at 450 nm on a Tecan Infinite M200 plate reader for 1 h with intermittent shaking. Slopes of the linear portions of the reaction progress curves were recorded and corrected for background by subtracting the average slope of control wells containing NAMPT inhibitor FK866.

A counterassay to confirm NAMPT-specific activation was performed for all compounds using the enzyme assay described above, but lacking NAMPT, NAM and PRPP, and with the addition of 5 uM NMNAT substrate NMN, and with a reduced NMNAT concentration (typically 1 nM).

### NAMPT Fluorescence Polarization (FP) Displacement Assay

All FP measurements were performed at room temperature in PBS buffer containing 0.01% Triton-X 100 and 1% DMSO in black 384-well plates. Measurements were performed on a Tecan F200 Pro plate reader fitted with polarized 485(20) nm emission filters and 535(25) nm emission filters. Initial titration of 20 nM FP probe with varying concentrations of NAMPT protein was performed to estimate a K_{d} value for the probe (˜750 nM), which defined the NAMPT concentration for all subsequent experiments. Anisotropy data from equilibrium competition binding experiments were fit to a four-parameter dose response equation in GraphPad prism.

All FP experiments included the following control sets: unbound probe (probe alone), bound probe (probe+NAMPT), and background signal (NAMPT alone). Results are provided in Table 2.

TABLE 2

| | EC₅₀ | Maximum |
|---|---|---|
| Compound | (μM) | activation (%) |
| 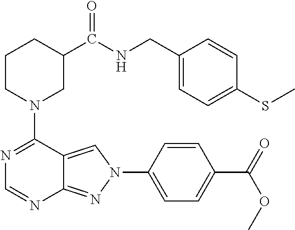 [SMILES: COC(=O)c1ccc(-n2cc3c(N4CCCC(C(=O)NCc5ccc(SC)cc5)C4)ncnc3n2)cc1] | 0.786 | 167 |
| 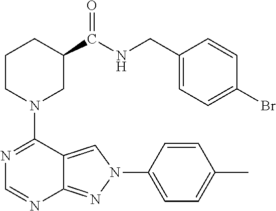 [SMILES: Cc1ccc(-n2cc3c(N4CCC[C@@H](C(=O)NCc5ccc(Br)cc5)C4)ncnc3n2)cc1] | 1.38 | 197 |
| 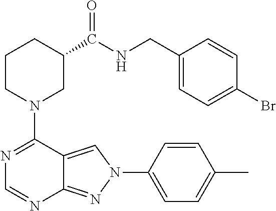 [SMILES: Cc1ccc(-n2cc3c(N4CCC[C@H](C(=O)NCc5ccc(Br)cc5)C4)ncnc3n2)cc1] | 4.6 | 161 |
| 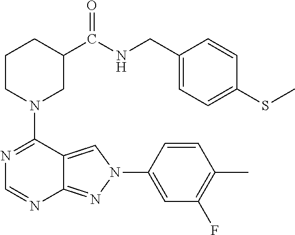 [SMILES: CSc1ccc(CNC(=O)C2CCCN(c3ncnc4nn(-c5ccc(C)c(F)c5)cc34)C2)cc1] | 0.008 | 140 |
| 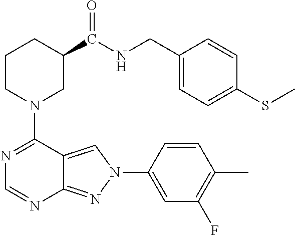 [SMILES: CSc1ccc(CNC(=O)[C@@H]2CCCN(c3ncnc4nn(-c5ccc(C)c(F)c5)cc34)C2)cc1] | <0.01 | 120 |
| 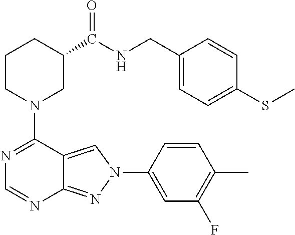 [SMILES: CSc1ccc(CNC(=O)[C@H]2CCCN(c3ncnc4nn(-c5ccc(C)c(F)c5)cc34)C2)cc1] | 0.18 | 216 |
| 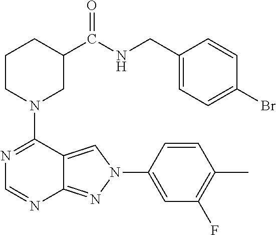 [SMILES: Cc1ccc(-n2cc3c(N4CCCC(C(=O)NCc5ccc(Br)cc5)C4)ncnc3n2)cc1F] | 0.118 | 166 |
| 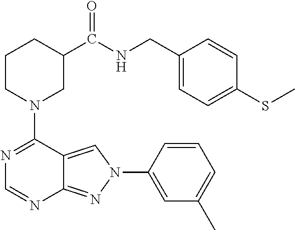 [SMILES: CSc1ccc(CNC(=O)C2CCCN(c3ncnc4nn(-c5cccc(C)c5)cc34)C2)cc1] | 1.12 | 141 |
| 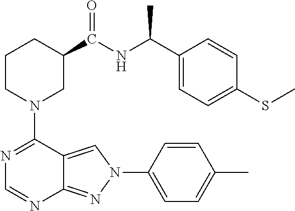 [SMILES: CSc1ccc([C@H](C)NC(=O)[C@@H]2CCCN(c3ncnc4nn(-c5ccc(C)cc5)cc34)C2)cc1] | 2.03 | 281 |
| 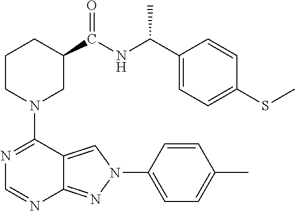 [SMILES: CSc1ccc([C@@H](C)NC(=O)[C@@H]2CCCN(c3ncnc4nn(-c5ccc(C)cc5)cc34)C2)cc1] | 1.00 | 192 |
| 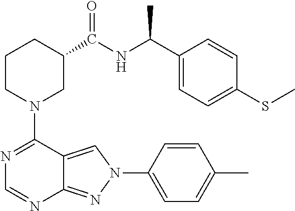 [SMILES: CSc1ccc([C@H](C)NC(=O)[C@H]2CCCN(c3ncnc4nn(-c5ccc(C)cc5)cc34)C2)cc1] | 2.15 | 222 |
| 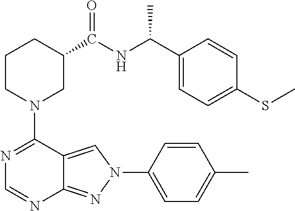 [SMILES: CSc1ccc([C@@H](C)NC(=O)[C@H]2CCCN(c3ncnc4nn(-c5ccc(C)cc5)cc34)C2)cc1] | 3.16 | 219 |
| 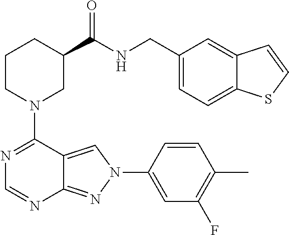 [SMILES: Cc1ccc(-n2cc3c(N4CCC[C@@H](C(=O)NCc5ccc6sccc6c5)C4)ncnc3n2)cc1F] | 0.002 | 136 |
| 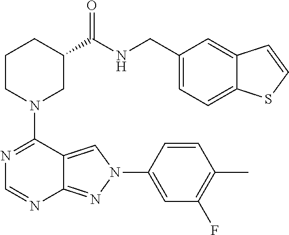 [SMILES: Cc1ccc(-n2cc3c(N4CCC[C@H](C(=O)NCc5ccc6sccc6c5)C4)ncnc3n2)cc1F] | 0.047 | 222 |
| 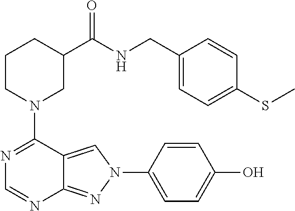 [SMILES: CSc1ccc(CNC(=O)C2CCCN(c3ncnc4nn(-c5ccc(O)cc5)cc34)C2)cc1] | 8.7 | 222 |
| 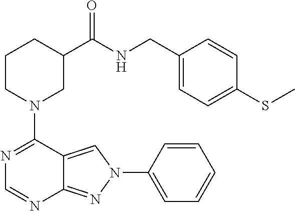 [SMILES: CSc1ccc(CNC(=O)C2CCCN(c3ncnc4nn(-c5ccccc5)cc34)C2)cc1] | 2.1 | 208 |
| 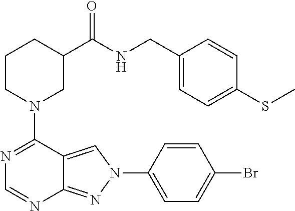 [SMILES: CSc1ccc(CNC(=O)C2CCCN(c3ncnc4nn(-c5ccc(Br)cc5)cc34)C2)cc1] | 0.081 | 1.91 |
| 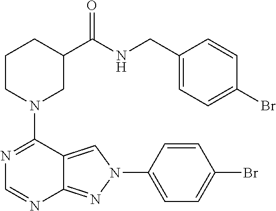 [SMILES: O=C(NCc1ccc(Br)cc1)C1CCCN(c2ncnc3nn(-c4ccc(Br)cc4)cc23)C1] | 0.199 | 200 |
| 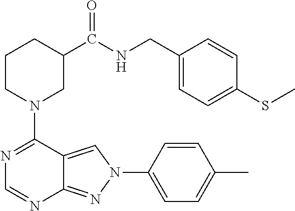 [SMILES: CSc1ccc(CNC(=O)C2CCCN(c3ncnc4nn(-c5ccc(C)cc5)cc34)C2)cc1] | 0.096 | 210 |
| 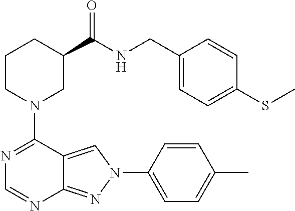 [SMILES: CSc1ccc(CNC(=O)[C@@H]2CCCN(c3ncnc4nn(-c5ccc(C)cc5)cc34)C2)cc1] | 0.04 | 186 |
| 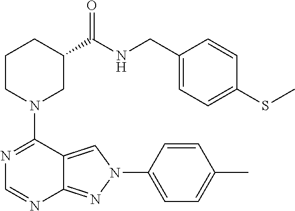 [SMILES: CSc1ccc(CNC(=O)[C@H]2CCCN(c3ncnc4nn(-c5ccc(C)cc5)cc34)C2)cc1] | 1.00 | 291 |
| 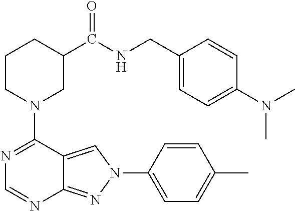 [SMILES: Cc1ccc(-n2cc3c(N4CCCC(C(=O)NCc5ccc(N(C)C)cc5)C4)ncnc3n2)cc1] | 0.16 | 186 |
| 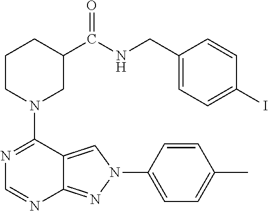 [SMILES: Cc1ccc(-n2cc3c(N4CCCC(C(=O)NCc5ccc(I)cc5)C4)ncnc3n2)cc1] | 0.40 | 217 |
| 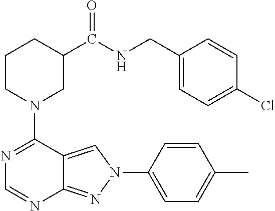 [SMILES: Cc1ccc(-n2cc3c(N4CCCC(C(=O)NCc5ccc(Cl)cc5)C4)ncnc3n2)cc1] | 1.07 | 220 |
| 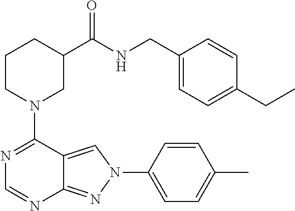 [SMILES: CCc1ccc(CNC(=O)C2CCCN(c3ncnc4nn(-c5ccc(C)cc5)cc34)C2)cc1] | 1.4 | 216 |
| 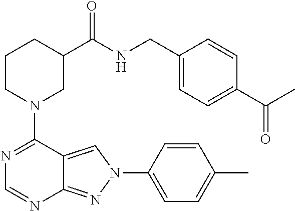 [SMILES: CC(=O)c1ccc(CNC(=O)C2CCCN(c3ncnc4nn(-c5ccc(C)cc5)cc34)C2)cc1] | 2.60 | 213 |
| 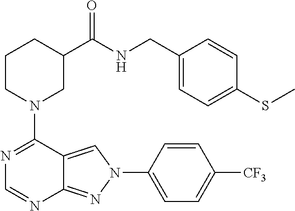 [SMILES: CSc1ccc(CNC(=O)C2CCCN(c3ncnc4nn(-c5ccc(C(F)(F)F)cc5)cc34)C2)cc1] | 0.116 | 235 |
| 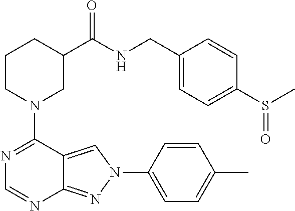 [SMILES: Cc1ccc(-n2cc3c(N4CCCC(C(=O)NCc5ccc(S(C)=O)cc5)C4)ncnc3n2)cc1] | >>20 | |
| 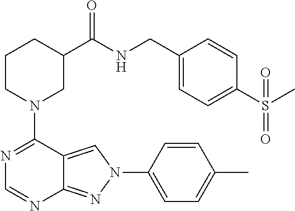 [SMILES: Cc1ccc(-n2cc3c(N4CCCC(C(=O)NCc5ccc(S(C)(=O)=O)cc5)C4)ncnc3n2)cc1] | >>20 | |
| 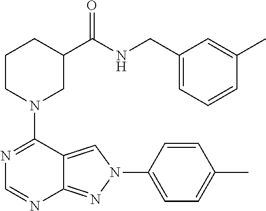 [SMILES: Cc1ccc(-n2cc3c(N4CCCC(C(=O)NCc5cccc(C)c5)C4)ncnc3n2)cc1] | >5 | |
| 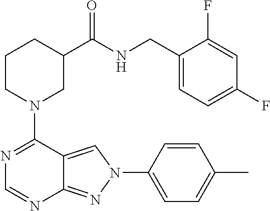 [SMILES: Cc1ccc(-n2cc3c(N4CCCC(C(=O)NCc5ccc(F)cc5F)C4)ncnc3n2)cc1] | >5 | |
| 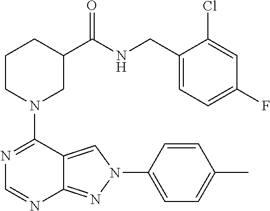 [SMILES: Cc1ccc(-n2cc3c(N4CCCC(C(=O)NCc5ccc(F)cc5Cl)C4)ncnc3n2)cc1] | 15.8 | 260 |
| 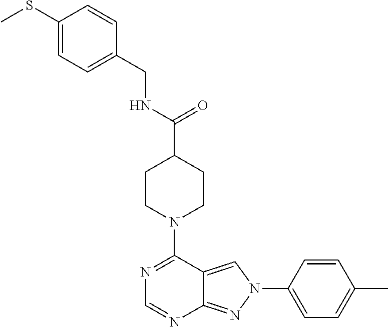 | 9.4 | 240 |
| 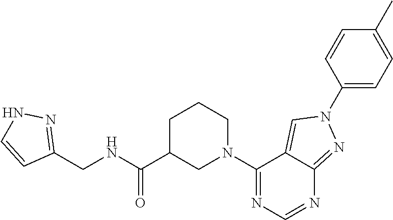 [SMILES: Cc1ccc(-n2cc3c(N4CCCC(C(=O)NCc5cc[nH]n5)C4)ncnc3n2)cc1] | >10 | |
| 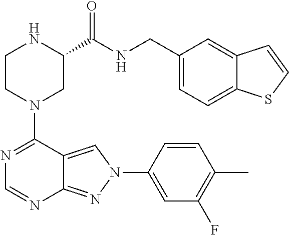 [SMILES: Cc1ccc(-n2cc3c(N4CCN[C@H](C(=O)NCc5ccc6sccc6c5)C4)ncnc3n2)cc1F] | 0.015 | 1.99 |
| 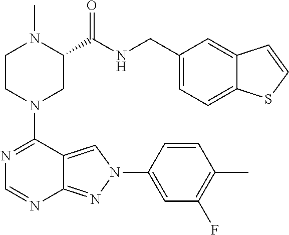 [SMILES: Cc1ccc(-n2cc3c(N4CCN(C)[C@H](C(=O)NCc5ccc6sccc6c5)C4)ncnc3n2)cc1F] | 0.018 | 1.71 |
| 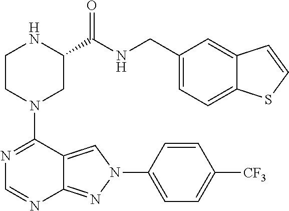 [SMILES: O=C(NCc1ccc2sccc2c1)[C@@H]1CN(c2ncnc3nn(-c4ccc(C(F)(F)F)cc4)cc23)CCN1] | 0.058 | 2.08 |
| 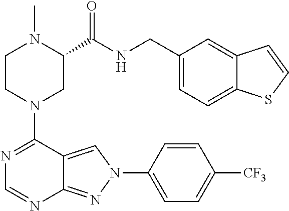 [SMILES: CN1CCN(c2ncnc3nn(-c4ccc(C(F)(F)F)cc4)cc23)C[C@H]1C(=O)NCc1ccc2sccc2c1] | 0.099 | 2.15 |

### NAD⁺ Levels in the THP-1 Cell-Based Assay

The human monocytic leukemia cell line (THP-1) was obtained from the American Type Culture Collection and were maintained in RPMI 1640 medium (ATCC) supplemented with 100 units of penicillin, 100 μg/ml streptomycin, and 10% Heat Inactived fetal bovine serum. All cells were grown at 37° C., under 5% CO₂ in a humidified incubator. Low passage THP-1 cells (37,500 cells/well) were seeded in 96-well plates and incubated at 37° C. and 5% CO₂ for 1½ h prior to a 24 h treatment. All compounds were dissolved in DMSO, and final DMSO concentrations never exceeded 1%. The NAD⁺ levels in the cells are measured using the NAD/NADH-Glo™ assay (Promega). The assays were performed in triplicate for each concentration and the IC₅₀ values were determined from non-linear regression analysis of the dose-response curve generated in GraphPad Prism 9. Results are provided in Table 3.

TABLE 3

| | EC₅₀ | Maximum |
|---|---|---|
| Compound | (μM) | activation (%) |
| 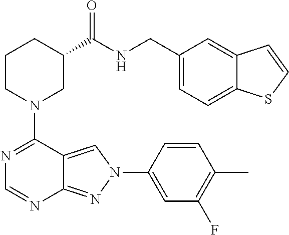 [SMILES: Cc1ccc(-n2cc3c(N4CCC[C@H](C(=O)NCc5ccc6sccc6c5)C4)ncnc3n2)cc1F] | 0.47 | 150.0 |
| 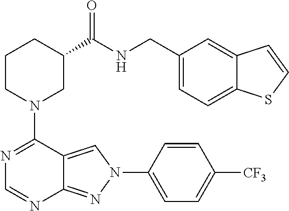 [SMILES: O=C(NCc1ccc2sccc2c1)[C@H]1CCCN(c2ncnc3nn(-c4ccc(C(F)(F)F)cc4)cc23)C1] | 0.74 | 150.0 |
| 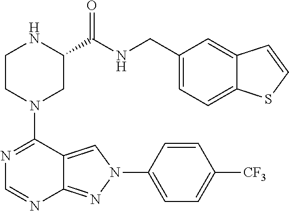 [SMILES: O=C(NCc1ccc2sccc2c1)[C@@H]1CN(c2ncnc3nn(-c4ccc(C(F)(F)F)cc4)cc23)CCN1] | 0.10 | 144.1 |
| 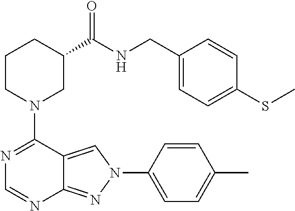 [SMILES: CSc1ccc(CNC(=O)[C@H]2CCCN(c3ncnc4nn(-c5ccc(C)cc5)cc34)C2)cc1] | 3.20 | 201.7 |

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Scheibye-Knudsen, M.; Mitchell, S. J.; Fang, E. F.; Iyama, T.; Ward, T.; Wang, J.; Dunn, C. A.; Singh, N.; Veith, S.; Hasan-Olive, M. M.; Mangerich, A.; Wilson, M. A.; Mattson, M. P.; Bergersen, L. H.; Cogger, V. C.; Warren, A.; Le Couteur, D. G.; Moaddel, R.; Wilson, D. M., 3rd; Croteau, D. L.; de Cabo, R.; Bohr, V. A. A high-fat diet and NAD(+) activate Sirt1 to rescue premature aging in cockayne syndrome. Cell Metab 2014, 20, 840-855.
2. Fang, E. F.; Scheibye-Knudsen, M.; Brace, L. E.; Kassahun, H.; SenGupta, T.; Nilsen, H.; Mitchell, J. R.; Croteau, D. L.; Bohr, V. A. Defective mitophagy in XPA via PARP-1 hyperactivation and NAD(+)/SIRT1 reduction. Cell 2014, 157, 882-896.
3. Belenky, P.; Racette, F. G.; Bogan, K. L.; McClure, J. M.; Smith, J. S.; Brenner, C. Nicotinamide riboside promotes Sir2 silencing and extends lifespan via Nrk and Urh1/Pnp1/Meu1 pathways to NAD⁺. Cell 2007, 129, 473-484.
4. Mouchiroud, L.; Houtkooper, R. H.; Moullan, N.; Katsyuba, E.; Ryu, D.; Canto, C.; Mottis, A.; Jo, Y. S.; Viswanathan, M.; Schoonjans, K.; Guarente, L.; Auwerx, J. The NAD(+)/Sirtuin Pathway Modulates Longevity through Activation of Mitochondrial UPR and FOXO Signaling. Cell 2013, 154, 430-441.
5. North, B. J.; Rosenberg, M. A.; Jeganathan, K. B.; Hafner, A. V.; Michan, S.; Dai, J.; Baker, D. J.; Cen, Y.; Wu, L. E.; Sauve, A. A.; van Deursen, J. M.; Rosenzweig, A.; Sinclair, D. A. SIRT2 induces the checkpoint kinase BubR1 to increase lifespan. EMBO J 2014, 33, 1438-1453.
6. Imai, S.; Guarente, L. NAD⁺ and sirtuins in aging and disease. Trends Cell Biol 2014, 24, 464-471.
7. Yoshino, J.; Baur, J. A.; Imai, S. I. NAD(+) Intermediates: The Biology and Therapeutic Potential of NMN and NR. Cell metabolism 2017.
8. Essuman, K.; Summers, D. W.; Sasaki, Y.; Mao, X.; DiAntonio, A.; Milbrandt, J. The SARM1 Toll/Interleukin-1 Receptor Domain Possesses Intrinsic NAD(+) Cleavage Activity that Promotes Pathological Axonal Degeneration. Neuron 2017, 93, 1334-1343 e1335.
9. DiAntonio, A. Axon degeneration: mechanistic insights lead to therapeutic opportunities for the prevention and treatment of peripheral neuropathy. Pain 2019, 160 Suppl 1, S17-S22.
10. Carty, M.; Bowie, A. G. SARM: From immune regulator to cell executioner. Biochem Pharmacol 2019, 161, 52-62.
11. Garten, A.; Schuster, S.; Penke, M.; Gorski, T.; de Giorgis, T.; Kiess, W. Physiological and pathophysiological roles of NAMPT and NAD metabolism. Nat Rev Endocrinol 2015, 11, 535-546.
12. Wang, T.; Zhang, X.; Bheda, P.; Revollo, J. R.; Imai, S.; Wolberger, C. Structure of Nampt/PBEF/visfatin, a mammalian NAD⁺ biosynthetic enzyme. Nat Struct Mol Biol 2006, 13, 661-662.
13. Wang, P.; Miao, C. Y. NAMPT as a Therapeutic Target against Stroke. Trends Pharmacol Sci 2015, 36, 891-905.
14. Zhao, Y.; Liu, X. Z.; Tian, W. W.; Guan, Y. F.; Wang, P.; Miao, C. Y. Extracellular visfatin has nicotinamide phosphoribosyltransferase enzymatic activity and is neuroprotective against ischemic injury. CNS neuroscience & therapeutics 2014, 20, 539-547.
15. Wang, S. N.; Xu, T. Y.; Li, W. L.; Miao, C. Y. Targeting Nicotinamide Phosphoribosyltransferase as a Potential Therapeutic Strategy to Restore Adult Neurogenesis. CNS neuroscience & therapeutics 2016, 22, 431-439.
16. Chang, Y. H.; Chang, D. M.; Lin, K. C.; Shin, S. J.; Lee, Y. J. Visfatin in overweight/obesity, type 2 diabetes mellitus, insulin resistance, metabolic syndrome and cardiovascular diseases: a meta-analysis and systemic review. Diabetes/metabolism research and reviews 2011, 27, 515-527.
17. Olszanecka-Glinianowicz, M.; Owczarek, A.; Bozentowicz-Wikarek, M.; Brzozowska, A.; Mossakowska, M.; Zdrojewski, T.; Grodzicki, T.; Wiecek, A.; Chudek, J. Relationship between circulating visfatin/NAMPT, nutritional status and insulin resistance in an elderly population—results from the PolSenior substudy. Metabolism 2014, 63, 1409-1418.
18. Kocelak, P.; Olszanecka-Glinianowicz, M.; Owczarek, A.; Bozentowicz-Wikarek, M.; Brzozowska, A.; Mossakowska, M.; Zdrojewski, T.; Grodzicki, T.; Wiecek, A.; Chudek, J. Plasma visfatin/nicotinamide phosphoribosyltransferase levels in hypertensive elderly—results from the PolSenior substudy. J Am Soc Hypertens 2015, 9, 1-8.
19. Yoshino, J.; Mills, K. F.; Yoon, M. J.; Imai, S. Nicotinamide mononucleotide, a key NAD(+) intermediate, treats the pathophysiology of diet- and age-induced diabetes in mice. Cell metabolism 2011, 14, 528-536.
20. Parihar, M. S.; Brewer, G. J. Mitoenergetic failure in Alzheimer disease. Am J Physiol Cell Physiol 2007, 292, C8-23.
21. Zhu, X. H.; Lu, M.; Lee, B. Y.; Ugurbil, K.; Chen, W. In vivo NAD assay reveals the intracellular NAD contents and redox state in healthy human brain and their age dependences. Proc Natl Acad Sci USA 2015, 112, 2876-2881.
22. Wang, X.; Xu, T. Y.; Liu, X. Z.; Zhang, S. L.; Wang, P.; Li, Z. Y.; Guan, Y. F.; Wang, S. N.; Dong, G. Q.; Zhuo, S.; Le, Y. Y.; Sheng, C. Q.; Miao, C. Y. Discovery of Novel Inhibitors and Fluorescent Probe Targeting NAMPT. Sci Rep 2015, 5, 12657.
23. Rongvaux, A.; Shea, R. J.; Mulks, M. H.; Gigot, D.; Urbain, J.; Leo, O.; Andris, F. Pre-B-cell colony-enhancing factor, whose expression is up-regulated in activated lymphocytes, is a nicotinamide phosphoribosyltransferase, a cytosolic enzyme involved in NAD biosynthesis. Eur J Immunol 2002, 32, 3225-3234.
24. Revollo, J. R.; Korner, A.; Mills, K. F.; Satoh, A.; Wang, T.; Garten, A.; Dasgupta, B.; Sasaki, Y.; Wolberger, C.; Townsend, R. R.; Milbrandt, J.; Kiess, W.; Imai, S. Nampt/PBEF/Visfatin regulates insulin secretion in beta cells as a systemic NAD biosynthetic enzyme. Cell metabolism 2007, 6, 363-375.
25. Stein, L. R.; Imai, S. Specific ablation of Nampt in adult neural stem cells recapitulates their functional defects during aging. EMBO J 2014, 33, 1321-1340.
26. Stein, L. R.; Wozniak, D. F.; Dearborn, J. T.; Kubota, S.; Apte, R. S.; Izumi, Y.; Zorumski, C. F.; Imai, S. Expression of Nampt in hippocampal and cortical excitatory neurons is critical for cognitive function. J Neurosci 2014, 34, 5800-5815.
27. Sperling, R.; Salloway, S.; Brooks, D. J.; Tampieri, D.; Barakos, J.; Fox, N. C.; Raskind, M.; Sabbagh, M.; Honig, L. S.; Porsteinsson, A. P.; Lieberburg, I.; Arrighi, H. M.; Morris, K. A.; Lu, Y.; Liu, E.; Gregg, K. M.; Brashear, H. R.; Kinney, G. G.; Black, R.; Grundman, M. Amyloid-related imaging abnormalities in patients with Alzheimer's disease treated with bapineuzumab: a retrospective analysis. Lancet Neurol 2012, 11, 241-249.
28. Zhang, W.; Xie, Y.; Wang, T.; Bi, J.; Li, H.; Zhang, L. Q.; Ye, S. Q.; Ding, S. Neuronal protective role of PBEF in a mouse model of cerebral ischemia. J Cereb Blood Flow Metab 2010, 30, 1962-1971.
29. Wang, P.; Xu, T. Y.; Guan, Y. F.; Tian, W. W.; Viollet, B.; Rui, Y. C.; Zhai, Q. W.; Su, D. F.; Miao, C. Y. Nicotinamide phosphoribosyltransferase protects against ischemic stroke through SIRT1-dependent adenosine monophosphate-activated kinase pathway. Ann Neurol 2011, 69, 360-374.
30. Zhao, Y.; Guan, Y. F.; Zhou, X. M.; Li, G. Q.; Li, Z. Y.; Zhou, C. C.; Wang, P.; Miao, C. Y. Regenerative Neurogenesis After Ischemic Stroke Promoted by Nicotinamide Phosphoribosyltransferase-Nicotinamide Adenine Dinucleotide Cascade. Stroke 2015, 46, 1966-1974.
31. Pittelli, M.; Cavone, L.; Lapucci, A.; Oteri, C.; Felici, R.; Niccolai, E.; Amedei, A.; Chiarugi, A. Nicotinamide phosphoribosyltransferase (NAMPT) activity is essential for survival of resting lymphocytes. Immunol Cell Biol 2014, 92, 191-199.
32. Pieper, A. A.; McKnight, S. L. Benefits of Enhancing Nicotinamide Adenine Dinucleotide Levels in Damaged or Diseased Nerve Cells. Cold Spring Harb Symp Quant Biol 2019.
33. Ding, C.; Hammarlund, M. Mechanisms of injury-induced axon degeneration. Curr Opin Neurobiol 2019, 57, 171-178.
34. Burgos, E. S.; Schramm, V. L. Weak coupling of ATP hydrolysis to the chemical equilibrium of human nicotinamide phosphoribosyltransferase. Biochemistry 2008, 47, 11086-11096.

1. A compound of Formula I 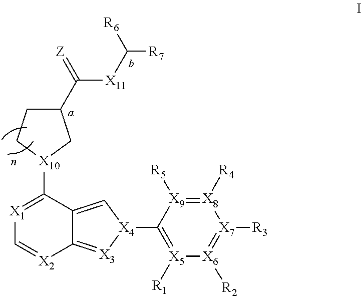
wherein X₁, X₂, X₃, an X₄ are N or CH;
X₅, X₆, X₇, X₈, and X₉ are N or C, wherein when X₅, X₆, X₇, X₈, or X₉ is N, R₁, R₂, R₃, R₄, R₅ and R₆ are not present;
X₁₀ is N or C(Y₁)₂;
X₁₁ is NY₁ or C(Y₁)₂;
Y₁ is independently selected from the group consisting of hydrogen, alkyl, alkoxy, haloalkyl, hydroxyalkyl, OH, CN, F, Cl, Br, and I;
Z is O or S;
R₁, R₂, R₃, R₄, R₅ and R₆ are each independently selected from —H, —OH, a halogen, —CN, alkyl, alkynyl, C3-C6 cycloalkyl, a C1-C3 haloalkyl, and —C(O)—O-alkyl;
R₇ is independently selected from: 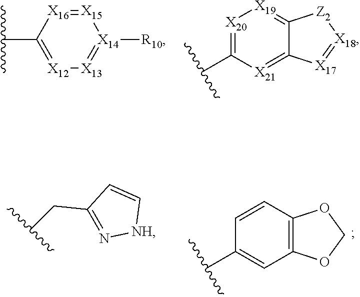
X₁₂, X₁₃, X₁₅, X₁₆, X₁₇, X₁₈, X₁₉, X₂₀, and X₂₁ are N or CY₂;
X₁₄ is N or C, wherein when X₁₄ is N, R₁₀ is not present;
Y₂ is selected from hydrogen, alkyl, alkoxy, haloalkyl, hydroxyalkyl, OH, CN, F, Cl, Br, and I;
Z₂ is S, NY₃ or O;
Y₃ is hydrogen or alky;
R₁₀ is independently selected from: hydrogen, alkyl, alkoxy, haloalkyl, hydroxyalkyl, OH, CN, F, Cl, Br, I, N(alkyl)₂, S-alkyl, S(O)-alkyl, and S(O)₂-alkyl;
n is an integer from 0 to 3; and
the stereochemistry at carbon a and carbon b is, independently, substantially R, substantially S, or racemic; or
a pharmaceutically acceptable salt thereof.

2. The compound of claim 1, wherein X₁, X₂, X₃, X₄, and X₁₀ are each N.

3. The compound of claim 1, wherein Z is O and X₁₁ is NH.

4. The compound of claim 1, wherein n is 2.

5. The compound of claim 1, wherein X₅, X₆, X₇, X₃, and X₉ are each C.

6. The compound of claim 1, wherein R₃ is a halogen, alkyl, or C1-C3 haloalkyl.

7. The compound of claim 1, wherein R₆ is hydrogen.

8. The compound of claim 1, wherein R₇ is

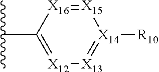

9. The compound of claim 8, wherein X₁₂, X₁₃, X₁₅, and X₁₆, are each CY₂ and X₁₄ is C.

10. The compound of claim 8, wherein R₁₀ is F, Cl, Br, I or S-alkyl.

11. The compound of claim 1, wherein R₇ is

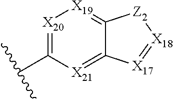

12. The compound of claim 11, wherein Z₂ is S and each of X₁₇, X₁₈, X₁₉, X₂₀, and X₂₁ are CY₂.

13. The compound of claim 1, wherein the compound is Formula II 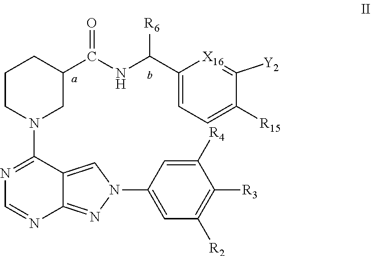
wherein X₁₆ is N or CH;
R₂, R₃, R₄, R₆, and Y₂ are independently selected from —H, —OH a halogen, —CN, alkynyl, C3-C6 cycloalkyl and C1-C3 haloalkyl; and
R₁₅ is selected from -SMe, -SEt, —I, —Br.

14. The compound of claim 1, wherein the compound is Formula III 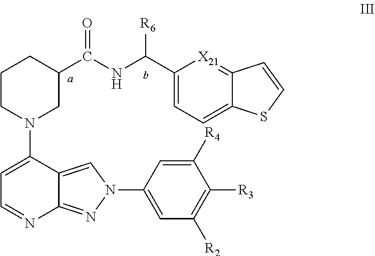
wherein X₂₁ is N or CH; and
R₂, R₃, R₄, and R₆ are independently selected from —H, —OH a halogen, —CN, alkynyl, C3-C6 cycloalkyl and C1-C3 haloalkyl.

15. The compound of claim 1, wherein R₆ is hydrogen and the stereochemistry at carbon a is substantially R or substantially S.

16. A compound of Formula IV or V 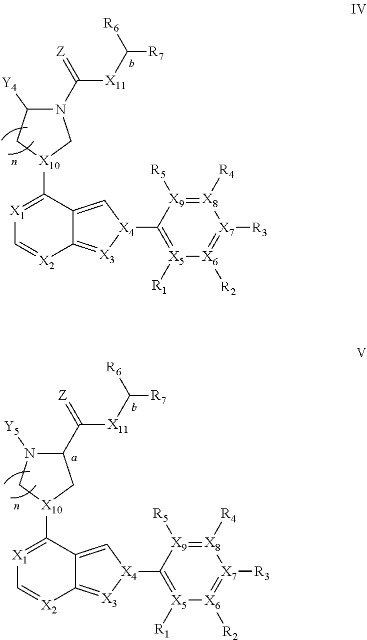
wherein X₁, X₂, X₃, and X₄ are N or CH;
X₅, X₆, X₇, X₈, and X₉ are N or C, wherein when X₅, X₆, X₇, X₈, or X₉ is N, R₁, R₂, R₃, R₄, R₅ and R₆ are not present;
X₁₀ is N or C(Y₁)₂;
X₁₁ is NY₁ or C(Y₁)₂;
Y₁, Y₄, and Y₅ are independently selected from the group consisting of hydrogen, alkyl, alkoxy, haloalkyl, hydroxyalkyl, OH, CN, F, C, Br, and I;
Z is O or S;
R₁, R₂, R₃, R₄, R₅ and R₆ are each independently selected from —H, —OH, a halogen, —CN, alkyl, alkynyl, C3-C6 cycloalkyl, a C1-C3 haloalkyl, and —C(O)—O-alkyl;
R₇ is independently selected from: 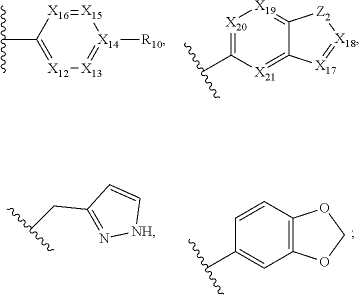
X₁₂, X₁₃, X₁₅, X₁₆, X₁₇, X₁₈, X₁₉, X₂₀, and X₂₁ are N or CY₂;
X₁₄ is N or C, wherein when X₁₄ is N, R₁₀ is not present;
Y₂ is selected from hydrogen, alkyl, alkoxy, haloalkyl, hydroxyalkyl, OH, CN, F, Cl, Br, and I;
Z₂ is S, NY₃ or O;
Y₃ is hydrogen or alkyl;
R₁₀ is independently selected from hydrogen, alkyl, alkoxy, haloalkyl, hydroxyalkyl, OH, CN, F, Cl, Br, I, N(alkyl)₂, S-alkyl, S(O)-alkyl, and S(O)₂-alkyl;
n is an integer from 0 to 3; and
the stereochemistry at carbon a and carbon b is, independently, substantially R, substantially S, or racemic; or
a pharmaceutically acceptable salt thereof.

17. The compound of claim 16, wherein n is 2, X₁, X₂, X₃, X₄, and X₁₀ are each N, Z is O, and X₁₁ is NH.

18. A method for activating NAMPT in a subject, the method comprising administering to the subject the compound of claim 1.

19. A method for increasing NAD+ in a subject, the method comprising administering to the subject the compound of claim 1.

20. A method for treating a neurological disorder, diabetes, or a metabolic disorder in a subject, the method comprising administering to the subject the compound of claim 1.
